(19) United States
(12) Patent Application Publication
(10) Pub. No.: US 2026/0207525 A1
(43) Pub. Date: Jul. 23, 2026

(54) LIPID NANOPARTICLES COMPRISING NUCLEIC ACIDS, IONIZABLE LIPIDS, STEROLS, LIPID ANCHORED POLYMERS AND HELPER LIPIDS, THEIR USES

(71) Applicant: Generation Bio Co., Cambridge, MA (US)

(72) Inventors: Matthew G. Stanton, Cambridge, MA (US); Nolan Gallagher, Cambridge, MA (US); Andrew Milstead, Cambridge, MA (US); Randall Newton Toy, Cambridge, MA (US); Nathaniel Silver, Cambridge, MA (US); Constance Martin, Cambridge, MA (US); Anshul Gupta, Cambridge, MA (US); Christian J. Slubowski, Cambridge, MA (US); Jimit G. Raghav, Cambridge, MA (US); Sandy Zhang, Cambridge, MA (US)

(21) Appl. No.: 19/134,444

(22) PCT Filed: Dec. 1, 2023
(86) PCT No.: PCT/US2023/082021

§ 371 Date: May 30, 2025

Related U.S. Application Data
(60) Provisional application No. 63/429,267, filed on Dec. 1, 2022.
(60) Provisional application No. 63/449,617, filed on Mar. 3, 2023.
(60) Provisional application No. 63/452,077, filed on Mar. 14, 2023.
(60) Provisional application No. 63/467,045, filed on May 17, 2023.

(51) Int. Cl. A61K 9/51; A61K 47/26; A61K 47/54; A61K 47/60; A61K 47/69; A61K 48/00

(52) U.S. Cl. CPC A61K 9/5123; A61K 9/5192; A61K 47/26; A61K 47/548; A61K 47/60; A61K 47/6929; A61K 48/0041; A61K 48/005

(57) ABSTRACT

Provided herein are lipid nanoparticle (LNP) compositions (e.g., pharmaceutical compositions) comprising a therapeutic nucleic acid (TNA), wherein the LNP comprises an ionizable lipid; a “helper” lipid, e.g., a ceramide or distearoylphosphatidylcholine (DSPC); a structural lipid, e.g., a sterol; and one or more types of lipid-anchored polymers, as well as uses thereof.

### RELATED APPLICATIONS

The instant application claims priority to U.S. Provisional Application No. 63/429,267, filed on Dec. 1, 2022; U.S. Provisional Application No. 63/449,617, filed Mar. 3, 2023; U.S. Provisional Application No. 63/452,077, filed Mar. 14, 2023; and U.S. Provisional Application No. 63/467,045, filed May 17, 2023. The entire contents of each of the foregoing applications are expressly incorporated by reference herein.

### BACKGROUND

Lipid-based nanoparticles have played a pivotal role in the successes of COVID-19 vaccines and many other nanomedicines, such as Doxil® and Onpattro®, and have therefore been considered as a frontrunner among nanoscale drug delivery systems. However, effective targeted delivery of biologically active substances, such as therapeutic nucleic acids, represents a continuing medical challenge. This has severely limited broad applications of nucleic acids such as mRNA and DNA in non-viral gene replacement therapy, gene therapy, gene editing, and vaccination.

Lack of effective methods and vehicles for non-viral delivery represents a major barrier to a broad use of nucleic acid therapeutics. Generally, non-viral delivery of the larger mRNA or DNA genetic cargoes is more challenging than that of very small oligonucleotides, in part due to the fact that mRNA and DNA molecules (which typically range from 300 kDa to 5,000 kDa in size, or ~1-15 kb) are significantly larger than other types of RNAs, such as small interfering RNAs or siRNA (which are typically ~14 kDa) or antisense oligonucleotides or ASOs (which typically range from 4 kDa to 10 kDa).

Furthermore, viral delivery of nucleic acid therapeutics to targeted cells is hindered greatly by the activation of the innate and/or adaptive immune responses. Whereas it is possible to avoid RNA sensing by myeloid dendritic cells (MDCs) by chemically modifying RNA cargo (e.g., with 1 ml, 2′OMe, etc.), there are no known chemical modifications to a DNA cargo that can limit pattern recognition receptor (PRR) sensing and still maintain transcriptional activity. An alternative approach to gene therapy is the recombinant adeno-associated virus (rAAV) vector platform that packages heterologous DNA in a viral capsid. However, there are several major disadvantages to using rAAV vectors as a gene delivery vector. One major drawback associated with rAAV is its limited viral packaging capacity of about 4.5 kb of heterologous DNA. Another major drawback is capsid immunogenicity that prevents re-administration to patients.

Thus, there remains a need for effective delivery vehicles that enable safe and effective non-viral delivery of nucleic acid therapeutics to desired cell populations.

### SUMMARY

The present disclosure provides lipid nanoparticles (LNPs) and LNP compositions (e.g., pharmaceutical compositions) comprising a therapeutic nucleic acid (TNA), e.g., a gene expression vector such as closed-ended DNA (ceDNA), single stranded DNA (ssDNA) vector, or messenger RNA (mRNA). The LNPs of the disclosure comprise structural LNP components which comprise an ionizable lipid; a “helper” lipid, e.g., a ceramide or distearoylphosphatidylcholine (DSPC); a structural lipid, e.g., a sterol; and one or more types of lipid-anchored polymers. The LNPs disclosed herein provide surprising and unexpected properties as compared to known LNPs. For example, the helper lipid of the LNP functions to increase the fusogenicity of the lipid bilayer of the LNP and to facilitate endosomal escape; the structural lipid of the LNP contributes to membrane integrity and stability of the LNP; and the lipid-anchored polymer of the LNP can inhibit aggregation of LNPs and provide steric stabilization (e.g., enhancing the stealth property of overall LNP characteristic in the circulation (e.g., the blood compartment) by minimizing interactions between opsonins present in the blood and the surface of the LNP). Moreover, the disclosed LNP compositions are characterized by a reduced LNP related toxicity, as is evidenced by serum levels of immune response markers (see Examples herein). Further, the disclosed LNPs with certain molecular percentage of sterol (e.g., 30%-45% molecular percentage of the total lipid) are characterized by a diameter of about 80 nm or less, making them particularly useful for therapeutic administration specifically directed to certain tissue/organ that has size limitations for effective delivery.

According to one aspect, the disclosure provides a lipid nanoparticle (LNP) comprising: a therapeutic nucleic acid (TNA);
an ionizable lipid;
a sterol;
a first lipid-anchored polymer; wherein the lipid-anchored polymer comprises:
   i) a polymer;
   ii) a lipid moiety comprising at least one hydrophobic tail; and
   iii) optionally a linker connecting the polymer to the lipid moiety;
   wherein the at least one hydrophobic tail comprises 12 to 22 carbon atoms in a single aliphatic chain backbone; and
a helper lipid represented by Formula (I):

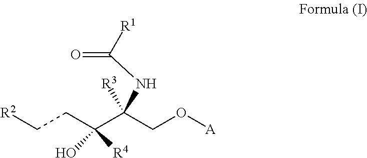

or a salt or an ester thereof, or a deuterated analogue of any of the foregoing, wherein:
   [symbol] is a single bond or a double bond;
   A is hydrogen,

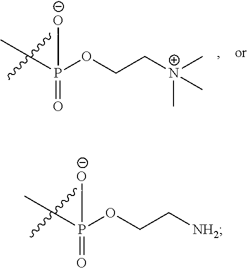 [SMILES: *P(=O)([O-])OCC[N+](C)(C)C ; *P(=O)([O-])OCCN]

and wherein
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

According to another aspect, the disclosure provides a lipid nanoparticle (LNP) comprising:
a therapeutic nucleic acid (TNA);
an ionizable lipid;
a sterol;
a first lipid-anchored polymer; wherein the lipid-anchored polymer comprises:
i) a polymer;
ii) a lipid moiety comprising at least two hydrophobic tails; and
iii) a linker connecting the polymer to the lipid moiety;
   wherein the at least two hydrophobic tails each comprise 16 to 22 carbon atoms in a single aliphatic chain backbone; and
a helper lipid represented by Formula (I):

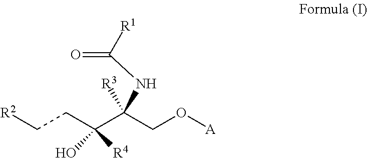

or a salt or an ester thereof, or a deuterated analogue of any of the foregoing, wherein:
   [symbol] is a single bond or a double bond;
   A is hydrogen,

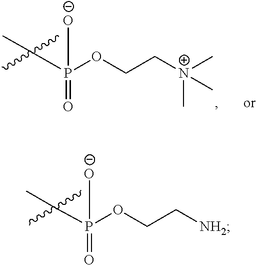 [SMILES: *P(=O)([O-])OCC[N+](C)(C)C ; *P(=O)([O-])OCCN]

and wherein
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

According to yet another aspect, the disclosure provides a lipid nanoparticle (LNP) comprising:
a therapeutic nucleic acid (TNA);
an ionizable lipid;
a sterol;
a first lipid-anchored polymer; wherein the lipid-anchored polymer comprises:
   i) a polymer;
   ii) a lipid moiety comprising at least two hydrophobic tails; and
   iii) a linker connecting the polymer to the lipid moiety;
      wherein the at least two hydrophobic tails each comprise 12 to 15 carbon atoms in a single aliphatic chain backbone; and
a helper lipid represented by Formula (I):

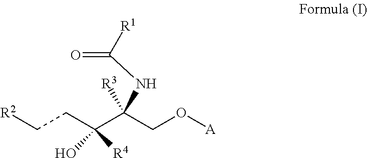

or a salt or an ester thereof, or a deuterated analogue of any of the foregoing, wherein:
   [symbol] is a single bond or a double bond;
   A is hydrogen,

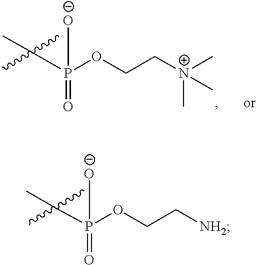 [SMILES: *P(=O)([O-])OCC[N+](C)(C)C ; *P(=O)([O-])OCCN]

R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

According to one further aspect, the disclosure provides a lipid nanoparticle (LNP) comprising:
a therapeutic nucleic acid (TNA);
an ionizable lipid;
a sterol;
a first lipid-anchored polymer; wherein the lipid-anchored polymer comprises:
   i) a polymer;
   ii) a lipid moiety comprising a single hydrophobic tail; and
   iii) a linker connecting the polymer to the lipid moiety;
   wherein the single hydrophobic tail comprises 18 to 22 carbon atoms in a single aliphatic chain backbone; and
a helper lipid represented by Formula (I):

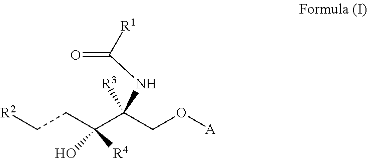

or a salt or an ester thereof, or a deuterated analogue of any of the foregoing, wherein:
   [symbol] is a single bond or a double bond;

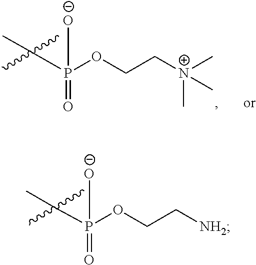 [SMILES: *P(=O)([O-])OCC[N+](C)(C)C ; *P(=O)([O-])OCCN]

A is hydrogen,
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments, the helper lipid in an LNP provided herein is represented by Formula (II)

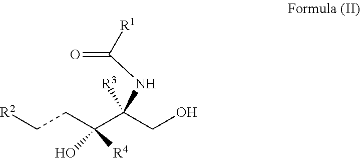

or a salt or an ester thereof, or a deuterated analogue of any of the foregoing.

In some embodiments, the helper lipid is represented by Formula (III):

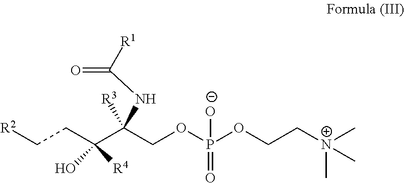

or a salt or an ester thereof, or a deuterated analogue of any of the foregoing.

In some embodiments, the helper lipid is represented by Formula (IV):

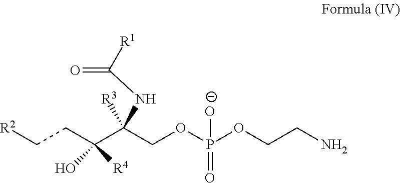

or a salt or an ester thereof, or a deuterated analogue of any of the foregoing.

In some embodiments, the LNP of this disclosure does not comprise distearoylphosphatidylcholine (DSPC), provided that a helper lipid represented by (I), (II), (III), or (IV), or a salt or an ester thereof, or a deuterated analogue of any of the foregoing is present. In some embodiments, the LNP of this disclosure does not comprise 1,2-dioleoyl-sn-glycero-3-phosphocholine (DOPC), provided that a helper lipid represented by (I), (II), (III), or (IV), or a salt or an ester thereof, or a deuterated analogue of any of the foregoing is present. In some embodiments, the LNP of this disclosure does not comprise 1,2-dioleoyl-sn-glycero-3-phosphoethanolamine (DOPE), provided that a helper lipid represented by (I), (II), (III), or (IV), or a salt or an ester thereof, or a deuterated analogue of any of the foregoing is present.

In some embodiments, R¹ is C₁-C₁₀ alkyl or C₂-C₁₀ alkenyl in Formula (I), (II), (III), or (IV); wherein R², R³, and R⁴ are as defined above.

In some embodiments, [symbol] is a double bond in Formula (I), (II), (III), or (IV); wherein R¹, R², R³, and R⁴ are as defined above.

In some embodiments, R¹ is C₁-C₈alkyl or C₂-C₈ alkenyl in Formula (I), (II), (III), or (IV); wherein R², R³, and R⁴ are as defined above. In some embodiments, R¹ is C₁-C₇ alkyl or C₂-C₇ alkenyl in Formula (I), (II), (III), or (IV); wherein R², R³, and R⁴ are as defined above. In some embodiments, R¹ is C₁ alkyl, C₃ alkyl, C₅ alkyl, or C₇ alkyl in Formula (I), (II), (III), or (IV); wherein R², R³, and R⁴ are as defined above. In some embodiments, R¹ is C₁ alkyl in Formula (I), (II), (III), or (IV); wherein R², R³, and R⁴ are as defined above.

In some embodiments, R² is C₃-C₁₅ alkyl or C₃-C₁₅ alkenyl in Formula (I), (II), (III), or (IV); wherein R¹, R³, and R⁴ are as defined above. In some embodiments, R² is C₉ alkyl, C₁₁ alkyl, C₁₂ alkyl, C₁₃ alkyl, or C₁₅ alkyl in Formula (I), (II), (III), or (IV); wherein R¹, R³, and R⁴ are as defined above. In some embodiments, R² is C₁₂ alkyl, C₁₃ alkyl, or C₁₄ alkyl; wherein R¹, R³, and R⁴ are as defined above. In some embodiments, R² is C₁₃ alkyl in Formula (I), (II), (III), or (IV); wherein R¹, R³, and R⁴ are as defined above.

In some embodiments, R³ is hydrogen in Formula (I), (II), (III), or (IV); wherein R¹, R², and R⁴ are as defined above. In some embodiments, R³ is C₁ alkyl in Formula (I), (II), (III), or (IV); wherein R¹, R², and R⁴ are as defined above.

In some embodiments, R⁴ is hydrogen in Formula (I), (II), (III), or (IV); wherein R¹, R², and R³ are as defined above. In some embodiments, R⁴ is C₁ alkyl; wherein R¹, R², and R³ are as defined above.

In some embodiments, the helper lipid represented by Formula (I) is selected from any of the helper lipids listed in Table 8, or a salt or an ester thereof, or a deuterated analogue of any of the foregoing.

In some embodiments, the helper lipid represented by Formula (I) is selected from:

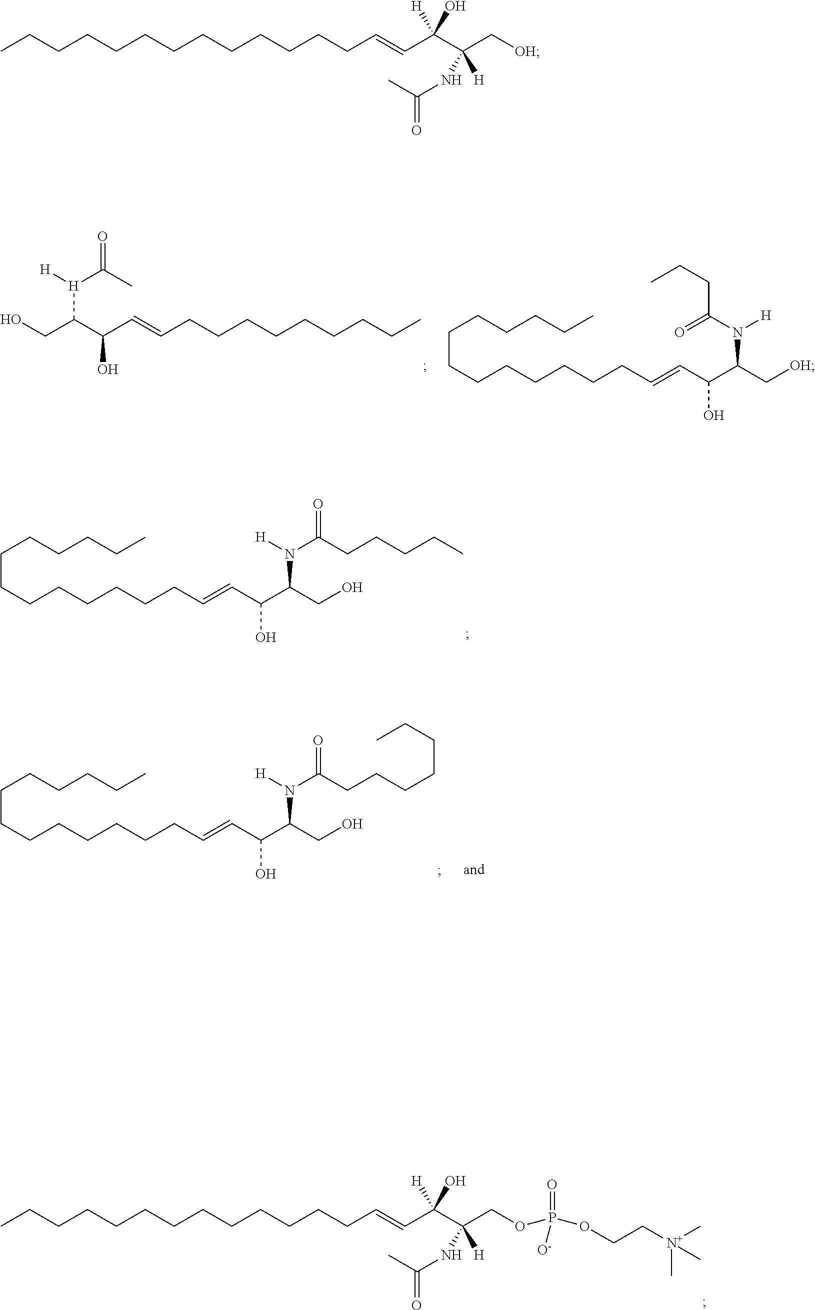

or a salt or an ester thereof, or a deuterated analogue of any of the foregoing.

In some embodiments, the helper lipid represented by Formula (I) or Formula (II) is:

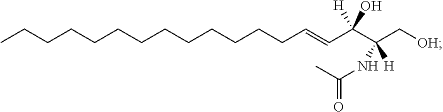 [SMILES: CCCCCCCCCCCCC/C=C/[C@@H](O)[C@H](CO)NC(C)=O]

or a salt or an ester thereof, or a deuterated analogue of any of the foregoing.

In some embodiments, the lipid represented by Formula (I), Formula (III), or Formula (IV) is:

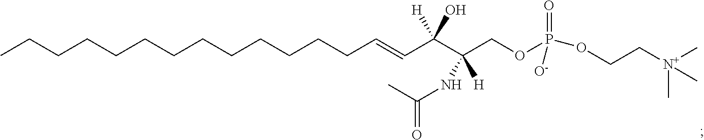 [SMILES: CCCCCCCCCCCCC/C=C/[C@@H](O)[C@H](COP(=O)([O-])OCC[N+](C)(C)C)NC(C)=O]

or a salt or an ester thereof, or a deuterated analogue of any of the foregoing.

According to another aspect, the present disclosure provides a lipid nanoparticle (LNP) comprising:
a therapeutic nucleic acid (TNA);
an ionizable lipid;
a sterol;
a first lipid-anchored polymer; wherein the first lipid-anchored polymer comprises:
   i) a polymer;
   ii) a lipid moiety comprising at least two hydrophobic tails; and
   iii) a linker connecting the polymer to the lipid moiety;
      wherein the at least two hydrophobic tails each comprise 16 to 22 carbon atoms in a single aliphatic chain backbone; and
a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE);
wherein the LNP has a whole blood half-life (t_{1/2}) of at least about 3 hours.

In some embodiments, the at least two hydrophobic tails of the first lipid-anchored polymer each have 18 to 22 carbon atoms in a single aliphatic chain backbone. In some embodiments, the at least two hydrophobic tails of the first lipid-anchored polymer each have 18 to 20 carbon atoms in a single aliphatic chain backbone. In some embodiments, the at least two hydrophobic tails of the first lipid-anchored polymer each have 18 carbon atoms in a single aliphatic chain backbone.

In some embodiments, the helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) is present in the LNP in an amount of about 2 mol % to about 40 mol %, or about 5 mol % to about 35 mol %, or about 5 mol % to about 30 mol %, or about 5 mol % to about 25 mol %, or about 5 mol % to about 20 mol %, or about 5 mol % to about 15 mol %, or about 5 mol % to about 10 mol %, or about 10 mol % to about 15 mol % of the total lipid present in the LNP. In some embodiments, the helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) is present in the LNP in an amount of about 10 mol %. In one embodiment, the helper lipid is DSPC. In one embodiment, the DSPC helper lipid is present in an amount of about 10 mol %.

In some embodiments, the LNP comprising a helper lipid selected from the group consisting of DSPC, DOPC, and DOPE has a whole blood half-life (t_{1/2}) of about 3 hours to about 24 hours, or about 3 hours to about 18 hours, or about 3 hours to about 15 hours, or about 3 hours to about 12 hours, or about 3 hours to about 10 hours, or about 3 hours to about 9 hours, or about 3 hours to about 8 hours, or about 3 hours to about 7.5 hours, or about 3 hours to about 6.5 hours, or about 3 hours to about 6 hours. In some embodiments, the LNP has a whole blood half-life (t_{1/2}) of about 3 hours to about 3.5 hours, or about 3 hours to about 4 hours, or about 3 hours to about 4.5 hours, or about 3 hours to about 5 hours, or about 3 hours to about 5.5 hours, or about 3.5 hours to about 4 hours, or about 3.5 hours to about 4.5 hours, or about 3.5 hours to about 5 hours, or about 3.5 hours to about 5.5 hours, or about 4 hours to about 4.5 hours, or about 4 hours to about 5 hours, or about 4 hours to about 5.5 hours, or about 4.5 hours to about 5 hours, or about 4.5 hours to about 5.5 hours, or about 5 hours to about 5.5 hours. In some embodiments, by comparison, a reference LNP has a whole blood half-life (t_{1/2}) of no greater than about 3 hours, e.g., about 10 minutes, about 15 minutes, about 20 minutes, about 30 minutes, about 1 hour, about 2 hours or about 2.5 hours. The reference LNP does not comprise a first lipid-anchored polymer having at least two hydrophobic tails with 16 to 22 carbon atoms in a single aliphatic chain backbone. In one embodiment, the reference LNP comprises a first lipid-anchored polymer comprising at least two hydrophobic tails each comprising 12 to 15 carbon atoms in a single aliphatic chain backbone. In one embodiment, the first lipid-anchored polymer is 1,2-dimyristoyl-rac-glycero-3-methoxypolyethylene glycol (DMG-PEG, or also referred to as PEG-DMG). In one embodiment, the reference LNP comprises DMG-PEG and a helper lipid selected from the group consisting of DSPC, DOPC, and DOPE.

In some embodiments, the LNP comprising a helper lipid selected from the group consisting of DSPC, DOPC, and DOPE has a whole blood clearance rate (Cl) of about 10 mL/min/kg to about 50 mL/min/kg, or about 10 mL/min/kg to about 45 mL/min/kg, or about 10 mL/min/kg to about 40 mL/min/kg. In some embodiments, the LNP has a whole blood clearance rate (Cl) of about 30 mL/min/kg to about 40 mL/min/kg, or about 35 mL/min/kg to about 40 mL/min/kg, or about 10 mL/min/kg to about 20 mL/min/kg, or about 10 mL/min/kg to about 18 mL/min/kg, or about 10 mL/min/kg to about 15 mL/min/kg. In some embodiments, by comparison, a reference LNP has a whole blood clearance rate (Cl) of at least twice the value of the whole blood clearance of the LNP comprising a helper lipid selected from the group consisting of DSPC, DOPC, and DOPE, as described above, e.g., greater than about 50 mL/min/kg, or about 50-100 mL/min/kg, or about 50-150 mL/min/kg, or about 50-200 mL/min/kg, or about 50-250 mL/min/kg, or about 50-300 mL/min/kg, or about 50-350 mL/min/kg, or about 100-150 mL/min/kg, or about 100-200 mL/min/kg, or about 100-250 mL/min/kg, or about 100-300 mL/min/kg, or about 100-350 mL/min/kg. In one embodiment, the reference LNP does not comprise a first lipid-anchored polymer having at least two hydrophobic tails with 16 to 22 carbon atoms in a single aliphatic chain backbone. In one embodiment, the reference LNP comprises a lipid-anchored polymer comprising at least two hydrophobic tails each comprising 12 to 15 carbon atoms in a single aliphatic chain backbone. In one embodiment, the reference lipid-anchored polymer is 1,2-dimyristoyl-rac-glycero-3-methoxypolyethylene glycol (DMG-PEG, also referred to as PEG-DMG). In one embodiment, the reference comprises DMG-PEG and a helper lipid selected from the group consisting of DSPC, DOPC, and DOPE.

In some embodiments, the LNP comprising a helper lipid selected from the group consisting of DSPC, DOPC, and DOPE has a whole blood terminal timepoint exposure (AUCₗₐₛₜ) of at least 50 hour*ng/mL. In one embodiment, the terminal timepoint is 24 hours. In other embodiments, the terminal timepoint is about 18 hours, about 20 hours, about 22 hours, about 26 hours, about 28 hours, about 30 hours, about 32 hours, about 34 hours, about 36 hours, about 38 hours, or about 40 hours. In some embodiments, the LNP has a whole blood terminal timepoint exposure (AUCₗₐₛₜ) of about 50 hour*ng/ml to about hour*ng/mL, or about 100 hour*ng/ml to about 750 hour*ng/ml, or about 150 hour*ng/mL to about 750 hour*ng/mL, or about 200 hour*ng/ml to about 700 hour*ng/mL.

In some embodiments, the LNP comprising a helper lipid selected from the group consisting of DSPC, DOPC, and DOPE has a whole blood terminal timepoint exposure (AUCₗₐₛₜ) of about 200 hour*ng/ml to about 250 hour*ng/ml, or about 200 hour*ng/mL to about 300 hour*ng/ml, or about 500 hour*ng/ml to about 700 hour*ng/mL, or about 500 hour*ng/ml to about 550 hour*ng/ml, or about 500 hour*ng/ml to about 600 hour*ng/mL, or about 550 hour*ng/ml to about 600 hour*ng/mL, or about 600 hour*ng/ml to about 700 hour*ng/mL, or about 600 hour*ng/mL to about 650 hour*ng/mL, or about 650 hour*ng/ml to about 700 hour*ng/mL. In some embodiments, by comparison, a reference LNP has a whole blood terminal timepoint exposure (AUCₗₐₛₜ) or no greater than 50 hour*ng/ml, e.g., about 40-45 hour*ng/ml, or about 35-40 hour*ng/ml, or about 30-35 hour*ng/mL, or about 25-30 hour*ng/ml, or about 20-25 hour*ng/ml, or about 15-20 hour*ng/ml, or about 10-15 hour*ng/mL, or about 5-10 hour*ng/mL. In one embodiment, the reference LNP does not comprise a first lipid-anchored polymer having the at least two hydrophobic tails with 16 to 22 carbon atoms in a single aliphatic chain backbone. In one embodiment, the reference LNP comprises a reference lipid-anchored polymer comprising at least two hydrophobic tails each comprised of 12 to 15 carbon atoms in a single aliphatic chain backbone. In one embodiment, the reference lipid-anchored polymer is 1,2-dimyristoyl-rac-glycero-3-methoxypolyethylene glycol (DMG-PEG). In one embodiment, the reference comprises DMG-PEG and a helper lipid selected from the group consisting of DSPC, DOPC, and DOPE.

In some embodiments, the first lipid-anchored polymer in an LNP of this disclosure comprises a lipid moiety comprising one hydrophobic tail or two hydrophobic tails. In one embodiment, the first lipid-anchored polymer in an LNP of this disclosure comprises a lipid moiety comprising two hydrophobic tails. In one embodiment, the two hydrophobic tails are each a fatty acid. In some embodiments, the two hydrophobic tails each independently comprise 16, 17, 18, 19, 20, 21, or 22 carbon atoms. In some embodiments, the two hydrophobic tails each independently comprise 16, 17, 18, 19, 20, or 21 carbon atoms. In some embodiments, the two hydrophobic tails each independently comprise 16, 17, 18, 19, or 20 carbon atoms. In some embodiments, the two hydrophobic tails each independently comprise 16, 17, 18, or 19 carbon atoms. In some embodiments, the two hydrophobic tails each independently comprise 16, 17, or 18 carbon atoms. In one embodiment, the two hydrophobic tails each comprise 16 carbon atoms. In one embodiment, the two hydrophobic tails each comprise 18 carbon atoms. In one embodiment, the two hydrophobic tails each comprise 20 carbon atoms. In some embodiments, the two hydrophobic tails are each independently selected from the group consisting of octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

In some embodiments, the two hydrophobic tails each independently comprise 12, 13, 14, or 15 carbon atoms. In some embodiments, the two hydrophobic tails each independently comprise 12, 13, or 14 carbon atoms. In one embodiment, the two hydrophobic tails each comprise 12 carbon atoms. In one embodiment, the two hydrophobic tails each comprise 14 carbon atoms. In some embodiments, the two hydrophobic tails are each independently selected from the group consisting of lauric acid, myristic acid, myristoleic acid, and a derivative thereof.

In one embodiment, the first lipid-anchored polymer in an LNP of this disclosure comprises a lipid moiety comprising a single hydrophobic tail. In one embodiment, the single hydrophobic tail is a fatty acid. In some embodiments, the single hydrophobic tail comprises 12, 14, 16, 18, 20, or 22 carbon atoms. In some embodiments, the single hydrophobic tail comprises 12, 14, 16, or 18 carbon atoms. In one embodiment, the single hydrophobic tail comprises 14 carbon atoms. In one embodiment, the single hydrophobic tail comprises 16 carbon atoms. In one embodiment, the single hydrophobic tail comprises 18 carbon atoms. In some embodiments, the single hydrophobic tail is selected from the group consisting of lauric acid, myristic acid, myristoleic acid, octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

In some embodiments, the first lipid-anchored polymer is a glycerolipid. In some embodiments, the first lipid-anchored polymer is a phospholipid. In some embodiments, the first lipid-anchored polymer does not comprise distearoylphosphatidylcholine (DSPC), provided that a helper lipid represented by (I), (II), (III), or (IV), or a salt or an ester thereof, or a deuterated analogue of any of the foregoing is present.

In some embodiments, the first lipid-anchored polymer comprises a linker-lipid moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), distearoyl-rac-glycerol (DSG), 1,2-dipalmitoyl-rac-glycerol (DPG), a derivative thereof, and a combination of any of the foregoing. In some embodiments, the linker-lipid moiety in the first lipid-anchored polymer is selected from the group consisting of DOPE, DSPE, DSG, DODA, DPG, a derivative thereof, and a combination of any of the foregoing.

In some embodiments, the linker-lipid moiety in the first lipid-anchored polymer is selected from the group consisting of 1,2-dimyristoyl-rac-glycero-3-methoxy (DMG), R-3-[(ω-methoxycarbamoyl)]-1,2-dimyristyloxl-propyl-3-amine, a derivative thereof, and a combination of any of the foregoing. In some embodiments, the first lipid-anchored polymer comprises DMG.

In some embodiments, the polymer is selected from the group consisting of polyethylene (PE), polypropylene (PP), polyethylene glycol (PEG), polyglycerol (PG), polyvinyl alcohol (PVOH), polysarcosine (pSar), and a combination thereof. In some embodiments, the polymer is selected from the group consisting of polyethylene glycol (PEG), polyglycerol (PG), polysarcosine (pSar), or a combination thereof.

In some embodiments, the polymer has a molecular weight of between about 1000 Da and about 5000 Da. In some embodiments, the polymer has a molecular weight of between about 2000 Da and about 5000 Da. In some embodiments, the polymer has a molecular weight of about 2000 Da. In some embodiments, the polymer has a molecular weight of about 3200 Da to about 3500 Da.

In some embodiments, the polymer is polyethylene glycol (PEG).

In some embodiments, the sterol is selected from the group consisting of cholesterol, beta-sitosterol, stigmasterol, beta-sitostanol, campesterol, brassicasterol, and a derivative of thereof, and a combination thereof. In some embodiments, the sterol is cholesterol. In some embodiments, the sterol is beta-sitosterol.

In some embodiments, the ionizable lipid is a lipid represented by:
a) Formula (A):

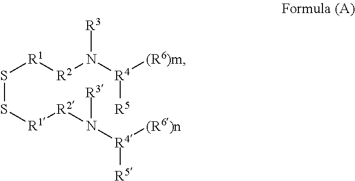

or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl;
or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or —[C(R^{a})₂]₂CR^{a};
R^{a}, for each occurrence, is independently H or C₁₋₃ alkyl;
or alternatively, when R⁴ is —C(R^{a})₂CR^{a}, or —[C(R^{a})₂]₂CR^{a} and when R^{a} is C₁₋₃ alkyl, R³ and R⁴, taken together with their intervening N atom, form a 4- to 8-membered heterocyclyl;
or alternatively, when R⁴ is —C(R^{a})₂CR^{a}, or —[C(R^{a})₂]₂CR^{a} and when R^{a} is C₁₋₃ alkyl, R^{3′} and R^{4′}, taken together with their intervening N atom, form a 4- to 8-membered heterocyclyl;
R⁵ and R^{5′} are each independently hydrogen, C₁₋₂₀ alkylene or C₂-20 alkenylene;
R⁶ and R^{6′}, for each occurrence, are independently C₁₋₂₀ alkylene, C₃-20 cycloalkylene, or C₂-20 alkenylene; and
m and n are each independently an integer selected from 1, 2, 3, 4, and 5; or
b) Formula (B):

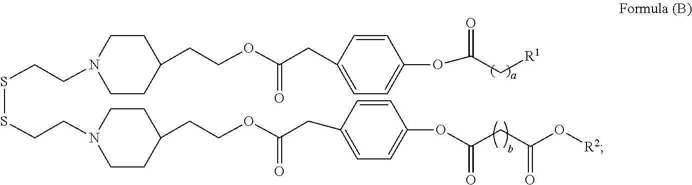

or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl; or
c) Formula (C):

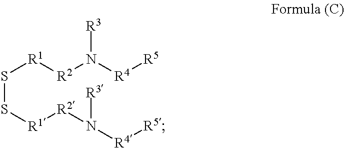

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆) cycloalkyl; and
R^{a} and R^{b} are each halo or cyano; or
d) Formula (D):

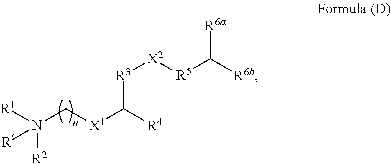

or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all positively charged;
R¹ and R² are each independently hydrogen, C₁-C₆ alkyl, or C₂-C₆ alkenyl;
   R³ is C₁-C₁₂ alkylene or C₂-C₁₂ alkenylene;
R⁴ is C₁-C₁₈ unbranched alkyl, C₂-C₁₈ unbranched alkenyl, or

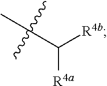

wherein: R^{4a} and R^{4b} are each independently C₁-C₁₆ unbranched alkyl or C₂-C₁₆ unbranched alkenyl; R⁵ is absent, C₁-C₈alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6;
wherein, in some embodiments, R⁴ is C₁-C₁₆ unbranched alkyl, C₂-C₁₆ unbranched alkenyl, or

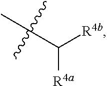

wherein R^{4a} and R^{4b} are as defined above; or
e) Formula (E):

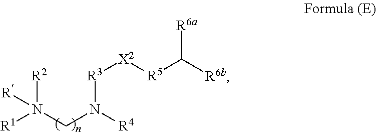

or a pharmaceutically acceptable salt thereof, wherein:

R′ is absent, hydrogen, or C₁-C₃ alkyl; provided that when R′ is hydrogen or C₁-C₃ alkyl, the nitrogen atom to which R′, R¹, and R² are all attached is positively charged;

R¹ and R² are each independently hydrogen or C₁-C₃ alkyl;
R³ is C₃-C₁₀ alkylene or C₃-C₁₀ alkenylene;
R⁴ is C₁-C₁₆ unbranched alkyl, C₂-C₁₆ unbranched alkenyl, or

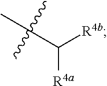

wherein: R^{4a} and R^{4b} are each independently C₁-C₁₆ unbranched alkyl or C₂-C₁₆ unbranched alkenyl; R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N—C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6; or
f) an ionizable lipid selected from any of the ionizable lipids in Table 1, 4, 5, 6 or 7.

In some embodiments, an LNP of this disclosure further comprises a targeting moiety.

In some embodiments, the LNP comprises a second lipid-anchored polymer and the targeting moiety is conjugated to the second lipid-anchored polymer. In some embodiments, the second lipid-anchored polymer comprises a linker-lipid moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), distearoyl-rac-glycerol (DSG), 1,2-dipalmitoyl-rac-glycerol (DPG), a derivative thereof, and a combination of any of the foregoing. In some embodiments, the linker-lipid moiety in the first lipid-anchored polymer is selected from the group consisting of DOPE, DSPE, DSG, DODA, DPG, a derivative thereof, and a combination of any of the foregoing.

In some embodiments, the first and the second lipid-anchored polymers are different lipid-anchored polymers; and the first and the second lipid-anchored polymers comprise one of the following combinations:
DSG (the first lipid-anchored polymer) and DSPE (the second lipid-anchored polymer);
DSPE (the first lipid-anchored polymer) and DSG (the second lipid-anchored polymer);
DODA (the first lipid-anchored polymer) and DSPE (the second lipid-anchored polymer);
DPG (the first lipid-anchored polymer) and DSPE (the second lipid-anchored polymer);
DMG (the first lipid-anchored polymer) and DSPE (the second lipid-anchored polymer);
DODA (the first lipid-anchored polymer) and DSG (the second lipid-anchored polymer);
DPG (the first lipid-anchored polymer) and DSG (the second lipid-anchored polymer);
DMG (the first lipid-anchored polymer) and DSG (the second lipid-anchored polymer);
DPG (the first lipid-anchored polymer) and DODA (the second lipid-anchored polymer);
DMG (the first lipid-anchored polymer) and DODA (the second lipid-anchored polymer); or
DMG (the first lipid-anchored polymer) and DPG (the second lipid-anchored polymer).

In some embodiments, the first and the second lipid-anchored polymers are the same lipid-anchored polymers; and the first and the second lipid-anchored polymers comprise one of the following combinations:
DSG (the first lipid-anchored polymer) and DSG (the second lipid-anchored polymer);
DSPE (the first lipid-anchored polymer) and DSPE (the second lipid-anchored polymer);
DODA (the first lipid-anchored polymer) and DODA (the second lipid-anchored polymer); or
DPG (the first lipid-anchored polymer) and DPG (the second lipid-anchored polymer).

In some embodiments, the targeting moiety is conjugated to a DSPE-anchored polymer. In some embodiments, the DSPE-anchored polymer is DSPE-PEG or a derivative thereof. In some embodiments, the targeting moiety is conjugated to a DSG-anchored polymer. In some embodiments, the DSG-anchored polymer is DSG-PEG or a derivative thereof.

In some embodiments, the targeting moiety is capable of binding to a liver cell. In some embodiments, the liver cell is a hepatocyte. In some embodiments, the targeting moiety is N-acetyl galactosamine (GalNAc) or a GalNAc derivative. In some embodiments, the targeting moiety is a tri-antennary GalNAc conjugate or a tetra-antennary GalNAc conjugate. In some embodiments, the targeting moiety is selected from the group consisting of an ApoE protein, an ApoE polypeptide, an ApoB protein, an ApoB polypeptide, a fragment thereof, and a derivative of any of the foregoing. In some embodiments, the targeting moiety is selected from the group consisting of an ApoE protein conjugate, an ApoE polypeptide conjugate, an ApoB protein conjugate, and an ApoB polypeptide conjugate. In one embodiment, the targeting moiety is a modified ApoE protein conjugate.

In one embodiment, the ionizable lipid in an LNP of the present disclosure in accordance with any of the foregoing embodiments is Ionizable Lipid 81:

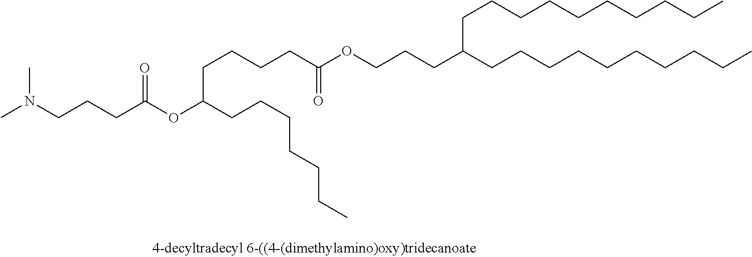 [SMILES: CCCCCCCCCCC(CCCCCCCCCC)CCCOC(=O)CCCCC(CCCCCCC)OC(=O)CCCN(C)C]

or a pharmaceutically acceptable salt thereof.

In one embodiment, the ionizable lipid in an LNP of the present disclosure in accordance with any of the foregoing embodiments is Ionizable Lipid 89:

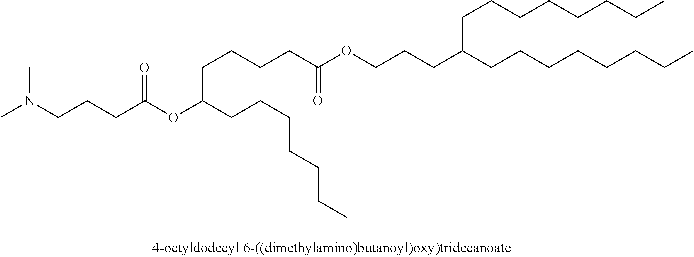 [SMILES: CCCCCCCCC(CCCCCCCC)CCCOC(=O)CCCCC(CCCCCCC)OC(=O)CCCN(C)C]

or a pharmaceutically acceptable salt thereof.

In one embodiment, the ionizable lipid in an LNP of the present disclosure in accordance with any of the foregoing embodiments is Ionizable Lipid 87:

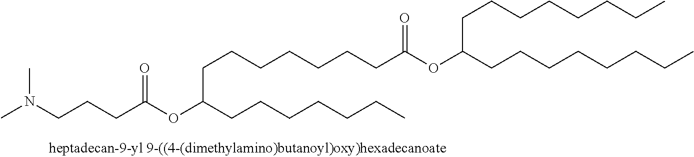 [SMILES: CCCCCCCCC(CCCCCCCC)OC(=O)CCCCCCCC(CCCCCCC)OC(=O)CCCN(C)C]

or a pharmaceutically acceptable salt thereof.

In some embodiments, the ionizable lipid is present in the LNP provided by the present disclosure in an amount of about 35 mol % to about 60 mol % of the total lipid present in the LNP. In some embodiments, the ionizable lipid is present in the LNP in an amount of about 20 mol % to about 50 mol % of the total lipid present in the LNP.

In some embodiments, the sterol is present in the LNP in an amount of about 20 mol % to about 45 mol % of the total lipid present in the LNP. In some embodiments, the sterol is present in the LNP in an amount of about 30 mol % to about 40 mol % of the total lipid present in the LNP.

In some embodiments, the first lipid-anchored polymer is present in the LNP in an amount of about 0.5 mol % to about 5 mol % of the total lipid present in the LNP. In some embodiments, the second lipid-anchored polymer is present in the LNP in an amount of about 0.005 mol % to about 5 mol % of the total lipid present in the LNP. In some embodiments, the first lipid-anchored polymer is present in the LNP in an amount of about 0.05 mol % to about 2 mol % of the total lipid present in the LNP.

In some embodiments, the second lipid-anchored polymer is present in the LNP in an amount of about 0.1 mol % to about 1 mol % of the total lipid present in the LNP. In some embodiments, the second lipid-anchored polymer is present in the LNP in an amount of about 0.5 mol % of the total lipid present in the LNP. In some embodiments, the first lipid-anchored polymer and the second lipid anchored polymer are present in the LNP in an amount of about 2.5 mol % and 0.5 mol %, respectively, of the total lipid present in the LNP.

In some embodiments, the helper lipid represented by Formula (I), (II), (III), or (IV), or a salt or an ester thereof, or a deuterated analogue of any of the foregoing, is present in the LNP in an amount of about 2 mol % to about 40 mol % of the total lipid present in the LNP. In some embodiments, the helper lipid represented by Formula (I), (II), (III), or (IV), or a salt or an ester thereof, or a deuterated analogue of any of the foregoing, is present in the LNP in an amount of about 5 mol % to about 30 mol % of the total lipid present in the LNP. In some embodiments, the helper lipid represented by Formula (I), (II), (III), or (IV), or a salt or an ester thereof, or a deuterated analogue of any of the foregoing, is present in the LNP in an amount of about 10 mol % to about 20 mol % of the total lipid present in the LNP. In some embodiments, the helper lipid represented by Formula (I), (II), (III), or (IV), or a salt or an ester thereof, or a deuterated analogue of any of the foregoing, is present in the LNP in an amount of about 10 mol % of the total lipid present in the LNP. In some embodiments, the helper lipid represented by Formula (I), (II), (III), or (IV), or a salt or an ester thereof, or a deuterated analogue of any of the foregoing, is present in the LNP in an amount of about 15 mol % of the total lipid present in the LNP. In some embodiments, the helper lipid represented by Formula (I), (II), (III), or (IV), or a salt or an ester thereof, or a deuterated analogue of any of the foregoing, is present in the LNP in an amount of about 20 mol % of the total lipid present in the LNP.

In some embodiments, the helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) is present in the LNP in an amount of about 2 mol % to about 40 mol %, or about 5 mol % to about 35 mol %, or about 5 mol % to about 30 mol %, or about 5 mol % to about 25 mol %, or about 5 mol % to about 20 mol %, or about 5 mol % to about 15 mol %, or about 5 mol % to about 10 mol %, or about 10 mol % to about 15 mol % of the total lipid present in the LNP.

In some embodiments, the LNP provided by the present disclosure is suitable for intravenous administration.

In some embodiments, the LNP is less immunogenic than a reference LNP; wherein the reference LNP: (i) does not comprise the helper lipid represented by Formula (I), (II), (III), or (IV), or a salt or an ester thereof, or a deuterated analogue of any of the foregoing; or (ii) comprises a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) and a reference lipid-anchored polymer comprising at least two hydrophobic tails each comprise 12 to 15 carbon atoms in a single aliphatic chain backbone. In one embodiment, the reference lipid-anchored polymer is 1,2-dimyristoyl-rac-glycero-3-methoxypolyethylene glycol (DMG-PEG).

In some embodiments, the LNP results in a lower uptake level of the TNA by a blood cell than that of the reference LNP.

In some embodiments, the LNP elicits a lower pro-inflammatory cytokine response than the reference LNP.

In some embodiments, the LNP results in an expression level of TNA in a blood cell that is lower than the expression level of TNA in a blood cell that results from a reference LNP. In some embodiments, the blood cell is a red blood cell, a macrophage, and a peripheral blood mononuclear cell.

In some embodiments, the therapeutic nucleic acid (TNA) is selected from the group consisting of a minigene, a plasmid, a minicircle, a small interfering RNA (siRNA), a microRNA (miRNA), a guide RNA (gRNA) an antisense oligonucleotide (ASO), a ribozyme, a closed-ended DNA (ceDNA), single-stranded DNA (ssDNA), a ministring, a Doggybone™, a protelomere closed ended DNA, a dumbbell linear DNA, a dicer-substrate dsRNA, a small hairpin RNA (shRNA), an asymmetrical interfering RNA (aiRNA), mRNA, tRNA, rRNA, gRNA, a DNA viral vector, a viral RNA vector, a non-viral vector and any combination thereof.

In some embodiments, the TNA is greater than about 200 bp or greater than about 200 nt in length. In some embodiments, the TNA is greater than about 500 bp or greater than about 500 nt in length. In some embodiments, the TNA is greater than about 1000 bp or greater than about 1000 nt in length. In some embodiments, the TNA is greater than about 4000 bp or greater than about 4000 nt in length.

In some embodiments, the TNA is a closed-ended DNA (ceDNA). In some embodiments, the TNA is a messenger RNA (mRNA). In some embodiments, the TNA is a single-stranded nucleic acid. In some embodiments, the TNA is a double-stranded nucleic acid.

In some aspects, the present disclosure provides a pharmaceutical composition comprising the LNP of the present disclosure and a pharmaceutically acceptable carrier.

In some aspects, the present disclosure also provides a method of producing the LNP of the disclosure, comprising combining: the therapeutic nucleic acid (TNA); the ionizable lipid; the sterol; the first lipid-anchored polymer; the helper lipid represented by Formula (I), (II), (III), or (IV), or a salt or ester thereof, or a deuterated analogue of any of the foregoing, or a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE); optionally the second lipid-anchored polymer; and optionally the targeting moiety.

In some aspects, the present disclosure also provides a method of treating a genetic disorder in a subject, said method comprising administering to said subject an effective amount of the LNP of the disclosure or the pharmaceutical composition of the disclosure.

In some embodiments, the subject is a human.

In some embodiments, the genetic disorder is selected from the group consisting of sickle cell anemia; melanoma; hemophilia A (clotting factor VIII (FVIII) deficiency); hemophilia B (clotting factor IX (FIX) deficiency); cystic fibrosis (CFTR); familial hypercholesterolemia (LDL receptor defect); hepatoblastoma; Wilson disease; phenylketonuria (PKU); congenital hepatic porphyria; an inherited disorder of hepatic metabolism; Lesch Nyhan syndrome; a thalassaemia; xeroderma pigmentosum; Fanconi's anemia; retinitis pigmentosa; ataxia telangiectasia; Bloom's syndrome; retinoblastoma; a mucopolysaccharide storage disease; a Niemann-Pick Disease; Fabry disease; Schindler disease; GM2-gangliosidosis Type II (Sandhoff Disease); Tay-Sachs disease; Metachromatic Leukodystrophy; Krabbe disease; a mucolipidosis (ML); Sialidosis Type II, a glycogen storage disease (GSD); Gaucher disease; cystinosis; Batten disease; Aspartylglucosaminuria; Salla disease; Danon disease (LAMP-2 deficiency); Lysosomal Acid Lipase (LAL) deficiency; a neuronal ceroid lipofuscinoses (NCL); a sphingolipidoses, galactosialidosis; amyotrophic lateral sclerosis (ALS); Parkinson's disease; Alzheimer's disease; Huntington's disease; spinocerebellar ataxia; spinal muscular atrophy (SMA); Friedreich's ataxia; Duchenne muscular dystrophy (DMD); a Becker muscular dystrophy (BMD), dystrophic epidermolysis bullosa (DEB); ectonucleotide pyrophosphatase 1 deficiency; generalized arterial calcification of infancy (GACI); Leber Congenital Amaurosis; Stargardt disease; wet macular degeneration (wet AMD); ornithine transcarbamylase (OTC) deficiency; Usher syndrome; alpha-1 antitrypsin deficiency; a progressive familial intrahepatic cholestasis (PFIC); and Cathepsin A deficiency.

In some embodiments, the genetic disorder is phenylketonuria (PKU). In some embodiments, the genetic disorder is hemophilia A (Factor VIII deficiency). In some embodiments, the genetic disorder is Wilson disease. In some embodiments, the genetic disorder is Gaucher disease. In some embodiments, the genetic disorder is Gaucher disease Type I, Gaucher disease Type II or Gaucher disease type III. In some embodiments, the genetic disorder is Leber congenital amaurosis (LCA). In some embodiments, the LCA is LCA10. In some embodiments, the genetic disorder is Stargardt disease. In some embodiments, the genetic disorder is wet macular degeneration (wet AMD).

In some aspects, the present disclosure also provides a method of providing anti-tumor immunity in a subject, the method comprising administering to the subject an effective amount of the LNP of the present disclosure or the pharmaceutical composition of the present disclosure. In some aspects, the present disclosure also provides a method of treating a subject having a disease, disorder or condition associated with an elevated expression of a tumor antigen, the method comprising administering to the subject an effective amount of the LNP of the present disclosure or the pharmaceutical composition of the present disclosure. In some embodiments, the subject is a human. In some embodiments, the TNA is retained in the spleen for at least about 6 hours, or at least about 9 hours, or at least about 12 hours, or at least about 15 hours, or at least about 18 hours, or at least about 21 hours, or at least about 24 hours, or at least about 27 hours, or at least about 30 hours, or at least about 33 hours, or at least about 36 hours after dosing. In some embodiments, the concentration of the TNA at the start of a 12, 18, or 24-hour time window post-dosing and the concentration of the TNA at the end of the time window are within the same order of magnitude. In one embodiment, the TNA is a messenger RNA (mRNA).

In some aspects, the present disclosure further provides a method of treating a blood disease, disorder or condition in a subject, the method comprising administering to the subject an effective amount of the LNP of the present disclosure or the pharmaceutical composition of the present disclosure. In some embodiments, the blood disease, disorder or condition is selected from the group consisting of acute myeloid leukemia (AML), acute lymphoblastic leukemia (ALL), chronic lymphocytic leukemia (CLL), chronic myelogenous leukemia (CML), Hodgkin lymphoma (HL), multiple myeloma, a myelodysplastic syndrome (MDS), non-Hodgkin lymphoma (NHL), adrenoleukodystrophy (ALD), Hurler syndrome, Krabbe disease (Globoid-cell leukodystrophy or GLD), metachromatic leukodystrophy (MLD), severe aplastic anemia (SAA), severe combined immunodeficiency (SCID), sickle cell disease (SCD), thalassemia, Wiskott-Aldrich syndrome, Diamond-Blackfan anemia, essential thrombocytosis, Fanconi anemia, hemophagocytic lymphohistiscytosis (HLH), juvenile myelomonocytic leukemia (JMML), myelofibrosis, polycythemia vera, and a combination thereof. In one embodiment, the TNA is a messenger RNA (mRNA).

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A shows the in vivo expression of luciferase from LNP D and C2 ceramide-containing LNP1 in CD-1 mice at Day 4 post-dosing. FIG. 1B shows the in vivo expression of luciferase from the same LNP formulations as described above for FIG. 1A, in mice at Day 7 post-dosing. FIG. 1C shows percent change in body weight of mice at Day 1 post-dosing.

FIG. 2A shows the in vivo expression of luciferase from LNP D, C2 ceramide-containing LNP1, C8 ceramide-containing LNP35, and C2 sphingomyelin-containing LNP36 in CD-1 mice at Day 4 post-dosing. FIG. 2B shows the in vivo expression of luciferase from the same LNP formulations as described above for FIG. 2A, in mice at Day 7 post-dosing. FIG. 2C shows percent change in body weight of mice at Day 1 post-dosing.

FIG. 3A shows the in vivo expression of luciferase from LNP C and C2 ceramide-containing LNP37 in CD-1 mice at Day 4 post-dosing. FIG. 3B shows the in vivo expression of luciferase, the same LNP formulations as described above for FIG. 3A, in mice at Day 7 post-dosing.

FIGS. 4A-4F depict the blood serum levels of the cytokines IFN-alpha (FIG. 4A), IL-6 (FIG. 4B), IFN-gamma (FIG. 4C), TNF-alpha (FIG. 4D), IL-18 (FIG. 4E), and IP-10 (FIG. 4F) measured in CD-1 mice at 6 hours post-dose following injection of LNP D and C2 ceramide-containing LNP1.

FIGS. 5A-5E depict the blood serum levels of the cytokines IFN-alpha (FIG. 5A), IL-6 (FIG. 5B), IFN-gamma (FIG. 5C), TNF-alpha (FIG. 5D), and IL-18 (FIG. 5E) measured in CD-1 mice at 6 hours post-dose following injection of LNP D, C2 ceramide-containing LNP1, C8 ceramide-containing LNP35, and C2 sphingomyelin-containing LNP36.

FIGS. 6A-6F depict the blood serum levels of the cytokines IFN-alpha (FIG. 6A), IL-6 (FIG. 6B), IFN-gamma (FIG. 6C), TNF-alpha (FIG. 6D), IL-18 (FIG. 6E), and IP-10 (FIG. 6F) in CD-1 mice at 6 hours pose-dose following injection of LNP A, and C2 ceramide-containing LNP23, LNP24, LNP25, LNP26, LNP27, and LNP28 1.

FIGS. 7A-7F depict the blood serum levels of the cytokines IFN-alpha (FIG. 7A), IL-6 (FIG. 7B), IFN-gamma (FIG. 7C), TNF-alpha (FIG. 7D), IL-18 (FIG. 7E), and IP-10 (FIG. 7F) measured in CD-1 mice at 6 hours post-dose following injection of LNP C and C2 ceramide-containing LNP37.

FIG. 8 depicts the whole blood and plasma levels of the ceDNA cargo in CD-1 mice at 1 hour, 3 hours, and 6 hours post-dose following injection of LNP E and C2 ceramide-containing LNP1.

FIG. 9A shows the in vitro expression of luciferase in primary mouse hepatocytes that were treated with C2 ceramide-containing LNP40 that carried an mRNA luciferase cargo. FIG. 9B shows the DiD signals that indicate the uptake of LNP40 into the primary mouse hepatocytes.

FIG. 10 compares the in vitro expression of luciferase in primary mouse hepatocytes that were treated with LNP F, C2 ceramide-containing LNP41, C4 ceramide-containing LNP42, C6 ceramide-containing LNP43, or C8 ceramide-containing LNP45, each carrying an mRNA luciferase cargo.

FIG. 11 shows and compares the 24-hour total IVIS fluorescence in the liver of CD-1 mice groups dosed with LNP101, LNP102, LNP103, LNP104, and LNP G, all of which carry luciferase mRNA as nucleic acid cargo.

FIG. 12A is a curve quantifying, via qPCR, concentrations of luciferase mRNA (μg/mL) in whole blood at 2 minutes, 1 hour, 6 hours, and 24 hours after dosing for CD-1 mice groups dosed with LNP101, LNP102, LNP103, LNP104, and LNP G.

FIG. 12B is a curve quantifying, via qPCR, copies of luciferase mRNA in the liver at 6 hours and 24 hours after dosing for CD-1 mice groups dosed with LNP101, LNP102, LNP103, LNP104, and LNP G.

FIG. 12C is a curve quantifying, via qPCR, copies of luciferase mRNA) in the spleen at 6 hours and 24 hours after dosing for CD-1 mice groups dosed with LNP101, LNP102, LNP103, LNP104, and LNP G.

FIG. 12D is a curve quantifying, via qPCR, copies of luciferase mRNA in the bone marrow at 6 hours and 24 hours after dosing for CD-1 mice groups dosed with LNP101, LNP102, LNP103, LNP104, and LNP G.

FIG. 13 is a curve quantifying, via qPCR, copies of ceDNA blood at 0 hour, 1 hour, 3 hours, 6 hours and 24 hours after dosing for CD-1 mice groups treated with LNP201, LNP202, and LNP203.

FIG. 14A depicts different retention times from HPLC-SEC readout for LNP formulations having incremental mol % of a first lipid-anchored polymer (i.e., LNPs having DSG-PEG2000-OMe at 1.5 mol %, 2 mol %, 2.5 mol %, 3 mol %, 5 mol %, and 7 mol %).

FIG. 14B depicts retention times for a LNP formulation having mol % of a lipid-anchored polymer (DSG-PEG2000-OMe) at 1.5 mol % (wavelength readout: 214 nm to track lipids and 260 nm to track nucleic acid cargo).

FIG. 14C depicts retention times for LNPs having mol % of a lipid-anchored polymer (DSG-PEG2000-OMe) at 7 mol % (wavelength readout: 214 nm to track lipids and 260 nm to track nucleic acid cargo).

### DETAILED DESCRIPTION

The present disclosure provides lipid nanoparticles (LNPs) and LNP compositions (e.g., pharmaceutical compositions) comprising a therapeutic nucleic acid (TNA), e.g., a gene expression vector such as closed ended DNA (ceDNA), single stranded DNA vector, or messenger RNA (mRNA). The structural components of an LNP provided by the present disclosure comprise an ionizable lipid; a “helper” lipid, e.g., C2 ceramide or C₂ sphingomyelin (“C2-C8 containing helper lipids”); a structural lipid, e.g., a sterol (e.g., cholesterol or betasitosterol); and one or more types of lipid-anchored polymers.

The LNPs and LNP compositions disclosed herein provide surprising and unexpected properties as compared to known LNPs. For example, the helper lipid of the LNP functions to increase the fusogenicity of the lipid bilayer of the LNP and to facilitate endosomal escape; the structural lipid of the LNP contributes to membrane integrity and stability of the LNP; and the lipid-anchored polymer of the LNP can inhibit aggregation of LNPs and provide steric stabilization (e.g., enhancing the stealth property of overall LNP characteristic in the blood compartment by minimizing any interaction between potential opsonins present in the blood and the surface of the LNP). Moreover, the disclosed LNPs and LNP compositions surprisingly are characterized by a reduced LNP related toxicity, as is evidenced by reduced serum levels of immune response markers (see, Examples herein). The present disclosure is based, at least in part, on the surprising observations that certain helper lipids, when present in an LNP together with a lipid-anchored polymer having at least two hydrophobic tails that each are of a certain length, e.g., each independently comprise 16 to 22 carbon atoms, may contribute to mitigation of LNP-related immunogenicity. Such helper lipids include ceramide, sphingomyelin and a fatty acid having a certain number of aliphatic carbon atoms in the fatty acid portion of the helper lipid, e.g., in C2-C8 ceramide. It was also found that a helper like DSPC can support stability and extended stealthiness of the LNPs of the present disclosure as measured by in vivo pharmacokinetics. Further, the disclosed LNPs comprising a certain molecular percentage of sterol (30%-45% molecular percentage of the total lipid) are characterized by an average diameter of about 70-100 nm, 70-80 nm or less, making them particularly useful for therapeutic administration. Hence, an LNP having desirable properties like an increased stealth property that could evade rapid cellular uptake by blood cells and enhanced tolerability can be achieved by combining LNP components having specific physical attributes of the helper lipids and the lipid-anchored polymers disclosed herein.

### I. Definitions

Unless otherwise defined herein, scientific and technical terms used in connection with the present application shall have meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6th Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al. Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used in this specification and the appended claims, the singular forms “a”, “an” and “the” include plural references unless the content clearly dictates otherwise.

The abbreviation, “e.g.” is derived from the Latin exempli gratia and is used herein to indicate a non-limiting example. Thus, the abbreviation “e.g.” is synonymous with the term “for example.”

The use of the alternative (e.g., “or”) should be understood to mean either one, both, or any combination thereof of the alternatives.

As used herein, the term “about,” when referring to a measurable value such as an amount, a temporal duration, and the like, is meant to encompass variations of ±20% or ±10%, more preferably ±5%, even more preferably ±1%, and still more preferably ±0.1% from the specified value, depending on the accuracy and precision of the methods available for determining such measurable values, or as such variations are appropriate to perform the disclosed methods.

As used herein, any concentration range, percentage range, ratio range, or integer range is to be understood to include the value of any integer within the recited range and, when appropriate, fractions thereof (such as one tenth and one hundredth of an integer), unless otherwise indicated.

As used herein, “comprise,” “comprising,” and “comprises” are meant to be synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

The term “consisting of” refers to compositions, methods, processes, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the disclosure.

As used herein, the terms “such as”, “for example” and the like are intended to refer to exemplary embodiments and not to limit the scope of the present disclosure.

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, preferred materials and methods are described herein.

As used herein the terms, “administration,” “administering” and variants thereof refers to introducing a composition or agent (e.g., nucleic acids, in particular ceDNA, ssDNA and mRNA) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

The term “immunogenicity of an LNP” or “immunogenicity of a composition comprising an LNP”, as used herein, refers to the ability of a composition comprising LNPs of the present disclosure to induce an undesired immune response in a subject to the LNP and its components after the LNPs of the disclosure or a composition comprising the LNPs of the disclosure are administered to the subject. In some embodiments, the immune response, e.g., before and after administration of a composition comprising LNPs of the present disclosure, may be measured by measuring levels of one or more pro-inflammatory cytokines. Exemplary pro-inflammatory cytokines that may be used to determine immunogenicity of LNPs of the present disclosure or a composition comprising LNPs of the present disclosure include, but are not limited to, granulocyte colony stimulating factor (G-CSF), interleukin 1 alpha (IL-1α), interleukin 1 beta (IL-1β), interleukin 6 (IL-6), interleukin 8 (IL-8 or CXCL8), interleukin 11 (IL-11), interleukin 17 (IL-17), interleukin 18 (IL-18), interferon α (IFN-α), interferon β (IFN-β), interferon γ (IFN-γ), C—X—C motif chemokine ligand 10 (CXCL10 or IP-10), monocyte chemoattractant protein 1 (MCP-1), CD40L, CCL2, CCL3, CCL4, CCL5, CCL11, tumor necrosis factor α (TNF-α), and combinations thereof. In some embodiments, the immune response, e.g., before and after administration of a composition comprising LNPs of the present disclosure, may be measured by measuring levels of specific antibodies to the LNP and components of the LNP.

The term “off-target delivery”, as used herein, refers to delivery of LNPs to non-target cells. After administration to a subject, an LNP may be delivered to a non-target cell, and may result in expression of a therapeutic nucleic acid (TNA) in the non-target cell.

In some embodiments, the non-target cell may be a liver sinusoidal endothelial cell (LSEC cell), a spleen cell or a Kupffer cell.

After administration to a subject, an LNP may be delivered to a non-target cell and may result in expression of a therapeutic nucleic acid (TNA) in the non-target cell, or may be degraded once engulfed by, e.g., a macrophage. In some embodiments, a reference LNP may be characterized by a higher rate of random delivery to or uptake by non-target cells, e.g., one or more of blood cells listed above, as compared to an LNP of the present disclosure. In some embodiments, an LNP of the present disclosure results in an uptake level of TNA (e.g., ceDNA, ssDNA or mRNA) in a blood cell that is lower than that of a reference LNP. In some embodiments, the reference LNP is an LNP that (i) does not comprise the helper lipid represented by Formula (I), (II), (III), or (IV), or a salt or an ester thereof, or a deuterated analogue of any of the foregoing; or (ii) comprises a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) and a reference lipid-anchored polymer comprising at least two hydrophobic tails each comprise 12 to 15 carbon atoms in a single aliphatic chain backbone, such as 1,2-dimyristoyl-rac-glycero-3-methoxypolyethylene glycol (DMG-PEG, also referred to as PEG-DMG).

As used herein, the term “aqueous solution” refers to a composition comprising in whole, or in part, water.

As used herein, the term “bases” includes purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the terms “carrier” and “excipient” are meant to include any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, the term “ceDNA” refers to capsid-free closed-ended linear double stranded (ds) duplex DNA for non-viral gene transfer, synthetic or otherwise. According to some embodiments, the ceDNA is a closed-ended linear duplex (CELiD) CELiD DNA. According to some embodiments, the ceDNA is a DNA-based minicircle. According to some embodiments, the ceDNA is a minimalistic immunological-defined gene expression (MIDGE)-vector. According to some embodiments, the ceDNA is a ministering DNA. According to some embodiments, the ceDNA is a dumbbell shaped linear duplex closed-ended DNA comprising two hairpin structures of ITRs in the 5′ and 3′ ends of an expression cassette. According to some embodiments, the ceDNA is a Doggybone™ DNA. Detailed description of ceDNA is described in International Patent Application No. PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International Patent Application Nos. PCT/US18/49996, filed Sep. 7, 2018, and PCT/US2018/064242, filed Dec. 6, 2018, each of which is incorporated herein in its entirety by reference. Certain methods for the production of synthetic ceDNA vectors comprising various ITR sequences and configurations are described, e.g., in International Application PCT/US2019/14122, filed on Jan. 18, 2019, the entire content of which is incorporated herein by reference.

As used herein, the term “closed-ended DNA vector” refers to a capsid-free DNA vector with at least one covalently closed end and where at least part of the vector has an intramolecular duplex structure. The terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “ceDNA genome” refers to an expression cassette that further incorporates at least one inverted terminal repeat (ITR) region. A ceDNA genome may further comprise one or more spacer regions. In some embodiments the ceDNA genome is incorporated as an intermolecular duplex polynucleotide of DNA into a plasmid or viral genome.

As used herein, the terms “DNA regulatory sequences,” “control elements,” and “regulatory elements,” are used interchangeably herein, and are meant to refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

As used herein, the terms “inverted terminal repeat” or “ITR” are meant to refer to a nucleic acid sequence located at the 5′ and/or 3′ terminus of the ssDNA vectors disclosed herein, which comprises at least one stem-loop structure comprising a partial duplex and at least one loop. According to some embodiments, the ITR may be an artificial sequence (e.g., contains no sequences derived from a virus). The ITR may further comprise one stem-loop structure (e.g., a “hairpin”), or more than one stem-loop structures. For example, the ITR may comprise two stem-loop structures (e.g., a “hammerhead”, “doggy-bone”, or “dumbbell”), three stem-loop structures (e.g., “cruciform”), or more complex structures. The ITR may comprise an aptamer sequence or one or more chemical modifications.

According to some embodiments, the “ITR” can be artificially synthesized using a set of oligonucleotides comprising one or more desirable functional sequences (e.g., palindromic sequence). The ITR sequence can be an artificial AAV ITR, an artificial non-AAV ITR, or an ITR physically derived from a viral AAV ITR (e.g., ITR fragments removed from a viral genome). For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. Typically, ITR sequences can be derived not only from AAV, but also from Parvovirus, lentivirus, goose virus, B19, in the configurations of wildtype, “doggy bone” and “dumbbell shape”, symmetrical or even asymmetrical ITR orientation. Although the ITRs are typically present in both 5′ and 3′ ends of an AAV vector, in a single-stranded DNA (ssDNA) molecule the ITR can be present in only one of end of the linear vector. For example, the ITR can be present on the 5′ end only. Some other cases, the ITR can be present on the 3′ end only in a single-stranded DNA (ssDNA) molecule. For convenience herein, an ITR located 5′ to (“upstream of”) an expression cassette in a single-stranded DNA (ssDNA) molecule is referred to as a “5′ ITR” or a “left ITR”, and an ITR located 3′ to (“downstream of”) an expression cassette in a single-stranded DNA (ssDNA) molecule is referred to as a “3′ ITR” or a “right ITR”.

As used herein, a “wild-type ITR” or “WT-ITR” refers to the sequence of a naturally occurring ITR sequence in an AAV genome or other dependovirus that remains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompasses for use herein include WT-ITR sequences as result of naturally occurring changes (e.g., a replication error).

As used herein, the term “substantially symmetrical WT-ITRs” or a “substantially symmetrical WT-ITR pair” refers to a pair of WT-ITRs within a synthetic AAV vector that are both wild type ITRs that have an inverse complement sequence across their entire length. For example, an ITR can be considered to be a wild-type sequence, even if it has one or more nucleotides that deviate from the canonical naturally occurring canonical sequence, so long as the changes do not affect the physical and functional properties and overall three-dimensional structure of the sequence (secondary and tertiary structures). In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (TRS) that pairs with the appropriate Rep protein. One can optionally test other functions, including transgene expression under permissive conditions.

As used herein, the phrases of “modified ITR” or “mod-ITR” or “mutant ITR” are used interchangeably and refer to an ITR with a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e., its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single synthetic AAV genome that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. According to some embodiments, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single stranded AAV genome that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a synthetic AAV vector is referred to as a “5′ ITR” or a “left ITR”, and an ITR located 3′ to (downstream of) an expression cassette in a synthetic AAV vector is referred to as a “3′ ITR” or a “right ITR”.

As used herein, the terms “substantially symmetrical modified-ITRs” or a “substantially symmetrical mod-ITR pair” refers to a pair of modified-ITRs within a synthetic AAV that are both that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. According to some embodiments, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, the term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. According to some embodiments, the term flanking refers to terminal repeats at each end of the linear single strand synthetic AAV vector.

As used herein, the term “spacer region” refers to an intervening sequence that separates functional elements in a vector or genome. In some embodiments, AAV spacer regions keep two functional elements at a desired distance for optimal functionality. In some embodiments, the spacer regions provide or add to the genetic stability of the vector or genome. In some embodiments, spacer regions facilitate ready genetic manipulation of the genome by providing a convenient location for cloning sites and a gap of design number of base pair. For example, in certain aspects, an oligonucleotide “polylinker” or “poly cloning site” containing several restriction endonuclease sites, or a non-open reading frame sequence designed to have no known protein (e.g., transcription factor) binding sites can be positioned in the vector or genome to separate the cis-acting factors, e.g., inserting a 6mer, 12mer, 18mer, 24mer, 48mer, 86mer, 176mer, etc., for example, between the terminal resolution site and the upstream transcriptional regulatory element as in an AAV vector or genome.

As used herein, the terms “Rep binding site” (“RBS”) and “Rep binding element” (“RBE”) are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are well known in the art, and include, for example, 5′-GCGCGCTCGCTCGCTC-3′, an RBS sequence identified in AAV2.

As used herein, the terms “terminal resolution site” and “TRS” are used interchangeably herein and refer to a region at which Rep forms a tyrosine-phosphodiester bond with the 5′ thymidine generating a 3′-OH that serves as a substrate for DNA extension via a cellular DNA polymerase, e.g., DNA pol delta or DNA pol epsilon. Alternatively, the Rep-thymidine complex may participate in a coordinated ligation reaction.

As used herein, the terms “sense” and “antisense” refer to the orientation of the structural element on the polynucleotide. The sense and antisense versions of an element are the reverse complement of each other.

As used herein, the term “synthetic AAV vector” and “synthetic production of AAV vector” refers to an AAV vector and synthetic production methods thereof in an entirely cell-free environment.

As used herein, the phrase an “effective amount” or “therapeutically effective amount” of an active agent or therapeutic agent, such as a therapeutic nucleic acid, is an amount sufficient to produce the desired effect, e.g., inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art.

As used herein, the term “expression” refers to the cellular processes involved in producing RNA and proteins and as appropriate, secreting proteins, including where applicable, but not limited to, for example, transcription, transcript processing, translation and protein folding, modification and processing. As used herein, the phrase “expression products” include RNA transcribed from a gene (e.g., transgene), and polypeptides obtained by translation of mRNA transcribed from a gene.

As used herein, the term “expression vector” refers to a vector that directs expression of an RNA or polypeptide from sequences linked to transcriptional regulatory sequences on the vector. The sequences expressed will often, but not necessarily, be heterologous to the host cell. An expression vector may comprise additional elements, for example, the expression vector may have two replication systems, thus allowing it to be maintained in two organisms, for example in human cells for expression and in a prokaryotic host for cloning and amplification. The expression vector may be a recombinant vector.

As used herein, the term “helper lipid” refers to a ceramide of the Formula (I), (II), (III), or (IV), or a salt or an ester thereof, or a deuterated analogue of any of the foregoing; or (ii) comprises a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE)

As used herein, the terms “expression cassette” and “expression unit” are used interchangeably and meant to refer to a heterologous DNA sequence that is operably linked to a promoter or other DNA regulatory sequence sufficient to direct transcription of a transgene of a DNA vector, e.g., synthetic AAV vector. Suitable promoters include, for example, tissue specific promoters. Promoters can also be of AAV origin.

As used herein, the phrase “genetic disease” or “genetic disorder” refers to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence.

As used herein, the term “polypeptide” refers to a polymeric sequence of amino acids. According to some embodiments, a polypeptide of the disclosure is an ApoE or an ApoB polypeptide. According to some embodiments, the ApoE polypeptide is a functional fragment (or a functional portion) of the full length ApoE polypeptide. According to some embodiments, the ApoE polypeptide is a functional fragment (or a functional portion) of the full length ApoB polypeptide. According to some embodiments, the ApoE polypeptide is 30 amino acids in length or less. According to some embodiments, the ApoB polypeptide is 30 amino acids in length or less.

As used herein, the term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

The term “lipid-anchored polymer” or “lipid polymer” or “lipid conjugate” refers to a conjugated lipid that inhibits aggregation of lipid particles. Such lipid conjugates include, but are not limited to, PEG-lipid conjugates such as PEG coupled to DSG (e.g., PEG-DSG conjugates), PEG coupled to DSPE (e.g., PEG-DSPE conjugates), and PEG conjugated to ceramides (see, e.g., U.S. Pat. No. 5,885,613), polyglycerol (PG)-lipid conjugate such as DODA-PG, and mixtures thereof. Examples of PG-lipid conjugates include DODA-PG45. Additional examples of POZ-lipid conjugates are described in PCT Publication No. WO 2010/006282. PEG, PG or POZ can be conjugated directly to the lipid or may be linked to the lipid via a linker moiety. Any linker moiety suitable for coupling the PEG, PG, or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

As used herein, the term “lipid-anchored polymer”, which may be used herein interchangeably with the term “lipid conjugate” or “lipid polymer” refers to a molecule comprising a lipid moiety covalently attached to a hydrophilic polymer via a linker. Without wishing to be bound by a specific theory, it is believed that a lipid-anchored polymer can inhibit aggregation of LNPs and provide steric stabilization and prolonged blood half-life (t_{1/2}) in vivo. The lipid moiety with a linker (“lipid-linker” or “linker-lipid moiety”) conjugated to a hydrophilic polymer (e.g., PEG, PG, or POZ) include, but are not limited to 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE), dioctadecylamine (DODA), distearoyl-rac-glycerol (DSG), 1,2-dipalmitoyl-rac-glycerol (DPG), a derivative thereof, and a combination any of the foregoing. In one embodiment, the lipid-anchored polymer comprises a linker-lipid moiety selected from the group consisting of DSPE, DSG, DODA, DPG, DOPE, and a derivative of thereof, and a combination of any of the foregoing. For example, PEG2000 coupled to DSG is a lipid-anchored polymer PEG2000-DSG (or DSG-PEG2000). PEG coupled to DSPE is a lipid-anchored polymer PEG-DSPE (or DSPE-PEG2000 or DSPE-PEG500)). An example of lipid-anchored PG polymer can include DODA-PG, wherein PG can be a multiunit ranging from about 5 to about 50 PG units.

As used herein, the term “lipid encapsulated” refers to a lipid particle that provides an active agent or therapeutic agent, such as a nucleic acid (e.g., a ceDNA, ssDNA, or mRNA), with full encapsulation, partial encapsulation, or both. In a preferred embodiment, the nucleic acid is fully encapsulated in the lipid particle (e.g., to form a nucleic acid containing lipid particle).

As used herein, the terms “lipid particle” or “lipid nanoparticle” refers to a lipid formulation that can be used to deliver a therapeutic agent such as nucleic acid therapeutics to a target site of interest (e.g., cell, tissue, organ, and the like). In one embodiment, the lipid particle of the disclosure is a nucleic acid containing lipid particle, which is typically formed from a cationic lipid, a non-cationic lipid, and optionally a conjugated lipid that prevents aggregation of the particle. In other preferred embodiments, a therapeutic agent such as a therapeutic nucleic acid may be encapsulated in the lipid portion of the particle, thereby protecting it from enzymatic degradation. In one embodiment, the lipid particle comprises a nucleic acid (e.g., ceDNA) and a lipid comprising one or more tertiary amino groups, one or more phenyl ester bonds and a disulfide bond.

According to some embodiments, lipid particles of the disclosure typically have a mean diameter of from about 20 nm to about 75 nm, about 20 nm to about 70 nm, about 25 nm to about 75 nm, about 25 nm to about 70 nm, about 30 nm to about 75 nm, about 30 nm to about 70 nm, about 35 nm to about 75 nm, about 35 nm to about 70 nm, about 40 nm to about 75 nm, about 40 nm to about 70 nm, about 45 nm to about 75 nm, about 50 nm to about 75 nm, about 50 nm to about 70 nm, about 60 nm to about 75 nm, about 60 nm to about 70 nm, about 65 nm to about 75 nm, about 65 nm to about 70 nm, about 65 nm to about 80 nm, about 65 nm to about 80 nm, about 60 nm to about 80 nm, about 65 nm to about 85 nm, or about 20 nm, about 25 nm, about 30 nm, about 35 nm, about 40 nm, about 45 nm, about 50 nm, about 51 nm, about 52 nm, about 53 nm, about 54 nm, about 55 nm, about 56 nm, about 57 nm, about 58 nm, about 59 nm about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, or about 85 nm (±3 nm) in size.

Generally, the LNPs of the disclosure have a mean diameter selected to provide an intended therapeutic effect. For example, the LNPs of the disclosure have a mean diameter that is compatible with delivery to a target organ, such that the LNPs of the disclosure are able to diffuse through the fenestrations of a target organ (e.g., liver) or a target cell subpopulation (e.g., hepatocytes).

According to some embodiments, the lipid particles of the disclosure typically have a mean diameter of less than about 85 nm, less than about 80 nm, less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, or less than about 20 nm in size.

As used herein, the term “cationic lipid” refers to any lipid that is positively charged at physiological pH. The cationic lipid in the lipid particles may comprise, e.g., one or more cationic lipids such as 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), “SS-cleavable lipid”, or a mixture thereof. In some embodiments, a cationic lipid can also be an ionizable lipid, i.e., an ionizable cationic lipid. The term “cationic lipids” also encompasses lipids that are positively charged at any pH, e.g., lipids comprising quaternary amine groups, i.e., quaternary lipids. Any cationic lipid described herein comprising a primary, secondary or tertiary amine group may be converted to a corresponding quaternary lipid, for example, by treatment with chloromethane (CH₃Cl) in acetonitrile (CH₃CN) and chloroform (CHCl₃).

As used herein, the term “ionizable lipid” refers to a lipid, e.g., cationic lipid, having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipids be present in the charged or neutral form. Generally, ionizable lipids have a pKa of the protonatable group in the range of about 4 to about 7. In some embodiments, ionizable lipid may include “cleavable lipid” or “SS-cleavable lipid”.

As used herein, the term “neutral lipid” refers to any number of lipid species that exists either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “cleavable lipid” or “SS-cleavable lipid” refers to an ionizable lipid comprising a disulfide bond cleavable unit. Cleavable lipids may include cleavable disulfide bond (“ss”) containing lipid-like materials that comprise a pH-sensitive amine, e.g., a tertiary amine, and self-degradable phenyl ester. For example, a SS-cleavable lipid can be an ss-OP lipid (COATSOME® SS-OP), an ss-M lipid (COATSOME® SS-M), an ss-E lipid (COATSOME® SS-E), an ss-EC lipid (COATSOME® SS-EC), an ss-LC lipid (COATSOME® SS-LC), an ss-OC lipid (COATSOME® SS-OC), and an ss-PalmE lipid (see, for example, Formulae I-IV), or a lipid described by Togashi et al., (2018) Journal of Controlled Release “A hepatic pDNA delivery system based on an intracellular environment sensitive vitamin E-scaffold lipid-like material with the aid of an anti-inflammatory drug” 279:262-270. Additional examples of cleavable lipids are described in U.S. Pat. Nos. 9,708,628, and 10,385,030, the entire contents of which are incorporated herein by reference. In one embodiment, cleavable lipids comprise a tertiary amine, which responds to an acidic compartment, e.g., an endosome or lysosome for membrane destabilization and a disulfide bond that can be cleaved in a reducing environment, such as the cytoplasm. In one embodiment, a cleavable lipid is a cationic lipid. In one embodiment, a cleavable lipid is an ionizable cationic lipid. Cleavable lipids are described in more detail herein.

As used herein, the term “organic lipid solution” refers to a composition comprising in whole, or in part, an organic solvent having a lipid.

As used herein, the term “liposome” refers to lipid molecules assembled in a spherical configuration encapsulating an interior aqueous volume that is segregated from an aqueous exterior. Liposomes are vesicles that possess at least one lipid bilayer. Liposomes are typically used as carriers for drug/therapeutic delivery in the context of pharmaceutical development. They work by fusing with a cellular membrane and repositioning its lipid structure to deliver a drug or active pharmaceutical ingredient. Liposome compositions for such delivery are typically composed of phospholipids, especially compounds having a phosphatidylcholine group, however these compositions may also include other lipids.

As used herein, the term “nucleic acid,” refers to a polymer containing at least two nucleotides (i.e., deoxyribonucleotides or ribonucleotides) in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), single-stranded DNA (ssDNA), Doggybone™ DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), messenger RNA (mRNA), rRNA, tRNA, gRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA) or guide RNA (gRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), single-stranded DNA (ssDNA), plasmids, bacmids, DOGGYBONE™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, “nucleotides” contain a sugar deoxyribose (DNA) or ribose (RNA), a base, and a phosphate group. Nucleotides are linked together through phosphate groups.

As used herein, the term “pharmaceutically acceptable carrier” includes any of the standard pharmaceutical carriers, such as a phosphate buffered saline solution, water, emulsions such as an oil/water or water/oil, and various types of wetting agents. The term also encompasses any of the agents approved by a regulatory agency of the U.S. federal government or listed in the US Pharmacopeia for use in animals, including humans, as well as any carrier or diluent that does not cause significant irritation to a subject and does not abrogate the biological activity and properties of the administered compound.

As used herein, the terms “gap” and “nick” are used interchangeably and refer to a discontinued portion of synthetic DNA vector of the present disclosure, creating a stretch of single stranded DNA portion in otherwise double stranded ceDNA. The gap can be 1 nucleotide (nt) to 100 nucleotides (nt) long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 nt to 10 nt long, 1 to 20 nt long, 1 to 30 nt long in length.

As used herein, the term “nick” refers to a discontinuity in a double stranded DNA molecule where there is no phosphodiester bond between adjacent nucleotides of one strand typically through damage or enzyme action. It is understood that one or more nicks allow for the release of torsion in the strand during DNA replication and that nicks are also thought to play a role in facilitating binding of transcriptional machinery.

By “receptor” means a polypeptide, or portion thereof, present on a cell membrane that selectively binds one or more ligands. The term “receptor” as used herein is intended to encompass the entire receptor or ligand-binding portions thereof. These portions of the receptor particularly include those regions sufficient for specific binding of the ligand to occur.

As used herein, the term “subject” refers to a human or animal, to whom treatment, including prophylactic treatment, with the therapeutic nucleic acid according to the present disclosure, is provided. Usually, the animal is a vertebrate such as, but not limited to, a primate, rodent, domestic animal or game animal. Primates include but are not limited to, chimpanzees, cynomolgus monkeys, spider monkeys, and macaques, e.g., rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human. A subject can be male or female. Additionally, a subject can be an infant or a child. In some embodiments, the subject can be a neonate or an unborn subject, e.g., the subject is in utero. Preferably, the subject is mammals. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Middle Eastern, etc. In some embodiments, the subject can be a patient or another subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the phrase “subject in need” refers to a subject that (i) will be administered a ceDNA lipid particle (or pharmaceutical composition comprising a ceDNA lipid particle) according to the described disclosure, (ii) is receiving a ceDNA lipid particle (or pharmaceutical composition comprising aceDNA lipid particle) according to the described disclosure; or (iii) has received a ceDNA lipid particle (or pharmaceutical composition comprising a ceDNA lipid particle) according to the described disclosure, unless the context and usage of the phrase indicates otherwise.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, the term “systemic delivery” refers to delivery of lipid particles that leads to a broad biodistribution of an active agent such as an interfering RNA (e.g., siRNA), mRNA, ceDNA, or ssDNA within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of LNPs can be by any means known in the art including, for example, intravenous, subcutaneous, and intraperitoneal. In a preferred embodiment, systemic delivery of LNPs is by intravenous delivery.

As used herein, the terms “effective amount”, which may be used interchangeably with the terms “therapeutic amount”, “therapeutically effective amount”, an “amount effective”, or “pharmaceutically effective amount” of an active agent (e.g., a ceDNA as described herein), refers to an amount that is sufficient to provide the intended benefit of treatment or effect, e.g., expression or inhibition of expression of a target sequence in comparison to the expression level detected in the absence of a therapeutic nucleic acid. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art. Dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “effective amount”, “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein. In one aspect of any of the aspects or embodiments herein, “therapeutic amount”, “therapeutically effective amounts” and “pharmaceutically effective amounts” refer to non-prophylactic or non-preventative applications.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “treat,” “treating,” and/or “treatment” include abrogating, inhibiting, slowing or reversing the progression of a condition, ameliorating clinical symptoms of a condition, or preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). In one aspect of any of the aspects or embodiments herein, the terms “treat,” “treating,” and/or “treatment” include abrogating, inhibiting, slowing or reversing the progression of a condition, or ameliorating clinical symptoms of a condition.

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “combination therapy” refers to treatment regimens for a clinical indication that comprise two or more therapeutic agents. Thus, the term refers to a therapeutic regimen in which a first therapy comprising a first composition (e.g., active ingredient) is administered in conjunction with a second therapy comprising a second composition (active ingredient) to a patient, intended to treat the same or overlapping disease or clinical condition. The first and second compositions may both act on the same cellular target, or discrete cellular targets. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

As used herein, the term “alkyl” refers to a saturated monovalent hydrocarbon radical of 1 to 20 carbon atoms (i.e., C₁₋₂₀ alkyl). “Monovalent” means that alkyl has one point of attachment to the remainder of the molecule. In one embodiment, the alkyl has 1 to 12 carbon atoms (i.e., C₁₋₁₂ alkyl) or 1 to 10 carbon atoms (i.e., C₁₋₁₀ alkyl). In one embodiment, the alkyl has 1 to 8 carbon atoms (i.e., C₁₋₈ alkyl), 1 to 7 carbon atoms (i.e., C₁₋₇ alkyl), 1 to 6 carbon atoms (i.e., C₁₋₆ alkyl), 1 to 4 carbon atoms (i.e., C₁₋₄ alkyl), or 1 to 3 carbon atoms (i.e., C₁₋₃ alkyl). Examples include, but are not limited to, methyl, ethyl, 1-propyl, 2-propyl, 1-butyl, 2-methyl-1-propyl, 2-butyl, 2-methyl-2-propyl, 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, 1-heptyl, 1-octyl, and the like. A linear or branched alkyl, such as a “linear or branched C₁₋₆ alkyl,” “linear or branched C₁₋₄ alkyl,” or “linear or branched C₁₋₃ alkyl” means that the saturated monovalent hydrocarbon radical is a linear or branched chain. As used herein, the term “linear” as referring to aliphatic hydrocarbon chains means that the chain is unbranched.

The term “alkylene” as used herein refers to a saturated divalent hydrocarbon radical of 1 to 20 carbon atoms (i.e., C₁₋₂₀ alkylene), examples of which include, but are not limited to, those having the same core structures of the alkyl groups as exemplified above. “Divalent” means that the alkylene has two points of attachment to the remainder of the molecule. In one embodiment, the alkylene has 1 to 12 carbon atoms (i.e., C₁₋₁₂ alkylene) or 1 to 10 carbon atoms (i.e., C₁₋₁₀ alkylene). In one embodiment, the alkylene has 1 to 8 carbon atoms (i.e., C₁₋₈ alkylene), 1 to 7 carbon atoms (i.e., C₁₋₇ alkylene), 1 to 6 carbon atoms (i.e., C₁₋₆ alkylene), 1 to 4 carbon atoms (i.e., C₁₋₄ alkylene), 1 to 3 carbon atoms (i.e., C₁₋₃ alkylene), ethylene, or methylene. A linear or branched alkylene, such as a “linear or branched C₁₋₆ alkylene,” “linear or branched C₁₋₄ alkylene,” or “linear or branched C₁₋₃ alkylene” means that the saturated divalent hydrocarbon radical is a linear or branched chain.

The term “alkenyl” refers to straight or branched aliphatic hydrocarbon radical with one or more (e.g., one or two) carbon-carbon double bonds, wherein the alkenyl radical includes radicals having “cis” and “trans” orientations, or by an alternative nomenclature, “E” and “Z” orientations.

“Alkenylene” as used herein refers to aliphatic divalent hydrocarbon radical of 2 to 20 carbon atoms (i.e., C₂₋₂₀ alkenylene) with one or two carbon-carbon double bonds, wherein the alkenylene radical includes radicals having “cis” and “trans” orientations, or by an alternative nomenclature, “E” and “Z” orientations. “Divalent” means that alkenylene has two points of attachment to the remainder of the molecule. In one embodiment, the alkenylene has 2 to 12 carbon atoms (i.e., C₂₋₁₆ alkenylene), 2 to 10 carbon atoms (i.e., C₂₋₁₀ alkenylene). In one embodiment, the alkenylene has 2 to four carbon atoms (C₂₋₄). Examples include, but are not limited to, ethylenylene or vinylene (—CH═CH—), allyl (—CH₂CH═CH—), and the like. A linear or branched alkenylene, such as a “linear or branched C₂₋₆ alkenylene,” “linear or branched C₂₋₄ alkenylene,” or “linear or branched C₂₋₃ alkenylene” means that the unsaturated divalent hydrocarbon radical is a linear or branched chain.

“Cycloalkylene” as used herein refers to a divalent saturated carbocyclic ring radical having 3 to 12 carbon atoms as a monocyclic ring, or 7 to 12 carbon atoms as a bicyclic ring. “Divalent” means that the cycloalkylene has two points of attachment to the remainder of the molecule. In one embodiment, the cycloalkylene is a 3- to 7-membered monocyclic or 3- to 6-membered monocyclic. Examples of monocyclic cycloalkyl groups include, but are not limited to, cyclopropylene, cyclobutylene, cyclopentylene, cyclohexylene, cycloheptylene, cyclooctylene, cyclononylene, cyclodecylene, cycloundecylene, cyclododecylene, and the like. In one embodiment, the cycloalkylene is cyclopropylene.

The terms “heterocycle,” “heterocyclyl,” heterocyclic and “heterocyclic ring” are used interchangeably herein and refer to a cyclic group which contains at least one N atom has a heteroatom and optionally 1-3 additional heteroatoms selected from N and S, and are non-aromatic (i.e., partially or fully saturated). It can be monocyclic or bicyclic (bridged or fused). Examples of heterocyclic rings include, but are not limited to, aziridinyl, diaziridinyl, thiaziridinyl, azetidinyl, diazetidinyl, triazetidinyl, thiadiazetidinyl, thiazetidinyl, pyrrolidinyl, pyrazolidinyl, imidazolinyl, isothiazolidinyl, thiazolidinyl, piperidinyl, piperazinyl, hexahydropyrimidinyl, azepanyl, azocanyl, and the like. The heterocycle contains 1 to 4 heteroatoms, which may be the same or different, selected from N and S. In one embodiment, the heterocycle contains 1 to 3 N atoms. In another embodiment, the heterocycle contains 1 or 2 N atoms. In another embodiment, the heterocycle contains 1 N atom. A “4- to 8-membered heterocyclyl” means a radical having from 4 to 8 atoms (including 1 to 4 heteroatoms selected from N and S, or 1 to 3 N atoms, or 1 or 2 N atoms, or 1 N atom) arranged in a monocyclic ring. A “5- or 6-membered heterocyclyl” means a radical having from 5 or 6 atoms (including 1 to 4 heteroatoms selected from N and S, or 1 to 3 N atoms, or 1 or 2 N atoms, or 1 N atom) arranged in a monocyclic ring. The term “heterocycle” is intended to include all the possible isomeric forms. Heterocycles are described in Paquette, Leo A., Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs (John Wiley & Sons, New York, 1950 to present), in particular Volumes 13, 14, 16, 19, and 28; and J. Am. Chem. Soc. (1960) 82:5566. The heterocyclyl groups may be carbon (carbon-linked) or nitrogen (nitrogen-linked) attached to the rest of the molecule where such is possible.

If a group is described as being “optionally substituted,” the group may be either (1) not substituted, or (2) substituted. If a carbon of a group is described as being optionally substituted with one or more of a list of substituents, one or more of the hydrogen atoms on the carbon (to the extent there are any) may separately and/or together be replaced with an independently selected optional substituent.

Suitable substituents for an alkyl, alkylene, alkenylene, cycloalkylene, and heterocyclyl, are those which do not significantly adversely affect the biological activity of the molecule. Unless otherwise specified, exemplary substituents for these groups include linear, branched or cyclic alkyl, alkenyl or alkynyl having from 1 to 10 carbon atoms, aryl, heteroaryl, heterocyclyl, halogen, guanidinium [—NH(C═NH)NH₂], —OR₁₀₀, NR₁₀₁R₁₀₂, —NO₂, —NR₁₀₁COR₁₀₂, —SR₁₀₀, a sulfoxide represented by —SOR₁₀₁, a sulfone represented by —SO₂R₁₀₁, a sulfonate —SO₃M, a sulfate —OSO₃M, a sulfonamide represented by —SO₂NR₁₀₁R₁₀₂, cyano, an azido, —COR₁₀₁, —OCOR₁₀₁, —OCONR₁₀₁R₁₀₂ and a polyethylene glycol unit (—OCH₂CH₂)ₙR₁₀₁ wherein M is H or a cation (such as Na⁺ or K⁺); R₁₀₁, R₁₀₂ and R₁₀₃ are each independently selected from H, linear, branched or cyclic alkyl, alkenyl or alkynyl having from 1 to 10 carbon atoms, a polyethylene glycol unit (—OCH₂CH₂) n-R₁₀₄, wherein n is an integer from 1 to 24, an aryl having from 6 to 10 carbon atoms, a heterocyclic ring having from 3 to 10 carbon atoms and a heteroaryl having 5 to 10 carbon atoms; and R₁₀₄ is H or a linear or branched alkyl having 1 to 4 carbon atoms, wherein the alkyl, alkenyl, alkynyl, aryl, heteroaryl and heterocyclyl in the groups represented by R₁₀₀, R₁₀₁, R₁₀₂, R₁₀₃ and R₁₀₄ are optionally substituted with one or more (e.g., 2, 3, 4, 5, 6 or more) substituents independently selected from halogen, —OH, —CN, —NO₂, and unsubstituted linear or branched alkyl having 1 to 4 carbon atoms. Preferably, the substituent for the optionally substituted alkyl, alkylene, alkenylene, cycloalkylene, and heterocyclyl described above is selected from the group consisting of halogen, —CN, —NR₁₀₁R₁₀₂, —CF₃, —OR₁₀₀, aryl, heteroaryl, heterocyclyl, —SR₁₀₁, —SOR₁₀₁, —SO₂R₁₀₁, and —SO₃M. Alternatively, the suitable substituent is selected from the group consisting of halogen, —OH, —NO₂, —CN, C₁₋₄ alkyl, —OR₁₀₀, NR₁₀₁R₁₀₂, —NR₁₀₁COR₁₀₂, —SR₁₀₀, —SO₂R₁₀₁, —SO₂NR₁₀₁R₁₀₂, —COR₁₀₁, —OCOR₁₀₁, and —OCONR₁₀₁R₁₀₂, wherein R₁₀₀, R₁₀₁, and R₁₀₂ are each independently —H or C₁₋₄ alkyl.

“Halogen” as used herein refers to F, Cl, Br or I. “Cyano” is —CN.

“Amine” or “amino” as used herein interchangeably refers to a functional group that contains a basic nitrogen atom with a lone pair.

The term “pharmaceutically acceptable salt” as used herein refers to pharmaceutically acceptable organic or inorganic salts of an ionizable lipid of the disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ions.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the disclosure.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited in any manner to the particular methodology, protocols, and reagents, etc., described herein and as such can vary. The terminology used herein is for the purpose of describing particular embodiments only and is not intended to limit the scope of the present disclosure, which is defined solely by the claims.

### II. Lipid Nanoparticles (LNPs)

Provided herein are lipid nanoparticles (LNPs) comprising a therapeutic nucleic acid (TNA); an ionizable lipid; a structural lipid (e.g., a sterol); one or more lipid-anchored polymers, e.g., a first lipid-anchored polymer and a second lipid-anchored polymer, and a ceramide or other helper lipid. Also provided herein are LNPs consisting essentially of a therapeutic nucleic acid (TNA); an ionizable lipid; a structural lipid (e.g., a sterol); one or more lipid-anchored polymers, e.g., a first lipid-anchored polymer and a second lipid-anchored polymer, and a ceramide or other helper lipid. Also provided herein are LNPs consisting of a therapeutic nucleic acid (TNA); an ionizable lipid; a structural lipid (e.g., a sterol); one or more lipid-anchored polymers, e.g., a first lipid-anchored polymer and a second lipid-anchored polymer, and a ceramide or other helper lipid.

As used herein, the term “lipid particle” or “lipid nanoparticle” (LNP) refers to a lipid formulation that can be used to deliver a therapeutic agent such as therapeutic nucleic acid to a target site of interest (e.g., cell, tissue, organ, and the like). In some embodiments, the lipid nanoparticle of the disclosure is typically formed from an ionizable lipid (e.g., cationic lipid), sterol (e.g., cholesterol), a conjugated lipid (e.g., lipid-anchored polymer) that prevents aggregation of the particle, and optionally a helper lipid (e.g., non-cationic lipid). In some other embodiments, a therapeutic agent such as a therapeutic nucleic acid (TNA) may be encapsulated in the lipid particle, thereby protecting it from degradation. In yet other embodiments, an immunosuppressant can be optionally included in the nucleic acid containing lipid nanoparticles. In one embodiment, the lipid particle comprises a nucleic acid (e.g., ceDNA, ssDNA and/or mRNA). The present disclosure provides LNPs where at least one of the lipids in the lipid anchored polymer contains either 16, 18 or 20 aliphatic carbons to more securely anchor the lipid anchored polymer to the LNP. In some embodiments, at least one lipid of the lipid anchored polymer having at least 18 aliphatic carbons is useful for creating stealth LNPs. In another embodiment, at least one lipid of the lipid anchored polymer having at least 20 aliphatic carbons is useful for creating stealth LNPs.

According to some embodiments, lipid nanoparticles of the disclosure typically have a mean diameter of from about 20 nm to about 90 nm, about 25 nm to about 80 nm, about 25 nm to about 75 nm, about 25 nm to about 70 nm, from about 30 nm to about 75 nm, from about 30 nm to about 70 nm, from about 35 nm to about 75 nm, from about 35 nm to about 70 nm, from about 40 nm to about 75 nm, from about 40 nm to about 70 nm, from about 45 nm to about 75 nm, from about 50 nm to about 75 nm, from about 50 nm to about 70 nm, from about 60 nm to about 75 nm, from about 60 nm to about 70 nm, from about 65 nm to about 75 nm, from about 65 nm to about 70 nm, or about 20 nm, about 25 nm, about 30 nm, about 35 nm, about 40 nm, about 45 nm, about 50 nm, about 51 nm, about 52 nm, about 53 nm, about 54 nm, about 55 nm, about 56 nm, about 57 nm, about 58 nm, about 59 nm about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, or about 75 nm (+3 nm) in size.

Generally, the LNPs of the disclosure have a mean diameter selected to provide an intended therapeutic effect. For example, the LNPs of the disclosure have a mean diameter that is compatible with a target organ, such that the LNPs of the disclosure are able to diffuse through the fenestrations of a target organ (e.g., liver) or a target cell subpopulation (e.g., hepatocytes).

According to some embodiments, the lipid particles of the disclosure typically have a mean diameter of less than about 100 nm, less than about 90 nm, less than about 80 nm, less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm in size.

In some embodiments, an LNP of the present disclosure does not comprise distearoylphosphatidylcholine (DSPC), provided that a helper lipid represented by (I), (II), (III), or (IV), or a salt or an ester thereof, or a deuterated analogue of any of the foregoing is present. In some embodiments, an LNP of the present disclosure does not comprise 1,2-dioleoyl-sn-glycero-3-phosphocholine (DOPC), provided that a helper lipid represented by (I), (II), (III), or (IV), or a salt or an ester thereof, or a deuterated analogue of any of the foregoing is present.

### A. Ionizable Lipids

In some embodiments, the ionizable lipid is present in the LNP provided by the present disclosure in an amount of about 20 mol % to about 70 mol %, about 20 mol % to about 65 mol %, about 20 mol % to about 60 mol %, about 20 mol % to about 55 mol %, about 20 mol % to about 50 mol %, about 25 mol % to about 70 mol %, about 25 mol % to about 65 mol %, about 25 mol % to about 60 mol %, about 25 mol % to about 55 mol %, about 25 mol % to about 50 mol %, about 30 mol % to about 70 mol %, about 30 mol % to about 65 mol %, about 30 mol % to about 60 mol %, about 30 mol % to about 55 mol %, about 30 mol % to about 50 mol %, about 35 mol % to about 70 mol %, about 35 mol % to about 65 mol %, about 35 mol % to about 60 mol %, about 35 mol % to about 55 mol %, about 35 mol % to about 50 mol %, 40 mol % to about 70 mol %, about 40 mol % to about 65 mol %, about 40 mol % to about 60 mol %, about 40 mol % to about 55 mol %, or about 40 mol % to about 50 mol %, of the total lipid present in the LNP.

In some embodiments, the LNPs provided by the present disclosure comprise an ionizable lipid. Exemplary ionizable lipids in the LNPs of the present disclosure are described in International Patent Application Publication Nos. WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US Patent Application Publication Nos. US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

### Formula (A)

In some embodiments, the ionizable lipid in the LNPs of the present disclosure is represented by Formula (A):

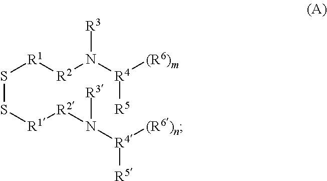

or a pharmaceutically acceptable salt thereof, wherein:
R¹ and R^{1′} are each independently C₁₋₃ alkylene;
R² and R^{2′} are each independently linear or branched C₁₋₆ alkylene, or C₃₋₆ cycloalkylene;
R³ and R^{3′} are each independently optionally substituted C₁₋₆ alkyl or optionally substituted C₃₋₆ cycloalkyl;
or alternatively, when R² is branched C₁₋₆ alkylene and when R³ is C₁₋₆ alkyl, R² and R³, taken together with their intervening N atom, form a 4- to 8-membered heterocyclyl;
or alternatively, when R^{2′} is branched C₁₋₆ alkylene and when R^{3′} is C₁₋₆ alkyl, R^{2′} and R³, taken together with their intervening N atom, form a 4- to 8-membered heterocyclyl;
R⁴ and R^{4′} are each independently —CH, —CH₂CH, or —(CH₂)₂CH;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5.

In some embodiments, R² and R^{2′} are each independently C₁₋₃ alkylene.

In some embodiments, the linear or branched C₁₋₃ alkylene represented by R¹ or R^{1′}, the linear or branched C₁₋₆ alkylene represented by R² or R^{2′}, and the optionally substituted linear or branched C₁₋₆ alkyl are each optionally substituted with one or more halo and cyano groups.

In some embodiments, R¹ and R² taken together are C₁₋₃ alkylene and R^{1′} and R^{2′} taken together are C₁₋₃ alkylene, e.g., ethylene.

In some embodiments, R³ and R^{3′} are each independently optionally substituted C₁₋₃ alkyl, e.g., methyl.

In some embodiments, R⁴ and R^{4′} are each —CH.

In some embodiments, R² is optionally substituted branched C₁₋₆ alkylene; and R² and R³, taken together with their intervening N atom, form a 5- or 6-membered heterocyclyl. In some embodiments, R^{2′} is optionally substituted branched C₁₋₆ alkylene; and R^{2′} and R^{3′}, taken together with their intervening N atom, form a 5- or 6-membered heterocyclyl, such as pyrrolidinyl or piperidinyl.

In some embodiments, R⁴ is —C(R^{a})₂CR^{a}, or —[C(R^{a})₂]₂CR^{a} and R^{a} is C₁₋₃ alkyl; and R³ and R⁴, taken together with their intervening N atom, form a 5- or 6-membered heterocyclyl. In some embodiments, R^{4′} is —C(R^{a})₂CR^{a}, or —[C(R^{a})₂]₂CR^{a} and R^{a} is C₁₋₃ alkyl; and R^{3′} and R^{4′}, taken together with their intervening N atom, form a 5- or 6-membered heterocyclyl, such as pyrrolidinyl or piperidinyl.

In some embodiments, R⁵ and R^{5′} are each independently C₁₋₁₀ alkylene or C₂₋₁₀ alkenylene. In one embodiment, R⁵ and R^{5′} are each independently C₁₋₈ alkylene or C₁₋₆ alkylene.

In some embodiments, R⁶ and R^{6′}, for each occurrence, are independently C₁₋₁₀ alkylene, C₃₋₁₀ cycloalkylene, or C₂₋₁₀ alkenylene. In one embodiment, C₁₋₆ alkylene, C₃₋₆ cycloalkylene, or C₂₋₆ alkenylene. In one embodiment the C₃₋₁₀ cycloalkylene or the C₃₋₆ cycloalkylene is cyclopropylene. In some embodiments, m and n are each 3.

In some embodiments, the ionizable lipid in the LNPs of the present disclosure may be selected from any one of the lipids listed in Table 1 below, or a pharmaceutically acceptable salt thereof.

TABLE 1
Exemplary ionizable lipids of Formula (A)

| Lipid No. | Structure and Name |
|---|---|
| 1 | 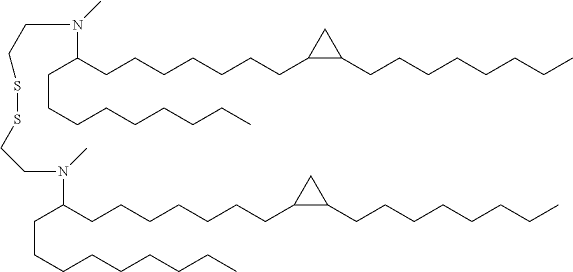 [SMILES: CCCCCCCCCC(CCCCCCCC1CC1CCCCCCCC)N(C)CCSSCCN(C)C(CCCCCCCCC)CCCCCCCC1CC1CCCCCCCC] |
| | N,N′-(disulfanediylbis(ethane-2,1-diyl))bis(N-methyl-1-(2-octylcyclopropyl)- |
| | heptadecan-8-amine) |
| 2 | 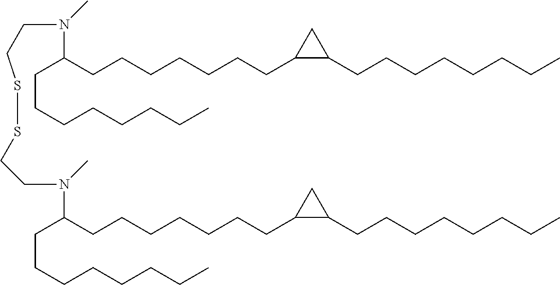 [SMILES: CCCCCCCCC1CC1CCCCCCCC(CCCCCCCC)N(C)CCSSCCN(C)C(CCCCCCCC)CCCCCCCC1CC1CCCCCCCC] |
| | N,N′-(disulfanediylbis(ethane-2,1-diyl))bis(N-methyl-1-(2-octylcyclopropyl)- |
| | hexadecan-8-amine) |
| 3 | 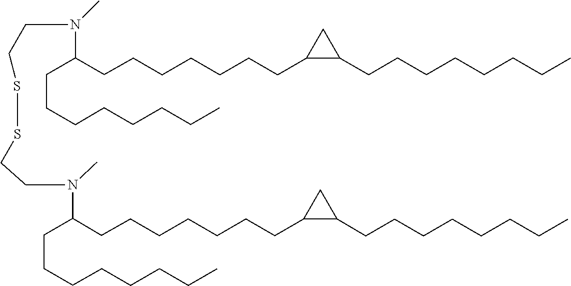 [SMILES: CCCCCCCCC1CC1CCCCCCCC(CCCCCCCC)N(C)CCSSCCN(C)C(CCCCCCCC)CCCCCCCC1CC1CCCCCCCC] |
| | N,N′-(disulfanediylbis(ethane-2,1-diyl))bis(N-methyl-1-(2-octylcyclopropyl)- |
| | hexadecan-8-amine) |
| 4 | 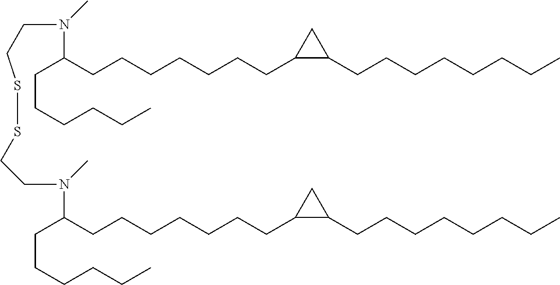 [SMILES: CCCCCCCCC1CC1CCCCCCCC(CCCCCC)N(C)CCSSCCN(C)C(CCCCCC)CCCCCCCC1CC1CCCCCCCC] |
| | N,N′-(disulfanediylbis(ethane-2,1-diyl))bis(N-methyl-14-(2-octylcyclopropyl)- |
| | tetradecan-7-amine) |
| 5 | 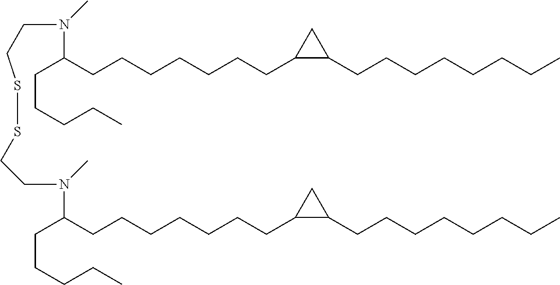 [SMILES: CCCCCCCCC1CC1CCCCCCCC(CCCCC)N(C)CCSSCCN(C)C(CCCCC)CCCCCCCC1CC1CCCCCCCC] |
| | N,N′-(disulfanediylbis(ethane-2,1-diyl))bis(N-methyl-13-(2-octylcyclopropyl)- |
| | tridecan-6-amine) |
| 6 | 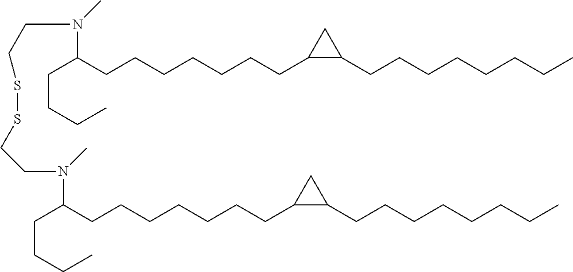 [SMILES: CCCCCCCCC1CC1CCCCCCCC(CCCC)N(C)CCSSCCN(C)C(CCCC)CCCCCCCC1CC1CCCCCCCC] |
| | N,N′-(disulfanediylbis(ethane-2,1-diyl))bis(N-methyl-12-(2-octylcyclopropyl)- |
| | dodecan-5-amine) |
| 7 | 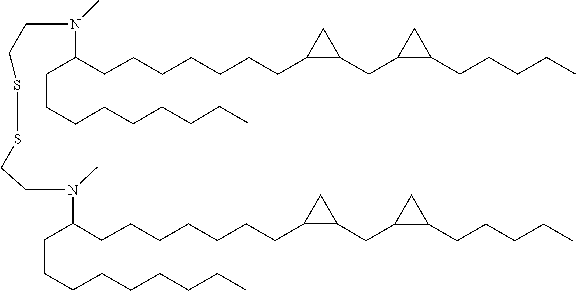 [SMILES: CCCCCCCCCC(CCCCCCCC1CC1CC1CC1CCCCC)N(C)CCSSCCN(C)C(CCCCCCCCC)CCCCCCCC1CC1CC1CC1CCCCC] |
| | N,N′-(disulfanediylbis(ethane-2,1-diyl))bis(N-methyl-1-(2-((2-pentylcyclopropyl)- |
| | methyl)cyclopropyl)heptadecan-8-amine) |
| 8 | 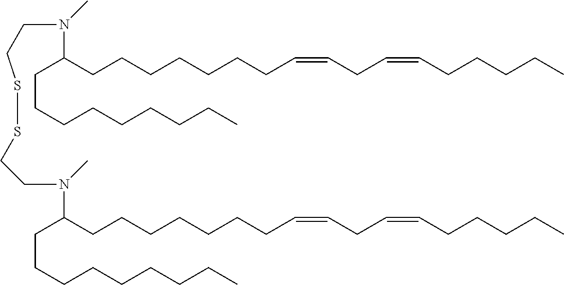 [SMILES: CCCCC/C=C\C/C=C\CCCCCCCC(CCCCCCCCC)N(C)CCSSCCN(C)C(CCCCCCC/C=C\C/C=C\CCCCC)CCCCCCCCC] |
| | (18Z,18′Z,21Z,21′Z)-N,N′-(disulfanediylbis(ethane-2,1-diyl))bis(N-methylheptacosa- |
| | 18,21-dien-10-amine) |
| 9 | 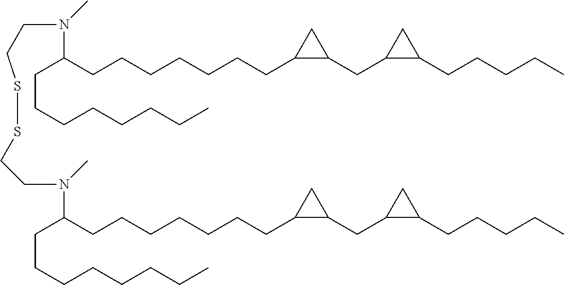 [SMILES: CCCCCCCCC(CCCCCCCC1CC1CC1CC1CCCCC)N(C)CCSSCCN(C)C(CCCCCCCC)CCCCCCCC1CC1CC1CC1CCCCC] |
| | N,N′-(disulfanediylbis(ethane-2,1-diyl))bis(N-methyl-1-(2-((2-pentylcyclopropyl)- |
| | methyl)cyclopropyl)hexadecan-8-amine) |
| 10 | 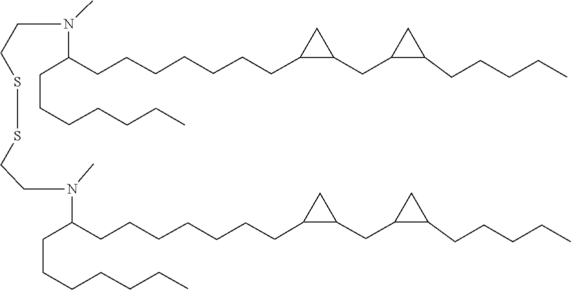 [SMILES: CCCCCCCC(CCCCCCCC1CC1CC1CC1CCCCC)N(C)CCSSCCN(C)C(CCCCCCC)CCCCCCCC1CC1CC1CC1CCCCC] |
| | N,N′-(disulfanediylbis(ethane-2,1-diyl))bis(N-methyl-1-(2-((2-pentylcyclopropyl)- |
| | methyl)cyclopropyl)pentadecan-8-amine) |
| 11 | 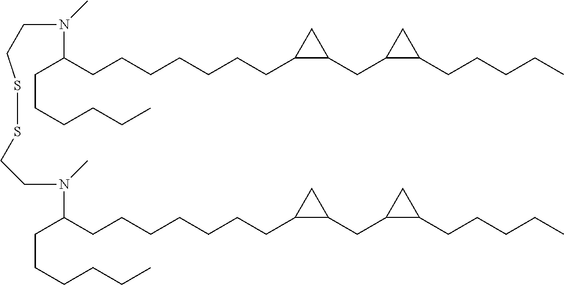 [SMILES: CCCCCCC(CCCCCCCC1CC1CC1CC1CCCCC)N(C)CCSSCCN(C)C(CCCCCC)CCCCCCCC1CC1CC1CC1CCCCC] |
| | N,N′-(disulfanediylbis(ethane-2,1-diyl))bis(N-methyl-14-(2-((2-pentylcyclopropyl)- |
| | methyl)cyclopropyl)tetradecan-7-amine) |
| 12 | 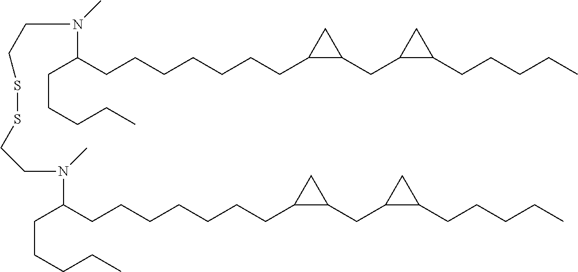 [SMILES: CCCCCC1CC1CC1CC1CCCCCCCC(CCCCC)N(C)CCSSCCN(C)C(CCCCC)CCCCCCCC1CC1CC1CC1CCCCC] |
| | N,N′-(disulfanediylbis(ethane-2,1-diyl))bis(N-methyl-13-(2-((2-pentylcyclopropyl)- |
| | methyl)cyclopropyl)tridecan-6-amine) |
| 13 | 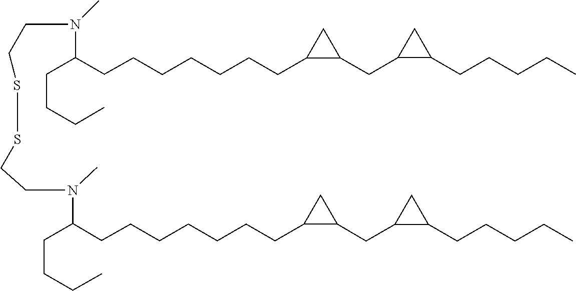 [SMILES: CCCCCC1CC1CC1CC1CCCCCCCC(CCCC)N(C)CCSSCCN(C)C(CCCC)CCCCCCCC1CC1CC1CC1CCCCC] |
| | N,N′-(disulfanediylbis(ethane-2,1-diyl))bis(N-methyl-12-(2-((2-pentylcyclopropyl)- |
| | methyl)cyclopropyl)dodecan-5-amine) |
| 14 | 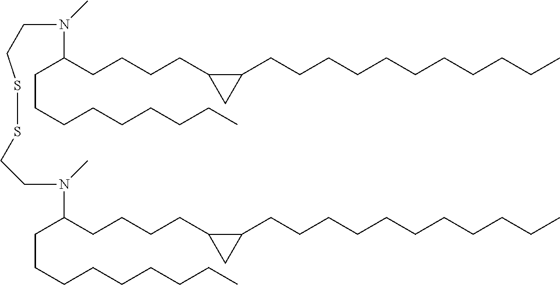 [SMILES: CCCCCCCCCCCC1CC1CCCCC(CCCCCCCCC)N(C)CCSSCCN(C)C(CCCCCCCCC)CCCCC1CC1CCCCCCCCCCC] |
| | N,N′-(disulfanediylbis(ethane-2,1-diyl))bis(N-methyl-1-(2-undecylcyclopropyl)- |
| | tetradecan-5-amine) |
| 15 | 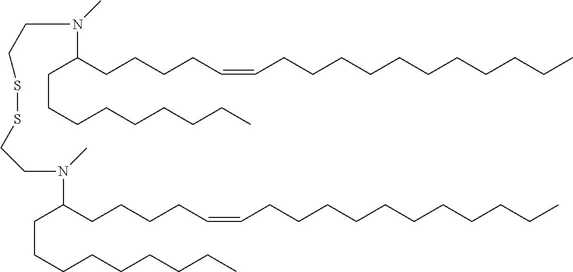 [SMILES: CCCCCCCCCCC/C=C\CCCCC(CCCCCCCCC)N(C)CCSSCCN(C)C(CCCC/C=C\CCCCCCCCCCC)CCCCCCCCC] |
| | (15Z,15′Z)-N,N′-(disulfanediylbis(ethane-2,1-diyl))bis(N-methylheptacos-15-en- |
| | 10-amine) |
| 16 | 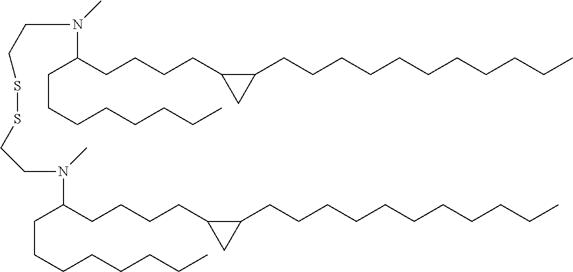 [SMILES: CCCCCCCCCCCC1CC1CCCCC(CCCCCCCC)N(C)CCSSCCN(C)C(CCCCCCCC)CCCCC1CC1CCCCCCCCCCC] |
| | N,N′-(disulfanediylbis(ethane-2,1-diyl))bis(N-methyl-1-(2-undecylcyclopropyl)- |
| | tridecan-5-amine) |
| 17 | 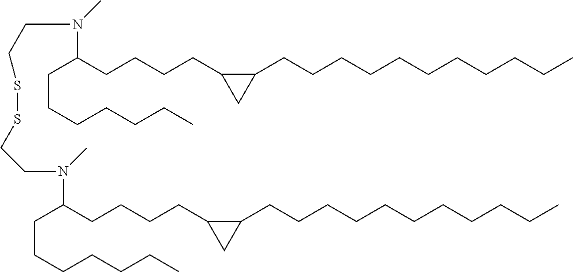 [SMILES: CCCCCCCCCCCC1CC1CCCCC(CCCCCCC)N(C)CCSSCCN(C)C(CCCCCCC)CCCCC1CC1CCCCCCCCCCC] |
| | N,N′-(disulfanediylbis(ethane-2,1-diyl))bis(N-methyl-1-(2-undecylcyclopropyl)- |
| | dodecan-5-amine) |
| 18 | 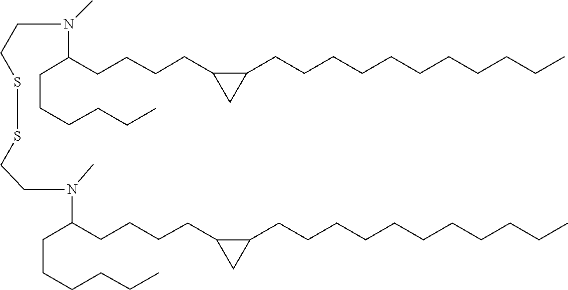 [SMILES: CCCCCCCCCCCC1CC1CCCCC(CCCCCC)N(C)CCSSCCN(C)C(CCCCCC)CCCCC1CC1CCCCCCCCCCC] |
| | N,N′-(disulfanediylbis(ethane-2,1-diyl))bis(N-methyl-1-(2-undecylcyclopropyl)- |
| | undecan-5-amine) |
| 19 | 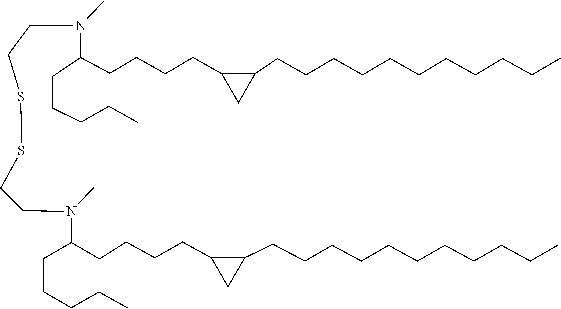 [SMILES: CCCCCCCCCCCC1CC1CCCCC(CCCCC)N(C)CCSSCCN(C)C(CCCCC)CCCCC1CC1CCCCCCCCCCC] |
| | N,N′-(disulfanediylbis(ethane-2,1-diyl))bis(N-methyl-1-(2-undecylcyclopropyl)- |
| | decan-5-amine) |
| 20 | 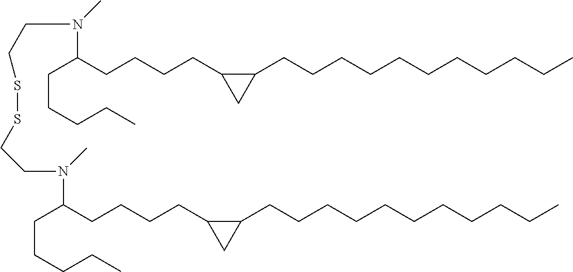 [SMILES: CCCCCCCCCCCC1CC1CCCCC(CCCCC)N(C)CCSSCCN(C)C(CCCCC)CCCCC1CC1CCCCCCCCCCC] |
| | N,N′-(disulfanediylbis(ethane-2,1-diyl))bis(N-methyl-1-(2-undecylcyclopropyl)- |
| | decan-5-amine) |
| 21 | 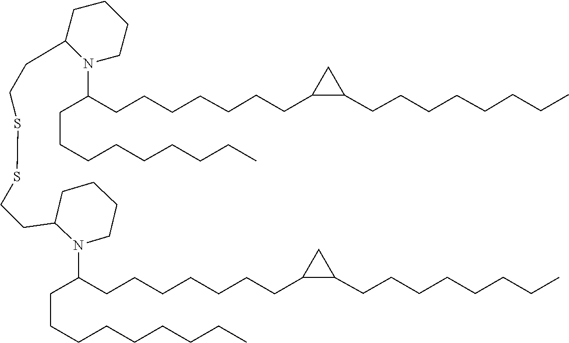 [SMILES: CCCCCCCCCC(CCCCCCCC1CC1CCCCCCCC)N1CCCCC1CCSSCCC1CCCCN1C(CCCCCCCCC)CCCCCCCC1CC1CCCCCCCC] |
| | 1,2-bis(2-(1-(1-(2-octylcyclopropyl)heptadecan-8-yl)piperidin-2-yl)ethyl)disulfane |
| 22 | 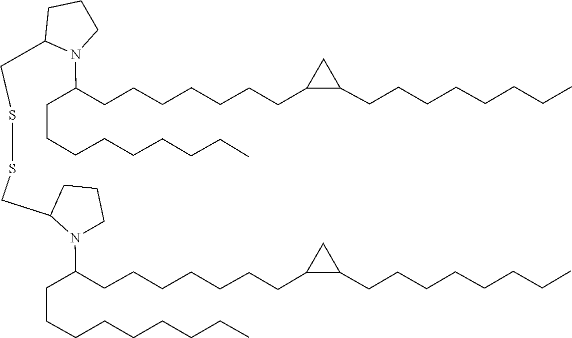 [SMILES: CCCCCCCCCC(CCCCCCCC1CC1CCCCCCCC)N1CCCC1CSSCC1CCCN1C(CCCCCCCCC)CCCCCCCC1CC1CCCCCCCC] |
| | 1,2-bis((1-(1-(2-octylcyclopropyl)heptadecan-8-yl)pyrrolidin-2-yl)methyl)disulfane |
| 23 | 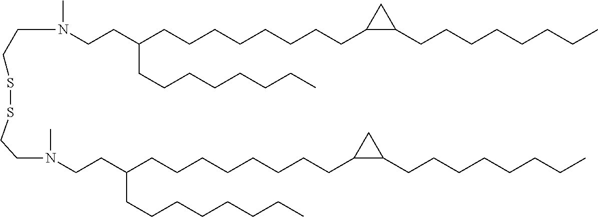 [SMILES: CCCCCCCCC(CCCCCCCCC1CC1CCCCCCCC)CCN(C)CCSSCCN(C)CCC(CCCCCCCC)CCCCCCCCC1CC1CCCCCCCC] |
| | N,N′-(disulfanediylbis(ethane-2,1-diyl))bis(N-methyl-3-octyl-11-(2-octylcyclopropyl)- |
| | undecan-1-amine) |
| 24 | 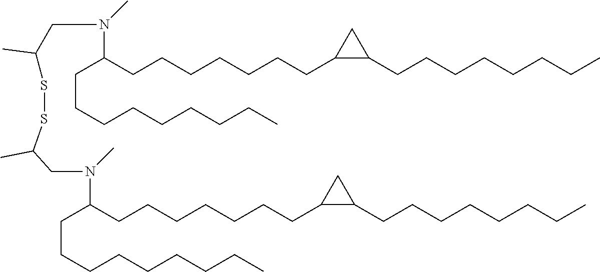 [SMILES: CCCCCCCCCC(CCCCCCCC1CC1CCCCCCCC)N(C)CC(C)SSC(C)CN(C)C(CCCCCCCCC)CCCCCCCC1CC1CCCCCCCC] |
| | N,N′-(disulfanediylbis(propane-2,1-diyl))bis(N-methyl-1-(2-octylcyclopropyl)- |
| | heptadecan-9-amine) |
| 25 | 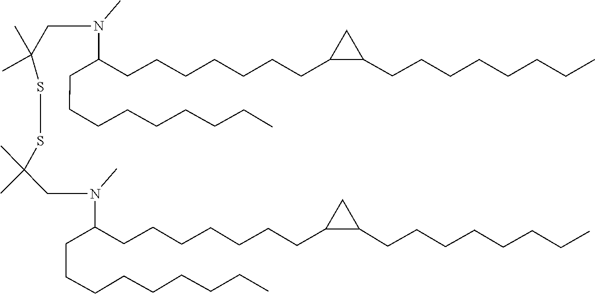 [SMILES: CCCCCCCCCC(CCCCCCCC1CC1CCCCCCCC)N(C)CC(C)(C)SSC(C)(C)CN(C)C(CCCCCCCCC)CCCCCCCC1CC1CCCCCCCC] |
| | N,N′-(disulfanediylbis(2-methylpropane-2,1-diyl))bis(N-methyl-1-(2-octylcyclopropyl)- |
| | heptadecan-8-amine) |
| 26 | 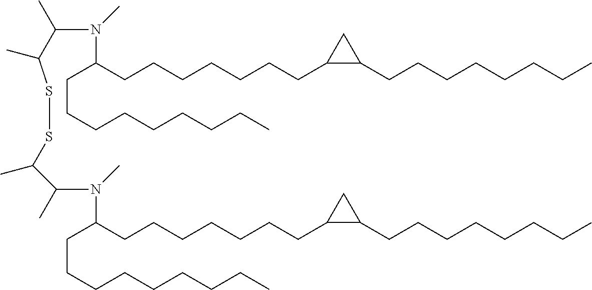 [SMILES: CCCCCCCCCC(CCCCCCCC1CC1CCCCCCCC)N(C)C(C)C(C)SSC(C)C(C)N(C)C(CCCCCCCCC)CCCCCCCC1CC1CCCCCCCC] |
| | N,N′-(disulfanediylbis(butane-3,2-diyl))bis(N-methyl-1-(2-octylcyclopropyl)heptadecan- |
| | 8-amine) |
| 27 | 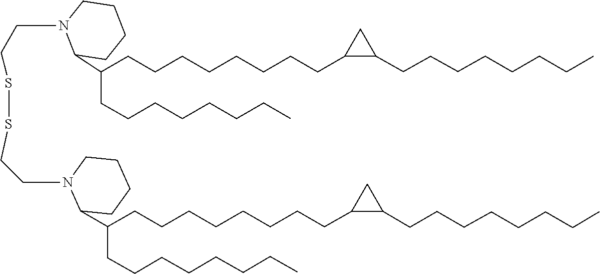 [SMILES: CCCCCCCCC1CC1CCCCCCCCC(CCCCCCCC)C1CCCCN1CCSSCCN1CCCCC1C(CCCCCCCC)CCCCCCCCC1CC1CCCCCCCC] |
| | 1,2-bis(2-(2-(1-(2-octylcyclopropyl)heptadecan-9-yl)piperidin-1-yl)ethyl)disulfane |
| 28 | 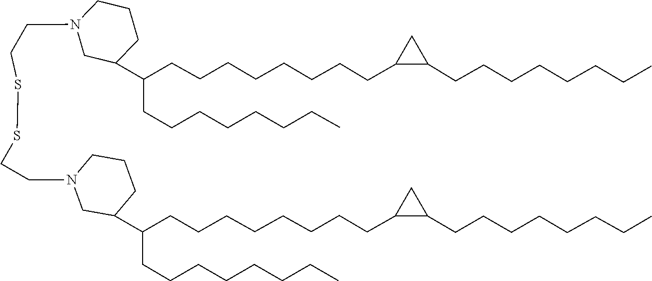 [SMILES: CCCCCCCCC(CCCCCCCCC1CC1CCCCCCCC)C1CCCN(CCSSCCN2CCCC(C(CCCCCCCC)CCCCCCCCC3CC3CCCCCCCC)C2)C1] |
| | 1,2-bis(2-(3-(1-(2-octylcyclopropyl)heptadecan-9-yl)piperidin-1-yl)ethyl)disulfane |
| 29 | 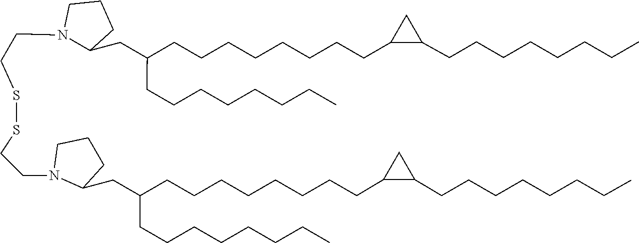 [SMILES: CCCCCCCCC(CCCCCCCCC1CC1CCCCCCCC)CC1CCCN1CCSSCCN1CCCC1CC(CCCCCCCC)CCCCCCCCC1CC1CCCCCCCC] |
| | 1,2-bis(2-(2-(2-octyl-10-(2-octylcyclopropyl)decyl)pyrrolidin-1-yl)ethyl)disulfane |
| 30 | 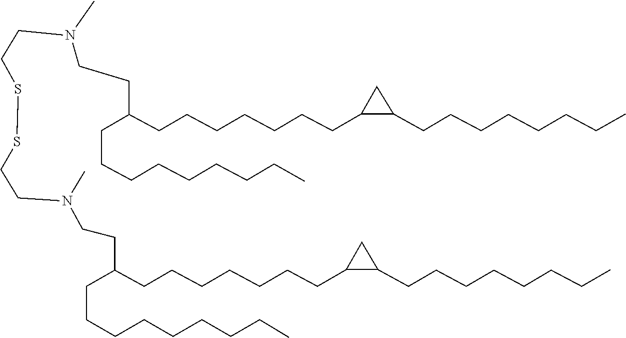 [SMILES: CCCCCCCCCC(CCCCCCCC1CC1CCCCCCCC)CCN(C)CCSSCCN(C)CCC(CCCCCCCCC)CCCCCCCC1CC1CCCCCCCC] |
| | N,N′-(disulfanediylbis(ethane-2,1-diyl))bis(N-methyl-3-(7-(2-octylcyclopropyl)heptyl)- |
| | dodecan-1-amine) |
| 31 | 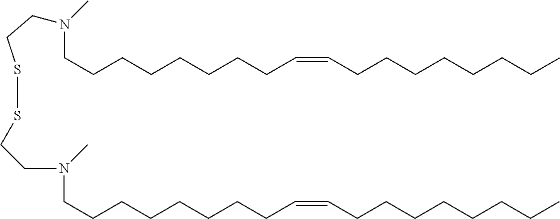 [SMILES: CCCCCCCC/C=C\CCCCCCCCN(C)CCSSCCN(C)CCCCCCCC/C=C\CCCCCCCC] |
| | (9Z,9′Z)-N,N′-(disulfanediylbis(ethane-2,1-diyl))bis(N-methyloctadec-9-en-1-amine) |
| 32 | 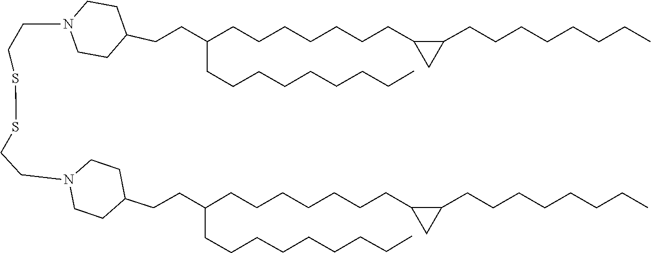 [SMILES: CCCCCCCCCC(CCCCCCCC1CC1CCCCCCCC)CCC1CCN(CCSSCCN2CCC(CCC(CCCCCCCCC)CCCCCCCC3CC3CCCCCCCC)CC2)CC1] |
| | 1,2-bis(2-(4-(1-(2-octylcyclopropyl)heptadecan-8-yl)piperidin-1-yl)ethyl)disulfane |
| 33 | 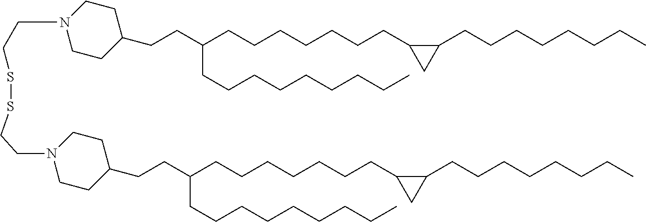 [SMILES: CCCCCCCCCC(CCCCCCCC1CC1CCCCCCCC)CCC1CCN(CCSSCCN2CCC(CCC(CCCCCCCCC)CCCCCCCC3CC3CCCCCCCC)CC2)CC1] |
| | 1,2-bis(2-(4-(3-(7-(2-octylcyclopropyl)heptyl)dodecyl)piperidin-1-yl)ethyl)disulfane |
| 34 | 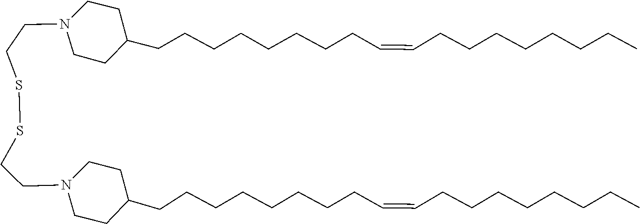 [SMILES: CCCCCCCC/C=C\CCCCCCCCC1CCN(CCSSCCN2CCC(CCCCCCCC/C=C\CCCCCCCC)CC2)CC1] |
| | 1,2-bis(2-(4-((Z)-octadec-9-en-1-yl)piperidin-1-yl)ethyl)disulfane |
| 35 | 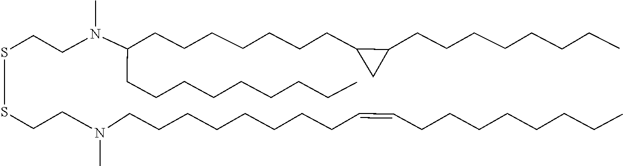 [SMILES: CCCCCCCC/C=C\CCCCCCCCN(C)CCSSCCN(C)C(CCCCCCCCC)CCCCCCCC1CC1CCCCCCCC] |
| | (Z)-N-methyl-N-(2-((2-(methyl(1-(2-octylcyclopropyl)heptadecan-8-yl)amino)ethyl)- |
| | disulfaneyl)ethyl)octadec-9-en-1-amine |
| 36 | 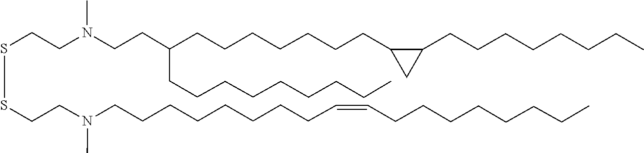 [SMILES: CCCCCCCC/C=C\CCCCCCCCN(C)CCSSCCN(C)CCC(CCCCCCCCC)CCCCCCCC1CC1CCCCCCCC] |
| | (Z)-N-methyl-N-(2-((2-(methyl(3-(7-(2-octylcyclopropyl)heptyl)dodecyl)amino)ethyl)disulfaneyl)- |
| | ethyl)octadec-9-en-1-amine |
| 37 | 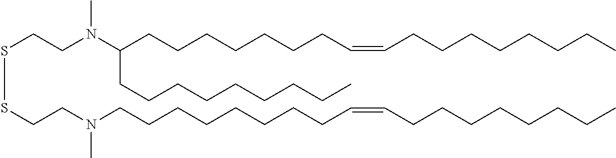 [SMILES: CCCCCCCC/C=C\CCCCCCCCN(C)CCSSCCN(C)C(CCCCCCC/C=C\CCCCCCCC)CCCCCCCCC] |
| | (Z)-N-methyl-N-(2-((2-(methyl((Z)-octadec-9-en-1-yl)amino)ethyl)disulfaneyl)ethyl)heptacos- |
| | 18-en-10-amine |
| 38 | 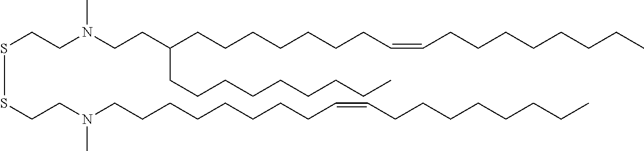 [SMILES: CCCCCCCC/C=C\CCCCCCCCN(C)CCSSCCN(C)CCC(CCCCCCC/C=C\CCCCCCCC)CCCCCCCCC] |
| | (Z)-N-methyl-N-(2-((2-(methyl((Z)-octadec-9-en-1-yl)amino)ethyl)disulfaneyl)ethyl)-3-nonylicos- |
| | 11-en-1-amine |
| 39 | 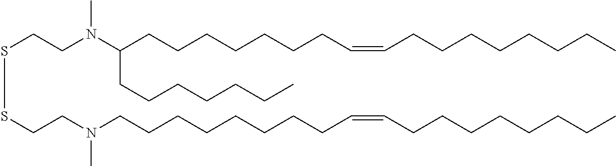 [SMILES: CCCCCCCC/C=C\CCCCCCCCN(C)CCSSCCN(C)C(CCCCCCC)CCCCCCC/C=C\CCCCCCCC] |
| | (Z)-N-methyl-N-(2-((2-(methyl((Z)-octadec-9-en-1-yl)amino)ethyl)disulfaneyl)ethyl)pentacos- |
| | 16-en-8-amine |
| 40 | 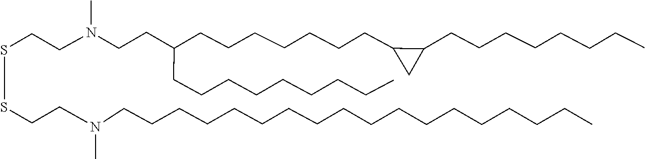 [SMILES: CCCCCCCCCCCCCCCCCCN(C)CCSSCCN(C)CCC(CCCCCCCCC)CCCCCCCC1CC1CCCCCCCC] |
| | N-methyl-N-(2-((2-(methyl(3-(7-(2-octylcyclopropyl)heptyl)dodecyl)amino)ethyl)disulfaneyl)- |
| | ethyl)octadecan-1-amine |
| 41 | 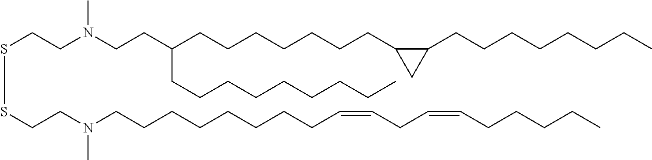 [SMILES: CCCCC/C=C\C/C=C\CCCCCCCCN(C)CCSSCCN(C)CCC(CCCCCCCCC)CCCCCCCC1CC1CCCCCCCC] |
| | (9Z,12Z)-N-methyl-N-(2-((2-(methyl(3-(7-(2-octylcyclopropyl)heptyl)dodecyl)amino)ethyl)- |
| | disulfaneyl)ethyl)octadeca-9,12-dien-1-amine |
| 42 | 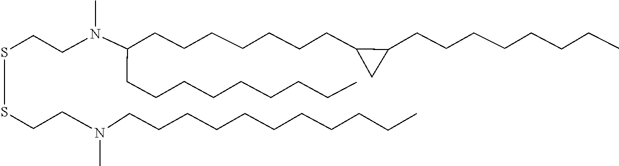 [SMILES: CCCCCCCCCCCN(C)CCSSCCN(C)C(CCCCCCCCC)CCCCCCCC1CC1CCCCCCCC] |
| | N-methyl-N-(2-((2-(methyl(undecyl)amino)ethyl)disulfaneyl)ethyl)-1-(2-octylcyclopropyl)- |
| | heptadecan-8-amine |
| 43 | 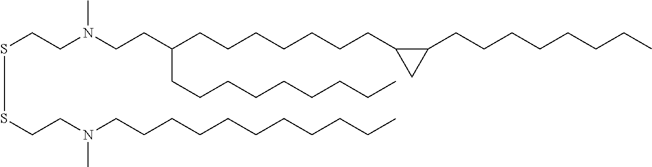 [SMILES: CCCCCCCCCCCN(C)CCSSCCN(C)CCC(CCCCCCCCC)CCCCCCCC1CC1CCCCCCCC] |
| | N-methyl-N-(2-((2-(methyl(undecyl)amino)ethyl)disulfaneyl)ethyl)-3-(7-(2-octylcyclopropyl)heptyl)- |
| | dodecan-1-amine |
| 44 | 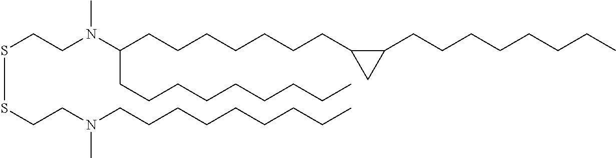 [SMILES: CCCCCCCCCC(CCCCCCCC1CC1CCCCCCCC)N(C)CCSSCCN(C)CCCCCCCCC] |
| | N-methyl-N-(2-((2-(methyl(nonyl)amino)ethyl)disulfaneyl)ethyl)-1-(2-octylcyclopropyl)- |
| | heptadecan-8-amine |
| 45 | 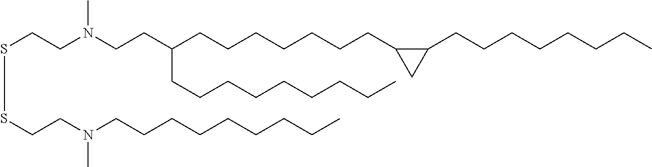 [SMILES: CCCCCCCCCC(CCCCCCCC1CC1CCCCCCCC)CCN(C)CCSSCCN(C)CCCCCCCCC] |
| | N-methyl-N-(2-((2-(methyl(nonyl)amino)ethyl)disulfaneyl)ethyl)-3-(7-(2-octylcyclopropyl)- |
| | heptyl)dodecan-1-amine |
| 46 | 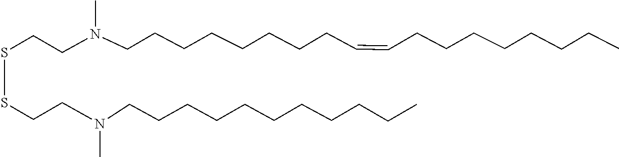 [SMILES: CCCCCCCC/C=C\CCCCCCCCN(C)CCSSCCN(C)CCCCCCCCCCC] |
| | (Z)-N-methyl-N-(2-((2-(methyl(undecyl)amino)ethyl)disulfaneyl)ethyl)octadec-9-en-1-amine |
| 47 | 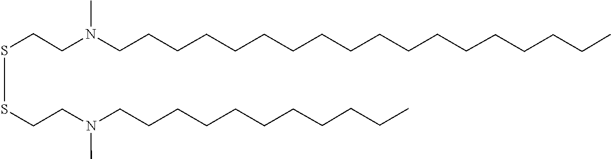 [SMILES: CCCCCCCCCCCCCCCCCCN(C)CCSSCCN(C)CCCCCCCCCCC] |
| | N-methyl-N-(2-((2-(methyl(undecyl)amino)ethyl)disulfaneyl)ethyl)octadecan-1-amine |
| 48 | 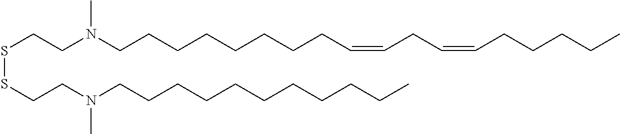 [SMILES: CCCCC/C=C\C/C=C\CCCCCCCCN(C)CCSSCCN(C)CCCCCCCCCCC] |
| | (9Z,12Z)-N-methyl-N-(2-((2-(methyl(undecyl)amino)ethyl)disulfaneyl)ethyl)octadeca-9,12- |
| | dien-1-amine |
| 49 | 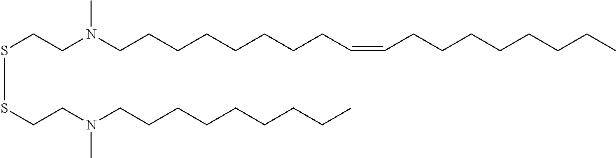 [SMILES: CCCCCCCC/C=C\CCCCCCCCN(C)CCSSCCN(C)CCCCCCCCC] |
| | (Z)-N-methyl-N-(2-((2-(methyl(nonyl)amino)ethyl)disulfaneyl)ethyl)octadec-9-en-1-amine |
| 50 | 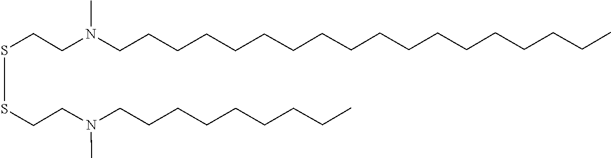 [SMILES: CCCCCCCCCCCCCCCCCCN(C)CCSSCCN(C)CCCCCCCCC] |
| | N-methyl-N-(2-((2-(methyl(nonyl)amino)ethyl)disulfaneyl)ethyl)octadecan-1-amine |
| 51 | 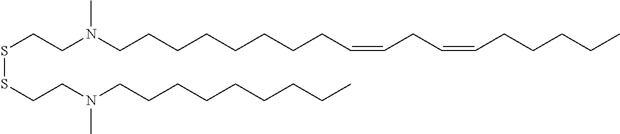 [SMILES: CCCCC/C=C\C/C=C\CCCCCCCCN(C)CCSSCCN(C)CCCCCCCCC] |
| | (9Z,12Z)-N-methyl-N-(2-((2-(methyl(nonyl)amino)ethyl)disulfaneyl)ethyl)octadeca-9,12- |
| | dien-1-amine |

### Formula (B)

In some embodiments, the ionizable lipid in the LNPs of the present disclosure is represented by Formula (B):

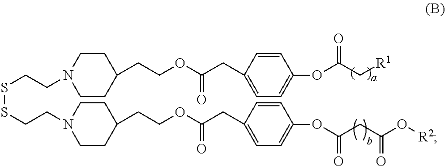

or a pharmaceutically acceptable salt thereof, wherein:
a is an integer ranging from 1 to 20 (e.g., a is 1, 2, 3, 4, 5, 6, 7, 8, 9, 10, 11, 12, 13, 14, 15, 16, 17, 18, 19, or 20);
b is an integer ranging from 2 to 10 (e.g., bis 2, 3, 4, 5, 6, 7, 8, 9, or 10);
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl.

In a second embodiment of Formula (B), the ionizable lipid of Formula (B) is represented by Formula (B-1):

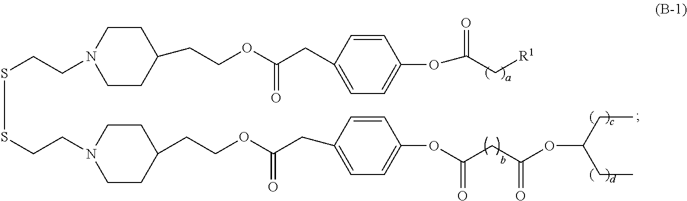

or a pharmaceutically acceptable salt thereof, wherein c and d are each independently integers ranging from 1 to 8 (e.g., 1, 2, 3, 4, 5, 6, 7, or 8), and wherein the remaining variables are as described for Formula (B).

In a third embodiment of Formula (B), c and d in Formula (B-1) are each independently integers ranging from 2 to 8, 3 to 8, 3 to 7, 3 to 6, 3 to 5, 4 to 8, 4 to 7, 4 to 6, 5 to 8, 5 to 7, or 6 to 8, wherein the remaining variables are as described for Formula (B-1).

In a fourth embodiment of Formula (B), c in Formula (B-1) is 2, 3, 4, 5, 6, 7, or 8, wherein the remaining variables are as described for Formula (B), or the second or third embodiment of Formula (B). Alternatively, c and d in Formula (B-1) are each independently 1, 3, 5, or 7, wherein the remaining variables are as described for Formula (B), or the second or third embodiment of Formula (B).

In a fifth embodiment of Formula (B), d in the cationic lipid of Formula (B-1) is 2, 3, 4, 5, 6, 7, or 8, wherein the remaining variables are as described for Formula (B), or the second, third or fourth embodiments of Formula (B). Alternatively, at least one of c and d in Formula (B-1) is 7, wherein the remaining variables are as described for Formula (B), or the second, third or fourth embodiments of Formula (B).

In a sixth embodiment of Formula (B), the ionizable lipid of Formula (B) or Formula (B-1) is represented by Formula (B-2):

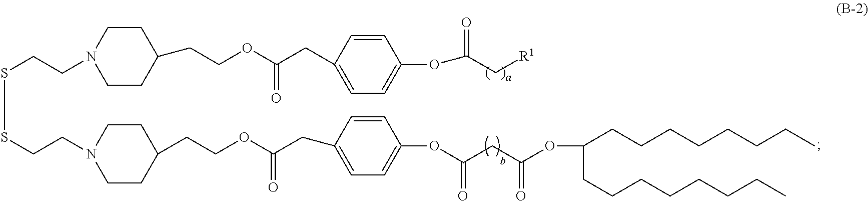

or a pharmaceutically acceptable salt thereof, wherein the remaining variables are as described for Formula (B) or Formula (B-1).

In a seventh embodiment of Formula (B), b in Formula (B), (B-1), or (B-2) is an integer ranging from 3 to 9, wherein the remaining variables are as described for Formula (B), or the second, third, fourth, fifth or sixth embodiments of Formula (B). Alternatively, b in Formula (B), (B-1), or (B-2) is an integer ranging from 3 to 8, 3 to 7, 3 to 6, 3 to 5, 4 to 9, 4 to 8, 4 to 7, 4 to 6, 5 to 9, 5 to 8, 5 to 7, 6 to 9, 6 to 8, or 7 to 9, wherein the remaining variables are as described for Formula (B), or the second, third, fourth, fifth or sixth embodiments of Formula (B). Alternatively, b in Formula (B), (B-1), or (B-2) is 3, 4, 5, 6, 7, 8, or 9, wherein the remaining variables are as described for Formula (B), or the second, third, fourth, fifth or sixth embodiments of Formula (B).

In an eighth embodiment of Formula (B), a in Formula (B), (B-1), or (B-2) is an integer ranging from 2 to 18, wherein the remaining variables are as described for Formula (B), or the second, third, fourth, fifth, sixth or seventh embodiment of Formula (B). Alternatively, a in Formula (B), (B-1), or (B-2) is an integer ranging from 2 to 18, 2 to 17, 2 to 16, 2 to 15, 2 to 14, 2 to 13, 2 to 12, 2 to 11, 2 to 10, 2 to 9, 2 to 8, 2 to 7, 2 to 6, 2 to 5, 2 to 4, 3 to 18, 3 to 17, 3 to 16, 3 to 15, 3 to 14, 3 to 13, 3 to 12, 3 to 11, 3 to 10, 3 to 9, 3 to 8, 3 to 7, 3 to 6, 3 to 5, 4 to 18, 4 to 17, 4 to 16, 4 to 15, 4 to 14, 4 to 13, 4 to 12, 4 to 11, 4 to 10, 4 to 9, 4 to 8, 4 to 7, 4 to 6, 5 to 18, 5 to 17, 5 to 16, 5 to 15, 5 to 14, 5 to 13, 5 to 12, 5 to 11, 5 to 10, 5 to 9, 25 to 8, 5 to 7, 6 to 18, 6 to 17, 6 to 16, 6 to 15, 6 to 14, 6 to 13, 6 to 12, 6 to 11, 6 to 10, 6 to 9, 6 to 8, 7 to 18, 7 to 17, 7 to 16, 7 to 15, 7 to 14, 7 to 13, 7 to 12, 7 to 11, 7 to 10, 7 to 9, 8 to 18, 8 to 17, 8 to 16, 8 to 15, 8 to 14, 8 to 13, 8 to 12, 8 to 11, 8 to 10, 9 to 18, 9 to 17, 9 to 16, 9 to 15, 9 to 14, 9 to 13, 9 to 12, 9 to 11, 10 to 18, 10 to 17, 10 to 16, 10 to 15, 10 to 14, 10 to 13, 11 to 18, 11 to 17, 11 to 16, 11 to 15, 11 to 14, 11 to 13, 12 to 18, 12 to 17, 12 to 16, 12 to 15, 12 to 14, 13 to 18, 13 to 17, 13 to 16, 13 to 15, 14 to 18, 14 to 17, 14 to 16, 15 to 18, 15 to 17, or 16 to 18, wherein the remaining variables are as described for Formula (B), or the second, third, fourth, fifth, sixth or seventh embodiment of Formula (B). Alternatively, a in Formula (B), (B-1), or (B-2) is 2, 3, 4, 5, 6, 7, 8, 9, 10, 11, 12, 13, 14, 15, 16, 17, or 18, wherein the remaining variables are as described for Formula (B), or the second, third, fourth, fifth, sixth or seventh embodiment of Formula (B).

In a ninth embodiment of Formula (B), R¹ in Formula (B), Formula (B-1), or Formula (B-2) is absent or is selected from (C₅-C₁₅)alkenyl, —C(O)O(C₄-C₁₈)alkyl, and cyclopropyl substituted with (C₄-C₁₆)alkyl, wherein the remaining variables are as described for Formula (B), or the second, third, fourth, fifth, sixth, seventh or eighth embodiments of Formula (B). Alternatively, R¹ in Formula (B), Formula (B-1), or Formula (B-2) is absent or is selected from (C₅-C₁₅)alkenyl, —C(O)O(C₄-C₁₆)alkyl, and cyclopropyl substituted with (C₄-C₁₆)alkyl, wherein the remaining variables are as described for Formula (B), or the second, third, fourth, fifth, sixth, seventh or eighth embodiments of Formula (B). Alternatively, R¹ in Formula (B), Formula (B-1), or Formula (B-2) is absent or is selected from (C₅-C₁₂)alkenyl, —C(O)O(C₄-C₁₂)alkyl, and cyclopropyl substituted with (C₄-C₁₂)alkyl, wherein the remaining variables are as described for Formula (B), or the second, third, fourth, fifth, sixth, seventh or eighth embodiments of Formula (B). In another alternative, R¹ in the cationic lipid of Formula (B), Formula (B-1), or Formula (B-2) is absent or is selected from (C₅-C₁₀)alkenyl, —C(O)O(C₄-C₁₀)alkyl, and cyclopropyl substituted with (C₄-C₁₀)alkyl, wherein the remaining variables are as described for Formula (B), or the second, third, fourth, fifth, sixth, seventh or eighth embodiments of Formula (B).

In a tenth embodiment of Formula (B), R¹ is C₁₀ alkenyl, wherein the remaining variables are as described for Formula (B), or the second, third, fourth, fifth, sixth, seventh or eighth embodiments of Formula (B).

In an eleventh embodiment of Formula (B), the alkyl in C(O)O(C₂-C₂₀)alkyl, —C(O)O(C₄-C₁₈)alkyl, —C(O)O(C₄-C₁₂)alkyl, or —C(O)O(C₄-C₁₀)alkyl of R¹ in Formula (B), Formula (B-1), or Formula (B-2) is an unbranched alkyl, wherein the remaining variables are as described for Formula (B), or the second, third, fourth, fifth, sixth, seventh, eighth or ninth embodiments of Formula (B). In one embodiment, R¹ is —C(O)O(C₉ alkyl). Alternatively, the alkyl in —C(O)O(C₄-C₁₈)alkyl, —C(O)O(C₄-C₁₂)alkyl, or —C(O)O(C₄-C₁₀)alkyl of R¹ in Formula (B), Formula (B-1), or Formula (B-2) is a branched alkyl, wherein the remaining variables are as described for Formula (B), Formula (B-1), or Formula (B-2), or the second, third, fourth, fifth, sixth, seventh, eighth or ninth embodiments of Formula (B). In one embodiment, R¹ is —C(O)O(C₁₇ alkyl), wherein the remaining variables are as described for Formula (B), Formula (B-1), or Formula (B-2), or the second, third, fourth, fifth, sixth, seventh, eighth or ninth embodiments of Formula (B).

In a twelfth embodiment of Formula (B), R¹ in Formula (B), Formula (B-1), or Formula (B-2) is selected from any group listed in Table 2 below, wherein the wavy bond in each of the groups indicates the point of attachment of the group to the rest of the ionizable lipid molecule, and wherein the remaining variables are as described for Formula (B), Formula (B-1), or Formula (B-2), or the second, third, fourth, fifth, sixth, seventh or eighth embodiments of Formula (B). The present disclosure further contemplates the combination of any one of the R¹ groups in Table 2 with any one of the R² groups in Table 3 in Formula (B), wherein the remaining variables are as described for Formula (B), Formula (B-1), or Formula (B-2), or the second, third, fourth, fifth, sixth, seventh or eighth embodiments of Formula (B).

TABLE 2
Exemplary R1 groups in Formula (B), Formula (B-1),
or Formula (B-2)

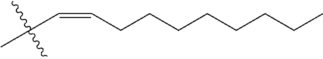 [SMILES: */C=C\CCCCCCCC]

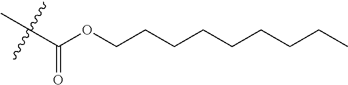 [SMILES: *C(=O)OCCCCCCCCC]

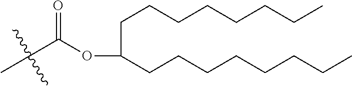 [SMILES: *C(=O)OC(CCCCCCCC)CCCCCCCC]

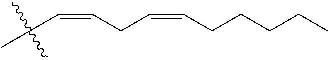 [SMILES: */C=C\C/C=C\CCCCC]

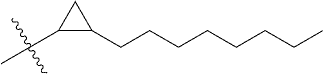 [SMILES: *C1CC1CCCCCCCC]

In a thirteenth embodiment, R² in Formula (B) or a pharmaceutically acceptable salt thereof is selected from any group listed in Table 3 below, wherein the wavy bond in each of the groups indicates the point of attachment of the group to the rest of the ionizable lipid molecule, and wherein the remaining variables are as described for Formula (B), Formula (B-1), or Formula (B-2), or the second, third, fourth, fifth, sixth, seventh or eighth, ninth, tenth, eleventh or twelfth embodiments of Formula (B).

TABLE 3
Exemplary R2 groups in Formula (B)

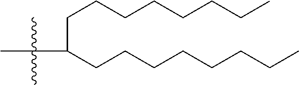 [SMILES: *C(CCCCCCCC)CCCCCCCC]

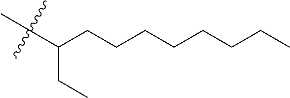 [SMILES: *C(CC)CCCCCCCC]

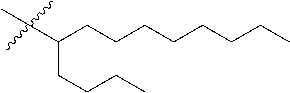 [SMILES: *C(CCCC)CCCCCCCC]

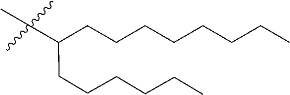 [SMILES: *C(CCCCCC)CCCCCCCC]

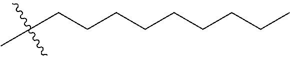 [SMILES: *CCCCCCCCC]

Table 4 below provides specific examples of ionizable lipids of Formula (B). Pharmaceutically acceptable salts as well as ionized and neutral forms are also included.

TABLE 4
Exemplary ionizable lipids of Formula (B), (B-1), or (B-2)

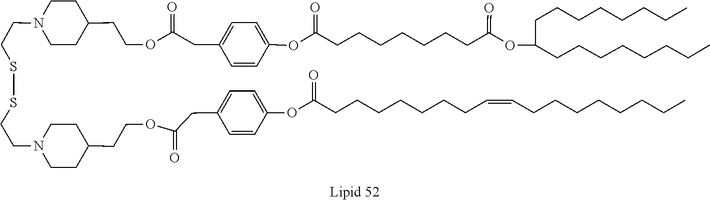 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-(oleoyloxy)phenyl)acetoxy)ethyl)piperidin-1-yl)ethyl)-
disulfaneyl)ethyl)piperidin-4-yl)ethoxy)-2-oxoethyl)phenyl) nonanedioate

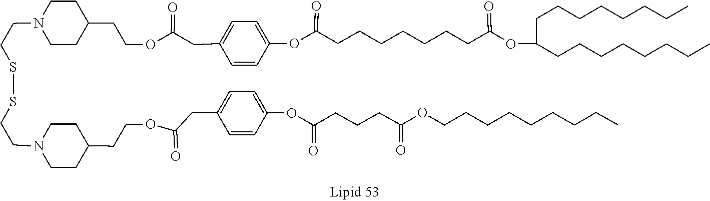 [SMILES: CCCCCCCCCOC(=O)CCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-((5-(nonyloxy)-5-oxopentanoyl)oxy)phenyl)acetoxy)-
ethyl) piperidin-1-yl)ethyl)disulfaneyl)ethyl) piperidin-4-yl)ethoxy)-2-oxoethyl)phenyl)nonanedioate

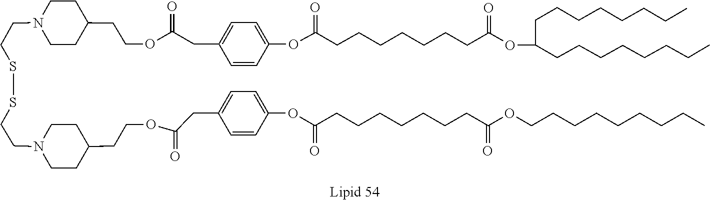 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-((9-(nonyloxy)-9-oxononanoyl)oxy)phenyl)acetoxy)-
ethyl)piperidin-1-yl)ethyl)disulfaneyl)ethyl)piperidin-4-yl)ethoxy)-2-oxoethyl)phenyl) nonanedioate

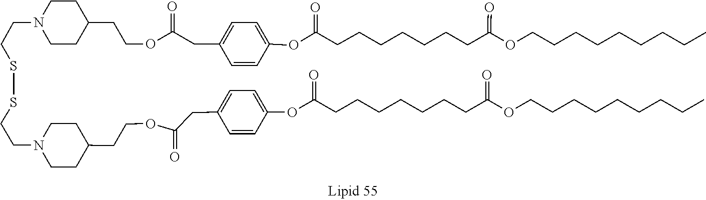 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCCCCCCCC)cc4)CC3)CC2)cc1]

1-(heptadecan-9-yl) 9-(4-(2-(2-(1-(2-((2-(4-(2-(2-(4-((5-(nonyloxy)-5-oxopentanoyl)oxy)phenyl)acetoxy)-
ethyl)piperidin-1-yl)ethyl)disulfaneyl)ethyl)piperidin-4-yl)ethoxy)-2-oxoethyl)phenyl)nonanedioate

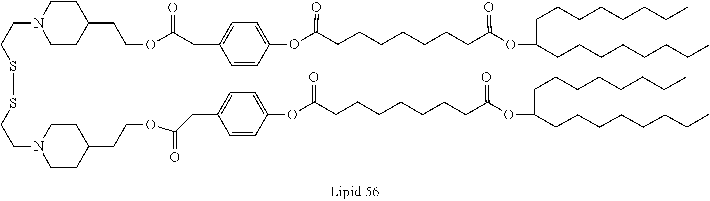 [SMILES: CCCCCCCCC(CCCCCCCC)OC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

O′1,01-((((((disulfanediylbis(ethane-2,1-diyl))bis(piperidine-1,4-diyl))bis(ethane-2,1-diyl))bis(oxy))bis(2-
oxoethane-2,1-diyl))bis(4,1-phenylene)) 9,9′-di(heptadecan-9-yl)di(nonanedioate)

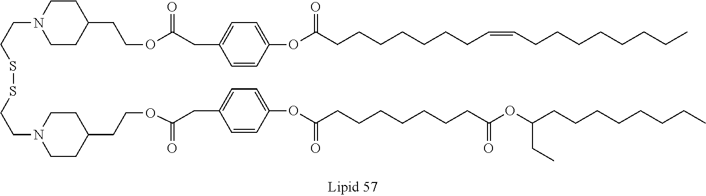 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

1-(4-(2-(2-(1-(2-((2-(4-(2-(2-(4-(oleoyloxy)phenyl)acetoxy)ethyl)piperidin-1-yl)ethyl)disulfaneyl)ethyl)-
piperidin-4-yl)ethoxy)-2-oxoethyl)phenyl) 9-(undecan-3-yl)nonanedioate

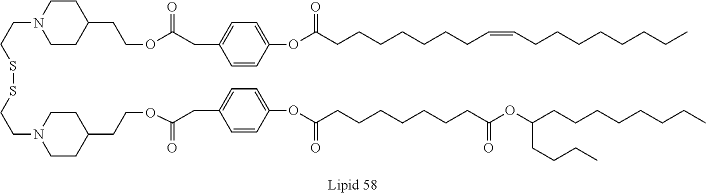 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

1-(4-(2-(2-(1-(2-((2-(4-(2-(2-(4-(oleoyloxy)phenyl)acetoxy)ethyl)piperidin-1-yl)ethyl)disulfaneyl)ethyl)-
piperidin-4-yl)ethoxy)-2-oxoethyl)phenyl) 9-(tridecan-5-yl)nonanedioate

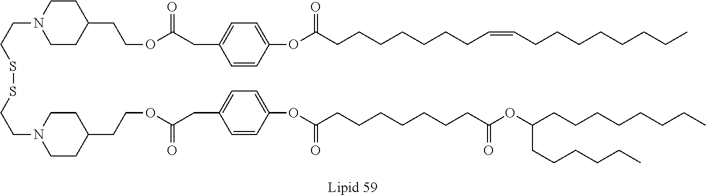 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

1-(4-(2-(2-(1-(2-((2-(4-(2-(2-(4-(oleoyloxy)phenyl)acetoxy)ethyl)piperidin-1-yl)ethyl)disulfaneyl)ethyl)-
piperidin-4-yl)ethoxy)-2-oxoethyl)phenyl) 9-(pentadecan-7-yl)nonanedioate

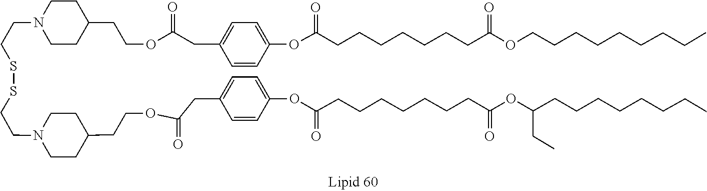 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CC)CCCCCCCC)cc4)CC3)CC2)cc1]

1-nonyl 9-(4-(2-oxo-2-(2-(1-(2-((2-(4-(2-(2-(4-((9-oxo-9-(undecan-3-yloxy)nonanoyl)oxy)phenyl)acetoxy)-
ethyl)piperidin-1-yl)ethyl)disulfaneyl)ethyl)piperidin-4-yl)ethoxy)ethyl)phenyl)nonanedioate

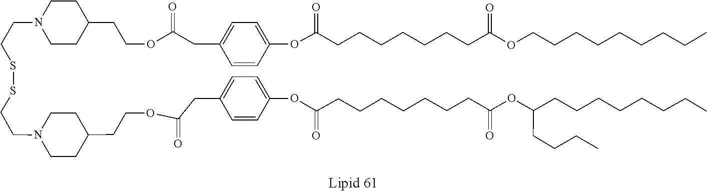 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCC)CCCCCCCC)cc4)CC3)CC2)cc1]

1-nonyl 9-(4-(2-oxo-2-(2-(1-(2-((2-(4-(2-(2-(4-((9-oxo-9-(tridecan-5-yloxy)nonanoyl)oxy)phenyl)acetoxy)-
ethyl)piperidin-1-yl)ethyl)disulfaneyl)ethyl)piperidin-4-yl)ethoxy)ethyl)phenyl) nonanedioate

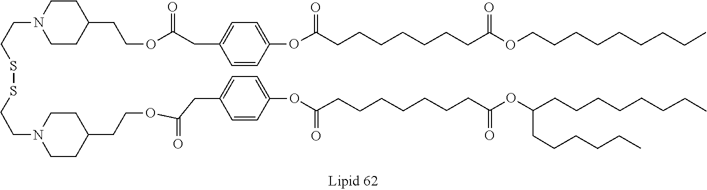 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OC(CCCCCC)CCCCCCCC)cc4)CC3)CC2)cc1]

1-nonyl 9-(4-(2-oxo-2-(2-(1-(2-((2-(4-(2-(2-(4-((9-oxo-9-(pentadecan-7-yloxy)nonanoyl)oxy)phenyl)acetoxy)-
ethyl)piperidin-1-yl)ethyl)disulfaneyl)ethyl)piperidin-4-yl)ethoxy)ethyl)phenyl) nonanedioate

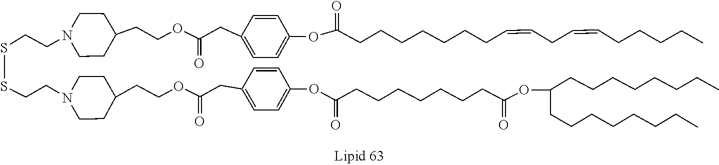 [SMILES: CCCCC/C=C\C/C=C\CCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-(((9Z,12Z)-octadeca-9,12-dienoyl)oxy)phenyl)acetoxy)-
ethyl)piperidin-1-yl)ethyl)disulfaneyl)ethyl)piperidin-4-yl)ethoxy)-2-oxoethyl)phenyl)nonanedioate

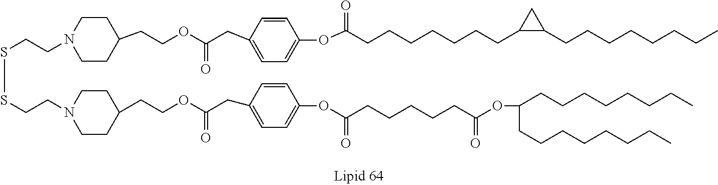 [SMILES: CCCCCCCCC(CCCCCCCC)OC(=O)CCCCCC(=O)Oc1ccc(CC(=O)OCCC2CCN(CCSSCCN3CCC(CCOC(=O)Cc4ccc(OC(=O)CCCCCCCC5CC5CCCCCCCC)cc4)CC3)CC2)cc1]

1-(heptadecan-9-yl) 9-(4-(2-(2-(1-(2-((2-(4-(2-(2-(4-((8-(2-octylcyclopropyl)octanoyl)oxy)phenyl)acetoxy)-
ethyl)piperidin-1-yl)ethyl)disulfaneyl)ethyl)piperidin-4-yl)ethoxy)-2-oxoethyl)phenyl)nonanedioate

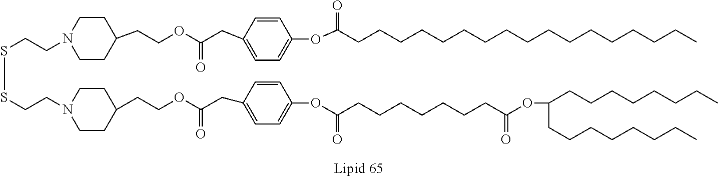 [SMILES: CCCCCCCCCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-oxo-2-(2-(1-(2-((2-(4-(2-(2-(4-(stearoyloxy)phenyl)acetoxy)ethyl)piperidin-1-yl)-
ethyl)disulfaneyl)ethyl)piperidin-4-yl)ethoxy)ethyl)phenyl)nonanedioate

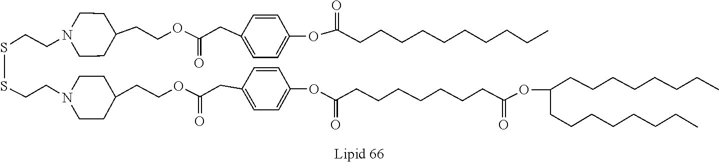 [SMILES: CCCCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-oxo-2-(2-(1-(2-((2-(4-(2-(2-(4-(undecanoyloxy)phenyl)acetoxy)ethyl)piperidin-
1-yl)ethyl)disulfaneyl)ethyl)piperidin-4-yl)ethoxy)ethyl)phenyl)nonanedioate

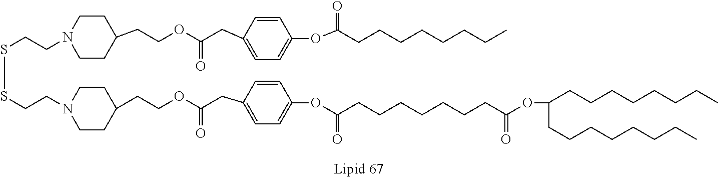 [SMILES: CCCCCCCCC(=O)Oc1ccc(CC(=O)OCCC2CCN(CCSSCCN3CCC(CCOC(=O)Cc4ccc(OC(=O)CCCCCCCC(=O)OC(CCCCCCCC)CCCCCCCC)cc4)CC3)CC2)cc1]

1-(heptadecan-9-yl) 9-(4-(2-(2-(1-(2-((2-(4-(2-(2-(4-(nonanoyloxy)phenyl)acetoxy)ethyl)piperidin-1-yl)ethyl)-
disulfaneyl)ethyl)piperidin-4-yl)ethoxy)-2-oxoethyl)phenyl) nonanedioate

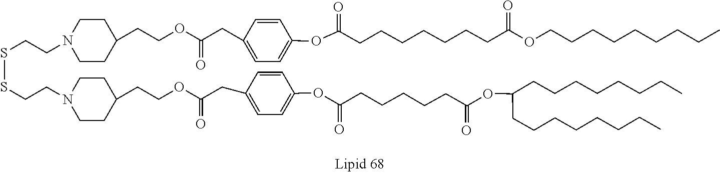 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]

1-nonyl 9-(4-(2-(2-(1-(2-((2-(4-(2-(2-(4-((9-((3-octylundecyl)oxy)-9-oxononanoyl)oxy)phenyl)acetoxy)ethyl)-
piperidin-1-yl)ethyl)disulfaneyl)ethyl)piperidin-4-yl)ethoxy)-2-oxoethyl)phenyl)nonanedioate

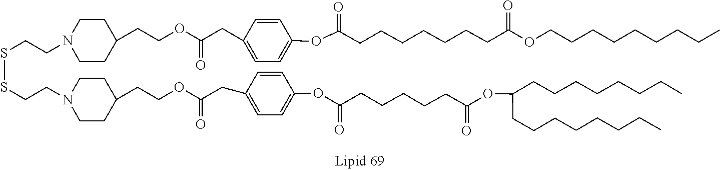 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OC(CCCCCCCC)CCCCCCCC)cc4)CC3)CC2)cc1]

1-(4-(2-(2-(1-(2-((2-(4-(2-(2-(4-((7-(heptadecan-9-yloxy)-7-oxoheptanoyl)oxy)phenyl)acetoxy)ethyl)piperidin-
1-yl)ethyl)disulfaneyl)ethyl)piperidin-4-yl)ethoxy)-2-oxoethyl)phenyl) 9-nonyl nonanedioate

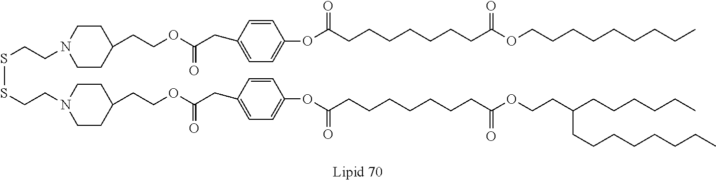 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCCCC(=O)OCCC(CCCCCC)CCCCCCCC)cc4)CC3)CC2)cc1]

1-nonyl 9-(4-(2-(2-(1-(2-((2-(4-(2-(2-(4-((9-((3-octylundecyl)oxy)-9-oxononanoyl)oxy)phenyl)acetoxy)ethyl)-
piperidin-1-yl)ethyl)disulfaneyl)ethyl)piperidin-4-yl)ethoxy)-2-oxoethyl)phenyl)nonanedioate

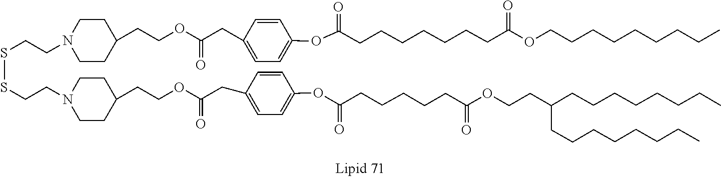 [SMILES: CCCCCCCCCOC(=O)CCCCCCCC(=O)Oc1ccc(CC(=O)OCCC2CCN(CCSSCCN3CCC(CCOC(=O)Cc4ccc(OC(=O)CCCCCC(=O)OCCC(CCCCCCCC)CCCCCCCC)cc4)CC3)CC2)cc1]

1-nonyl 9-(4-(2-(2-(1-(2-((2-(4-(2-(2-(4-((7-((3-octylundecyl)oxy)-7-oxoheptanoyl)oxy)phenyl)acetoxy)ethyl)-
piperidin-1-yl)ethyl)disulfaneyl)ethyl)piperidin-4-yl)ethoxy)-2-oxoethyl)phenyl)nonanedioate

### Formula (C)

In some embodiments, the ionizable lipid in the LNPs of the present disclosure are represented by Formula (C):

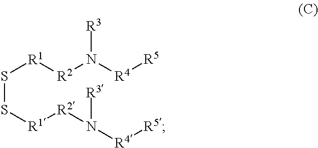

or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆) cycloalkyl; and
R^{a} and R^{b} are each halo or cyano.

In a second embodiment of Formula (C), R¹ and R¹ are each independently (C₁-C₆)alkylene, wherein the remaining variables are as described above for Formula (C). Alternatively, R¹ and R^{1′} are each independently (C₁-C₃)alkylene, wherein the remaining variables are as described above for Formula (C).

In a third embodiment of Formula (C), the ionizable lipid of the Formula (C) is represented by Formula (C-1):

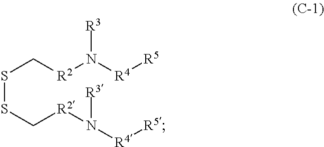

or a pharmaceutically acceptable salt thereof, wherein R² and R^{2′}, R³ and R^{3′}, R⁴ and R^{4′} and R⁵ and R^{5′} are as described above for Formula (C) or the second embodiment of Formula (C).

In a fourth embodiment, the ionizable lipid of Formula (C) is represented by Formula (C-2) or Formula (C-3):

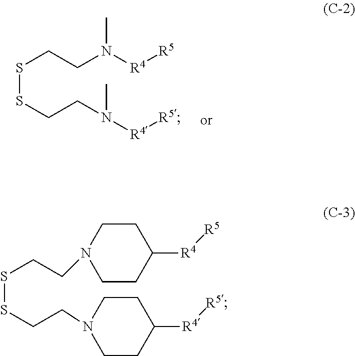

or a pharmaceutically acceptable salt thereof, wherein R⁴ and R^{4′} and R⁵ and R^{5′} are as described above for Formula (C).

In a fifth embodiment of Formula (C), the ionizable lipid of Formula (C) is represented by Formula (C-4) or (C-5):

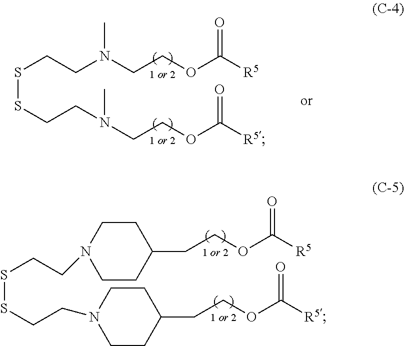

or a pharmaceutically acceptable salt thereof, wherein R⁵ and R^{5′} are as described above for Formula (C).

In a sixth embodiment of Formula (C), the ionizable lipid of Formula (C) is represented by Formula (C-6), (C-7), (C-8), or (C-9):

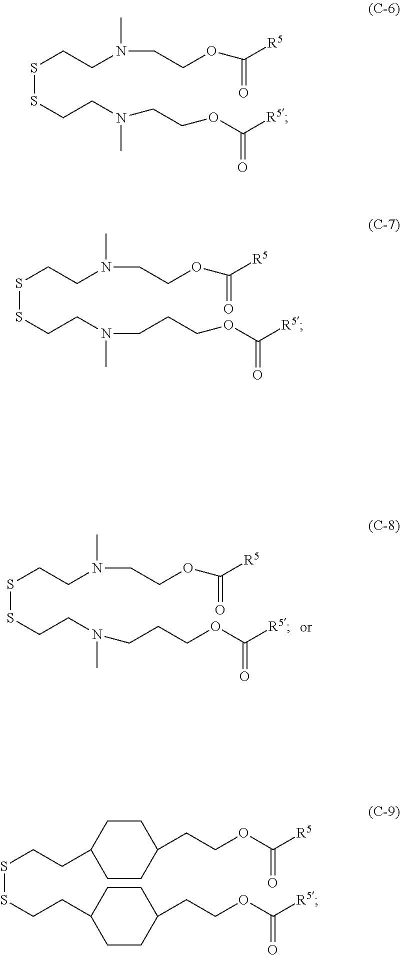

or a pharmaceutically acceptable salt thereof, wherein R⁵ and R^{5′} are as described above for Formula (XV).

In a seventh embodiment of Formula (C), at least one of R⁵ and R^{5′} in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C). Alternatively, one of R⁵ and R^{5′} in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C). Alternatively, R^{5′} in Formula (C), (C-1), (C-2), (C-3), (C-4), (C-5), (C-6), (C-7), (C-8), or (C-9) is a branched alkyl or branched alkenyl, and the remaining variables are as described above for Formula (C) or the second embodiment of Formula (C).

In an eighth embodiment of Formula (C), R⁵ in Formula (C), (C-1), (C-2), (C-3), (C-4), (C-5), (C-6), (C-7), (C-8), or (C-9) is a (C₆-C₂₆)alkyl or (C₆-C₂₆)alkenyl, each of which are optionally interrupted with —C(O)O— or (C₃-C₆) cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₆-C₂₆)alkyl or (C₆-C₂₆)alkenyl, each of which are optionally interrupted with —C(O)O— or (C₃-C₅) cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₇-C₂₆)alkyl or (C₇-C₂₆)alkenyl, each of which are optionally interrupted with —C(O)O— or (C₃-C₅) cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₈-C₂₆)alkyl or (C₈-C₂₆)alkenyl, each of which are optionally interrupted with —C(O)O— or (C₃-C₅) cycloalkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₆-C₂₄)alkyl or (C₆-C₂₄)alkenyl, each of which are optionally interrupted with —C(O)O— or cyclopropyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₈-C₂₄)alkyl or (C₈-C₂₄)alkenyl, wherein said (C₈-C₂₄)alkyl is optionally interrupted with —C(O)O— or cyclopropyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₈-C₁₀)alkyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₁₄-C₁₆)alkyl interrupted with cyclopropyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₁₀-C₂₄)alkyl interrupted with —C(O)O—, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is a (C₁₆-C₁₈)alkenyl, and the remaining variables are as described above for Formula (C) or the second embodiment of Formula (C). Alternatively, R⁵ in Formula (C), (C-1), (C-2), (C-3), (C-4), (C-5), (C-6), (C-7), (C-8), or (C-9) is —(CH₂)₃C(O)O(CH₂)₈CH₃, —(CH₂)₅C(O)O(CH₂)₈CH₃, —(CH₂)₇C(O)O(CH₂)₈CH₃, —(CH₂)₇C(O)OCH[(CH₂)₇CH₃]₂, —(CH₂)₇—C₃H₆—(CH₂)₇CH₃, —(CH₂)₇CH₃, —(CH₂)₉CH₃, —(CH₂)₁₆CH₃, —(CH₂)₇CH═CH(CH₂)₇CH₃, or —(CH₂)₇CH═CHCH₂CH═CH(CH₂)₄CH₃, and the remaining variables are as described above for Formula (C) or the second embodiment of Formula (C).

In a ninth embodiment, R^{5′} in Formula (C), (C-1), (C-2), (C-3), (C-4), (C-5), (C-6), (C-7), (C-8), or (C-9) is a (C₁₅-C₂₈)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₇-C₂₈)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₉-C₂₈)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₇-C₂₆)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₁₉-C₂₆)alkyl interrupted with —C(O)O—, and the remaining variables are as described above for Formula (C) or the second or eighth embodiments of Formula (C). Alternatively, R^{5′} in Formula (C), (C-1), (C-2), (C-3), (C-4), (C-5), (C-6), (C-7), (C-8), or (C-9) is a (C₂₀-C₂₆)alkyl interrupted with —C(O)O—, and the remaining variables are as described above for Formula (C) or the second or eighth embodiments of Formula (C). Alternatively, R^{5′} is a (C₂₂-C₂₄)alkyl interrupted with —C(O)O—, and the remaining variables are as described above for Formula (C) or the second or eighth embodiments of Formula (C). Alternatively, R^{5′} is —(CH₂)₅C(O)OCH[(CH₂)₇CH₃]₂, —(CH₂)—C(O)OCH[(CH₂)₇CH₃]₂, —(CH₂)₅C(O)OCH(CH₂)₂[(CH₂)₇CH₃]₂, or —(CH₂)₇C(O)OCH(CH₂)₂[(CH₂)₇CH₃]₂, and the remaining variables are as described above for Formula (C) or the second or eighth embodiments of Formula (C).

In some embodiments, the ionizable lipid of Formula (C), (C-1), (C-3), (C-3), (C-4), (C-5), (C-7), (C-8), or (C-9) may be selected from any of the lipids listed in Table 5 below, or pharmaceutically acceptable salts thereof.

TABLE 5
Exemplary ionizable lipids of Formula (C), (C-1), (C-2), (C-3), (C-4), (C-5), (C-6), (C-7), (C-8), or (C-9)

| Lipid No. | Lipid Structure and Name |
|---|---|
| 72 | 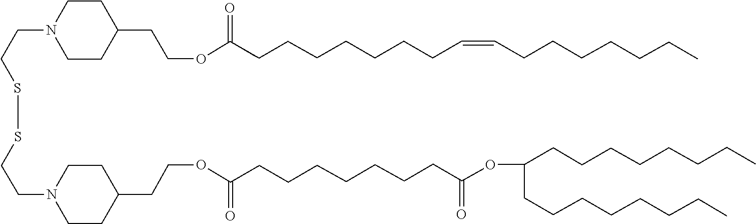 |
| | (Z)-1-(2-(1-(2-((2-(4-(2-(heptadec-9-enoyloxy)ethyl)piperidin-1- |
| | yl)ethyl)disulfaneyl)ethyl)piperidin-4-yl)ethyl) 9-(heptadecan-9-yl) |
| | nonanedioate |
| 73 | 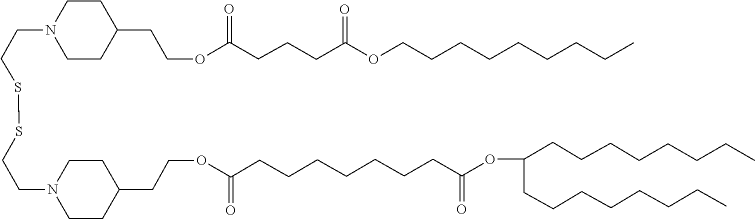 |
| | 1-(heptadecan-9-yl) 9-(2-(1-(2-((2-(4-(2-((5-(nonyloxy)-5- |
| | oxopentanoyl)oxy)ethyl)piperidin-1-yl)ethyl)disulfaneyl)ethyl)piperidin-4- |
| | yl)ethyl) nonanedioate |
| 74 | 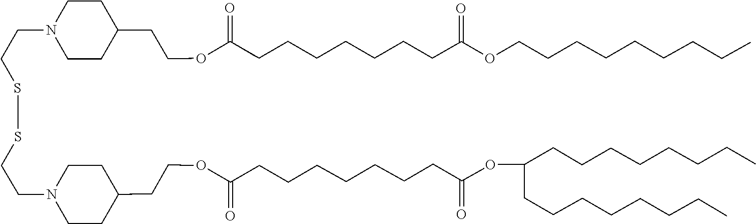 |
| | 1-(heptadecan-9-yl) 9-(2-(1-(2-((2-(4-(2-((9-(nonyloxy)-9- |
| | oxononanoyl)oxy)ethyl)piperidin-1-yl)ethyl)disulfaneyl)ethyl)piperidin-4- |
| | yl)ethyl) nonanedioate |
| 75 | 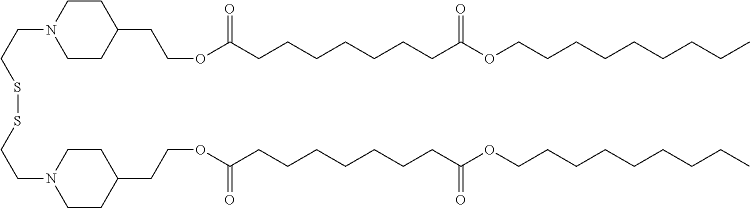 [SMILES: CCCCCCCCCOC(=O)CCCCCCCC(=O)OCCC1CCN(CCSSCCN2CCC(CCOC(=O)CCCCCCCC(=O)OCCCCCCCCC)CC2)CC1] |
| | O′1,O1-(((disulfanediylbis(ethane-2,1-diyl))bis(piperidine-1,4- |
| | diyl))bis(ethane-2,1-diyl)) 9,9′-dinonyl di(nonanedioate) |
| 76 | 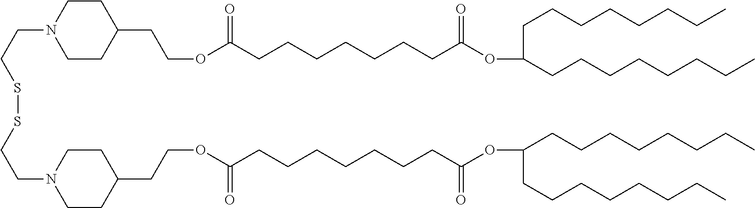 [SMILES: CCCCCCCCC(CCCCCCCC)OC(=O)CCCCCCCC(=O)OCCC1CCN(CCSSCCN2CCC(CCOC(=O)CCCCCCCC(=O)OC(CCCCCCCC)CCCCCCCC)CC2)CC1] |
| | O′1,01-(((disulfanediylbis(ethane-2,1-diyl))bis(piperidine-1,4- |
| | diyl))bis(ethane-2,1-diyl)) 9,9′-di(heptadecan-9-yl) di(nonanedioate) |

### Formula (D)

In some embodiments, the ionizable lipid, e.g., cationic lipid, in the LNPs of the present disclosure is represented by Formula (D):

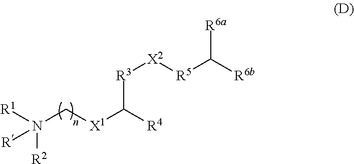

or a pharmaceutically acceptable salt thereof, wherein:

R′ is absent, hydrogen, or C₁-C₆ alkyl; provided that when R′ is hydrogen or C₁-C₆ alkyl, the nitrogen atom to which R′, R¹, and R² are all attached is positively charged;
R¹ and R² are each independently hydrogen, C₁-C₆ alkyl, or C₂-C₆ alkenyl;
R³ is C₁-C₁₂ alkylene or C₂-C₁₂ alkenylene;

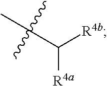

R⁴ is C₁-C₁₈ unbranched alkyl, C₂-C₁₈ unbranched alkenyl, or wherein:
   R^{4a} and R^{4b} are each independently C₁-C₁₆ unbranched alkyl or C₂-C₁₆ unbranched alkenyl;
R⁵ is absent, C₁-C₈ alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment of Formula (D), X¹ and X² are the same; and all other remaining variables are as described for Formula (C).

In a third embodiment of Formula (D), X¹ and X² are each independently —OC(═O)—, —SC(═O)—, —OC(═S)—, —C(═O)O—, —C(—O)S—, or —S—S—; or X¹ and X² are each independently —C(═O)O—, —C(═O)S—, or —S—S—; or X¹ and X² are each independently —C(═O)O— or —S—S—; and all other remaining variables are as described for Formula (D) or the second embodiment of Formula (D).

In a fourth embodiment of Formula (D), the ionizable lipid, e.g., cationic lipid, in the LNPs of the present disclosure, is represented by Formula (D-1):

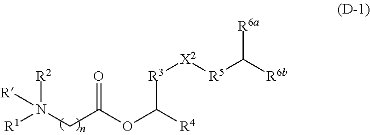

or a pharmaceutically acceptable salt thereof, wherein n is an integer selected from 1, 2, 3, and 4; and all other remaining variables are as described for Formula (D) or the second or third embodiments of Formula (D).

In a fifth embodiment of Formula (D), the ionizable lipid, e.g., cationic lipid, in the LNPs of the present disclosure, is represented by Formula (D-2):

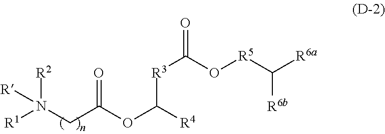

or a pharmaceutically acceptable salt thereof, wherein n is an integer selected from 1, 2, and 3; and all other remaining variables are as described for Formula (D) or the second or third embodiments of Formula (D).

In a sixth embodiment of Formula (D), the ionizable lipid, e.g., cationic lipid, in the LNPs of the present disclosure is represented by Formula (D-3):

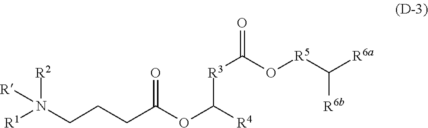

or a pharmaceutically acceptable salt thereof; and all other remaining variables are as described for Formula (D) or the second or third embodiments of Formula (D).

In a seventh embodiment of Formula (D), in the ionizable lipid, e.g., cationic lipid, according to Formula (D), Formula (D-1), Formula (D-2), Formula (D-3), or the second or third embodiments of Formula (D), R¹ and R² are each independently hydrogen, C₁-C₆ alkyl or C₂-C₆ alkenyl, or C₁-C₅ alkyl or C₂-C₅ alkenyl, or C₁-C₄ alkyl or C₂-C₄ alkenyl, or C₆ alkyl, or C₅ alkyl, or C₄ alkyl, or C₃ alkyl, or C₂ alkyl, or C₁ alkyl, or C₆ alkenyl, or C₅ alkenyl, or C₄ alkenyl, or C₃ alkenyl, or C₂ alkenyl; and all other remaining variables are as described for Formula (D), Formula (D-1), Formula (D-2), Formula (D-3) or the second or third embodiments of Formula (D).

In an eighth embodiment of Formula (D), the ionizable lipid, e.g., cationic lipid, in the LNPs of the present disclosure is represented by Formula (D-4):

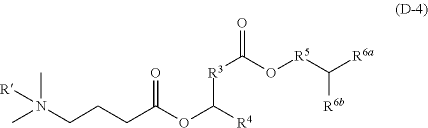

or a pharmaceutically acceptable salt thereof; and all other remaining variables are as described for Formula (D), Formula (D-1), Formula (D-2), Formula (D-3) or the second, third or seventh embodiments of Formula (D).

In a ninth embodiment of Formula (D), in the ionizable lipid, e.g., cationic lipid, according to Formula (D), Formula (D-1), Formula (D-2), Formula (D-3), Formula (D-4) or the second, third or seventh embodiments of Formula (D), R³ is C₁-C₉ alkylene or C₂-C₉ alkenylene, C₁-C₇alkylene or C₂-C₇alkenylene, C₁-C₅ alkylene or C₂-C₅ alkenylene, or C₂-C₈ alkylene or C₂-C₈ alkenylene, or C₃-C₇ alkylene or C₃-C₇alkenylene, or C₅-C₇alkylene or C₅-C₇alkenylene; or R³ is C₁₂ alkylene, C₁₁ alkylene, C₁₀ alkylene, C₉ alkylene, or C₈ alkylene, or C₇alkylene, or C₆ alkylene, or C₅ alkylene, or C₄ alkylene, or C₃ alkylene, or C₂ alkylene, or C₁ alkylene, or C₁₂ alkenylene, C₁₁ alkenylene, C₁₀ alkenylene, C₉ alkenylene, or C₈ alkenylene, or C₇ alkenylene, or C₆ alkenylene, or C₅ alkenylene, or C₄ alkenylene, or C₃ alkenylene, or C₂ alkenylene; and all other remaining variables are as described for Formula (D), Formula (D-1), Formula (D-2), Formula (D-3), Formula (D-4) or the second, third or seventh embodiments of Formula (D).

In a tenth embodiment of Formula (D), in the ionizable lipid, e.g., cationic lipid, according to Formula (D), Formula (D-1), Formula (D-2), Formula (D-3), Formula (D-4) or the second, third or seventh embodiments of Formula (D), R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₅ alkenylene, C₄ alkenylene, C₃ alkenylene, or C₂ alkenylene; and all other remaining variables are as described for Formula (D), Formula (D-1), Formula (D-2), Formula (D-3), Formula (D-4) or the second, third, seventh, or ninth embodiments of Formula (D).

In an eleventh embodiment of Formula (D), in the ionizable lipid, e.g., cationic lipid, according to Formula (D), Formula (D-1), Formula (D-2), Formula (D-3), Formula (D-4) or the second, third, seventh, ninth or tenth embodiments of Formula (D), R⁴ is C₁-C₁₄ unbranched alkyl, C₂-C₁₄ unbranched alkenyl, or

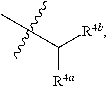

wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₇ unbranched alkyl or C₅-C₇ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is

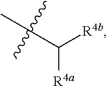

wherein R^{4a} and R^{4b} are each independently C₂-C₁₀ unbranched alkyl or C₂-C₁₀ unbranched alkenyl; or R⁴ is

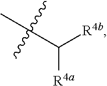

wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; and all other remaining variables are as described for Formula (D), Formula (D-1), Formula (D-2), Formula (D-3), Formula (D-4) or the second, third, seventh, ninth or tenth embodiments of Formula (D).

In a twelfth embodiment, in the ionizable lipid, e.g., cationic lipid, according to Formula (D), Formula (D-1), Formula (D-2), Formula (D-3), Formula (D-4), or the second, third, seventh, ninth, tenth or eleventh embodiments of Formula (D), R^{6a} and R^{6b} are each independently C₆-C₁₄ alkyl or C₆-C₁₄ alkenyl; or R^{6a} and R^{6b} are each independently C₈-C₁₂ alkyl or C₈-C₁₂ alkenyl; or R^{6a} and R^{6b} are each independently C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇ alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (D), Formula (D-1), Formula (D-2), Formula (D-3), Formula (D-4), or the second, third, seventh, ninth, tenth or eleventh embodiments of Formula (D).

In a thirteenth embodiment of Formula (D), in the ionizable lipid, e.g., cationic lipid, according to Formula (D), Formula (D-1), Formula (D-2), Formula (D-3), Formula (D-4), or the second, third, seventh, ninth, tenth, eleventh or twelfth embodiments of Formula (D), or a pharmaceutically acceptable salt thereof, R^{6a} and R^{6b} contain an equal number of carbon atoms with each other; or R^{6a} and R^{6b} are the same; or R^{6a} and R^{6b} are both C₁₆ alkyl, C₁₅ alkyl, C₁₄ alkyl, C₁₃ alkyl, C₁₂ alkyl, C₁₁ alkyl, C₁₀ alkyl, C₉ alkyl, C₈ alkyl, C₇alkyl, C₁₆ alkenyl, C₁₅ alkenyl, C₁₄ alkenyl, C₁₃ alkenyl, C₁₂ alkenyl, C₁₁ alkenyl, C₁₀ alkenyl, C₉ alkenyl, C₈ alkenyl, or C₇ alkenyl; provided that the total number of carbon atoms in R^{6a} and R^{6b} as combined is greater than 15; and all other remaining variables are as described for Formula (D), Formula (D-1), Formula (D-2), Formula (D-3), Formula (D-4) or the second, third, seventh, ninth, tenth, eleventh or twelfth embodiments of Formula (D).

In a fourteenth embodiment of Formula (D), in the ionizable lipid, e.g., cationic lipid, according to Formula (D), Formula (D-1), Formula (D-2), Formula (D-3), Formula (D-4), or the second, third, seventh, ninth, tenth, eleventh, twelfth or thirteenth embodiments of Formula (D), R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇alkyl and R^{6a} is C₈ alkyl, R^{6a} is C₈ alkyl and R^{6a} is C₇alkyl, R^{6a} is C₉ alkyl and R^{6a} is C₈ alkyl, R^{6a} is C₉ alkyl and R^{6a} is C₈ alkyl, R^{6a} is C₉ alkyl and R^{6a} is C₁₀ alkyl, R^{6a} is C₁₀ alkyl and R^{6a} is C₉ alkyl, R^{6a} is C₁₀ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂ alkyl and R^{6a} is C₁₁ alkyl, R^{6a} is C₇alkyl and R^{6a} is C₉ alkyl, R^{6a} is C₉ alkyl and R^{6a} is C₇alkyl, R^{6a} is C₈ alkyl and R^{6a} is C₁₀ alkyl, R^{6a} is C₁₀ alkyl and R^{6a} is C₈ alkyl, R^{6a} is C₉ alkyl and R^{6a} is C₁₁ alkyl, R^{6a} is C₁₁ alkyl and R^{6a} is C₉ alkyl, R^{6a} is C₁₀ alkyl and R^{6a} is C₁₂ alkyl, R^{6a} is C₁₂ alkyl and R^{6a} is C₁₀ alkyl, R^{6a} is C₁₁ alkyl and R^{6a} is C₁₃ alkyl, or R^{6a} is C₁₃ alkyl and R^{6a} is C₁₁ alkyl, etc.; and all other remaining variables are as described for Formula I, Formula II, Formula III, Formula IV, Formula V, or the second, third, seventh, ninth, tenth, eleventh, twelfth or thirteenth embodiments of Formula (D).

In a fifteenth embodiment of Formula (D), R⁴ is C₁-C₁₆ unbranched alkyl, C₂-C₁₆ unbranched alkenyl, or

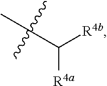

wherein R^{4a} and R^{4b} are as described above for the second, third, fourth, fifth, sixth, seventh, eighth, ninth, tenth, eleventh, twelfth, thirteenth or fourteenth embodiments of Formula (D).

In one embodiment, the ionizable lipid, e.g., cationic lipid, of the present disclosure or the ionizable lipid of Formula (D), Formula (D-1), Formula (D-2), Formula (D-3), or Formula (D-4) is any one lipid selected from the lipids listed in Table 6 below, or a pharmaceutically acceptable salt thereof:

TABLE 6
Exemplary lipids of Formula (D), Formula (D-1), Formula (D-2), Formula (D-3) or Formula (D-4)

| Lipid | |
|---|---|
| No. | Lipid Structure and Name |
| 77 | 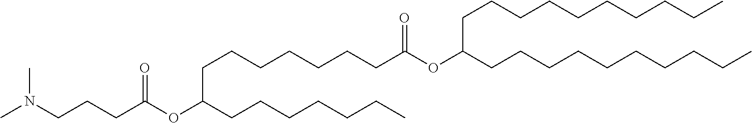 [SMILES: CCCCCCCCCCC(CCCCCCCCCC)OC(=O)CCCCCCCC(CCCCCCC)OC(=O)CCCN(C)C] |
| | henicosan-11-yl 9-((4-(dimethylamino)butanoyl)oxy)hexadecanoate |
| 78 | 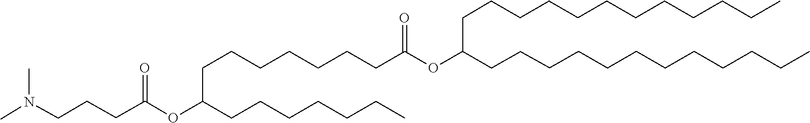 [SMILES: CCCCCCCCCCCCC(CCCCCCCCCCCC)OC(=O)CCCCCCCC(CCCCCCC)OC(=O)CCCN(C)C] |
| | pentacosan-13-yl 9-((4-(dimethylamino)butanoyl)oxy)hexadecanoate |
| 79 | 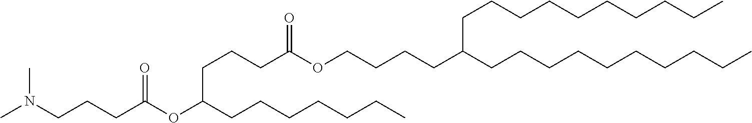 [SMILES: CCCCCCCCCCC(CCCCCCCCCC)CCCCOC(=O)CCCC(CCCCCCC)OC(=O)CCCN(C)C] |
| | 5-decylpentadecyl 5-((4-(dimethylamino)butanoyl)oxy)dodecanoate |
| 80 | 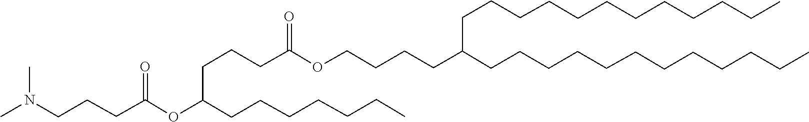 [SMILES: CCCCCCCCCCCCC(CCCCCCCCCCCC)CCCCOC(=O)CCCC(CCCCCCC)OC(=O)CCCN(C)C] |
| | 5-dodecylheptadecyl 5-((4-(dimethylamino)butanoyl)oxy)dodecanoate |
| 81 | 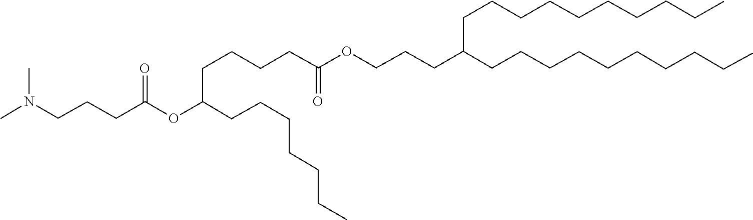 [SMILES: CCCCCCCCCCC(CCCCCCCCCC)CCCOC(=O)CCCCC(CCCCCCC)OC(=O)CCCN(C)C] |
| | 4-decyltetradecyl 6-((4-(dimethylamino)butanoyl)oxy)tridecanoate |
| 82 | 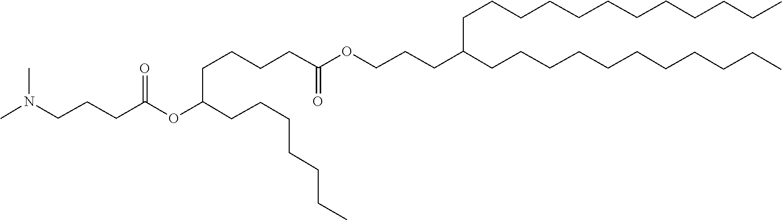 [SMILES: CCCCCCCCCCCCC(CCCCCCCCCCC)CCCOC(=O)CCCCC(CCCCCCC)OC(=O)CCCN(C)C] |
| | 4-dodecylhexadecyl 6-((4-(dimethylamino)butanoyl)oxy)tridecanoate |
| 83 | 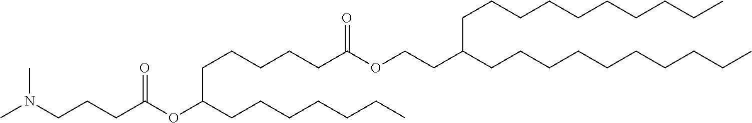 [SMILES: CCCCCCCCCCC(CCCCCCCCCC)CCOC(=O)CCCCCC(CCCCCCC)OC(=O)CCCN(C)C] |
| | 3-decyltridecyl 7-((4-(dimethylamino)butanoyl)oxy)tetradecanoate |
| 84 | 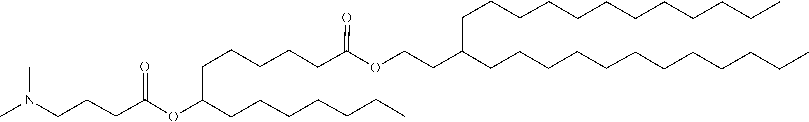 [SMILES: CCCCCCCCCCCCC(CCCCCCCCCCCC)CCOC(=O)CCCCCC(CCCCCCC)OC(=O)CCCN(C)C] |
| | 3-dodecylpentadecyl 7-((4-(dimethylamino)butanoyl)oxy)tetradecanoate |
| 85 | 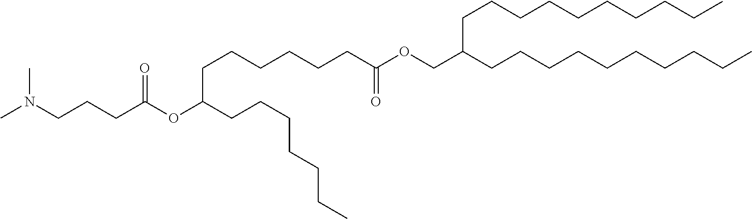 [SMILES: CCCCCCCCCCC(CCCCCCCCCC)COC(=O)CCCCCCC(CCCCCCC)OC(=O)CCCN(C)C] |
| | 2-decyldodecyl 8-((4-(dimethylamino)butanoyl)oxy)pentadecanoate |
| 86 | 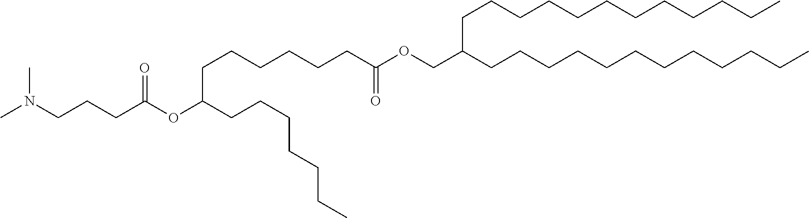 [SMILES: CCCCCCCCCCCCC(CCCCCCCCCCCC)COC(=O)CCCCCCC(CCCCCCC)OC(=O)CCCN(C)C] |
| | 2-dodecyltetradecyl 8-((4-(dimethylamino)butanoyl)oxy)pentadecanoate |
| 87 | 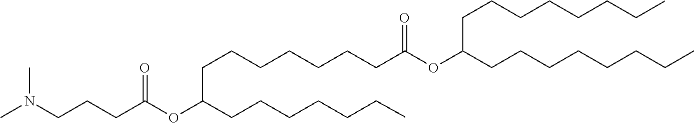 [SMILES: CCCCCCCCC(CCCCCCCC)OC(=O)CCCCCCCC(CCCCCCC)OC(=O)CCCN(C)C] |
| | heptadecan-9-yl 9-((4-(dimethylamino)butanoyl)oxy)hexadecanoate |
| 88 | 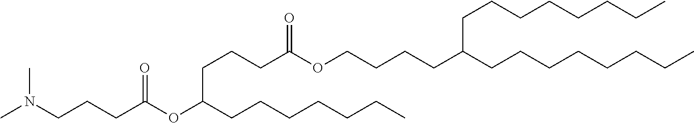 [SMILES: CCCCCCCCC(CCCCCCCC)CCCCOC(=O)CCCC(CCCCCCC)OC(=O)CCCN(C)C] |
| | 5-octyltridecyl 5-((4-(dimethylamino)butanoyl)oxy)dodecanoate |
| 89 | 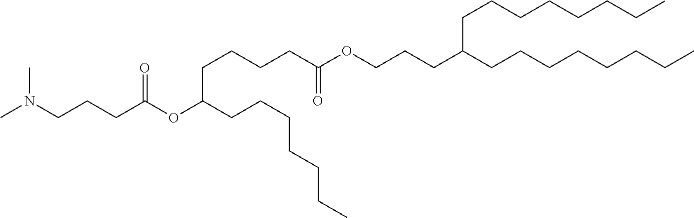 [SMILES: CCCCCCCCC(CCCCCCCC)CCCOC(=O)CCCCC(CCCCCCC)OC(=O)CCCN(C)C] |
| | 4-octyldodecyl 6-((4-(dimethylamino)butanoyl)oxy)tridecanoate |
| 91 | 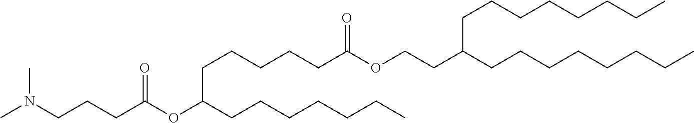 [SMILES: CCCCCCCCC(CCCCCCCC)CCOC(=O)CCCCCC(CCCCCCC)OC(=O)CCCN(C)C] |
| | 3-octylundecyl 7-((4-(dimethylamino)butanoyl)oxy)tetradecanoate |
| 92 | 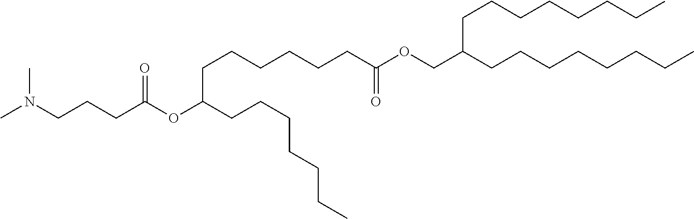 [SMILES: CCCCCCCCC(CCCCCCCC)COC(=O)CCCCCCC(CCCCCCC)OC(=O)CCCN(C)C] |
| | 2-octyldecyl 8-((4-(dimethylamino)butanoyl)oxy)pentadecanoate |
| 93 | 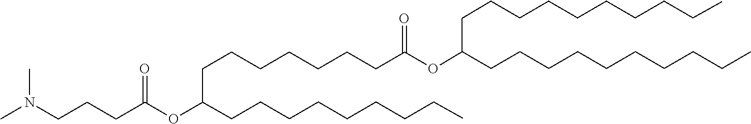 [SMILES: CCCCCCCCCCC(CCCCCCCCCC)OC(=O)CCCCCCCC(CCCCCCCCC)OC(=O)CCCN(C)C] |
| | henicosan-11-yl 9-((4-(dimethylamino)butanoyl)oxy)octadecanoate |
| 94 | 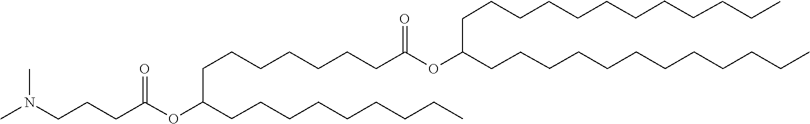 [SMILES: CCCCCCCCCCCCC(CCCCCCCCCCCC)OC(=O)CCCCCCCC(CCCCCCCCC)OC(=O)CCCN(C)C] |
| | pentacosan-13-yl 9-((4-(dimethylamino)butanoyl)oxy)octadecanoate |
| 95 | 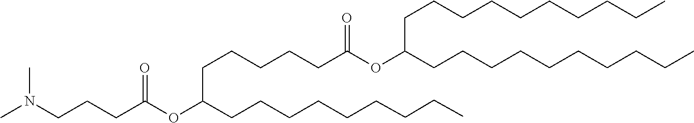 [SMILES: CCCCCCCCCCC(CCCCCCCCCC)OC(=O)CCCCCC(CCCCCCCCC)OC(=O)CCCN(C)C] |
| | henicosan-11-yl 7-((4-(dimethylamino)butanoyl)oxy)hexadecanoate |
| 96 | 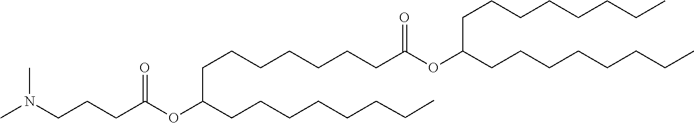 [SMILES: CCCCCCCCC(CCCCCCCC)OC(=O)CCCCCCCC(CCCCCCCC)OC(=O)CCCN(C)C] |
| | heptadecan-9-yl 9-((4-(dimethylamino)butanoyl)oxy)heptadecanoate |
| 97 | 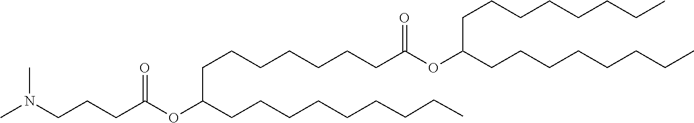 [SMILES: CCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)octadecanoate |
| 98 | 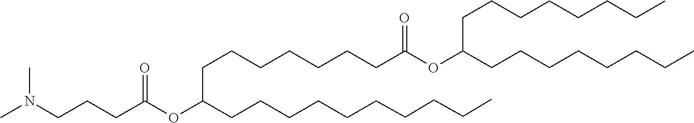 [SMILES: CCCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)nonadecanoate |
| 99 | 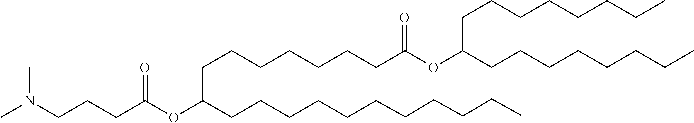 [SMILES: CCCCCCCCCCCC(CCCCCCCC(=O)OC(CCCCCCCC)CCCCCCCC)OC(=O)CCCN(C)C] |
| | heptadecan-9-yl 9-((4-(dimethylamino)butanoyl)oxy)icosanoate |
| 100 | 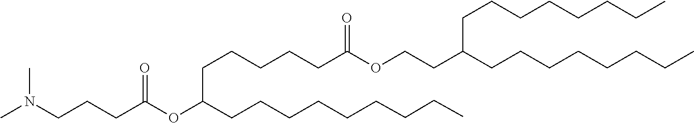 [SMILES: CCCCCCCCCC(CCCCCC(=O)OCCC(CCCCCCCC)CCCCCCCC)OC(=O)CCCN(C)C] |
| | 3-octylundecyl 7-((4-(dimethylamino)butanoyl)oxy)hexadecanoate |
| 101 | 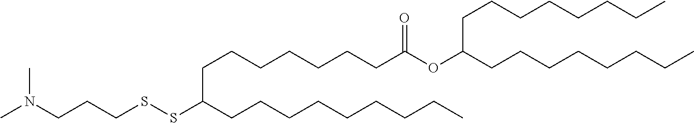 [SMILES: CCCCCCCCCC(CCCCCCCC(=O)OC(CCCCCCCC)CCCCCCCC)SSCCCN(C)C] |
| | heptadecan-9-yl 9-((3-(dimethylamino)propyl)disulfaneyl)octadecanoate |
| 113 | 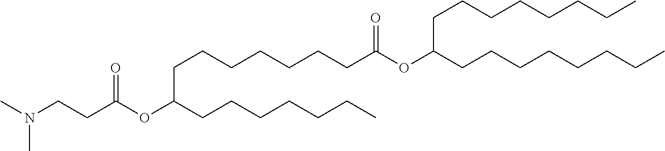 [SMILES: CCCCCCCCC(CCCCCCCC)OC(=O)CCCCCCCC(CCCCCCC)OC(=O)CCN(C)C] |
| 114 | 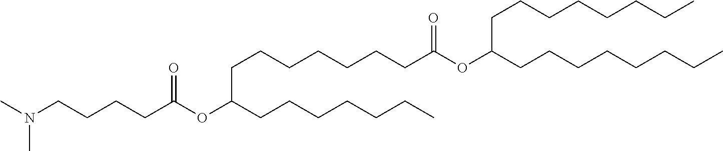 [SMILES: CCCCCCCCC(CCCCCCCC)OC(=O)CCCCCCCC(CCCCCCC)OC(=O)CCCCN(C)C] |
| 115 | 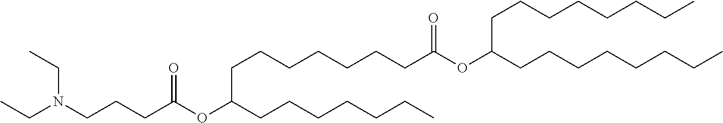 [SMILES: CCCCCCCCC(CCCCCCCC)OC(=O)CCCCCCCC(CCCCCCC)OC(=O)CCCN(CC)CC] |
| 116 | 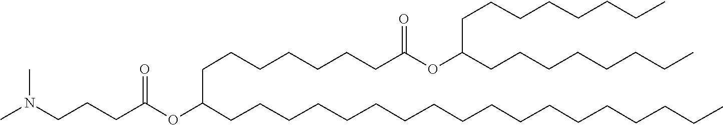 [SMILES: CCCCCCCCCCCCCCCCCCC(CCCCCCCC(=O)OC(CCCCCCCC)CCCCCCCC)OC(=O)CCCN(C)C] |
| 117 | 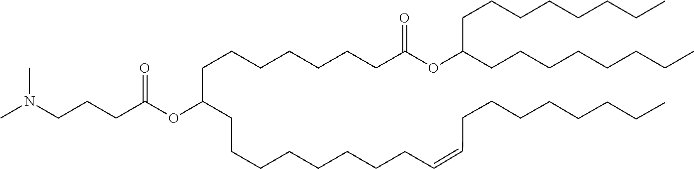 [SMILES: CCCCCCCC/C=C\CCCCCCCCC(CCCCCCCC(=O)OC(CCCCCCCC)CCCCCCCC)OC(=O)CCCN(C)C] |
| 118 | 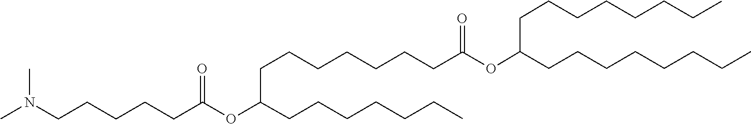 [SMILES: CCCCCCCCC(CCCCCCCC)OC(=O)CCCCCCCC(CCCCCCC)OC(=O)CCCCCN(C)C] |

In one embodiment, the ionizable lipid in the LNPs of the present disclosure comprises Lipid No. 87:

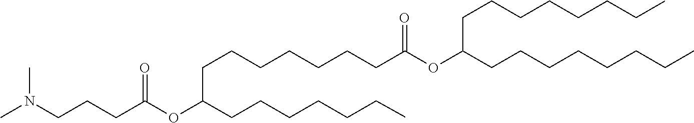 [SMILES: CCCCCCCCC(CCCCCCCC)OC(=O)CCCCCCCC(CCCCCCC)OC(=O)CCCN(C)C]

heptadecan-9-yl 9-((4-(dimethylamino)butanoyl)oxy) hexadecanoate
   or a pharmaceutically acceptable salt or ester thereof, or a deuterated analogue thereof.

### Formula (E)

In some embodiments, the ionizable lipid, e.g., cationic lipid, in the LNPs of the present disclosure is represented by Formula (E):

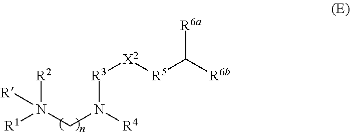

### (E)

or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is positively charged;
R¹ and R² are each independently hydrogen or C₁-C₃ alkyl;
R³ is C₃-C₁₀ alkylene or C₃-C₁₀ alkenylene;

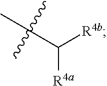

R⁴ is C₁-C₁₆ unbranched alkyl, C₂-C₁₆ unbranched alkenyl, or wherein:
   R^{4a} and R^{4b} are each independently C₁-C₁₆ unbranched alkyl or C₂-C₁₆ unbranched alkenyl;
R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
   R^{a}, for each occurrence, is independently hydrogen or C₁-C₆ alkyl; and
n is an integer selected from 1, 2, 3, 4, 5, and 6.

In a second embodiment of Formula (E), in the ionizable lipid, e.g., cationic lipid, according to the first embodiment, or a pharmaceutically acceptable salt thereof, X is —OC(═O)—, —SC(═O)—, —OC(═S)—, —C(═O)O—, —C(═O)S—, or —S—S—; and all other remaining variables are as described for Formula I or the first embodiment.

In a third embodiment of Formula (E), the ionizable lipid, e.g., cationic lipid, in the LNPs of the present disclosure is represented by Formula (E-1):

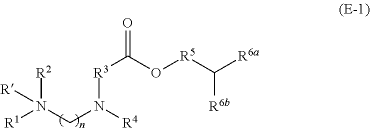

or a pharmaceutically acceptable salt thereof, wherein n is an integer selected from 1, 2, 3, and 4; and all other remaining variables are as described for Formula (E) or the second embodiment of Formula (E). Alternatively, n is an integer selected from 1, 2, and 3; and all other remaining variables are as described for Formula (E) or the second embodiment of Formula (E).

In a fourth embodiment of Formula (E), the ionizable lipid, e.g., cationic lipid, in the LNPs of the present disclosure is represented by Formula (E-2):

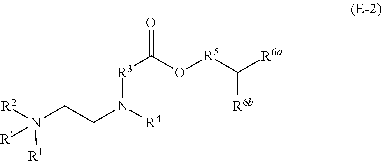

or a pharmaceutically acceptable salt thereof; and all other remaining variables are as described for Formula (E), Formula (E-1) or the second embodiment of Formula (E).

In a fifth embodiment of Formula (E), in the ionizable lipid, e.g., cationic lipid, in the LNPs of the present disclosure, R¹ and R² are each independently hydrogen or C₁-C₂ alkyl, or C₂-C₃ alkenyl; or R′, R¹, and R² are each independently hydrogen, C₁-C₂ alkyl; and all other remaining variables are as described for Formula (E), Formula (E-1) or the second embodiment of Formula (E).

In a sixth embodiment of Formula (E), the ionizable lipid, e.g., cationic lipid, in the LNPs of the present disclosure is represented by Formula (E-3):

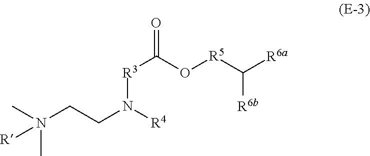

or a pharmaceutically acceptable salt thereof; and all other remaining variables are as described for Formula (E), Formula (E-1), Formula (E-2) or the second or fifth embodiments of Formula (E).

In a seventh embodiment of Formula (E), in the ionizable lipid, e.g., cationic lipid, according to Formula (E), Formula (E-1), Formula (E-2), Formula (E-3) or the second or firth embodiments of Formula (E), R⁵ is absent or C₁-C₈ alkylene; or R⁵ is absent, C₁-C₆ alkylene, or C₂-C₆ alkenylene; or R⁵ is absent, C₁-C₄ alkylene, or C₂-C₄ alkenylene; or R⁵ is absent; or R⁵ is C₈ alkylene, C₇alkylene, C₆ alkylene, C₅ alkylene, C₄ alkylene, C₃ alkylene, C₂ alkylene, C₁ alkylene, C₈ alkenylene, C₇ alkenylene, C₆ alkenylene, C₅ alkenylene, C₄ alkenylene, C₃ alkenylene, or C₂ alkenylene; and all other remaining variables are as described for Formula (E), Formula (E-1), Formula (E-2), Formula (E-3) or the second or fifth embodiments of Formula (E).

In an eighth embodiment of Formula (E), he ionizable lipid, e.g., cationic lipid, in the LNPs of the present disclosure is represented by Formula (E-4):

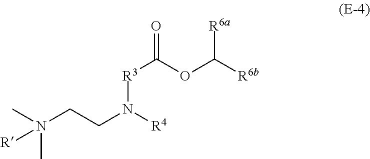

or a pharmaceutically acceptable salt thereof; and all other remaining variables are as described for Formula (E), Formula (E-1), Formula (E-2), Formula (E-3) or the second, fifth or seventh embodiments of Formula (E).

In a ninth embodiment, in the ionizable lipid, e.g., cationic lipid, according to Formula (E), Formula (E-1), Formula (E-2), Formula (E-3), Formula (E-4) or the second, fifth or seventh embodiments of Formula (E), or a pharmaceutically acceptable salt thereof, R⁴ is C₁-C₁₄ unbranched alkyl, C₂-C₁₄ unbranched alkenyl, or

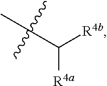

wherein R^{4a} and R^{4b} are each independently C₁-C₁₂ unbranched alkyl or C₂-C₁₂ unbranched alkenyl; or R⁴ is C₂-C₁₂ unbranched alkyl or C₂-C₁₂ unbranched alkenyl; or R⁴ is C₅-C₁₂ unbranched alkyl or C₅-C₁₂ unbranched alkenyl; or R⁴ is C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ unbranched alkyl, C₁ unbranched alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; or R⁴ is

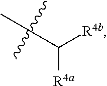

wherein R^{4a} and R^{4b} are each independently C₂-C₁₀ unbranched alkyl or C₂-C₁₀ unbranched alkenyl; or R⁴ is

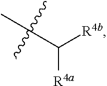

wherein R^{4a} and R^{4b} are each independently C₁₆ unbranched alkyl, C₁₅ unbranched alkyl, C₁₄ unbranched alkyl, C₁₃ unbranched alkyl, C₁₂ unbranched alkyl, C₁₁ unbranched alkyl, C₁₀ unbranched alkyl, C₉ unbranched alkyl, C₈ unbranched alkyl, C₇ unbranched alkyl, C₆ unbranched alkyl, C₅ unbranched alkyl, C₄ unbranched alkyl, C₃ unbranched alkyl, C₂ alkyl, C₁ alkyl, C₁₆ unbranched alkenyl, C₁₅ unbranched alkenyl, C₁₄ unbranched alkenyl, C₁₃ unbranched alkenyl, C₁₂ unbranched alkenyl, C₁₁ unbranched alkenyl, C₁₀ unbranched alkenyl, C₉ unbranched alkenyl, C₈ unbranched alkenyl, C₇ unbranched alkenyl, C₆ unbranched alkenyl, C₅ unbranched alkenyl, C₄ unbranched alkenyl, C₃ unbranched alkenyl, or C₂ alkenyl; and all other remaining variables are as described for Formula (E), Formula (E-1), Formula (E-2), Formula (E-3), Formula (E-4) or the second, fifth or seventh embodiments of Formula (E).

In a tenth embodiment, in the ionizable lipid, e.g., cationic lipid, according to Formula (E), Formula (E-1), Formula (E-2), Formula (E-3), Formula (E-4) or the second, fifth, seventh or ninth embodiments of Formula (E), R³ is C₃-C₈ alkylene or C₃-C₈ alkenylene, C₃-C₇alkylene or C₃-C₇ alkenylene, or C₃-C₅ alkylene or C₃-C₅ alkenylene; or R³ is C₈ alkylene, or C₇ alkylene, or C₆ alkylene, or C₅ alkylene, or C₄ alkylene, or C₃ alkylene, or C₁ alkylene, or C₈ alkenylene, or C₇ alkenylene, or C₆ alkenylene, or C₅ alkenylene, or C₄ alkenylene, or C₃ alkenylene; and all other remaining variables are as described for Formula (E), Formula (E-1), Formula (E-2), Formula (E-3), Formula (E-4) or the second, fifth, seventh or ninth embodiments of Formula (E).

In an eleventh embodiment, in the ionizable lipid, e.g., cationic lipid, according to Formula (E), Formula (E-1), Formula (E-2), Formula (E-3), Formula (E-4) or the second, fifth, seventh, ninth or tenth embodiments of Formula (E), R^{6a} and R^{6b} are each independently C₇-C₁₂ alkyl or C₇-C₁₂ alkenyl; or R^{6a} and R^{6b} are each independently C₈-C₁₀ alkyl or C₈-C₁₀ alkenyl; or R^{6a} and R^{6b} are each independently C₁₂ alkyl, C₁₁ alkyl, C₁₀ alkyl, C₉ alkyl, C₅ alkyl, C₇alkyl, C₁₂ alkenyl, C₁₁ alkenyl, C₁₀ alkenyl, C₉ alkenyl, C₈ alkenyl, or C₇ alkenyl; and all other remaining variables are as described for Formula (E), Formula (E-1), Formula (E-2), Formula (E-3), Formula (E-4) or the second, fifth, seventh, ninth or tenth embodiments of Formula (E).

In a twelfth embodiment, in the ionizable lipid, e.g., cationic lipid, according to Formula (E), Formula (E-1), Formula (E-2), Formula (E-3), Formula (E-4) or the second, fifth, seventh, ninth, tenth or eleventh embodiments of Formula (E), R^{6a} and R^{6b} contain an equal number of carbon atoms with each other; or R^{6a} and R^{6b} are the same; or R^{6a} and R^{6b} are both C₁₂ alkyl, C₁₁ alkyl, C₁₀ alkyl, C₉ alkyl, C₈ alkyl, C₇ alkyl, C₁₂ alkenyl, C₁₁ alkenyl, C₁₀ alkenyl, C₉ alkenyl, C₈ alkenyl, or C₇ alkenyl; and all other remaining variables are as described for Formula (E), Formula (E-1), Formula (E-2), Formula (E-3), Formula (E-4) or the second, fifth, seventh, ninth, tenth or eleventh embodiments of Formula (E).

In a thirteenth embodiment, in the ionizable lipid, e.g., cationic lipid, according to Formula (E), Formula (E-1), Formula (E-2), Formula (E-3), Formula (E-4), R^{6a} and R^{6b} as defined in any one of the preceding embodiments each contain a different number of carbon atoms with each other; or the number of carbon atoms R^{6a} and R^{6b} differs by one or two carbon atoms; or the number of carbon atoms R^{6a} and R^{6b} differs by one carbon atom; or R^{6a} is C₇alkyl and R^{6a} is C₈ alkyl, R^{6a} is C₈ alkyl and R^{6a} is C₇alkyl, R^{6a} is C₈ alkyl and R^{6a} is C₉ alkyl, R^{6a} is C₉ alkyl and R^{6a} is C₈ alkyl, R^{6a} is C₉ alkyl and R^{6a} is C₁₀ alkyl, R^{6a} is C₁₀ alkyl and R^{6a} is C₉ alkyl, R^{6a} is C₁₀ alkyl and R^{6a} is C₁₁ alkyl, R^{6a} is C₁₁ alkyl and R^{6a} is C₁₀ alkyl, R^{6a} is C₁₁ alkyl and R^{6a} is C₁₂ alkyl, R^{6a} is C₁₂ alkyl and R^{6a} is C₁₁ alkyl, R^{6a} is C₇ alkyl and R^{6a} is C₉ alkyl, R^{6a} is C₉ alkyl and R^{6a} is C₇alkyl, R^{6a} is C₈ alkyl and R^{6a} is C₁₀ alkyl, R^{6a} is C₁₀ alkyl and R^{6a} is C₈ alkyl, R^{6a} is C₉ alkyl and R^{6a} is C₁₁ alkyl, R^{6a} is C₁₁ alkyl and R^{6a} is C₉ alkyl, R^{6a} is C₁₀ alkyl and R^{6a} is C₁₂ alkyl, R^{6a} is C₁₂ alkyl and R^{6a} is C₁₀ alkyl, etc.; and all other remaining variables are as described for Formula (E), Formula (E-1), Formula (E-2), Formula (E-3), Formula (E-4) or the second, fifth, seventh, ninth, tenth, eleventh or twelfth embodiments of Formula (E).

In a fourteenth embodiment, in the ionizable lipid, e.g., cationic lipid, according to Formula (E), Formula (E-1), Formula (E-2), Formula (E-3), Formula (E-4) or the second, fifth, seventh, ninth, tenth, eleventh, twelfth or thirteenth embodiments of Formula (E), R′ is absent; and all other remaining variables are as described for Formula (E), Formula (E-1), Formula (E-2), Formula (E-3), Formula (E-4) or the second, fifth, seventh, ninth, tenth, eleventh, twelfth or thirteenth embodiments of Formula (E).

In one embodiment, the ionizable lipid, e.g., cationic lipid, in the LNPs of the present disclosure or the cationic lipid of Formula (E), Formula (E-1), Formula (E-2), Formula (E-3), Formula (E-4) is any one lipid selected from the lipids in Table 7 or a pharmaceutically acceptable salt thereof:

TABLE 7
Exemplary lipids of Formula (E), Formula (E-1), Formula (E-2), Formula (E-3), Formula (E-4)

| Lipid | |
|---|---|
| No. | Lipid Structure and Name |
| 102 | 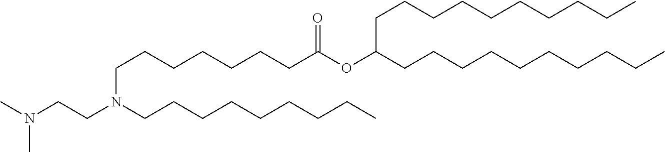 [SMILES: CCCCCCCCCCC(CCCCCCCCCC)OC(=O)CCCCCCCN(CCCCCCCCC)CCN(C)C] |
| | henicosan-11-yl 8-((2-(dimethylamino)ethyl)(nonyl)amino)octanoate |
| 103 | 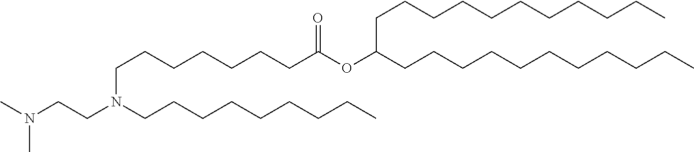 [SMILES: CCCCCCCCCCCC(CCCCCCCCCCC)OC(=O)CCCCCCCN(CCCCCCCCC)CCN(C)C] |
| | tricosan-12-yl 8-((2-(dimethylamino)ethyl)(nonyl)amino)octanoate |
| 104 | 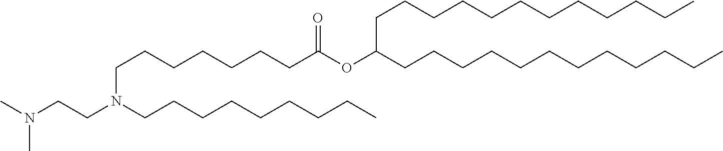 [SMILES: CCCCCCCCCCCCC(CCCCCCCCCCCC)OC(=O)CCCCCCCN(CCCCCCCCC)CCN(C)C] |
| | pentacosan-13-yl 8-((2-(dimethylamino)ethyl)(nonyl)amino)octanoate |
| 105 | 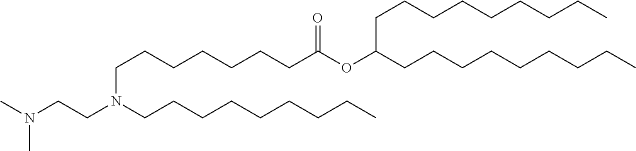 [SMILES: CCCCCCCCCC(CCCCCCCCC)OC(=O)CCCCCCCN(CCCCCCCCC)CCN(C)C] |
| | nonadecan-10-yl 8-((2-(dimethylamino)ethyl)(nonyl)amino)octanoate |
| 106 | 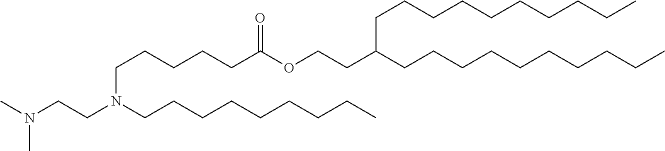 [SMILES: CCCCCCCCCCC(CCCCCCCCCC)CCOC(=O)CCCCCN(CCCCCCCCC)CCN(C)C] |
| | 3-decyltridecyl 6-((2-(dimethylamino)ethyl)(nonyl)amino)hexanoate |
| 107 | 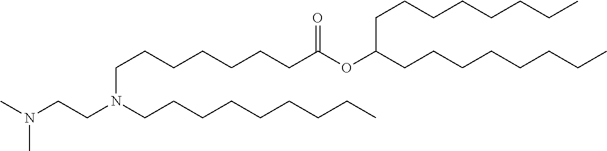 [SMILES: CCCCCCCCCN(CCCCCCCC(=O)OC(CCCCCCCC)CCCCCCCC)CCN(C)C] |
| | heptadecan-9-yl 8-((2-(dimethylamino)ethyl)(nonyl)amino)octanoate |
| 108 | 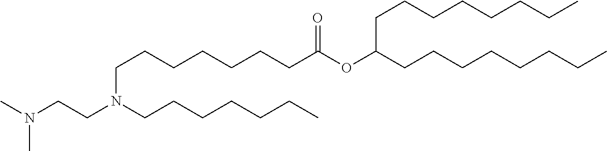 [SMILES: CCCCCCCCC(CCCCCCCC)OC(=O)CCCCCCCN(CCCCCCC)CCN(C)C] |
| | heptadecan-9-yl 8-((2-(dimethylamino)ethyl)(heptyl)amino)octanoate |
| 109 | 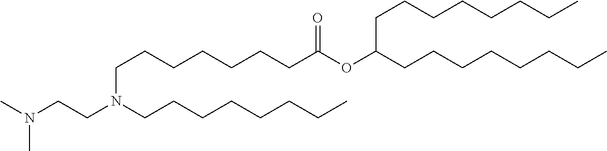 [SMILES: CCCCCCCCC(CCCCCCCC)OC(=O)CCCCCCCN(CCCCCCCC)CCN(C)C] |
| | heptadecan-9-yl 8-((2-(dimethylamino)ethyl)(octyl)amino)octanoate |
| 110 | 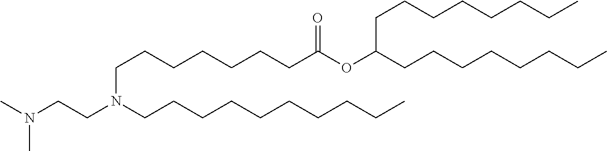 [SMILES: CCCCCCCCCCN(CCCCCCCC(=O)OC(CCCCCCCC)CCCCCCCC)CCN(C)C] |
| | heptadecan-9-yl 8-(decyl(2-(dimethylamino)ethyl)amino)octanoate |
| 111 | 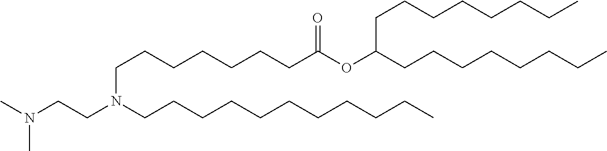 [SMILES: CCCCCCCCCCCN(CCCCCCCC(=O)OC(CCCCCCCC)CCCCCCCC)CCN(C)C] |
| | heptadecan-9-yl 8-((2-(dimethylamino)ethyl)(undecyl)amino)octanoate |
| 112 | 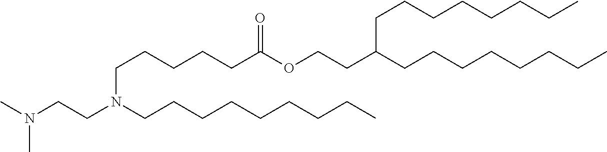 [SMILES: CCCCCCCCCN(CCCCCC(=O)OCCC(CCCCCCCC)CCCCCCCC)CCN(C)C] |
| | 3-octylundecyl 6-((2-(dimethylamino)ethyl)(nonyl)amino)hexanoate |

Specific examples are provided in the exemplification section below and are included as part of the cationic or ionizable lipids described herein. Pharmaceutically acceptable salts as well as neutral forms are also included.

### Cleavable Lipids

In some embodiments, the LNPs provided by the present disclosure comprise an ionizable lipid that is also a cleavable lipid. As used herein, the term “cleavable lipid”, which may be used interchangeably with the term “SS-cleavable lipid” refers to an ionizable lipid comprising a disulfide bond (“SS”). The SS in the cleavable lipid is a cleavable unit. In one embodiment, a cleavable lipid comprises an amine, e.g., a tertiary amine, and a disulfide bond. In this cleavable lipid, an amine can become protonated in an acidic compartment (e.g., an endosome or a lysosome), leading to LNP destabilization, and the cleavable lipid can become cleaved in a reductive environment (e.g., the cytoplasm). Cleavable lipids also include pH-activated lipid-like materials, such as ss-OP lipids, ssPalm lipids, ss-M lipids, ss-E lipids, ss-EC lipids, ss-LC lipids and ss-OC lipids, etc.

According to some embodiments, SS-cleavable lipids are described in International Patent Application Publication No. WO2019188867, incorporated by reference in its entirety herein.

In one embodiment, a cleavable lipid may comprise three components: an amine head group, a linker group, and a hydrophobic tail(s). In one embodiment, the cleavable lipid comprises one or more phenyl ester bonds, one of more tertiary amino groups, and a disulfide bond. The tertiary amine groups provide pH responsiveness and induce endosomal escape, the phenyl ester bonds enhance the degradability of the structure (self-degradability) and the disulfide bond becomes cleaved in a reductive environment.

In one embodiment, the cleavable lipid is an ss-OP lipid. In one embodiment, an ss-OP lipid comprises the structure of Lipid A shown below:

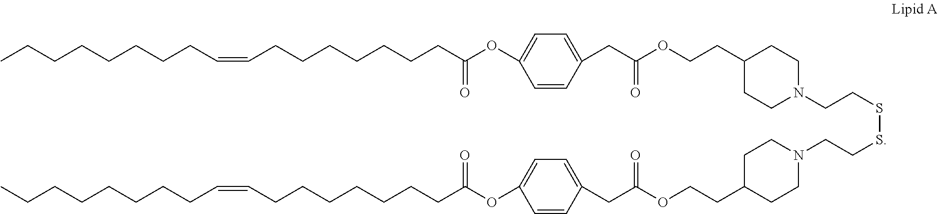 [SMILES: CCCCCCCC/C=C\CCCCCCCC(=O)Oc1ccc(CC(=O)OCCC2CCN(CCSSCCN3CCC(CCOC(=O)Cc4ccc(OC(=O)CCCCCCC/C=C\CCCCCCCC)cc4)CC3)CC2)cc1]

In one embodiment, the SS-cleavable lipid is an SS-cleavable and pH-activated lipid-like material (ssPalm). ssPalm lipids are well known in the art. For example, see Togashi et al., Journal of Controlled Release, 279 (2018) 262-270, the entire contents of which are incorporated herein by reference. In one embodiment, the ssPalm is an ssPalmM lipid comprising the structure of Lipid B shown below:

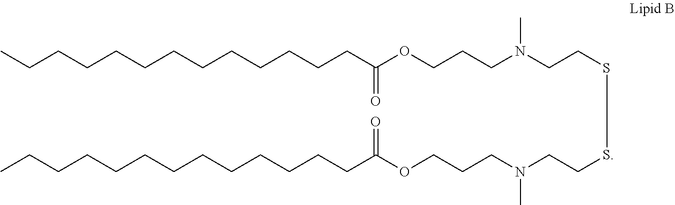 [SMILES: CCCCCCCCCCCCCC(=O)OCCCN(C)CCSSCCN(C)CCCOC(=O)CCCCCCCCCCCCC]

In one embodiment, the ssPalmE lipid is a ssPalmE-P4-C₂ lipid comprising the structure of Lipid C below:

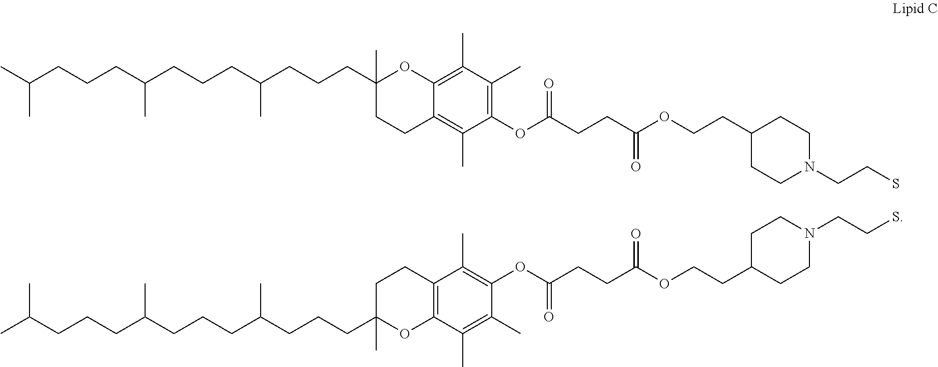

In one embodiment, the ssPalmE lipid is a ssPalmE-Paz4-C₂ lipid, comprising the structure of Lipid D below:

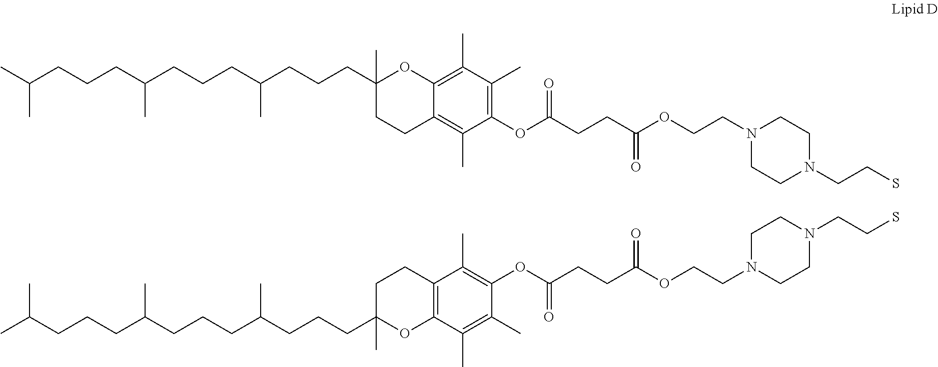

In one embodiment, the cleavable lipid is an ss-M lipid. In one embodiment, an ss-M lipid comprises the structure shown in Lipid E below:

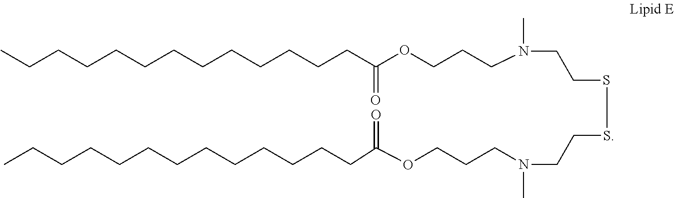 [SMILES: CCCCCCCCCCCCCC(=O)OCCCN(C)CCSSCCN(C)CCCOC(=O)CCCCCCCCCCCCC]

In one embodiment, the cleavable lipid is an ss-E lipid. In one embodiment, an ss-E lipid comprises the structure shown in Lipid F below:

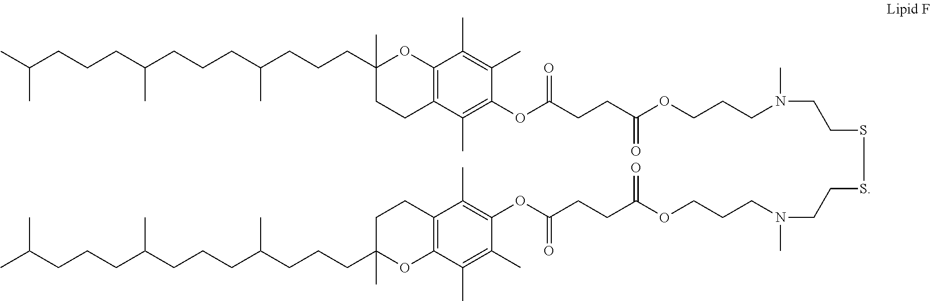 [SMILES: Cc1c(C)c2c(c(C)c1OC(=O)CCC(=O)OCCCN(C)CCSSCCN(C)CCCOC(=O)CCC(=O)Oc1c(C)c(C)c3c(c1C)CCC(C)(CCCC(C)CCCC(C)CCCC(C)C)O3)CCC(C)(CCCC(C)CCCC(C)CCCC(C)C)O2]

In one embodiment, the cleavable lipid is an ss-EC lipid. In one embodiment, an ss-EC lipid comprises the structure shown for Lipid G below:

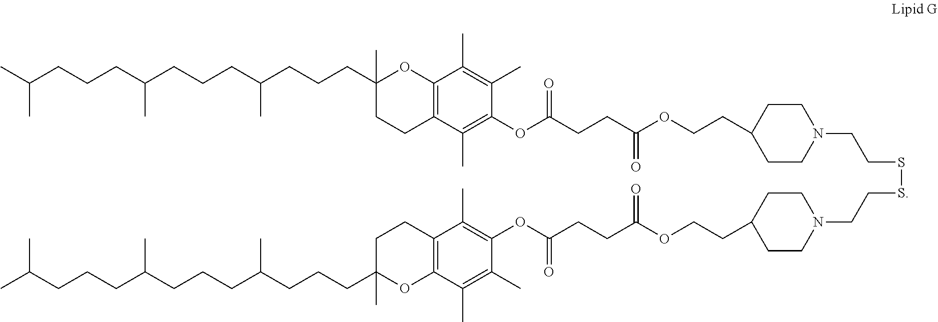 [SMILES: Cc1c(C)c2c(c(C)c1OC(=O)CCC(=O)OCCC1CCN(CCSSCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2]

In one embodiment, the cleavable lipid is an ss-LC lipid. In one embodiment, an ss-LC lipid comprises the structure shown for Lipid H below:

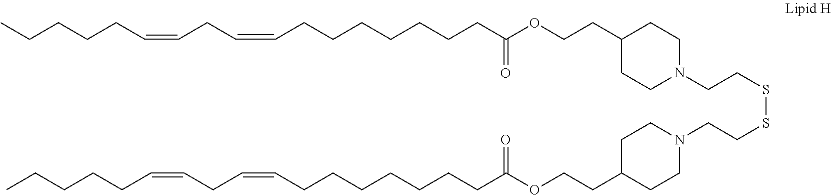 [SMILES: CCCCC/C=C\C/C=C\CCCCCCCC(=O)OCCC1CCN(CCSSCCN2CCC(CCOC(=O)CCCCCCC/C=C\C/C=C\CCCCC)CC2)CC1]

In one embodiment, the cleavable lipid is an ss-OC lipid. In one embodiment, an ss-OC lipid comprises the structure shown for Lipid J below:

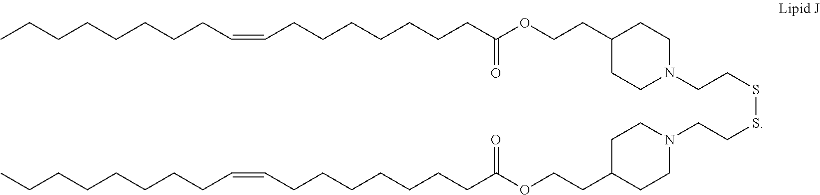 [SMILES: CCCCCCCC/C=C\CCCCCCCC(=O)OCCC1CCN(CCSSCCN2CCC(CCOC(=O)CCCCCCC/C=C\CCCCCCCC)CC2)CC1]

### Other Lipids

In some embodiments, the ionizable lipid in the LNPs of the present disclosure is selected from the group consisting of N-[1-(2,3-dioleyloxy)propyll-N,N,N-trimethylammonium chloride (DOTMA); N-[1-(2,3-dioleoyloxy)propyll-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1), N1-[2-((1S)-1-[(3-aminopropyl)amino]-4-[di(3-amino-propyl) aminolbutylc arboxamidoiethyl 1-3,4-di[oleyloxy]-benzamide (MVL5); Dioctadecylamido-glycylspermine (DOGS); 3b-[N—(N′,N′-dimethylaminoethyl) carb amoyl] cholesterol (DC-Chol); Dioctadecyldimethylammonium Bromide (DDAB); a Saint lipid (e.g., SAINT-2, N-methyl-4-(dioleyl)methylpyridinium); 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropy 1-3-dimethylhydroxyethyl ammonium chloride (DORI); Di-alkylated Amino Acid (DILA2) (e.g., C18:1-norArg-C16); Dioleyldimethylammonium chloride (DODAC); 1-palmitoyl-2-oleoyl-sn-glycero-3-ethylpho sphocholine (POEPC); and 1,2-dimyristoleoyl-sn-glycero-3-ethylphosphocholine (MOEPC). In some variations, the condensing agent, e.g., a cationic lipid, is a lipid such as, e.g., Dioctadecyldimethylammonium bromide (DDAB), 1,2-dilinoleyloxy-3-dimethylaminopropane (DLinDMA), 2,2-dilinoleyl-4-(2dimethylaminoethyl)-[1,31-dioxolane (DLin-KC2-DMA), heptatriaconta-6,9,28,31-tetraen-19-yl-4-(dimethylamino)butanoate (Dlin-MC3-DMA), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diyl dioleate hydrochloride (DODAPen-C₁), (R)-5-guanidinopentane-1,2-diyl dioleate hydrochloride (DOPen-G), and (R)-N,N,N-trimethyl-4,5-bis(oleoyloxy) pentan-1-aminium chloride (DOTAPen).

In some embodiments, the ionizable lipid in the LNP of the present disclosure is represented by the following structure:

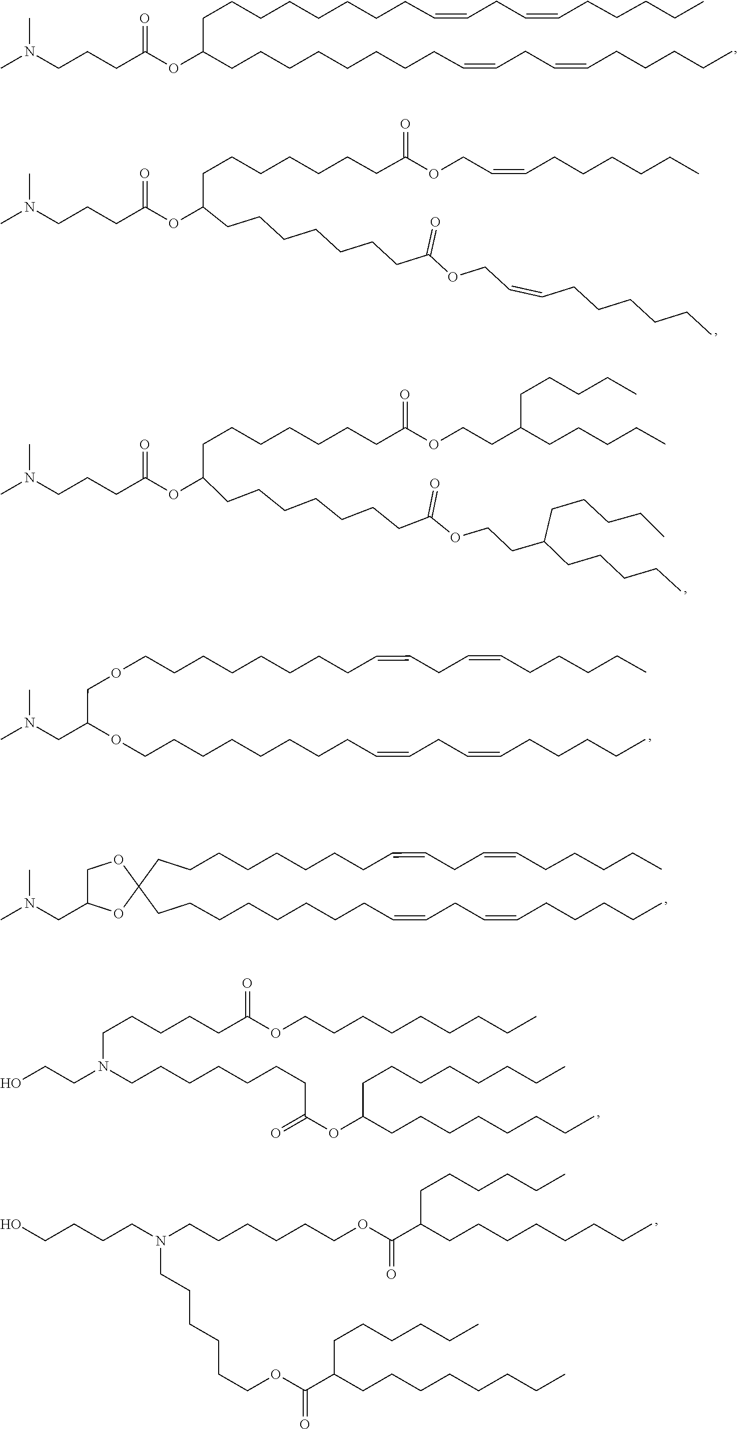

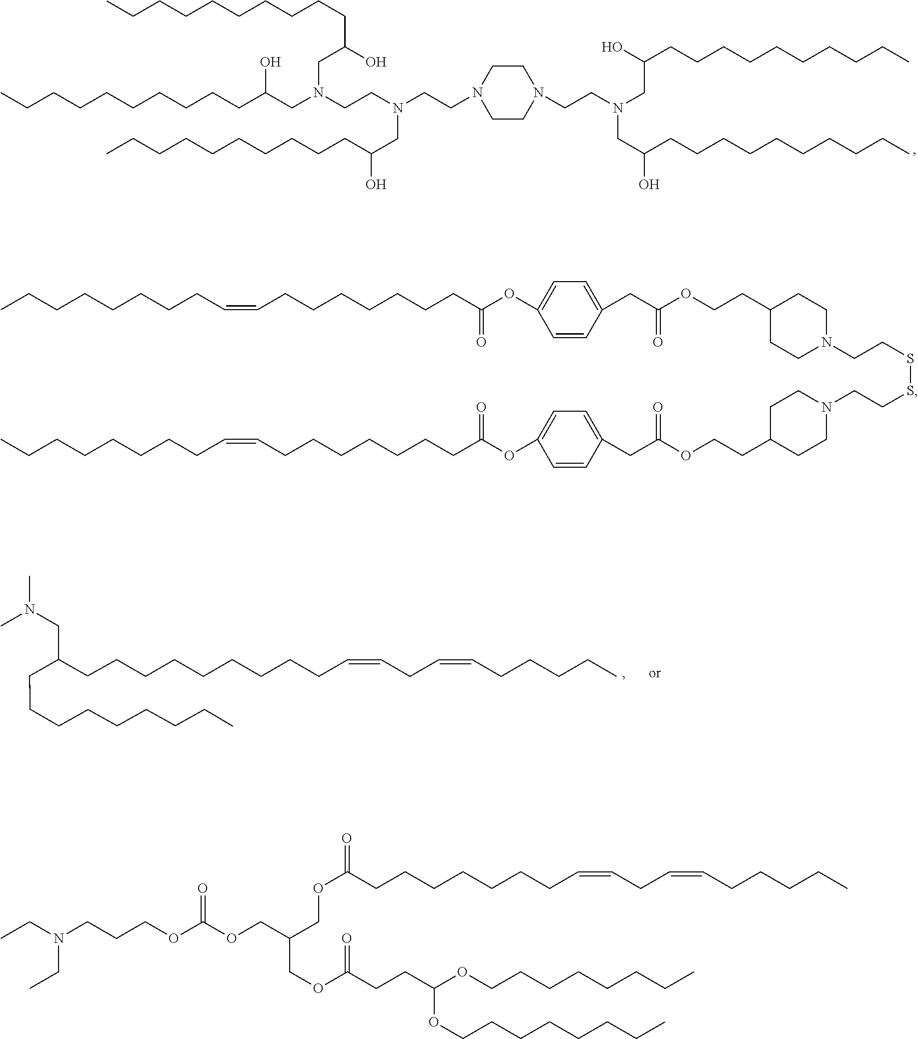

or a pharmaceutically acceptable salt or ester thereof, or a deuterated analogue thereof.

### B. Structural Lipids

In some embodiments, the LNPs provided by the present disclosure comprise a structural lipid. Without wishing to be bound by a specific theory, it is believed that a structural lipid, when present in an LNP, contributes to membrane integrity and stability of the LNP.

In some embodiments, the structural lipid is a sterol, e.g., cholesterol, or a derivative thereof. In one embodiment, the structural lipid is cholesterol. In another embodiment, the structural lipid is a derivative of cholesterol. Non-limiting examples of cholesterol derivatives include polar analogues such as 5α-cholestanol, 5β-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5β-cholestanone, and cholesteryl decanoate; and mixtures thereof. In some embodiments, the cholesterol derivative is a polar analogue such as cholesteryl-(4′-hydroxy)-butyl ether. In some embodiments, cholesterol derivative is cholestryl hemisuccinate (CHEMS).

Exemplary cholesterol derivatives are described in International Patent Application Publication No. WO2009/127060 and U.S. Patent Application Publication No. US2010/0130588, contents of both of which are incorporated herein by reference in their entirety.

In some embodiments, the sterol in the LNPs of the present disclosure is selected from the group consisting of cholesterol, beta-sitosterol, stigmasterol, beta-sitostanol, campesterol, brassicasterol, and derivatives thereof, and any combination thereof. In one embodiment, the sterol is cholesterol. In another embodiment, the sterol is beta-sitosterol.

In some embodiments, the structural lipid constitutes about 20 mol % to about 45 mol % of the total lipid present in the LNP. In some embodiments, the structural lipid constitutes about 25 mol % to about 45 mol % of the total lipid content of the LNP. In some embodiments, the structural lipid constitutes about 30 to about 45% of the total lipid present in the LNP. In some embodiments, the structural lipid constitutes about 30 mol % to about 40 mol % of the total lipid present in the LNP. In some embodiments, such a component is about 40 mol % of the total lipid present in the LNP. In some embodiments, the structural lipid, e.g., a sterol, constitutes about 20 mol % to about 45 mol % of the total lipid present in the LNP. In some embodiments, the structural lipid, e.g., a sterol, constitutes about 30 mol % to about 40 mol % of the total lipid present in the LNP.

In some embodiments, the structural lipid is cholesterol and constitutes about 30 mol % to about 45 mol % of the total lipid present in the LNP. In some embodiments, the structural lipid is cholesterol and constitutes about 35 mol % to about 45 mol % of the total lipid present in the LNP. In some embodiments, the structural lipid is cholesterol and constitutes about 40 mol % to about 45 mol % of the total lipid present in the LNP. In some embodiments, the structural lipid is cholesterol and constitutes about 40 mol % of the total lipid present in the LNP. In some embodiments, the structural lipid is cholesterol and constitutes about 45 mol % of the total lipid present in the LNP. In some embodiments, the structural lipid is cholesterol and constitutes about 40 mol % to about 45 mol % of the total lipid present in the LNP, wherein the encapsulation efficiency (“Enc. Eff.”) of TNA is greater than 95% and/or the average size of the LNP ranges about 70 nm to 90 nm in diameter.

In some embodiments, the structural lipid is dexamethasone or dexamethasone-palmitate.

### C. Helper Lipids

The LNPs provided by the present disclosure comprise a helper lipid. In some embodiments, the helper lipid is ceramide or sphingomyelin. Both ceramides and sphingomyelins are sphingolipids which is a class of cell membrane lipids. Structurally, both ceramides and sphingomyelins both contain an N-acetylsphingosine (i.e., (E)-N-(1,3-dihydroxyoctadec-4-en-2-yl) acetamide) backbone and a fatty acid linked to the amide group. In sphingomyelins, the N-acetylsphingosine backbone is further linked to a phosphocholine or phosphoethanolamine group. In some embodiments, the LNPs provided by the present disclosure comprise a ceramide or a sphingomyelin or a combination thereof, whereby the fatty acid portion of the ceramide or sphingomyelin is of a certain length or is a fatty acid having a certain number of carbon atoms as described below. As used herein, the term “helper lipid” refers to an amphiphilic lipid comprising at least one non-polar chain and at least one polar moiety. Without wishing to be bound by a specific theory, it is believed that a helper lipid functions to evade off-targeting of the LNP to the blood compartment, to increase the fusogenicity of the lipid bilayer of the LNP, to stabilize the LNP structure, and to facilitate endosomal escape.

In some embodiments, the ceramide or sphingomyelin in the LNPs of the present disclosure as a helper lipid is represented by a helper lipid represented by Formula (I):

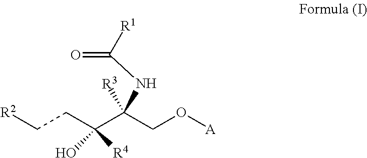

or a salt or an ester thereof, or a deuterated analogue of any of the foregoing, wherein:
[symbol] is a single bond or a double bond;

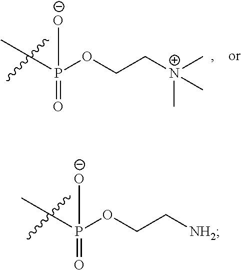 [SMILES: *P(=O)([O-])OCC[N+](C)(C)C ; *P(=O)([O-])OCCN]

A is hydrogen,
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (I), R¹ is C₁-C₁₀ alkyl or C₂-C₁₀ alkenyl.

In some embodiments of Formula (I),
R¹ is C₁-C₁₀ alkyl or C₂-C₁₀ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (I), R³ and R⁴ are both hydrogens. In some embodiments of Formula (I), R³ and R⁴ are independently hydrogen or C₁ alkyl.

In some embodiments of Formula (I), R¹ is C₁-C₇ alkyl or C₂-C₇ alkenyl. In one embodiment, R¹ is C₁-C₇ alkyl. In one embodiment, R¹ is C₁ alkyl.

In some embodiments, the helper lipid is not distearoylphosphatidylcholine (DSPC), provided that a helper lipid represented by (I), (II), (III), or (IV), or a salt or an ester thereof, or a deuterated analogue of any of the foregoing is present.

In some embodiments, the helper lipid is not 1,2-dioleoyl-sn-glycero-3-phosphocholine (DOPC), provided that a helper lipid represented by (I), (II), (III), or (IV), or a salt or an ester thereof, or a deuterated analogue of any of the foregoing is present.

In some embodiments, the helper lipid is not DOPE, provided that a helper lipid represented by (I), (II), (III), or (IV), or a salt or an ester thereof, or a deuterated analogue of any of the foregoing is present.

In some embodiments, the helper lipid is represented by Formula (II):

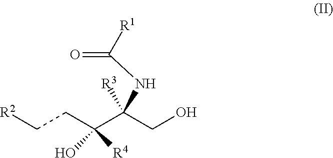

or a salt or an ester thereof, or a deuterated analogue of any of the foregoing, wherein R¹, R², R³ and R⁴ are as defined above in Formula (I).

In some embodiments of Formula (II), R³ and R⁴ are both hydrogens.

In some embodiments of Formula (II), R³ and R⁴ are independently hydrogen or C₁ alkyl.

In some embodiments of Formula (II), R¹ is C₁-C₇ alkyl or C₂-C₇ alkenyl. In one embodiment, R¹ is C₁-C₇ alkyl. In one embodiment, R¹ is C₁ alkyl.

In some embodiments, the helper lipid is represented by Formula (III):

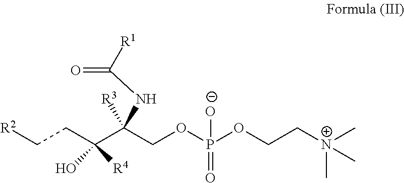

or a salt or an ester thereof, or a deuterated analogue of any of the foregoing, wherein R¹, R², R³ and R⁴ are as defined above in Formula (I).

In some embodiments of Formula (III), R³ and R⁴ are both hydrogens.

In some embodiments of Formula (III), R¹ is C₁-C₁₀ alkyl or C₂-C₁₀ alkenyl. In one embodiment, R¹ is C₁-C₁₀ alkyl.

In some embodiments, the helper lipid is represented by Formula (IV):

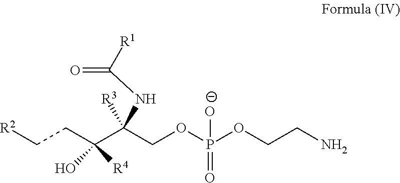

or a salt or an ester thereof, or a deuterated analogue of any of the foregoing, wherein R¹, R², R³ and R⁴ are as defined above in Formula (I).

As used herein, the term “salt” when referring to a helper lipid represented by Formula (I), Formula (II), Formula (III) or Formula (IV) means a pharmaceutically acceptable salt of a helper lipid represented by Formula (I), Formula (II), Formula (III) or Formula (IV), including both acid and base addition salts. A salt of a helper lipid represented by Formula (I), Formula (II), Formula (III) or Formula (IV) retains the biological effectiveness and properties of the free acid forms or free base forms of the helper lipid represented by Formula (I), Formula (II), Formula (III) or Formula (IV), which are not biologically or otherwise undesirable, and which are formed with inorganic acids or organic acids, or inorganic bases or organic bases. Examples of inorganic acids include, but are not limited to, hydrochloric acid, hydrobromic acid, sulfuric acid, nitric acid, phosphoric acid and the like; and examples of organic acids include, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

As used herein, the term “ester” when referring to a helper lipid represented by Formula (I), Formula (II), Formula (III) or Formula (IV) means an ester of a helper lipid represented by Formula (I), Formula (II), Formula (III) or Formula (IV). As a non-limiting example, a hydroxyl group of the helper lipid represented by Formula (I), Formula (II), Formula (III) or Formula (IV) may be linked to an organic acid such as phosphoric acid or carboxylic acid via the process of esterification to form an ester (e.g., a carboxylate or a phosphate) of a helper lipid represented by Formula (I), Formula (II), Formula (III) or Formula (IV).

As used herein, a “deuterated analogue” when referring to a helper lipid represented by Formula (I), Formula (II), Formula (III) or Formula (IV) means an analogue of a helper lipid represented by Formula (I), Formula (II), Formula (III) or Formula (IV), whereby any one or more hydrogen atoms of the lipid are substituted with deuterium, which is an isotope of hydrogen.

In some embodiments, an LNP of the present disclosure does not contain or comprise distearoylphosphatidylcholine (DSPC), provided that a helper lipid represented by (I), (II), (III), or (IV), or a salt or an ester thereof, or a deuterated analogue of any of the foregoing is present. In some embodiments, an LNP of the present disclosure does not contain or comprise 1,2-dioleoyl-sn-glycero-3-phosphocholine (DOPC), provided that a helper lipid represented by (I), (II), (III), or (IV), or a salt or an ester thereof, or a deuterated analogue of any of the foregoing is present. In some embodiments, an LNP of the present disclosure does not contain or comprise 1,2-dioleoyl-sn-glycero-3-phosphoethanolamine (DOPE), provided that a helper lipid represented by (I), (II), (III), or (IV), or a salt or an ester thereof, or a deuterated analogue of any of the foregoing is present.

In one embodiment of Formula (I), Formula (II), Formula (III) and Formula (IV), or a salt or an ester thereof, or a deuterated analogue of any of the foregoing, [symbol] is a double bond; R¹, R², R³ and R⁴ are as defined above. In an alternative embodiment of Formula (I), Formula (II), Formula (III) and Formula (IV), or a salt or an ester thereof, or a deuterated analogue of any of the foregoing, [symbol] is a single bond; R¹, R², R³ and R⁴ are as defined above.

In some embodiments of Formula (I), Formula (II), Formula (III) and Formula (IV), or a salt or an ester thereof, or a deuterated analogue of any of the foregoing, R¹ is C₁-C₁₅ alkyl or C₂-C₁₅ alkenyl.

In some embodiments of Formula (I), Formula (II), Formula (III) and Formula (IV), or a salt or an ester thereof, or a deuterated analogue of any of the foregoing:
R¹ is C₁-C₁₅ alkyl or C₂-C₁₅ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (I), Formula (II), Formula (III) and Formula (IV), or a salt or an ester thereof, or a deuterated analogue of any of the foregoing, R¹ is C₁-C₁₀ alkyl or C₂-C₁₀ alkenyl.

In some embodiments of Formula (I), Formula (II), Formula (III) and Formula (IV), or a salt or an ester thereof:
R¹ is C₁-C₁₀ alkyl or C₂-C₁₀ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (I), Formula (II), Formula (III) and Formula (IV), or a salt or an ester thereof, or a deuterated analogue of any of the foregoing, R¹ is C₁-C₈ alkyl or C₂-C₈ alkenyl. In one embodiment, R¹ is C₁-C₈ alkyl. In some embodiments of Formula (I)), Formula (II), Formula (III) and Formula (IV), or a salt or an ester thereof, or a deuterated analogue of any of the foregoing, R¹ is C₁-C₇ alkyl or C₂-C₇ alkenyl. In one embodiment, R¹ is C₁-C₇ alkyl. In one embodiment of Formula (I), Formula (II), Formula (III) and Formula (IV), or a salt or an ester thereof, or a deuterated analogue of any of the foregoing:
R¹ is C₁-C₇ alkyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

In some embodiments of Formula (I), Formula (II), Formula (III) and Formula (IV), or a salt or an ester thereof, or a deuterated analogue of any of the foregoing, R¹ is C₁ alkyl, C₂ alkyl, C₃ alkyl, C₄ alkyl, C₅ alkyl, C₆ alkyl, or C₇ alkyl. In some embodiments of Formula (I), Formula (II), Formula (III) and Formula (IV), or a salt or an ester thereof, or a deuterated analogue of any of the foregoing, R¹ is C alkyl, C₂ alkyl, C₃ alkyl, C₄ alkyl, C₅ alkyl, C₆ alkyl, or C₇ alkyl. In some embodiments of Formula (I), Formula (II), Formula (III) and Formula (IV), or a salt or an ester thereof, or a deuterated analogue of any of the foregoing, R¹ is C₁ alkyl, C₃ alkyl, C₅ alkyl, or C₇ alkyl. In one embodiment of Formula (I), Formula (II), Formula (III) and Formula (IV), or a salt or an ester thereof, or a deuterated analogue of any of the foregoing, R¹ is C₁ alkyl. In one embodiment of Formula (I), Formula (II), Formula (III) and Formula (IV), or a salt or an ester thereof, or a deuterated analogue of any of the foregoing, R¹ is C₃ alkyl. In one embodiment of Formula (I), Formula (II), Formula (III) and Formula (IV), or a salt or an ester thereof, or a deuterated analogue of any of the foregoing, R¹ is C₅ alkyl. In one embodiment of Formula (I), Formula (II), Formula (III) and Formula (IV), or a salt or an ester thereof, or a deuterated analogue of any of the foregoing, R¹ is C₇ alkyl.

In some embodiments of Formula (I), Formula (II), Formula (III) and Formula (IV), or a salt or an ester thereof, or a deuterated analogue of any of the foregoing, R² is C₃-C₁₅ alkyl or C₃-C₁₅ alkenyl; and R¹, R³ and R⁴ are as defined above. In some embodiments of Formula (I), Formula (II), Formula (III) and Formula (IV), or a salt or an ester thereof, or a deuterated analogue of any of the foregoing, R² is C₅-C₁₅ alkyl or C₃-C₁₅ alkenyl; and R¹, R³ and R⁴ are as defined above. In some embodiments of Formula (I), Formula (II), Formula (III) and Formula (IV), or a salt or an ester thereof, or a deuterated analogue of any of the foregoing, R² is C₇-C₁₅ alkyl or C₃-C₁₅ alkenyl; and R¹, R³ and R⁴ are as defined above. In some embodiments of Formula (I), Formula (II), Formula (III) and Formula (IV), or a salt or an ester thereof, or a deuterated analogue of any of the foregoing, R² is C₉-C₁₅ alkyl or C₉-C₁₅ alkenyl; and R¹, R³ and R⁴ are as defined above. In some embodiments of Formula (I), Formula (II), Formula (III) and Formula (IV), or a salt or an ester thereof, or a deuterated analogue of any of the foregoing, R² is C₉ alkyl, C₁₀ alkyl, C₁₁ alkyl, C₁₂ alkyl, C₁₃ alkyl, C₁₄ alkyl, or C₁₅ alkyl; and R¹, R³ and R⁴ are as defined above. In some embodiments of Formula (I), Formula (II), Formula (III) and Formula (IV), or a salt or an ester thereof, or a deuterated analogue of any of the foregoing, R² is C₉ alkyl; and R¹, R³ and R⁴ are as defined above. In some embodiments of Formula (I), Formula (II), Formula (III) and Formula (IV), or a salt or an ester thereof, or a deuterated analogue of any of the foregoing, R² is C₁₁ alkyl; and R¹, R³ and R⁴ are as defined above. In some embodiments of Formula (I), Formula (II), Formula (III) and Formula (IV), or a salt or an ester thereof, or a deuterated analogue of any of the foregoing, R² is C₁₃ alkyl; and R¹, R³ and R⁴ are as defined above.

In some embodiments of Formula (I), Formula (II), Formula (III) and Formula (IV), or a salt or an ester thereof, or a deuterated analogue of any of the foregoing, R³ is hydrogen or C₁ alkyl; and R¹, R² and R⁴ are as defined above. In one embodiment of Formula (I), Formula (II), Formula (III) and Formula (IV), or a salt or an ester thereof, or a deuterated analogue of any of the foregoing, R³ is hydrogen; and R¹, R² and R⁴ are as defined above. In one embodiment of Formula (I), Formula (II), Formula (III) and Formula (IV), or a salt or an ester thereof, or a deuterated analogue of any of the foregoing, R³ is C₁ alkyl; and R¹, R² and R⁴ are as defined above.

In some embodiments of Formula (I), Formula (II), Formula (III) and Formula (IV), or a salt or an ester thereof, or a deuterated analogue of any of the foregoing, R⁴ is hydrogen or C₁ alkyl; and R¹, R² and R³ are as defined above. In one embodiment of Formula (I), Formula (II), Formula (III) and Formula (IV), or a salt or an ester thereof, or a deuterated analogue of any of the foregoing, R⁴ is hydrogen; and R¹, R² and R³ are as defined above. In one embodiment of Formula (I), Formula (II), Formula (III) and Formula (IV), or a salt or an ester thereof, or a deuterated analogue of any of the foregoing, R⁴ is C₁ alkyl; and R¹, R² and R³ are as defined above.

In some embodiments of Formula (I), Formula (II), Formula (III) and Formula (IV), R¹ is C₁-C₇ alkyl or C₂-C₇ alkenyl.

In some embodiments, R¹ is C₁ alkyl, C₃ alkyl, C₅ alkyl, or C₇ alkyl. In some embodiments, R¹ is C₁ alkyl.

In some embodiments of Formula (I), Formula (II), Formula (III) and Formula (IV), R² is C₃-C₁₅ alkyl or C₃-C₁₅ alkenyl. In some embodiments, R² is C₁₀ alkyl, C₁₁ alkyl, C₁₂ alkyl, C₁₃ alkyl, C₁₄ alkyl, or C₁₅ alkyl. In some embodiments, R² is C₁₂ alkyl, C₁₃ alkyl, or C₁₄ alkyl. In some embodiments, R² is C₁₃ alkyl. In some embodiments, R² is C₁₂ alkyl. In some embodiments, R² is C₁₁ alkyl.

In some embodiments of Formula (I), Formula (II), Formula (III) and Formula (IV), both R¹ and R² are hydrogen; and [symbol] is a double bond.

In some embodiments of Formula (I), Formula (II), Formula (III) and Formula (IV), both R¹ and R² are hydrogen and [symbol] is a double bond; and R¹ is C₁ alkyl, C₃ alkyl, C₅ alkyl or C₇ alkyl. In one embodiment, R¹ is C₁ alkyl. In another embodiment, R¹ is C₃ alkyl. In yet another embodiment, R¹ is C₅ alkyl. In yet another embodiment, R¹ is C₇ alkyl.

In some embodiments of Formula (I), Formula (II), Formula (III) and Formula (IV), both R¹ and R² are hydrogen and [symbol] is a double bond; R¹ is C₁ alkyl, C₃ alkyl, C₅ alkyl or C₇ alkyl and R² is C₉ alkyl, C₁₁, or C₁₃ alkyl. In one embodiment, R² is C₉ alkyl. In one embodiment, R² is C₁₁ alkyl. In another embodiment, R² is C₁₃ alkyl.

In some embodiments of Formula (I), Formula (II), Formula (III) and Formula (IV), R³ is hydrogen. In some embodiments of Formula (I), Formula (II), Formula (III) and Formula (IV), R³ is C₁ alkyl.

In some embodiments of Formula (I), Formula (II), Formula (III) and Formula (IV), R⁴ is hydrogen. In some embodiments of Formula (I), Formula (II), Formula (III) and Formula (IV), R⁴ is C₁ alkyl.

In some embodiments, the helper lipid represented by Formula (I), Formula (II), Formula (III) or Formula (IV) (e.g., ceramide or sphingomyelin) in the LNPs of the present disclosure are as in Table 8 below, or a salt or an ester thereof, or a deuterated analogue of any of the foregoing.

TABLE 8
Exemplary helper lipids (e.g., ceramide or sphingomyelin) in the LNPs of the disclosure

| Name | Structure |
|---|---|
| C2 Ceramide (d18:1/2:0) | 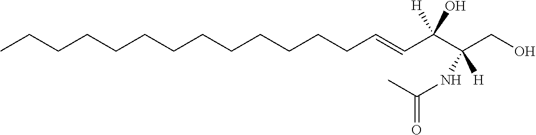 [SMILES: CCCCCCCCCCCCC/C=C/[C@@H](O)[C@H](CO)NC(C)=O] |
| C2 Ceramide (d14:1/2:0) | 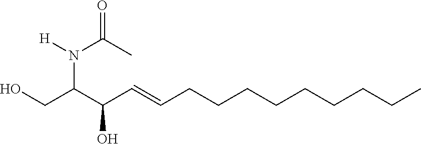 [SMILES: CCCCCCCCC/C=C/[C@@H](O)C(CO)NC(C)=O] |
| C2 Dihydroceramide (d18:0/2:0) | 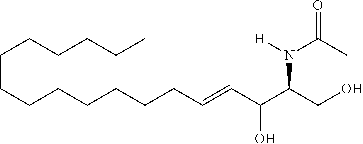 [SMILES: CCCCCCCCCCCCC/C=C/C(O)[C@H](CO)NC(C)=O] |
| C4 Ceramide (d18:1/4:0) | 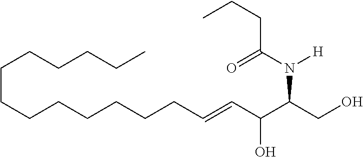 [SMILES: CCCCCCCCCCCCC/C=C/C(O)[C@H](CO)NC(=O)CCC] |
| C6 Ceramide (d18:1/6:0) | 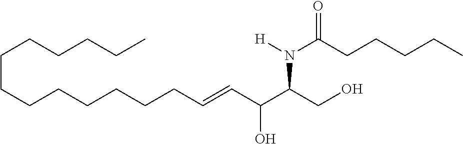 [SMILES: CCCCCCCCCCCCC/C=C/C(O)[C@H](CO)NC(=O)CCCCC] |
| C6 Dihydroceramide (d18:0/6:0) | 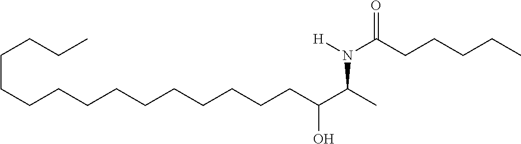 [SMILES: CCCCCCCCCCCCCCCC(O)[C@H](C)NC(=O)CCCCC] |
| C8 Ceramide (d18:1/8:0) | 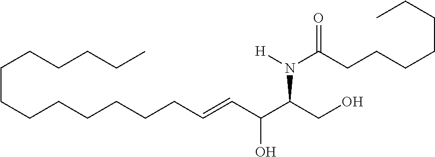 [SMILES: CCCCCCCCCCCCC/C=C/C(O)[C@H](CO)NC(=O)CCCCCCC] |
| C8 Dihydroceramide (d18:0/8:0) | 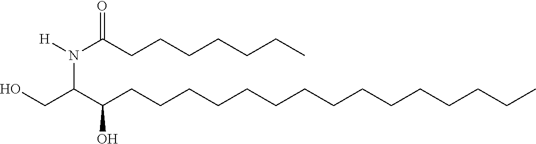 [SMILES: CCCCCCCCCCCCCCC[C@@H](O)C(CO)NC(=O)CCCCCCC] |
| C2 Sphingomyelin (d18:1/2:0) | 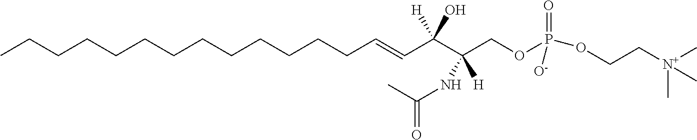 [SMILES: CCCCCCCCCCCCC/C=C/[C@@H](O)[C@H](COP(=O)([O-])OCC[N+](C)(C)C)NC(C)=O] |

In some embodiments, the helper lipid is DSPC, a salt or an ester thereof, or a deuterated analogue of any of the foregoing. In some embodiments, the helper lipid is DOPE, or a salt or an ester thereof, or a deuterated analogue of any of the foregoing. In some embodiments, the helper lipid is ceramide, a salt or an ester thereof, or a deuterated analogue of any of the foregoing.

As used herein, the term “salt” means a pharmaceutically acceptable salt of a helper lipid including both acid and base addition salts. A salt of a helper lipid retains the biological effectiveness and properties of the free acid forms or free base forms of the helper lipid.

As used herein, the term “ester” when referring to a helper lipid means an ester of a helper lipid. As a non-limiting example, a hydroxyl group of the helper lipid may be linked to an organic acid such as phosphoric acid or carboxylic acid via the process of esterification to form an ester (e.g., a carboxylate or a phosphate) of a helper lipid.

As used herein, a “deuterated analogue” when referring to a helper lipid means an analogue of a helper lipid that any one or more hydrogen atoms of the helper lipid are substituted with deuterium.

In some embodiments, an LNP of the present disclosure does not contain or comprise a helper lipid (e.g., distearoylphosphatidylcholine (DSPC), 1,2-dioleoyl-sn-glycero-3-phosphocholine (DOPC), or 1,2-dioleoyl-sn-glycero-3-phosphoethanolamine (DOPE).

In some embodiments, the helper lipid (e.g., ceramide of this disclosure constitutes about 2 mol % to about 40 mol % of the total lipid present in the LNP, or about 5 mol % to about 40 mol %, or about 5 mol % to about 35 mol %, or about 5 mol % to about 30 mol %, or about 5 mol % to about 25 mol %, or about 5 mol % to about 20 mol %, or about 5 mol % to about 15 mol %, or 10 mol % to about 40 mol %, or about 10 mol % to about 35 mol %, or about 10 mol % to about 30 mol %, or about 10 mol % to about 25 mol %, or about 10 mol % to about 20 mol %, or 15 mol % to about 40 mol %, or about 15 mol % to about 35 mol %, or about 15 mol % to about 30 mol %, or about 15 mol % to about 25 mol %, or about 15 mol % to about 20 mol %, or 20 mol % to about 40 mol %, or about 20 mol % to about 35 mol %, or about 20 mol % to about 30 mol %, or about 20 mol % to about 25 mol %, or 25 mol % to about 40 mol %, or about 25 mol % to about 35 mol %, or about 25 mol % to about 30 mol %, or 30 mol % to about 40 mol %, or about 30 mol % to about 35 mol %, or about 35 mol % to about 40 mol %, or about 5 mol %, or about 10 mol %, or about 15 mol %, or about 20%, or about 25 mol %, or about 30 mol %, or about 35 mol %, or about 40 mol %. In some embodiments, the helper lipid (e.g., DSPC, DOPE, ceramide, etc.) constitutes about 10% mol to about 20 mol % of the total lipid present in the LNP and such LNP having about 10% mol to about 20 mol % of the total lipid present in the LNP demonstrate overall increased tolerability (e.g., as demonstrated in body weight loss profiles in a subject and reduced cytokine response), as compared to the LNP comprising less than 10% of the same helper lipid.

### D. Lipid-Anchored Polymers

In some embodiments, the LNPs provided by the present disclosure comprise at least one type of lipid-anchored polymer, i.e., a first lipid-anchored polymer. As used herein, the term “lipid-anchored polymer” refers to a molecule comprising a lipid moiety covalently attached to a polymer, optionally via a linker. Without wishing to be bound by a specific theory, it is believed that a lipid-anchored polymer can inhibit aggregation of LNPs and provide steric stabilization. In some embodiments, the LNPs provided by the present disclosure comprise two lipid-anchored polymers, i.e., a first lipid-anchored polymer and a second lipid-anchored polymer.

### Lipid Moieties in Lipid-Anchored Polymers

More specifically, in one embodiment, a lipid-anchored polymer, e.g., a first lipid-anchored polymer in accordance with the present disclosure comprises:
(i) a polymer;
(ii) a lipid moiety comprising at least one hydrophobic tail (which may be linear or branched); and
(iii) optionally a linker connecting the polymer to the lipid moiety;
   wherein the at least one hydrophobic tail (which may be linear or branched) comprises 16 to 22 carbon atoms in a single aliphatic chain backbone, i.e., 16, 17, 18, 19, 20, 21, or 22 carbon atoms in a single aliphatic chain backbone. In one embodiment, the lipid-anchored polymer, e.g., a first lipid-anchored polymer comprises a lipid moiety comprising a single or two hydrophobic tails, wherein the single or two hydrophobic tails each comprise 16 to 22 carbon atoms in a single aliphatic chain backbone, i.e., 16, 17, 18, 19, 20, 21, or 22 carbon atoms in a single aliphatic chain backbone. In one embodiment, the single or two hydrophobic tails each comprise between 18 to 22 carbon atoms in a single aliphatic chain backbone. In another embodiment, the single or two hydrophobic tails each comprise between 18 to 20 carbon atoms in a single aliphatic chain backbone. In a particular embodiment, the single or two hydrophobic tails each comprise 18 carbon atoms in a single aliphatic chain backbone. In another embodiment, the single or two hydrophobic tails each comprise at least 18 carbon atoms in a single aliphatic chain backbone.

The term “linker-lipid moiety”, as used herein, refers to a lipid moiety comprising at least two hydrophobic tails, e.g., two hydrophobic tails, covalently attached to a linker. In some embodiments, the linker-lipid moiety may be a part of a lipid-anchored polymer.

In one embodiment, the at least one (e.g., single or two) hydrophobic tail is a fatty acid. Non-limiting examples of the at least one (e.g., single or two) hydrophobic tail comprising 16 to 22 carbon atoms in a single aliphatic chain backbone include octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

The term “derivative,” when used herein in reference to hydrophobic tails in a lipid-anchored polymer, refers to a hydrophobic tail that has been modified as compared to the original or native hydrophobic tail. In some embodiments, the derivative contains one or more of the following modifications as compared to the original or native hydrophobic tail: a) carboxylate group has been replaced with an amine group, an amide group, an ether group, or a carbonate group; b) one or more points of saturation, e.g., double bonds, have been introduced into (e.g., via dehydrogenation) the hydrophobic tail; c) one or more points of saturation, e.g., double bonds, have been removed from (e.g., via hydrogenation) the hydrophobic tail; and d) configuration of one or more double bonds, if present, has been changed, e.g., from a cis configuration to a trans configuration, or from a trans configuration to a cis configuration. The derivative contains the same number of carbon atoms as its original or native hydrophobic tail.

As used herein the term “a single aliphatic chain backbone” when referring to a hydrophobic tail in a lipid-anchored polymer refers to the main linear aliphatic chain or carbon chain, i.e., the longest continuous linear aliphatic chain or carbon chain. For example, the alkyl chain below that has several branchings contains 18 carbon atoms in a single aliphatic chain backbone, i.e., the longest continuous linear alkyl chain contains 18 carbon atoms. Note that the one or two carbon atoms (all indicated with *) in the several branching points are not included in the carbon atom count in the single aliphatic chain backbone.

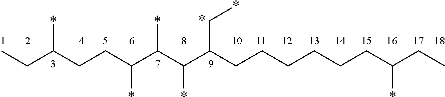 [SMILES: *CC(CCCCCCC(*)CC)C(*)C(*)C(*)CCC(*)CC]

In one embodiment, a lipid-anchored polymer or a first lipid-anchored polymer in accordance with the present disclosure comprises:
(i) a polymer;
(ii) a lipid moiety comprising at least two hydrophobic tails (which may be linear or branched); and
(iii) optionally a linker connecting the polymer to the lipid moiety;
   wherein the at least two hydrophobic tails (which may be linear or branched) comprise 16 to 22 carbon atoms in a single aliphatic chain backbone, i.e., 16, 17, 18, 19, 20, 21, or 22 carbon atoms in a single aliphatic chain backbone. In one embodiment, the lipid-anchored polymer or first lipid-anchored polymer comprises a lipid moiety comprising two hydrophobic tails, wherein the two hydrophobic tails each independently comprise 16 to 22 carbon atoms in a single aliphatic chain backbone, i.e., 16, 17, 18, 19, 20, 21, or 22 carbon atoms in a single aliphatic chain backbone. In one embodiment, the two hydrophobic tails each independently comprise 16 to 21 carbon atoms in a single aliphatic chain backbone, i.e., 16, 17, 18, 19, 20, or 21 carbon atoms in a single aliphatic chain backbone. In one embodiment, the two hydrophobic tails each independently comprise 16 to 20 carbon atoms in a single aliphatic chain backbone, i.e., 16, 17, 18, 19, or 20 carbon atoms in a single aliphatic chain backbone. In one embodiment, the two hydrophobic tails each independently comprise 16 to 19 carbon atoms in a single aliphatic chain backbone, i.e., 16, 17, 18, or 19 carbon atoms in a single aliphatic chain backbone. In one embodiment, the two hydrophobic tails each independently comprise 16 to 18 carbon atoms in a single aliphatic chain backbone, i.e., 16, 17, or 18 carbon atoms in a single aliphatic chain backbone. In one embodiment, the two hydrophobic tails each independently comprise 16 or 18 carbon atoms in a single aliphatic chain backbone. In one embodiment, the two hydrophobic tails each independently comprise 16 or 20 carbon atoms in a single aliphatic chain backbone. In one embodiment, the two hydrophobic tails each independently comprise 18 or 20 carbon atoms in a single aliphatic chain backbone. In one embodiment, the two hydrophobic tails each comprise 16 carbon atoms in a single aliphatic chain backbone. In one embodiment, the two hydrophobic tails each comprise 17 carbon atoms in a single aliphatic chain backbone. In one embodiment, the two hydrophobic tails each comprise 18 carbon atoms in a single aliphatic chain backbone. In one embodiment, the two hydrophobic tails each comprise 19 carbon atoms in a single aliphatic chain backbone. In one embodiment, the two hydrophobic tails each comprise 20 carbon atoms in a single aliphatic chain backbone.

In one embodiment, the at least two hydrophobic tails (e.g., two) are each a fatty acid. Non-limiting examples of the at least two hydrophobic tails comprising 16 to 22 carbon atoms in a single aliphatic chain backbone include octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

In one embodiment, a lipid-anchored polymer or a first lipid-anchored polymer in accordance with the present disclosure comprises:
(i) a polymer;
(ii) a lipid moiety comprising at least two hydrophobic tails (which may be linear or branched); and
(iii) optionally a linker connecting the polymer to the lipid moiety;
   wherein the at least two hydrophobic tails (which may be linear or branched) comprise 12 to 15 carbon atoms in a single aliphatic chain backbone, i.e., 12, 13, 14, or 15 carbon atoms in a single aliphatic chain backbone. In one embodiment, the lipid-anchored polymer or first lipid-anchored polymer comprises a lipid moiety comprising two hydrophobic tails, wherein the two hydrophobic tails each independently comprise 12 to 15 carbon atoms in a single aliphatic chain backbone, i.e., 12, 13, 14, or 15 carbon atoms in a single aliphatic chain backbone. In one embodiment, the two hydrophobic tails each independently comprise 12 to 14 carbon atoms in a single aliphatic chain backbone, i.e., 12, 13, or 14 carbon atoms in a single aliphatic chain backbone. In one embodiment, the two hydrophobic tails each independently comprise 12 or 14 carbon atoms in a single aliphatic chain backbone. In one embodiment, the two hydrophobic tails each comprise 12 carbon atoms in a single aliphatic chain backbone. In one embodiment, the two hydrophobic tails each comprise 13 carbon atoms in a single aliphatic chain backbone. In one embodiment, the two hydrophobic tails each comprise 14 carbon atoms in a single aliphatic chain backbone. In one embodiment, the two hydrophobic tails each comprise 15 carbon atoms in a single aliphatic chain backbone.

In one embodiment, one of the two hydrophobic tails is a fatty acid. Non-limiting examples of the at least two hydrophobic tails comprising 12 to 15 carbon atoms in a single aliphatic chain backbone include lauric acid, myristic acid, myristoleic acid, and a derivative thereof.

In one embodiment, a lipid-anchored polymer or a first lipid-anchored polymer in accordance with the present disclosure comprises:
(i) a polymer;
(ii) a lipid moiety comprising a single hydrophobic tail (which may be linear or branched); and optionally
(iii) a linker connecting the polymer to the lipid moiety;
   wherein the single hydrophobic tail (which may be linear or branched) comprises 12 to 22 carbon atoms in a single aliphatic chain backbone, i.e., 12, 13, 14, 15, 16, 17, 18, 19, 20, 21, or 22 carbon atoms in a single aliphatic chain backbone. In one embodiment, the lipid-anchored polymer or first lipid-anchored polymer comprises a lipid moiety comprising a single hydrophobic tail, wherein the single hydrophobic tail comprises 12 to 22 carbon atoms in a single aliphatic chain backbone, i.e., 12, 13, 14, 15, 16, 17, 18, 19, 20, 21, or 22 carbon atoms in a single aliphatic chain backbone. In one embodiment, the single hydrophobic tail comprises 12, 14, 16, 18, 20, or 22 carbon atoms in a single aliphatic chain backbone. In one embodiment, the single hydrophobic tail comprises 16 to 20 carbon atoms in a single aliphatic chain backbone, i.e., 16, 17, 18, 19, or 20 carbon atoms in a single aliphatic chain backbone. In one embodiment, the single hydrophobic tail comprises 16 to 19 carbon atoms in a single aliphatic chain backbone, i.e., 16, 17, 18, or 19 carbon atoms in a single aliphatic chain backbone. In one embodiment, the single hydrophobic tail comprises 16 to 18 carbon atoms in a single aliphatic chain backbone, i.e., 16, 17, or 18 carbon atoms in a single aliphatic chain backbone. In one embodiment, the single hydrophobic tail comprises 12 carbon atoms in a single aliphatic chain backbone. In one embodiment, the single hydrophobic tail comprises 13 carbon atoms in a single aliphatic chain backbone. In one embodiment, the single hydrophobic tail comprises 14 carbon atoms in a single aliphatic chain backbone. In one embodiment, the single hydrophobic tail comprises 15 carbon atoms in a single aliphatic chain backbone. In one embodiment, the single hydrophobic tail comprises 16 carbon atoms in a single aliphatic chain backbone. In one embodiment, the single hydrophobic tail comprises 17 carbon atoms in a single aliphatic chain backbone. In one embodiment, the single hydrophobic tail comprises 18 carbon atoms in a single aliphatic chain backbone. In one embodiment, the single hydrophobic tail comprises 19 carbon atoms in a single aliphatic chain backbone. In one embodiment, the single hydrophobic tail comprises 20 carbon atoms in a single aliphatic chain backbone. In one embodiment, the single hydrophobic tail comprises 21 carbon atoms in a single aliphatic chain backbone. In one embodiment, the single hydrophobic tail comprises 22 carbon atoms in a single aliphatic chain backbone.

In one embodiment, the single hydrophobic tail is a fatty acid. Non-limiting examples of the single hydrophobic tail comprising 12 to 22 carbon atoms in a single aliphatic chain backbone include lauric acid, myristic acid, myristoleic acid, octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

### Linkers in Lipid-Anchored Polymers

In some embodiments, in a lipid-anchored polymer of the present disclosure, a lipid moiety is covalently directly attached to a polymer or optionally via a linker. In some embodiments, the linker in the lipid-anchored polymer of the present disclosure is a glycerol linker, a phosphate linker, an ether linker, an amide linker, an amine linker, a peptide linker, a phosphoethanolamine linker, a phosphocholine linker, or any combination thereof. In some embodiments, the linker in the lipid-anchored polymer in the LNPs of the present disclosure a glycerol linker. Accordingly, in some embodiments, the lipid-anchored polymer in the LNPs of the present disclosure is a glycerolipid, wherein the glycerolipid comprises glycerol as a linker and one or more two lipid moieties as described above, e.g., distearoyl-rac-glycerol (DSG).

In some embodiments, the linker in the lipid-anchored polymer in the LNPs of the present disclosure is a phosphate linker. Accordingly, in some embodiments, the lipid-anchored polymer in the LNPs of the present disclosure is a phospholipid, wherein the phospholipid comprises a phosphate group as a linker and one or more lipid moieties as described above.

In some embodiments, the lipid-anchored polymer in an LNP of the present disclosure is both a glycerolipid and a phospholipid, such as 1,2-distearoyl-sn-glycero-3-phosphoethanolamine (DSPE).

In some embodiments, the first lipid-anchored polymer comprises a linker-lipid moiety (i.e., with one or more hydrophobic tails containing 16 to 22 carbon atoms in a single aliphatic chain) selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), distearoyl-rac-glycerol (DSG), 1,2-dipalmitoyl-rac-glycerol (DPG), a derivative thereof, and a combination any of the foregoing.

As used herein, the term “derivative” when used in reference to a linker-lipid moiety means a linker-lipid moiety containing one or more of the following modifications: a) a phosphatidylethanolamine (PE) head group, if present, is modified to convert an amino group into a methylamino group or a dimethylamino group; b) the modified linker-lipid moiety comprises one or more additional functional groups or moieties, such as —OH, —OCH₃, —NH₂, a maleimide, an azide or a cyclooctyne such as dibonzeocyclooctyne (DBCO).

In one embodiment, the first lipid-anchored polymer comprises a linker-lipid moiety (i.e., with one or more hydrophobic tails containing 16 to 22 carbon atoms in a single aliphatic chain) selected from the group consisting of DOPE, DSPE, DSG, DODA, DPG, a derivative thereof, and a combination of any of the foregoing.

In some embodiments, the first lipid-anchored polymer comprises a linker-lipid moiety (i.e., with one or more hydrophobic tails containing 12 to 15 carbon atoms in a single aliphatic chain) selected from the group consisting of 1,2-dimyristoyl-rac-glycero-3-methoxy (DMG), R-3-[(ω-methoxycarbamoyl)]-1,2-dimyristyloxl-propyl-3-amine, a derivative thereof, and a combination of any of the foregoing. In one embodiment, the first lipid-anchored polymer comprises DMG.

### Polymers in Lipid-Anchored Polymers

In some embodiments, the polymer in the lipid-anchored polymer is selected from the group consisting of polyethylene (PE), polypropylene (PP), polyethylene glycol (PEG), polyglycerol (PG), polyvinyl alcohol (PVOH), polysarcosine (pSar), and a combination thereof. In one embodiment, the polymer is selected from the group consisting of polyethylene glycol (PEG), polyglycerol (PG), polysarcosine (pSar), and a combination thereof.

In one embodiment, the polymer is polyethyelene glycol (PEG). In another embodiment, the polymer is polyglycerol (PG).

In some embodiments, the polymer in the lipid-anchored polymer has a molecular weight of about 5000 Da or less, e.g., about 4500 Da or less, about 4000 Da or less, about 3500 Da or less, about 3200 Da or less, about 3000 Da or less, about 2500 Da or less, about 2000 Da or less, about 1500 Da or less, about 1000 Da or less, about 500 Da or less, about 100 Da or less or about 50 Da or less. In some embodiments, the polymer in the lipid-anchored polymer has an average molecular weight of about 20 Da to about 100 Da, about 50 Da to about 500 Da, about 500 Da to about 2000 Da, about 1000 Da to about 5000 Da, e.g., about 2000 Da to about 5000 Da, about 1000 Da to about 3000 Da, about 1500 Da to about 2500 Da, about 2000 Da to about 4000 Da or about 2000 Da to about 5000 Da. In some embodiments, the polymer in the lipid-anchored polymer has an average molecular weight of about 1000 Da, about 1500 Da, about 2000 Da, about 2500 Da, about 3000 Da, about 3200 Da, about 3300 Da, about 3350 Da, about 3400 Da, about 3500 Da, about 4000 Da, about 4500 Da or about 5000 Da. In some embodiments, the polymer in the lipid-anchored polymer has an average molecular weight of about 2000 Da. In some embodiments, the polymer in the lipid-anchored polymer has an average molecular weight of about 2000 Da. In some embodiments, the polymer in the lipid-anchored polymer has an average molecular weight of about 3200 Da to about 3500 Da. In some embodiments, the polymer in the lipid-anchored polymer has an average molecular weight of about 3300 Da. In some embodiments, the polymer in the lipid-anchored polymer has an average molecular weight of about 3350 Da. In some embodiments, the polymer in the lipid-anchored polymer has an average molecular weight of about 3400 Da. In some embodiments, the polymer in the lipid-anchored polymer has an average molecular weight of about 3500 Da.

### Targeting Moiety and Second Lipid-Anchored Polymer

In some embodiments, an LNP of the present disclosure further comprises one or more targeting moieties. The targeting moiety targets the LNP for delivery to a specific cell type or a tissue in a subject, e.g., liver, bone marrow, spleen, blood, etc. In some embodiments, the targeting moiety is capable of binding to specific cell types e.g., hepatocytes, T-cells, B cells, NK cell, dendritic cells, etc. In some embodiments, the one or more targeting moieties are conjugated to a second lipid-anchored polymer. In some embodiments, the one or more targeting moieties conjugated to the second lipid-anchored polymer can be an antibody.

The antibodies may be intact monoclonal or polyclonal antibodies, and immunologically active fragments (e.g., a Fab or (Fab) 2 fragment), an antibody heavy chain, an antibody light chain, humanized antibodies, a genetically engineered single chain Fv (scFv) molecule, or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art. In one embodiment the targeting moiety is an antibody or an antibody fragment, e.g., an antibody or an antibody fragment that is capable of specifically binding to an antigen present on the surface of a cell In one embodiment the antibody or an antibody fragment is a monoclonal antibody (mAb), a single chain variable fragment (scFv), a heavy chain antibody (hcAb), a nanobody (Nb), a heavy-chain-only immunoglobulin (HCIg), an immunoglobulin new antigen receptor (IgNAR), variable domain of immunoglobulin new antigen receptor (VNAR), a single-domain antibody, or a variable heavy chain-only antibody (VHH). In one embodiment, the antibody target moiety is scFv. In another embodiment, the antibody targeting moiety is IgG. In yet another embodiment, the antibody targeting moiety is VHH (e.g., nanobody). In some embodiments, the targeting moiety is an antibody directed to an epitope present on a target cell. In some embodiments, the target cell is selected from the group consisting of T cell, B cell, NK cell, dendritic cell, hematopoietic cells, neuronal cell, and hepatocytes. In some embodiments, the target cell is T cell. In some embodiments, the antibody targeting moiety binds an epitope of T cell receptor (TCR), CD3, CD4, CD5, CD6, CD7, CD8, CD9, CD10, CD11, CD19, CD21, CD28, or PD-1.

In some embodiments, an LNP of the present disclosure further comprises one or more targeting moieties capable of binding to specific liver cells, such as hepatocytes. In one embodiment, the targeting moiety is capable of binding to the asialoglycoprotein receptor (ASGPR), i.e., hepatocyte-specific ASGPR. In one embodiment, the targeting moiety comprises an N-acetylgalactosamine molecule (GalNAc) or a GalNAc derivative thereof. As used herein, a “GalNAc derivative” refers to a modified GalNAc molecule or a conjugate of one or more GalNAc molecules (modified or unmodified) covalently linked to, for example, a lipid-anchored polymer as defined herein. In one embodiment, the targeting moiety is a tri-antennary or tri-valent GalNAc conjugate (i.e., GalNAc3) which is a ligand conjugate having three GalNAc molecules or three GalNAc derivatives. In one embodiment, the targeting moiety is a tri-antennary GalNAc represented by the following structural formula:

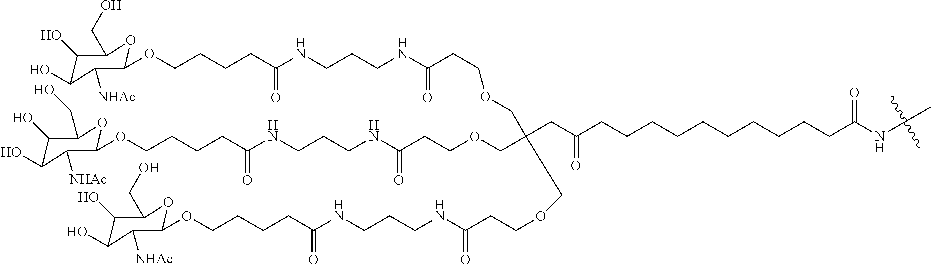 [SMILES: *NC(=O)CCCCCCCCCCC(=O)CC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In one embodiment, the targeting moiety is a tetra-antennary GalNAc conjugate. In one embodiment, the targeting moiety is a tetra-antennary or tetra-valent GalNAc conjugate (i.e., GalNAc4) which is a ligand having four GalNAc molecules or four GalNAc derivatives.

In one embodiment, the targeting moiety is capable of binding to low-density lipoprotein receptors (LDLRs), e.g., hepatocyte-specific LDLRs. In one embodiment, the targeting moiety comprises an apolipoprotein E (ApoE) protein, an ApoE polypeptide (or peptide), an apolipoprotein B (ApoB) protein, an ApoB polypeptide (or peptide), a fragment of any of the foregoing, or a derivative of any of the foregoing. In one embodiment, the ApoE polypeptide, ApoB polypeptide, or a fragment thereof is a ApoE polypeptide, ApoB polypeptide, or a fragment thereof as disclosed in International Patent Application Publication No. WO2022/261101, which is incorporated herein by reference in its entirety. In one embodiment, the ApoE protein is a modified ApoE protein and the ApoB protein is a modified ApoB protein. In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVCGRLVQYR GEVQAMLGQSTEELRVRLASHLRKLRKRLLRDADDLQKRLAVYQAGAREGAERGLSAIRER LGPLVEQGRVR (SEQ ID NO: 1). In one embodiment, the ApoE protein comprises, or consists of, the amino acid sequence set forth in SEQ ID NO: 1. In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVCGRLVQYR GEVQAMLGQSTEELRVRLASHLRKLRKRLLRDADDLQKRLAVYQAGAREGAERGLSAIRER LGPLVEQGRVRHHHHHH (SEQ ID NO: 2). In one embodiment, the ApoE protein comprises the amino acid sequence set forth in SEQ ID NO: 2. In one embodiment, the ApoE protein consists of the amino acid sequence set forth in SEQ ID NO: 2. In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVSGRLVQYR GEVQAMLGQSTEELRVRLASHLRKLRKRLLRDADDLQKRLAVYQAGAREGAERGLSAIRER LGPLVEQGRVR (SEQ ID NO: 3). In one embodiment, the ApoE protein comprises, or consists of, the amino acid sequence set forth in SEQ ID NO: 3. In one embodiment, the ApoE protein has an amino acid sequence having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98, or at least about 99% sequence identity to the following amino acid sequence: MKVEQAVETEPEPELRQQTEWQSGQRWELALGRFWDYLRWVQTLSEQVQEELLSSQVTQE LRALMDETMKELKAYKSELEEQLTPVAEETRARLSKELQAAQARLGADMEDVSGRLVQYR GEVQAMLGQSTEELRVRLASHLRKLRKRLLRDADDLQKRLAVYQAGAREGAERGLSAIRER LGPLVEQGRVRHHHHHHGGSSGSGC (SEQ ID NO: 4). In one embodiment, the ApoE protein comprises the amino acid sequence set forth in SEQ ID NO: 4. In one embodiment, the ApoE protein consists of the amino acid sequence set forth in SEQ ID NO: 4.

As used herein, the term “sequence identity” refers to the ratio of the number of identical amino acids between the 2 aligned sequences over the aligned length, expressed as a percentage. In some embodiments, the 2 aligned sequences are identical in length, i.e., have the same number of amino acids.

In one embodiment, the targeting moiety in an LNP of the present disclosure is an ApoE protein conjugate in an ApoB protein conjugate, which is a conjugate of one or more ApoE and/or ApoB protein molecules (native or modified) or a fragment thereof covalently linked to, for example, a lipid-anchored polymer as defined herein. In one embodiment, the targeting moiety in an LNP of the present disclosure is an ApoE polypeptide conjugate in an ApoB polypeptide conjugate, which is a conjugate of one or more ApoE and/or ApoB polypeptide molecules or a fragment thereof covalently linked to, for example, a lipid-anchored polymer as defined herein.

Accordingly, one key embodiment of an LNP of the present disclosure is that the LNP comprises a second lipid-anchored polymer and the targeting moiety as defined herein (and including GalNAc, ApoE protein, ApoB protein, ApoE polypeptide, ApoB polypeptide) is conjugated to the second lipid-anchored polymer. The second lipid-anchored polymer is structurally similar to the first lipid-anchored polymer as described herein in that the second lipid-anchored polymer also contains a lipid moiety covalently attached to a polymer via a linker. In one embodiment, the second lipid-anchored polymer comprises a linker-lipid moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), distearoyl-rac-glycerol (DSG), 1,2-dipalmitoyl-rac-glycerol (DPG), a derivative thereof, and a combination any of the foregoing. In one embodiment, the second lipid-anchored polymer comprises a linker-lipid moiety selected from the group consisting of DSPE, DSG, DODA, DPG, DOPE, and a derivative of thereof, and a combination of any of the foregoing.

In one embodiment, the ApoE protein, ApoB protein, ApoE polypeptide, ApoB polypeptide, or a fragment thereof, is covalently linked to a lipid-anchored polymer (e.g., second lipid-anchored polymer) or to an LNP of the present disclosure via strain promoted alkyne-azide cycloaddition (SPAAC) chemistry, such as via an azide-modified lipid-anchored polymer (e.g., DSG-PEG2000-azide, DSPE-PEG2000-azide, DSG-PEG3400-azide, DSPE-PEG3400-azide, DSG-PEG5000-azide, DSPE-PEG5000-azide) and a dibenzocyclooctyne (DBCO)-functionalized ApoE protein, ApoB protein, ApoE polypeptide, ApoB polypeptide, or a fragment thereof.

In some embodiments, the LNPs of the present disclosure may comprise a first lipid-anchored polymer and a second lipid-anchored polymer. For example, the LNPs of the present disclosure may comprise a first lipid-anchored polymer that does not comprise a targeting moiety, and a second type of lipid-anchored polymer that comprises a targeting moiety, such as GalNAc. For example, the LNPs of the present disclosure may comprise DSG-PEG2000 modified to comprise an additional OCH₃ group (DSG-PEG2000-OMe) as a first lipid-anchored polymer and DSPE-PEG2000-GalNAc3 as a second lipid-anchored polymer.

In some embodiments, the LNPs of the present disclosure may comprise a first lipid-anchored polymer and a second lipid-anchored polymer, wherein the second lipid-anchored polymer comprises a targeting moiety. In some embodiments, the second lipid-anchored polymer comprises a lipid moiety selected from the group consisting of DSPE, DSG, DODA, DPG, DOPE, and a derivative of thereof. In some embodiments, the first lipid-anchored polymer is any lipid-anchored polymer as described hereinabove.

In one embodiment, the LNP of the present disclosure comprises a second lipid-anchored polymer and the targeting moiety as defined herein (e.g., mAb, IgG, scFv, VHH, GalNAc, ApoE protein or peptide, ApoB protein or peptide) is conjugated to the second lipid-anchored polymer. The second lipid-anchored polymer is structurally similar to the first lipid-anchored polymer in that the second lipid-anchored polymer also contains a lipid moiety comprising a hydrophobic fatty acid tail with a single aliphatic chain backbone of C₁₈-C₂₂ covalently attached to a polymer via a linker. In one embodiment, the second lipid-anchored polymer comprises a lipid-linker moiety (also referred to as “linker-lipid moiety”) selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE), dioctadecylamine (DODA), distearoyl-rac-glycerol (DSG), 1,2-dipalmitoyl-rac-glycerol (DPG), a derivative thereof, and a combination any of the foregoing. In one embodiment, the second lipid-anchored polymer comprises a lipid-linker moiety (linker-lipid moiety) selected from the group consisting of DSPE, DSG, DODA, DPG, DOPE, and a derivative of thereof, and a combination of any of the foregoing.

A lipid-anchored polymer of the present disclosure may also comprise a reactive species. In some embodiments, the reactive species is conjugated to the polymer in the lipid-anchored polymer. The reactive species present in a lipid-anchored polymer of the present disclosure may be used for conjugation, e.g., to a targeting moiety which has been functionalized with a complementary reactive species, i.e., a reactive species capable of reacting with the reactive species comprised in the lipid-anchored polymer of the present disclosure. In some embodiments, the reactive species conjugated to the lipid-anchored polymer of the present disclosure may be a thiol reagent, a maleimide reagent, or click chemistry reagent, e.g., a reagent selected from the group consisting of an alkyne reagent, such as a dibenzocyclooctyne (DBCO) reagent, a transcyclooctene (TCO) reagent, a tetrazine (TZ) reagent and an azide (AZ) reagent.

In one embodiment, the antibody or fragment thereof, e.g., IgG, scFv, VHH, is covalently linked to a lipid-anchored polymer (e.g., second lipid-anchored polymer) via strain promoted alkyne-azide cycloaddition (SPAAC) chemistry, such as via an azide-modified lipid-anchored polymer (e.g., DSG-PEG2000-azide, DSPE-PEG2000-azide, DSG-PEG3400-azide, DSPE-PEG3400-azide, DSG-PEG5000-azide, DSPE-PEG5000-azide; DODA-PG46-azide) and a dibenzocyclooctyne (DBCO)-functionalized scFv, VHH, IgG or a fragment thereof.

In an exemplary embodiment, the second lipid-anchored polymer conjugated to a targeting moiety is represented by the following structure:

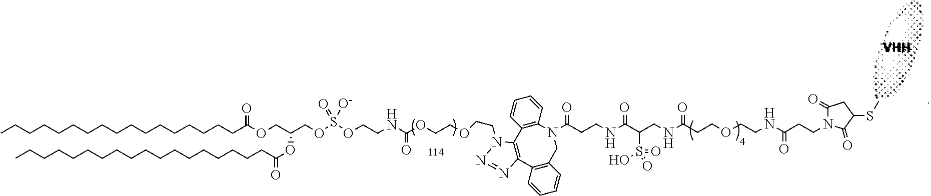

In another exemplary embodiment, the second lipid-anchored polymer conjugated to a targeting moiety is represented by the following structure:

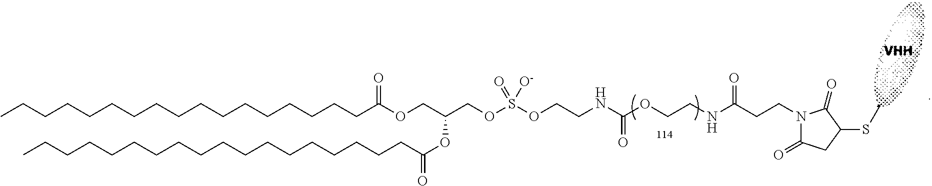

In one embodiment, the ApoE protein, ApoB protein, ApoE polypeptide, ApoB polypeptide, or a fragment thereof, is covalently linked to a lipid-anchored polymer (e.g., second lipid-anchored polymer) via strain promoted alkyne-azide cycloaddition (SPAAC) chemistry, such as via an azide-modified lipid-anchored polymer (e.g., DSG-PEG2000-azide, DSPE-PEG2000-azide, DSG-PEG3400-azide, DSPE-PEG3400-azide, DSG-PEG5000-azide, DSPE-PEG5000-azide, DODA-PG-azide) and a dibenzocyclooctyne (DBCO)-functionalized ApoE protein, ApoB protein, ApoE polypeptide, ApoB polypeptide, or a fragment thereof.

In some embodiments, the LNPs of the present disclosure may comprise a first lipid-anchored polymer and a second lipid-anchored polymer. For example, the LNPs of the present disclosure may comprise a first lipid-anchored polymer that does not comprise a targeting moiety, and a second type of lipid-anchored polymer that comprises a targeting moiety, such as scFv, VHH, GalNAc, ApoE protein/peptide, ApoB protein/peptide. For example, the LNPs of the present disclosure may comprise DSG-PEG2000 modified to comprise an additional OCH₃ group (DSG-PEG2000-OMe) as a first lipid-anchored polymer and DSPE-PEG2000-scFv as a second lipid-anchored polymer.

In one specific embodiment, the first lipid-anchored polymer is the polymer-conjugated lipid of the present disclosure, e.g., DODA-PG34, DODA-PG45, DODA-PG46, or DODA-PG58. For example, the LNPs of the present disclosure may comprise DODA-PG45 as a first lipid-anchored polymer and DSPE-PEG2000-scFv as the second lipid-anchored polymer.

In some embodiments, the LNPs of the present disclosure may comprise a first lipid-anchored polymer and a second lipid-anchored polymer, wherein the second lipid-anchored polymer comprises a targeting moiety. In some embodiments, the second lipid-anchored polymer comprises a lipid-linker moiety (linker-lipid moiety) selected from the group consisting of DSPE, DSG, DODA, DPG, DOPE, and a derivative of thereof. In some embodiments, the first lipid-anchored polymer is any lipid-anchored polymer as described hereinabove.

In some embodiments, the LNPs of the present disclosure may comprise a first lipid-anchored polymer and a second lipid-anchored polymer, wherein the second lipid-anchored polymer comprises a targeting moiety, and the first lipid-anchored polymer and the second lipid-anchored polymer are the same in their lipid-linkers but different in their hydrophilic polymers.

In some embodiments, the LNPs of the present disclosure may comprise a first lipid-anchored polymer and a second lipid-anchored polymer, wherein the second lipid-anchored polymer comprises a targeting moiety, and the first lipid-anchored polymer and the second lipid-anchored polymer are different in their lipid-linker (linker-lipid moiety) as shown below:
DSG-PEG (the first lipid-anchored polymer) and DSPE-PEG (the second lipid-anchored polymer);
DSPE-PEG (the first lipid-anchored polymer) and DSG-PEG (the second lipid-anchored polymer);
DODA-PG (the first lipid-anchored polymer) and DSPE-PEG (the second lipid-anchored polymer);
DPG-PEG (the first lipid-anchored polymer) and DSPE-PEG (the second lipid-anchored polymer);
DODA-PG (the first lipid-anchored polymer) and DSG-PEG (the second lipid-anchored polymer);
DPG-PEG (the first lipid-anchored polymer) and DSG-PEG (the second lipid-anchored polymer); and
DPG-PEG (the first lipid-anchored polymer) and DODA-PG (the second lipid-anchored polymer).

In some embodiments, the LNPs of the present disclosure may comprise a first lipid-anchored polymer and a second lipid-anchored polymer, wherein the second lipid-anchored polymer comprises a targeting moiety, and the first lipid-anchored polymer and the second lipid-anchored polymer are the same lipid-anchored polymers and are selected from one of the following combinations:
DSG-PEG (the first lipid-anchored polymer) and DSG-PEG (the second lipid-anchored polymer);
DSPE-PEG (the first lipid-anchored polymer) and DSPE-PEG (the second lipid-anchored polymer);
DODA-PG (the first lipid-anchored polymer) and DODA-PG (the second lipid-anchored polymer); and
DPG-PEG (the first lipid-anchored polymer) and DPG-PEG (the second lipid-anchored polymer).

In some embodiments, the targeting moiety is conjugated to a DSPE-anchored polymer. In some embodiments, the DSPE-anchored polymer is DSPE-PEG or a derivative thereof.

In some embodiments, the targeting moiety is conjugated to a DSG-anchored polymer. In some embodiments, the DSG-anchored polymer is DSG-PEG or a derivative thereof.

In some embodiments, the LNPs provided by the present disclosure comprise a therapeutic nucleic acid (TNA); an ionizable lipid; helper lipid (e.g., DSPC, DOPE, ceramide); cholesterol; DODA-PG; and DSPE-PEG-IgG. In some embodiments, the LNPs provided by the present disclosure consist essentially of a therapeutic nucleic acid (TNA); an ionizable lipid; helper lipid (e.g., DSPC, DOPE, ceramide); cholesterol; DODA-PG; and DSPE-PEG-IgG. In some embodiments, the LNPs provided by the present disclosure consist of a therapeutic nucleic acid (TNA); an ionizable lipid; helper lipid (e.g., DSPC, DOPE, ceramide); cholesterol; DODA-PG; and DSPE-PEG-IgG.

In some embodiments, the LNPs provided by the present disclosure comprise a therapeutic nucleic acid (TNA); an ionizable lipid; helper lipid (e.g., DSPC, DOPE, ceramide); cholesterol; DODA-PG; and DSPE-PEG-VHH. In some embodiments, the LNPs provided by the present disclosure consist essentially of a therapeutic nucleic acid (TNA); an ionizable lipid; helper lipid (e.g., DSPC, DOPE, ceramide); cholesterol; DODA-PG; and DSPE-PEG-VHH. In some embodiments, the LNPs provided by the present disclosure consist of a therapeutic nucleic acid (TNA); an ionizable lipid; helper lipid (e.g., DSPC, DOPE, ceramide); cholesterol; DODA-PG; and DSPE-PEG-VHH.

In some embodiments, the LNPs provided by the present disclosure comprise a therapeutic nucleic acid (TNA); an ionizable lipid; helper lipid (e.g., DSPC, DOPE, ceramide); cholesterol; DODA-PG; and DODA-PG-scFv. In some embodiments, the LNPs provided by the present disclosure consist essentially of a therapeutic nucleic acid (TNA); an ionizable lipid; helper lipid (e.g., DSPC, DOPE, ceramide); cholesterol; DODA-PG; and DODA-PG-scFv. In some embodiments, the LNPs provided by the present disclosure consist of a therapeutic nucleic acid (TNA); an ionizable lipid; helper lipid (e.g., DSPC, DOPE, ceramide); cholesterol; DODA-PG; and DODA-PG-scFv.

In some embodiments, the LNPs provided by the present disclosure comprise a therapeutic nucleic acid (TNA); an ionizable lipid; helper lipid (e.g., DSPC, DOPE, ceramide); cholesterol; DODA-PG; and DODA-PG-VHH. In some embodiments, the LNPs provided by the present disclosure consist essentially of a therapeutic nucleic acid (TNA); an ionizable lipid; helper lipid (e.g., DSPC, DOPE, ceramide); cholesterol; and DODA-PG; and DODA-PG-VHH. In some embodiments, the LNPs provided by the present disclosure consist of a therapeutic nucleic acid (TNA); an ionizable lipid; helper lipid (e.g., DSPC, DOPE, ceramide); cholesterol; DODA-PG; and DODA-PG-VHH.

In some embodiments, the LNPs provided by the present disclosure comprise a therapeutic nucleic acid (TNA); an ionizable lipid; helper lipid (e.g., DSPC, DOPE, ceramide); cholesterol; and DODA-PG46 (i.e., polyglycerol having an average of 46 glycerol repeating units). In some embodiments, the LNPs provided by the present disclosure consist essentially of a therapeutic nucleic acid (TNA); an ionizable lipid; helper lipid (e.g., DSPC, DOPE, ceramide); cholesterol; and bis-DODA-PG46 (e.g., d18:½:0 or d14:1/2:0). In some embodiments, the LNPs provided by the present disclosure consist of a therapeutic nucleic acid (TNA); an ionizable lipid; helper lipid (e.g., DSPC, DOPE, ceramide); cholesterol; and DODA-PG46.

In some embodiments, the LNPs provided by the present disclosure comprise a therapeutic nucleic acid (TNA); an ionizable lipid; helper lipid (e.g., DSPC, DOPE, ceramide); cholesterol; and DODA-PG34 (i.e., polyglycerol having an average of 34 glycerol units). In some embodiments, the LNPs provided by the present disclosure consist essentially of a therapeutic nucleic acid (TNA); an ionizable lipid; helper lipid (e.g., DSPC, DOPE, ceramide); cholesterol; and DODA-PG34. In some embodiments, the LNPs provided by the present disclosure consist of a therapeutic nucleic acid (TNA); an ionizable lipid; helper lipid (e.g., DSPC, DOPE, ceramide); cholesterol; and DODA-PG34.

In some embodiments, the LNPs provided by the present disclosure comprise a therapeutic nucleic acid (TNA); an ionizable lipid; helper lipid (e.g., DSPC, DOPE, ceramide); cholesterol; DODA-PG46; and DODA-PG46-VHH. In some embodiments, the LNPs provided by the present disclosure consist essentially of a therapeutic nucleic acid (TNA); an ionizable lipid; helper lipid (e.g., DSPC, DOPE, ceramide); cholesterol; DODA-PG46; and DODA-PG46-VHH. In some embodiments, the LNPs provided by the present disclosure consist of a therapeutic nucleic acid (TNA); an ionizable lipid; helper lipid (e.g., DSPC, DOPE, ceramide); cholesterol; DODA-PG46; and DODA-PG46-VHH.

In some embodiments, the LNPs provided by the present disclosure comprise a therapeutic nucleic acid (TNA); an ionizable lipid; helper lipid (e.g., DSPC, DOPE, ceramide); cholesterol; DODA-PG46; and DODA-PG46-scFv. In some embodiments, the LNPs provided by the present disclosure consist essentially of a therapeutic nucleic acid (TNA); an ionizable lipid; helper lipid (e.g., DSPC, DOPE, ceramide); cholesterol; DODA-PG46; and DODA-PG46-scFv. In some embodiments, the LNPs provided by the present disclosure consist of a therapeutic nucleic acid (TNA); an ionizable lipid; helper lipid (e.g., DSPC, DOPE, ceramide); cholesterol; DODA-PG46; and DODA-PG46-scFv.

In some embodiments, the LNPs provided by the present disclosure comprise a therapeutic nucleic acid (TNA); an ionizable lipid; helper lipid (e.g., DSPC, DOPE, ceramide); cholesterol; DSG-PEG2000-OMe; and DODA-PG-VHH. In some embodiments, the LNPs provided by the present disclosure consist essentially of a therapeutic nucleic acid (TNA); an ionizable lipid; helper lipid (e.g., DSPC, DOPE, ceramide); cholesterol; DSG-PEG2000-OMe; and DODA-PG-VHH. In some embodiments, the LNPs provided by the present disclosure consist of a therapeutic nucleic acid (TNA); an ionizable lipid; helper lipid (e.g., DSPC, DOPE, ceramide); cholesterol; DSG-PEG2000-OMe; and DODA-PG-VHH.

In some embodiments, the LNPs provided by the present disclosure comprise a therapeutic nucleic acid (TNA); an ionizable lipid; helper lipid (e.g., DSPC, DOPE, ceramide); cholesterol; DSG-PEG2000-OH; and DODA-PG-VHH. In some embodiments, the LNPs provided by the present disclosure consist essentially of a therapeutic nucleic acid (TNA); an ionizable lipid; helper lipid (e.g., DSPC, DOPE, ceramide); cholesterol; DSG-PEG2000-OH; and DODA-PG-VHH. In some embodiments, the LNPs provided by the present disclosure consist of a therapeutic nucleic acid (TNA); an ionizable lipid; helper lipid (e.g., DSPC, DOPE, ceramide); cholesterol; DSG-PEG2000-OH; and DODA-PG-VHH.

In some embodiments, the LNPs provided by the present disclosure comprise a therapeutic nucleic acid (TNA); an ionizable lipid; helper lipid (e.g., DSPC, DOPE, ceramide); cholesterol; and DSG-PEG2000-OMe. In some embodiments, the LNPs provided by the present disclosure consist essentially of a therapeutic nucleic acid (TNA); an ionizable lipid; helper lipid (e.g., DSPC, DOPE, ceramide); cholesterol; and DSG-PEG2000-OMe. In some embodiments, the LNPs provided by the present disclosure consist of a therapeutic nucleic acid (TNA); an ionizable lipid; helper lipid (e.g., DSPC, DOPE, ceramide); cholesterol; DSG-PEG2000-OMe; and DSPE-PEG2000-VHH.

In some embodiments, the LNPs provided by the present disclosure comprise a therapeutic nucleic acid (TNA); an ionizable lipid; helper lipid (e.g., DSPC, DOPE, ceramide); cholesterol; and DSG-PEG2000-OH. In some embodiments, the LNPs provided by the present disclosure consist essentially of a therapeutic nucleic acid (TNA); an ionizable lipid; helper lipid (e.g., DSPC, DOPE, ceramide); cholesterol; DSG-PEG2000-OH; and DSPE-PEG2000-VHH. In some embodiments, the LNPs provided by the present disclosure consist of a therapeutic nucleic acid (TNA); an ionizable lipid; helper lipid (e.g., DSPC, DOPE, ceramide) cholesterol; DSG-PEG2000-OH; and DSPE-PEG2000-VHH.

In some embodiments, the LNPs provided by the present disclosure comprise a therapeutic nucleic acid (TNA); an ionizable lipid; helper lipid (e.g., DSPC, DOPE, ceramide); cholesterol; DSG-PEG2000-OMe and DSPE-PEG2000-scFv. In some embodiments, the LNPs provided by the present disclosure consist essentially of a therapeutic nucleic acid (TNA); an ionizable lipid; helper lipid (e.g., DSPC, DOPE, ceramide); cholesterol; DSG-PEG2000-OMe and DSPE-PEG2000-scFv. In some embodiments, the LNPs provided by the present disclosure consist of a therapeutic nucleic acid (TNA); an ionizable lipid; helper lipid (e.g., DSPC, DOPE, ceramide); cholesterol; DSG-PEG2000-OMe and DSPE-PEG2000-scFv.

In some embodiments, the LNPs provided by the present disclosure comprise a therapeutic nucleic acid (TNA); an ionizable lipid; helper lipid (e.g., DSPC, DOPE, ceramide); cholesterol; DSG-PEG2000-OH and DSPE-PEG2000-scFv. In some embodiments, the LNPs provided by the present disclosure consist essentially of a therapeutic nucleic acid (TNA); an ionizable lipid; helper lipid (e.g., DSPC, DOPE, ceramide); cholesterol; DSG-PEG2000-OH and DSPE-PEG2000-scFv. In some embodiments, the LNPs provided by the present disclosure consist of a therapeutic nucleic acid (TNA); an ionizable lipid; helper lipid (e.g., DSPC, DOPE, ceramide); cholesterol; DSG-PEG2000-OH and DSPE-PEG2000-scFv.

In some embodiments, the LNPs provided by the present disclosure comprise a therapeutic nucleic acid (TNA); an ionizable lipid; helper lipid (e.g., DSPC, DOPE, ceramide); cholesterol; bis-DSG-PEG2000 and DSPE-PEG2000-scFv. In some embodiments, the LNPs provided by the present disclosure consist essentially of a therapeutic nucleic acid (TNA); an ionizable lipid; helper lipid (e.g., DSPC, DOPE, ceramide); cholesterol; bis-DSG-PEG2000 and DSPE-PEG2000-scFv. In some embodiments, the LNPs provided by the present disclosure consist of a therapeutic nucleic acid (TNA); an ionizable lipid; helper lipid (e.g., DSPC, DOPE, ceramide); cholesterol; bis-DSG-PEG2000 and DSPE-PEG2000-scFv.

In some embodiments, the LNPs provided by the present disclosure comprise a therapeutic nucleic acid (TNA); an ionizable lipid; helper lipid (e.g., DSPC, DOPE, ceramide); cholesterol; DODA-PG and DSPE-PEG-scFv. In some embodiments, the LNPs provided by the present disclosure consist essentially of a therapeutic nucleic acid (TNA); an ionizable lipid; helper lipid (e.g., DSPC, DOPE, ceramide); cholesterol; DODA-PG45 and DSPE-PEG2000-scFv. In some embodiments, the LNPs provided by the present disclosure consist of a therapeutic nucleic acid (TNA); an ionizable lipid; helper lipid (e.g., DSPC, DOPE, ceramide); cholesterol; DODA-PG45 and DSPE-PEG2000-scFv.

In some embodiments, the lipid-anchored polymers (first and second lipid-anchored polymers in combination) constitute about 0.1 mol % to about 20 mol % of the total lipid present in the LNP. In some embodiments, the lipid-anchored polymers constitute about 0.5 mol % to about 10 mol % present in the LNP. In some embodiments, the lipid-anchored polymers constitute about 1 mol % to about 10 mol % present in the LNP. In some embodiments, the lipid-anchored polymers constitute about 2 mol % to about 10 mol % present in the LNP. In some embodiments, the lipid-anchored polymers constitute more than about 2 mol % (e.g., 2.1 mol %, 2.2 mol %, 2.3 mol %, 2.4 mol %, 2.5 mol %, 2.6 mol %, 2.7 mol %, 2.8 mol %, 2.9 mol %, 3.0 mol %) to about 10 mol % present in the LNP. In some embodiments, the lipid-anchored polymers constitute about 3 mol % to about 8 mol % present in the LNP. In some embodiments, the lipid-anchored polymers constitute about 3 mol % to about 7 mol % present in the LNP. In some embodiments, the lipid-anchored polymers constitute about 3 mol % to about 5 mol % present in the LNP. In some embodiments, the lipid-anchored polymers constitute about 2 mol % to about 4 mol % present in the LNP. In some embodiments, the lipid-anchored polymers constitute about 2% to about 3% present in the LNP. In some embodiments, the lipid-anchored polymers constitute about 2 mol % present in the LNP. In some embodiments, the lipid-anchored polymers constitute about 2.5 mol % present in the LNP. In some embodiments, the lipid-anchored polymers constitute about 3 mol % present in the LNP. In some embodiments, the lipid-anchored polymers constitute about 3.5 mol % present in the LNP. In some embodiments, the lipid-anchored polymers constitute about 4 mol % present in the LNP. In some embodiments, the lipid-anchored polymers constitute about 5 mol % present in the LNP. In some embodiments, the lipid-anchored polymers constitute about 6 mol % present in the LNP. In some embodiments, the lipid-anchored polymers constitute about 7 mol % present in the LNP. In some embodiments, the lipid-anchored polymers constitute about 8 mol % present in the LNP. In some embodiments, the lipid-anchored polymers constitute about 9 mol % present in the LNP. In some embodiments, the lipid-anchored polymers constitute about 10 mol % present in the LNP.

In some embodiments, the first lipid-anchored polymer is present in about 0.1 mol % to about 10 mol % of the total lipid present in the LNP, or about 0.2 mol % to about 8 mol %, or about 0.2 mol % to about 7 mol %, or about 0.2% mol % to about 5 mol %, or about 0.3 mol to about 4 mol %, or about 0.4 mol % to about 4 mol %, or about 0.5 mol % to about 5 mol %, or about 0.5 mol % to about 4 mol %, or about 0.5 mol % to about 3.5 mol %, or about 0.5 mol % to about 3 mol %, or about 0.7 mol % to about 5 mol %, or about 0.7 mol % to about 4 mol %, or about 0.7 mol % to about 3.5 mol %, or about 0.7 mol % to about 3 mol %, or about 1 mol % to about 5 mol %, or about 1 mol % to about 4 mol %, or about 1 mol % to about 3.5 mol %, or about 1 mol % to about 3 mol %, or about 1.5 mol % to about 5 mol %, or about 1.5 mol % to about 4 mol %, or about 1.5 mol % to about 3.5 mol %, or about 1.5 mol % to about 3 mol %, or about 2 mol % to about 5 mol %, or about 2 mol % to about 4 mol %, or about 2 mol % to about 3.5 mol %, or about 2 mol % to about 3 mol %, or about 2.5 mol % to about 5 mol %, or about 2.5 mol % to about 4 mol %, or about 2.5 mol % to about 3.5 mol %, or about 2.5 mol % to about 3 mol %, or about 3 mol % to about 5 mol %, or about 3 mol % to about 4.5 mol % or about 3 mol % to about 4 mol %, or about 3 mol % to about 3.5 mol %, or about 3.5 mol % to about 5 mol %, or about 3.5 mol % to about 4.5 mol % or about 3.5 mol % to about 4 mol % or about 3 mol % to about 7 mol %.

In some embodiments, the second lipid-anchored polymer, if present, is present in about 0.005 mol % to about 5 mol % of the total lipid present in the LNP, or about 0.005 mol % to about 3 mol %, or about 0.005 mol % to about 2 mol %, or about 0.005 mol % to about 1 mol %, or about 0.005 mol % to about 0.5 mol %, or about 0.01 mol % to about 3 mol %, or about 0.01 mol % to about 2 mol %, or about 0.01 mol % to about 1 mol %, or about 0.01 mol % to about 0.5 mol %, or about 0.025 mol % to about 3 mol %, or about 0.025 mol % to about 2 mol %, or about 0.025 mol % to about 1 mol %, or about 0.025 mol % to about 0.5 mol %, or about 0.05 mol % to about 3 mol %, or about 0.05 mol % to about 2 mol %, or about 0.05 mol % to about 1 mol %, or about 0.05 mol % to about 0.5 mol %, or about 0.01 mol % to about 0.4 mol %, or about 0.01 mol % to about 0.3 mol %, or about 0.01 mol % to about 0.25 mol %, or about 0.01 mol % to about 0.2 mol %, or about 0.01 mol % to about 0.1 mol %, or about 0.025 mol % to about 0.4 mol %, or about 0.025 mol % to about 0.3 mol %, or about 0.025 mol % to about 0.25 mol %, or about 0.025 mol % to about 0.2 mol %, or about 0.025 mol % to about 0.1 mol %, or about 0.05 mol % to about 0.4 mol %, or about 0.05 mol % to about 0.3 mol %, or about 0.05 mol % to about 0.25 mol %, or about 0.05 mol % to about 0.2 mol %, or about 0.05 mol % to about 0.1 mol %. In some embodiments, the second lipid-anchored polymer is present in about 0.5 mol %

In some embodiments, the targeting moiety is conjugated to a DSPE-anchored polymer. In some embodiments, the DSPE-anchored polymer is DSPE-PEG or a derivative thereof.

In some embodiments, the targeting moiety is conjugated to a DSG-anchored polymer. In some embodiments, the DSG-anchored polymer is DSG-PEG or a derivative thereof.

In some embodiments, the LNPs provided by the present disclosure comprise a therapeutic nucleic acid (TNA); an ionizable lipid; C2 ceramide (e.g., d18:½:0 or d14:1/2:0); cholesterol; and DSG-PEG2000-OMe. In some embodiments, the LNPs provided by the present disclosure consist essentially of a therapeutic nucleic acid (TNA); an ionizable lipid; C2 ceramide (e.g., d18:½:0 or d14:1/2:0); cholesterol; and DSG-PEG2000-OMe. In some embodiments, the LNPs provided by the present disclosure consist of a therapeutic nucleic acid (TNA); an ionizable lipid; C2 ceramide (e.g., d18:½:0 or d14:1/2:0); cholesterol; and DSG-PEG2000-OMe.

In some embodiments, the LNPs provided by the present disclosure comprise a therapeutic nucleic acid (TNA); an ionizable lipid; C2 ceramide (e.g., d18:½:0 or d14:1/2:0); cholesterol; and DSG-PEG2000-OH. In some embodiments, the LNPs provided by the present disclosure consist essentially of a therapeutic nucleic acid (TNA); an ionizable lipid; C2 ceramide (e.g., d18:½:0 or d14:1/2:0); cholesterol; and DSG-PEG2000-OH. In some embodiments, the LNPs provided by the present disclosure consist of a therapeutic nucleic acid (TNA); an ionizable lipid; C2 ceramide (e.g., d18:½:0 or d14:1/2:0); cholesterol; and DSG-PEG2000-OH.

In some embodiments, the LNPs provided by the present disclosure comprise a therapeutic nucleic acid (TNA); an ionizable lipid; C2 ceramide (e.g., d18:½:0 or d14:1/2:0); cholesterol; and bis-DSG-PEG2000-OMe. In some embodiments, the LNPs provided by the present disclosure consist essentially of a therapeutic nucleic acid (TNA); an ionizable lipid; C2 ceramide (e.g., d18:½:0 or d14:1/2:0); cholesterol; and bis-DSG-PEG2000-OMe. In some embodiments, the LNPs provided by the present disclosure consist of a therapeutic nucleic acid (TNA); an ionizable lipid; C2 ceramide (e.g., d18:½:0 or d14:1/2:0); cholesterol; and bis-DSG-PEG2000-OMe.

In some embodiments, the LNPs provided by the present disclosure comprise a therapeutic nucleic acid (TNA); an ionizable lipid; C2 ceramide (e.g., d18:½:0 or d14:1/2:0); cholesterol; and DODA-PG46 (i.e., polyglycerol having an average of 46 glycerol repeating units). In some embodiments, the LNPs provided by the present disclosure consist essentially of a therapeutic nucleic acid (TNA); an ionizable lipid; C2 ceramide (e.g., d18:½:0 or d14:1/2:0); cholesterol; and bis-DODA-PG46 (e.g., d18:½:0 or d14:1/2:0). In some embodiments, the LNPs provided by the present disclosure consist of a therapeutic nucleic acid (TNA); an ionizable lipid; C2 ceramide (e.g., d18:½:0 or d14:1/2:0); cholesterol; and DODA-PG46.

In some embodiments, the LNPs provided by the present disclosure comprise a therapeutic nucleic acid (TNA); an ionizable lipid; C2 ceramide (e.g., d18:½:0 or d14:1/2:0); cholesterol; and DODA-PG34 (i.e., polyglycerol having an average of 34 glycerol units). In some embodiments, the LNPs provided by the present disclosure consist essentially of a therapeutic nucleic acid (TNA); an ionizable lipid; C2 ceramide (e.g., d18:½:0 or d14:1/2:0); cholesterol; and bis-DODA-PG34. In some embodiments, the LNPs provided by the present disclosure consist of a therapeutic nucleic acid (TNA); an ionizable lipid; C2 ceramide (e.g., d18:½:0 or d14:1/2:0); cholesterol; and DODA-PG34. In some embodiments, the LNPs provided by the present disclosure comprise a therapeutic nucleic acid (TNA); an ionizable lipid; DSPC; cholesterol; and DSG-PEG2000-OMe. In some embodiments, the LNPs provided by the present disclosure consist essentially of a therapeutic nucleic acid (TNA); an ionizable lipid; DSPC; cholesterol; and DSG-PEG2000-OMe. In some embodiments, the LNPs provided by the present disclosure consist of a therapeutic nucleic acid (TNA); an ionizable lipid; DSPC; cholesterol; and DSG-PEG2000-OMe.

In some embodiments, the LNPs provided by the present disclosure comprise a therapeutic nucleic acid (TNA); an ionizable lipid; DSPC; cholesterol; and DSG-PEG2000-OH. In some embodiments, the LNPs provided by the present disclosure consist essentially of a therapeutic nucleic acid (TNA); an ionizable lipid; DSPC; cholesterol; and DSG-PEG2000-OH. In some embodiments, the LNPs provided by the present disclosure consist of a therapeutic nucleic acid (TNA); an ionizable lipid; DSPC; cholesterol; and DSG-PEG2000-OH.

In some embodiments, the LNPs provided by the present disclosure comprise a therapeutic nucleic acid (TNA); an ionizable lipid; DOPE; cholesterol; and DSG-PEG2000-OMe. In some embodiments, the LNPs provided by the present disclosure consist essentially of a therapeutic nucleic acid (TNA); an ionizable lipid; DOPE; cholesterol; and DSG-PEG2000-OMe. In some embodiments, the LNPs provided by the present disclosure consist of a therapeutic nucleic acid (TNA); an ionizable lipid; DOPE; cholesterol; and DSG-PEG2000-OMe.

In some embodiments, the LNPs provided by the present disclosure comprise a therapeutic nucleic acid (TNA); an ionizable lipid; DSPC; cholesterol; and DSPE-PEG2000-OH. In some embodiments, the LNPs provided by the present disclosure consist essentially of a therapeutic nucleic acid (TNA); an ionizable lipid; DSPC; cholesterol; and DSPE-PEG2000-OH. In some embodiments, the LNPs provided by the present disclosure consist of a therapeutic nucleic acid (TNA); an ionizable lipid; DSPC; cholesterol; and DSPE-PEG2000-OH.

In some embodiments, the LNPs provided by the present disclosure comprise a therapeutic nucleic acid (TNA); an ionizable lipid; DOPE; cholesterol; and DSG-PEG2000-OMe. In some embodiments, the LNPs provided by the present disclosure consist essentially of a therapeutic nucleic acid (TNA); an ionizable lipid; DOPE; cholesterol; and DSG-PEG2000-OMe. In some embodiments, the LNPs provided by the present disclosure consist of a therapeutic nucleic acid (TNA); an ionizable lipid; DOPE; cholesterol; and DSG-PEG2000-OMe.

In some embodiments, the LNPs provided by the present disclosure comprise a therapeutic nucleic acid (TNA); an ionizable lipid; DOPE; cholesterol; and DSPE-PEG2000-OH. In some embodiments, the LNPs provided by the present disclosure consist essentially of a therapeutic nucleic acid (TNA); an ionizable lipid; DOPE; cholesterol; and DSPE-PEG2000-OH. In some embodiments, the LNPs provided by the present disclosure consist of a therapeutic nucleic acid (TNA); an ionizable lipid; DOPE; cholesterol; and DSPE-PEG2000-OH.

In some embodiments, the LNPs provided by the present disclosure comprise a therapeutic nucleic acid (TNA); an ionizable lipid; DSPC; cholesterol; DSG-PEG2000-OMe and DSPE-PEG2000-GalNAc3. In some embodiments, the LNPs provided by the present disclosure consist essentially of a therapeutic nucleic acid (TNA); an ionizable lipid; DSPC; cholesterol; DSG-PEG2000-OMe and DSPE-PEG2000-GalNAc3. In some embodiments, the LNPs provided by the present disclosure consist of a therapeutic nucleic acid (TNA); an ionizable lipid; DSPC; cholesterol; DSG-PEG2000-OMe and DSPE-PEG2000-GalNAc3.

In some embodiments, the LNPs provided by the present disclosure comprise a therapeutic nucleic acid (TNA); an ionizable lipid; C2 ceramide (e.g., d18:½:0 or d14:1/2:0); cholesterol; DSG-PEG2000-OH and DSPE-PEG2000-GalNAc3. In some embodiments, the LNPs provided by the present disclosure consist essentially of a therapeutic nucleic acid (TNA); an ionizable lipid; C2 ceramide (e.g., d18:½:0 or d14:1/2:0); cholesterol; DSG-PEG2000-OH and DSPE-PEG2000-GalNAc3. In some embodiments, the LNPs provided by the present disclosure consist of a therapeutic nucleic acid (TNA); an ionizable lipid; C2 ceramide (e.g., d18:½:0 or d14:1/2:0); cholesterol; DSG-PEG2000-OH and DSPE-PEG2000-GalNAc3.

In some embodiments, the LNPs provided by the present disclosure comprise a therapeutic nucleic acid (TNA); an ionizable lipid; DSPC; cholesterol; bis-DSG-PEG2000 and DSPE-PEG2000. In some embodiments, the LNPs provided by the present disclosure consist essentially of a therapeutic nucleic acid (TNA); an ionizable lipid; DSPC; cholesterol; bis-DSG-PEG2000 and DSPE-PEG2000. In some embodiments, the LNPs provided by the present disclosure consist of a therapeutic nucleic acid (TNA); an ionizable lipid; DSPC; cholesterol; bis-DSG-PEG2000 and DSPE-PEG2000.

In some embodiments, the LNPs provided by the present disclosure comprise a therapeutic nucleic acid (TNA); an ionizable lipid; DSPC; cholesterol; DODA-PG46 and DSPE-PEG2000-GalNAc3. In some embodiments, the LNPs provided by the present disclosure consist essentially of a therapeutic nucleic acid (TNA); an ionizable lipid; DSPC; cholesterol; DODA-PG46 and DSPE-PEG2000-GalNAc3. In some embodiments, the LNPs provided by the present disclosure consist of a therapeutic nucleic acid (TNA); an ionizable lipid; DSPC; cholesterol; DODA-PG46 and DSPE-PEG2000-GalNAc3.

In some embodiments, the lipid-anchored polymers (first and second lipid-anchored polymers in combination) constitute about 0.1 mol % to about 20 mol % of the total lipid present in the LNP. In some embodiments, the lipid-anchored polymers constitute about 0.5 mol % to about 10 mol % present in the LNP. In some embodiments, the lipid-anchored polymers constitute about 1 mol % to about 10 mol % present in the LNP. In some embodiments, the lipid-anchored polymers constitute about 2 mol % to about 10 mol % present in the LNP. In some embodiments, the lipid-anchored polymers constitute more than about 2 mol % (e.g., 2.1 mol %, 2.2 mol %, 2.3 mol %, 2.4 mol %, 2.5 mol %, 2.6 mol %, 2.7 mol %, 2.8 mol %, 2.9 mol %) to about 10 mol % present in the LNP. In some embodiments, the lipid-anchored polymers constitute about 3 mol % to about 8 mol % present in the LNP. In some embodiments, the lipid-anchored polymers constitute about 3 mol % to about 7 mol % present in the LNP. In some embodiments, the lipid-anchored polymers constitute about 3 mol % to about 5 mol % present in the LNP. In some embodiments, the lipid-anchored polymers constitute about 2 mol % to about 4 mol % present in the LNP. In some embodiments, the lipid-anchored polymers constitute about 2% to about 3% present in the LNP. In some embodiments, the lipid-anchored polymers constitute about 2 mol % present in the LNP. In some embodiments, the lipid-anchored polymers constitute about 2.5 mol % present in the LNP. In some embodiments, the lipid-anchored polymers constitute about 3 mol % present in the LNP. In some embodiments, the lipid-anchored polymers constitute about 3.5 mol % present in the LNP. In some embodiments, the lipid-anchored polymers constitute about 4 mol % present in the LNP.

In some embodiments, the first lipid-anchored polymer is present in about 0.1 mol % to about 10 mol % of the total lipid present in the LNP, or about 0.2 mol % to about 8 mol %, or about 0.2 mol % to about 7 mol %, or about 0.2% mol % to about 5 mol %, or about 0.3 mol to about 4 mol %, or about 0.4 mol % to about 4 mol %, or about 0.5 mol % to about 5 mol %, or about 0.5 mol % to about 4 mol %, or about 0.5 mol % to about 3.5 mol %, or about 0.5 mol % to about 3 mol %, or about 0.7 mol % to about 5 mol %, or about 0.7 mol % to about 4 mol %, or about 0.7 mol % to about 3.5 mol %, or about 0.7 mol % to about 3 mol %, or about 1 mol % to about 5 mol %, or about 1 mol % to about 4 mol %, or about 1 mol % to about 3.5 mol %, or about 1 mol % to about 3 mol %, or about 1.5 mol % to about 5 mol %, or about 1.5 mol % to about 4 mol %, or about 1.5 mol % to about 3.5 mol %, or about 1.5 mol % to about 3 mol %, or about 2 mol % to about 5 mol %, or about 2 mol % to about 4 mol %, or about 2 mol % to about 3.5 mol %, or about 2 mol % to about 3 mol %, or about 2.5 mol % to about 5 mol %, or about 2.5 mol % to about 4 mol %, or about 2.5 mol % to about 3.5 mol %, or about 2.5 mol % to about 3 mol %, or about 3 mol % to about 5 mol %, or about 3 mol % to about 4.5 mol % or about 3 mol % to about 4 mol %, or about 3 mol % to about 3.5 mol %, or about 3.5 mol % to about 5 mol %, or about 3.5 mol % to about 4.5 mol % or about 3.5 mol % to about 4 mol %.

In some embodiments, the second lipid-anchored polymer, if present, is present in about 0.005 mol % to about 5 mol % of the total lipid present in the LNP, or about 0.005 mol % to about 3 mol %, or about 0.005 mol % to about 2 mol %, or about 0.005 mol % to about 1 mol %, or about 0.005 mol % to about 0.5 mol %, or about 0.01 mol % to about 3 mol %, or about 0.01 mol % to about 2 mol %, or about 0.01 mol % to about 1 mol %, or about 0.01 mol % to about 0.5 mol %, or about 0.025 mol % to about 3 mol %, or about 0.025 mol % to about 2 mol %, or about 0.025 mol % to about 1 mol %, or about 0.025 mol % to about 0.5 mol %, or about 0.05 mol % to about 3 mol %, or about 0.05 mol % to about 2 mol %, or about 0.05 mol % to about 1 mol %, or about 0.05 mol % to about 0.5 mol %, or about 0.01 mol % to about 0.4 mol %, or about 0.01 mol % to about 0.3 mol %, or about 0.01 mol % to about 0.25 mol %, or about 0.01 mol % to about 0.2 mol %, or about 0.01 mol % to about 0.1 mol %, or about 0.025 mol % to about 0.4 mol %, or about 0.025 mol % to about 0.3 mol %, or about 0.025 mol % to about 0.25 mol %, or about 0.025 mol % to about 0.2 mol %, or about 0.025 mol % to about 0.1 mol %, or about 0.05 mol % to about 0.4 mol %, or about 0.05 mol % to about 0.3 mol %, or about 0.05 mol % to about 0.25 mol %, or about 0.05 mol % to about 0.2 mol %, or about 0.05 mol % to about 0.1 mol %.

Lipid nanoparticles (LNPs) comprising ceDNA are disclosed in International Patent Application No. PCT/US2018/050042, filed on Sep. 7, 2018, which is incorporated herein in its entirety and envisioned for use in the methods and compositions as disclosed herein.

The size of LNPs can be determined by quasi-elastic light scattering using a Malvern Zetasizer Nano ZS (Malvern, UK). In some embodiments, LNPs of the present disclosure have a mean diameter as determined by light scattering of less than about 90 nm, e.g., less than about 80 nm or less than about 75 nm. According to some embodiments, LNPs of the present disclosure have a mean diameter as determined by light scattering of between about 50 nm and about 75 nm or between about 50 nm and about 70 nm.

The pKa of formulated cationic lipids can be correlated with the effectiveness of the LNPs for delivery of nucleic acids (see Jayaraman et al., Angewandte Chemie, International Edition (2012), 51 (34), 8529-8533; Semple et al., Nature Biotechnology 28, 172-176 (2010), both of which are incorporated by reference in their entireties). In one embodiment, the pKa of each cationic lipid is determined in lipid nanoparticles using an assay based on fluorescence of 2-(p-toluidino)-6-napthalene sulfonic acid (TNS). LNPs in PBS at a concentration of 0.4 mM total lipid can be prepared using the in-line process as described herein and elsewhere. TNS can be prepared as a 100 mM stock solution in distilled water. Vesicles can be diluted to 24 mM lipid in 2 mL of buffered solutions containing, 10 mM HEPES, 10 mM MES, 10 mM ammonium acetate, 130 mM NaCl, where the pH ranges from 2.5 to 11. An aliquot of the TNS solution can be added to give a final concentration of 1 mM and following vortex mixing fluorescence intensity is measured at room temperature in a SLM Aminco Series 2 Luminescence Spectrophotometer using excitation and emission wavelengths of 321 nm and 445 nm. A sigmoidal best fit analysis can be applied to the fluorescence data and the pKa is measured as the pH giving rise to half-maximal fluorescence intensity.

In one embodiment, relative activity can be determined by measuring luciferase expression in the liver 4 hours following administration via tail vein injection. The activity is compared at a dose of 0.3 and 1.0 mg ceDNA/kg and expressed as ng luciferase/g liver measured 4 hours after administration.

Without limitations, LNP of the present disclosure includes a lipid formulation that can be used to deliver a capsid-free, non-viral DNA vector to a target site of interest (e.g., cell, tissue, organ, specific cell types and the like). Generally, the LNP comprises capsid-free, non-viral DNA vector and a cationic lipid or a salt thereof.

Further exemplary lipid-anchored polymers include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEGylated lipid, for example, a (methoxy polyethylene glycol)-conjugated lipid. PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, a pegylated phosphatidylethanoloamine (PEG-PE), PEG succinate diacylglycerol (PEGS-DAG) (such as 4-0-(2′,3′-di(tetradecanoyloxy)propyl-1-0-w-methoxy (polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, or a mixture thereof. Additional exemplary PEG-lipid conjugates are described, for example, in International Patent Application Publication Nos. WO 1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, U.S. Patent Application Publication Nos. US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and U.S. Pat. Nos. 5,885,613, 6,287,591, 6,320,017, and 6,586,559, the contents of all of which are incorporated herein by reference in their entireties.

Additional examples of PEG-DAA PEGylated lipids include, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3 [beta]-oxy) carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy (polyethylene glycol)-2000]. In one embodiment, the PEG-lipid can be selected from the group consisting of PEG-DMG, 1,2-dimyristoyl-sn-glycero-3-phosphoethanolamine-N-[methoxy (polyethylene glycol)-2000],

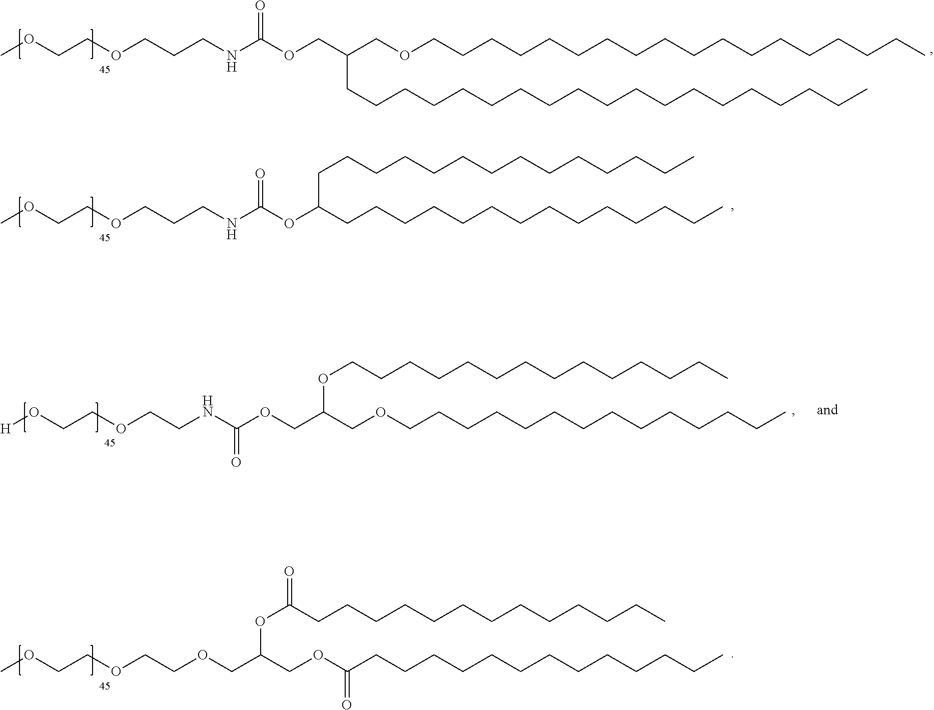

Yet further exemplary lipid-anchored polymers include N-(Carbonyl-methoxypolyethyleneglycoln)-1,2-dimyristoyl-sn-glycero-3-phosphoethanolamine (DMPE-PEGₙ, where n is 350, 500, 750, 1000 or 2000), N-(Carbonyl-methoxypolyethyleneglycoln)-1,2-distearoyl-sn-glycero-3-phosphoethanolamine (DSPE-PEGₙ, where n is 350, 500, 750, 1000 or 2000), DSPE-polyglycelin-cyclohexyl-carboxylic acid, DSPE-polyglycelin-2-methylglutar-carboxylic acid, 1,2-Distearoyl-sn-Glycero-3-Phosphoethanolamine (DSPE) conjugated Polyethylene Glycol (DSPE-PEG-OH), polyethylene glycol-dimyristolglycerol (PEG-DMG), or polyethylene glycol-distearoyl glycerol (PEG-DSG). In some examples of DMPE-PEGₙ, where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some examples of DSPE-PEGₙ, where n is 350, 500, 750, 1000 or 2000, the PEG-lipid is N-(Carbonyl-methoxypolyethyleneglycol 2000)-1,2-distearoyl-sn-glycero-3-phosphoethanolamine (DSPE-PEG 2,000). In some embodiments, the PEG-lipid is DSPE-PEG-OH. In some embodiments, the PEG-lipid is PEG-DMG having two C₁₄ hydrophobic tails and PEG2000.

### E. Therapeutic Nucleic Acids

The LNPs provided by the present disclosure also comprise a therapeutic nucleic acid (TNA). According to embodiments, also provided are pharmaceutical compositions comprising the LNPs of the disclosure.

Illustrative therapeutic nucleic acids in the LNPs of the present disclosure can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, ssDNA, CELiD, linear covalently closed DNA (“ministring”), Doggybone™, protelomere closed ended DNA, or dumbbell linear DNA), dicer-substrate dsRNA, small hairpin RNA (shRNA), asymmetrical interfering RNA (aiRNA), mRNA, tRNA, rRNA, gRNA, and DNA viral vectors, viral RNA vector, and any combination thereof.

In any of the aspects and embodiments provided herein, the therapeutic nucleic acid can be a therapeutic DNA. Said therapeutic DNA can be ceDNA, ssDNA. CELiD, linear covalently closed DNA (“ministring” or otherwise), Doggybone™, protelomere closed ended DNA, dumbbell linear DNA, minigenes, plasmids, or minicircles.

siRNA or miRNA that can downregulate the intracellular levels of specific proteins through a process called RNA interference (RNAi) are also contemplated by the present disclosure to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson-capable of binding to the mRNA by Crick base pairing. This binding prevents translation of a target mRNA, and/or triggers RNaseH degradation of the mRNA transcript. As a result, the antisense oligonucleotide has increased specificity of action (i.e., down-regulation of a specific disease-related protein).

In any of the aspects and embodiments provided herein, the therapeutic nucleic acid can be a therapeutic RNA. The therapeutic RNA can be messenger RNA (mRNA) encoding a protein or peptide, an inhibitor of mRNA translation, agent of RNA interference (RNAi), catalytically active RNA molecule (ribozyme), transfer RNA (tRNA), an RNA that binds an mRNA transcript (ASO), protein or other molecular ligand (aptamer), or a guide RNA (gRNA). In any of the methods provided herein, the agent of RNAi can be a double-stranded RNA, single-stranded RNA, microRNA, short interfering RNA, short hairpin RNA, or a triplex-forming oligonucleotide. In one embodiment, the TNA is mRNA.

Single Stranded DNA (ssDNA)

As described herein, the present disclosure relates to synthetic single-stranded (ssDNA) molecules. According to some aspects, the disclosure provides a single stranded deoxyribonucleic acid (ssDNA) molecule comprising at least one nucleic acid sequence of interest flanked by at least one stem-loop structure at the 3′ end. According to some embodiments, the ssDNA molecule further comprises a 5′ end, comprising at least one stem-loop structure.

i) 3′ End Stem-Loop Structure of ssDNA

As described herein, according to some aspects, the disclosure provides a ssDNA molecule comprising at least one nucleic acid sequence of interest flanked by at least one stem-loop structure at the 3′ end. As described herein, the stem structure comprises a partial DNA duplex (e.g., with a free 3′—OH group) to prime replication or transcription. The partial DNA duplex functions, in part, to hold the stem-loop structure together.

According to some embodiments, the partial DNA duplex comprises between 4-500 nucleotides, for example between 4-10 nucleotides, between 4-25 nucleotides, between 4-50 nucleotides, between 4-100 nucleotides, between 4-200 nucleotides, between 4-300 nucleotides, between 4-400 nucleotides, between 20-25 nucleotides, between 20-50 nucleotides, between 20-100 nucleotides, between 20-200 nucleotides, between 20-300 nucleotides, between 20-400 nucleotides, between 20-500 nucleotides, between 50-100 nucleotides, between 50-200 nucleotides, between 50-300 nucleotides, between 50-400 nucleotides, between 50-500 nucleotides, 150-200 nucleotides, between 150-300 nucleotides, between 150-400 nucleotides, between 150-500 nucleotides, between 200-300 nucleotides, between 200-400 nucleotides, between 200-500 nucleotides, between 250-300 nucleotides, between 250-400 nucleotides, between 250-500 nucleotides, between 300-400 nucleotides, between 300-500 nucleotides, between 350-400 nucleotides, between 350-500 nucleotides, between 400-500 nucleotides, or between 450-500 nucleotides, and at least one loop on the 3′ end. According to some embodiments, the DNA duplex comprises at least 4, 5, 6, 7, 8, 9, 10, 20, 30, 40, 50, 60, 70, 80, 90, 100, 150, 200, 250, 300, 350, 400, 450 or 500 nucleotides, and at least one loop on the 3′ end.

According to some embodiments, the loop structure at the 3′ end comprises a minimum of between 3-500 unbound nucleotides, for example between 3-450 nucleotides, between 3-400 nucleotides, between 3-350 nucleotides, between 3-300 nucleotides, between 3-250 nucleotides, between 3-200 nucleotides, between 3-150 nucleotides, between 3-100 nucleotides, between 3-90 nucleotides, between 3-80 nucleotides, between 3-70 nucleotides, between 3-60 nucleotides, between 3-50 nucleotides, between 3-40 nucleotides, between 3-30 nucleotides, between 3-20 nucleotides, between 3-10 nucleotides, between 3-5 nucleotides, between 10-450 nucleotides, between 10-400 nucleotides, between 10-350 nucleotides, between 10-300 nucleotides, between 10-250 nucleotides, between 10-200 nucleotides, between 10-150 nucleotides, between 10-100 nucleotides, between 10-90 nucleotides, between 10-80 nucleotides, between 10-70 nucleotides, between 10-60 nucleotides, between 10-50 nucleotides, between 10-40 nucleotides, between 10-30 nucleotides, between 10-20 nucleotides, between 50-450 nucleotides, between 50-400 nucleotides, between 50-350 nucleotides, between 50-300 nucleotides, between 50-250 nucleotides, between 50-200 nucleotides, between 50-150 nucleotides, between 50-100 nucleotides, between 50-90 nucleotides, between 50-80 nucleotides, between 50-70 nucleotides, between 50-60 nucleotides, between 100-450 nucleotides, between 100-400 nucleotides, between 100-350 nucleotides, between 100-300 nucleotides, between 100-250 nucleotides, between 100-200 nucleotides, between 150-450 nucleotides, between 150-400 nucleotides, between 150-350 nucleotides, between 150-300 nucleotides, between 150-250 nucleotides, between 150-200 nucleotides, between 200-450 nucleotides, between 200-400 nucleotides, between 200-350 nucleotides, between 200-300 nucleotides, between 200-250 nucleotides, between 250-450 nucleotides, between 250-400 nucleotides, between 250-350 nucleotides, between 250-300 nucleotides, between 300-450 nucleotides, between 300-400 nucleotides, between 300-350 nucleotides, between 350-450 nucleotides, between 350-400 nucleotides, or between 400-450 nucleotides.

According to some embodiments, the stem portion of the stem-loop is 4-500 nucleotides in length and the loop portion of the stem-loop is 3-500 nucleotides in length. According to some embodiments, the stem portion of the stem-loop is 4-50 nucleotides in length and the loop portion of the stem-loop is 3-50 nucleotides in length. According to some embodiments, the stem portion of the stem-loop is 4-20 nucleotides in length and the loop portion of the stem-loop is 3-20 nucleotides in length. According to some embodiments, the stem portion of the stem-loop is 4-10 nucleotides in length and the loop portion of the stem-loop is 3-10 nucleotides in length.

According to some embodiments, the loop further comprises one or more nucleic acids or that are used to stabilize the ends. According to other embodiments, the loop further comprises one or more nucleic acids that may be employed in therapeutic methods. According to other embodiments, the loop further comprises one or more nucleic acids that may be employed in diagnostic methods. According to other embodiments, the loop further comprises one or more nucleic acids that that may be employed for research purposes.

According to some embodiments, the minimal nucleic acid structure that is necessary at the 3′ end of the ssDNA is any structure that loops back on itself, i.e., a hairpin structure. However, it is to be understood that a variety of structures are envisioned at the 3′ end, as long as there is at least one stem and one loop. For example, in some embodiments, the ssDNA described herein may comprise at least one stem-loop structure at the 3′ end. In some embodiments, the ssDNA may comprise at least at least two stem-loop structures at the 3′ end. In some embodiments, the ssDNA may comprise at least at least three stem-loop structures at the 3′ end. In some embodiments, the ssDNA may comprise at least at least four stem-loop structures at the 3′ end. In some embodiments, the ssDNA may comprise at least at least five stem-loop structures at the 3′ end.

According to some embodiments, the nucleotides at the 3′ end form a cruciform DNA structure. A DNA cruciform structure can be formed when both strands form a stem-loop structure at the same location in the molecule, and comprises a four-way junction and two closed hairpin-shaped points.

According to some embodiments, the nucleotides at the 3′ end form a hairpin DNA structure. Hairpin loop structures in nucleic acids consist of a base-paired stem structure and a loop sequence with unpaired or non-Watson-Crick-paired nucleotides.

According to some embodiments, the nucleotides at the 3′ end form a hammerhead DNA structure, made up of three base paired helices, separated by short linkers of conserved sequence.

According to some embodiments, the nucleotides at the 3′ end form a quadraplex DNA structure. G-quadruplexes are four-stranded DNA secondary structures (G4s) that form from certain guanine-rich sequences.

According to some embodiments, the nucleotides at the 3′ end form a bulged DNA structure.

According to some embodiments, the nucleotides at the 3′ end form a multibranched loop.

According to some embodiments, the nucleotides at the 3′ end do not form a 2 stem-loop structure. In one embodiment, the nucleotides at the 3′ end do not form an AAV ITR structure.

According to some embodiments, the at least one stem-loop structure at the 3′ end does not comprise the A, A′, D, and D′ regions that would be present in a wild-type AAV ITR.

According to some embodiments, the at least one stem-loop structure at the 3′ end does not comprise the A, A′, B, B′, C, C′, D, and D′ regions that would be present in a wild-type AAV ITR.

According to some embodiments, the at least one stem-loop structure at the 3′ end does not comprise a rep binding element (RBE) that would be present in a wild-type ITR. According to some embodiments, the at least one stem-loop structure at the 3′ end does not comprise a terminal resolution site (trs) that would be present in a wild-type ITR. According to some embodiments, the at least one stem loop structure at the 3′ end is devoid of any viral capsid protein coding sequences.

According to some embodiments, the stem structure at the 3′ end comprises one or more nucleotides that are modified to be exonuclease resistant. According to some embodiments, the stem structure at the 3′ end comprises two or more, 3 or more, 4 or more, 5 or more, 6 or more, 7 or more, 8 or more, 9 or more, 10 or more, or 20 or more nucleotides that are modified to be exonuclease resistant.

According to some embodiments, the stem structure at the 3′ end comprises one or more phosphorothioate-modified nucleotides. According to some embodiments, the stem structure at the 3′ end comprises about 4 to about 10 phosphorothioate-modified nucleotides, e.g., about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10 or about 9 to about 10. According to some embodiments, the stem structure comprises more than 10 phosphorothioate-modified nucleotides.

According to some embodiments, the phosphorothioate-modified nucleotides are located adjacent to each other.

According to some embodiments, the one or more phosphorothioate-modified nucleotides of the 3′ end are resistant to exonuclease degradation. Boranophosphate modified DNA is also resistant to nuclease degradation, and may be considered as an alternative to phosphorothioate modification.

According to further embodiments, the stem structure may comprise at least one functional moiety. In one embodiment, the at least one functional moiety is an aptamer sequence. In further embodiments, the aptamer sequence has a high binding affinity to a nuclear localized protein.

According to some embodiments, the nucleotides in the loop are chemically modified with functional groups in order to alter their properties.

According to some embodiments, the loop further comprises one or more aptamers. According to some embodiments, the aptamer is identified from the Apta-index database of aptamers available to the public (aptagen.com/apta-index).

According to some embodiments, the loop further comprises one or more synthetic ribozymes.

According to some embodiments, the loop further comprises one or more antisense oligonucleotides (ASOs).

According to some embodiments, the loop further comprises one or more short-interfering RNAs (siRNAs).

According to some embodiments, the loop further comprises one or more antiviral nucleoside analogues (ANAs).

According to some embodiments, the loop further comprises one or more triplex forming oligonucleotides.

According to some embodiments, the loop further comprises one or more gRNAs or gDNAs.

According to some embodiments, the loop further comprises one or more molecular probes, for example nucleic acid based fluorescent probes.

According to some embodiments, “click” azide-alkyne cycloaddition (Kolb et al., Angew. Chem. Int. Ed. Engl. 2001, 40, 2004-2021) is used to modify the nucleotides in the loop. Click chemistry was developed to join together organic molecules under mild conditions in the presence of a diverse range of functional groups. Most click-mediated modifications are performed on the nitrogenous bases by introducing novel base analogues, attaching fluorophores or isotopic elements for molecular imaging, forming inter-strand linkages between oligonucleotides, and for the bioconjugation of molecules. The best example of click chemistry is the Cu¹ catalyzed version of Huisgen's [3+2] azide-alkyne cycloaddition reaction (Angew. Chem., Int. Ed. 1963, 2, 633-645), discovered independently by Sharpless and Meldal (the CuAAC reaction) (Angew. Chem., Int. Ed. 2002, 41, 2596-2599).

According to some embodiments, the introduction of active amino or thiol groups into synthesized oligonucleotides provides acceptors for, e.g., subsequent chemical fluorescent labeling.

According to some embodiments, the stem-loop structure may comprise alternative or modified nucleotides, including, but not limited to, ribonucleic acids (RNA), peptide-nucleic acids (PNA), locked nucleic acids (LNA). According to some embodiments, the loop portion of the stem-loop structure may comprise a chemical structure that does not comprise nucleic acids.

ii) 5′ End Stem-Loop Structure of ssDNA

As described herein, according to some aspects, the disclosure provides a ssDNA molecule comprising at least one nucleic acid sequence of interest flanked by at least one stem-loop structure at the 3′ end, as set forth in detail above. According to some embodiments, the ssDNA molecule further comprises a 5′ end, comprising at least one stem-loop structure. According to some embodiments, the DNA structure at the 5′ end is the same as the DNA structure at the 3′ end. According to some embodiments, the DNA structure at the 5′ end is different from the DNA structure at the 3′ end.

For example, in some embodiments, the ssDNA described herein may comprise at least one stem-loop structure at the 5′ end. According to some embodiments, ssDNA may comprise at least at least two stem-loop structures at the 5′ end. According to some embodiments, the ssDNA may comprise at least at least three stem-loop structures at the 5′ end. According to some embodiments, the ssDNA may comprise at least at least four stem-loop structures at the 5′ end. According to some embodiments, the ssDNA may comprise at least at least five stem-loop structures at the 5′ end.

According to some embodiments, the nucleotides at the 5′ end form a cruciform DNA structure.

According to some embodiments, the nucleotides at the 5′ end form a hairpin structure.

According to some embodiments, the nucleotides at the 5′ end form a hammerhead structure.

According to some embodiments, the nucleotides at the 5′ end form a quadraplex structure.

According to some embodiments, the nucleotides at the 5′ end form a bulging structure.

According to some embodiments, the nucleotides at the 5′ end form a multibranched loop.

According to some embodiments, the nucleotides at the 5′ end do not form a 2 stem-loop structure. In one embodiment, the nucleotides at the 5′ end do not form an AAV ITR structure.

According to some embodiments, the at least one stem-loop structure at the 5′ end does not comprise the A, A′, D, and D′ regions that would be present in a wild-type AAV ITR.

According to some embodiments, the at least one stem-loop structure at the 5′ end does not comprise the A, A′, B, B′, C, C′, D, and D′ regions that would be present in a wild-type AAV ITR.

According to some embodiments, the at least one stem-loop structure at the 5′ end does not comprise a rep binding element (RBE) that would be present in a wild-type ITR. According to some embodiments, the at least one stem-loop structure at the 5′ end does not comprise a terminal resolution site (trs) that would be present in a wild-type ITR. According to some embodiments, the at least one stem loop structure at the 5′ end is devoid of any viral capsid protein coding sequences.

According to some embodiments, the stem structure at the 5′ end comprises one or more nucleotides that are modified to be exonuclease resistant. According to some embodiments, the stem structure at the 5′ end comprises two or more, 3 or more, 4 or more, 5 or more, 6 or more, 7 or more, 8 or more, 9 or more, 10 or more, or 20 or more nucleotides that are modified to be exonuclease resistant.

According to some embodiments, the stem structure comprises one or more phosphorothioate-modified nucleotides. According to some embodiments, the stem structure comprises about 4 to about 10 phosphorothioate-modified nucleotides, e.g., about 4 to about 5, about 4 to about 6, about 4 to about 7, about 4 to about 8, about 4 to about 9, about 4 to about 10, about 5 to about 6, about 5 to about 7, about 5 to about 8, about 5 to about 9, about 5 to about 10, about 6 to about 7, about 6 to about 8, about 6 to about 9, about 6 to about 10, about 7 to about 8, about 7 to about 9, about 7 to about 10, about 8 to about 9, about 8 to about 10 or about 9 to about 10. According to some embodiments, the stem structure comprises more than 10 phosphorothioate-modified nucleotides.

According to some embodiments, the phosphorothioate-modified nucleotides are located adjacent to each other. According to some embodiments, the one or more phosphorothioate-modified nucleotides of the are resistant to exonuclease degradation.

According to some embodiments, the loop further comprises one or more nucleic acids or that are used to stabilize the ends. According to other embodiments, the loop further comprises one or more nucleic acids that may be employed in therapeutic methods. According to other embodiments, the loop further comprises one or more nucleic acids that may be employed in diagnostic methods. According to other embodiments, the loop further comprises one or more nucleic acids that that may be employed for research purposes.

According to some embodiments, the nucleotides in the loop are chemically modified with functional groups in order to alter their properties.

According to some embodiments, the loop further comprises one or more aptamers. According to some embodiments, the aptamer is identified from the Apta-index database of aptamers available to the public (aptagen.com/apta-index).

According to some embodiments, the loop further comprises one or more synthetic ribozymes.

According to some embodiments, the loop further comprises one or more antisense oligonucleotides (ASOs).

According to some embodiments, the loop further comprises one or more short-interfering RNAs (siRNAs).

According to some embodiments, the loop further comprises one or more antiviral nucleoside analogues (ANAs).

According to some embodiments, the loop further comprises one or more triplex forming oligonucleotides.

According to some embodiments, the loop further comprises one or more gRNAs or gDNAs.

According to some embodiments, the loop further comprises one or more molecular probes, for example nucleic acid based fluorescent probes.

According to some embodiments, “click” azide-alkyne cycloaddition (Kolb et al., Angew. Chem. Int. Ed. Engl. 2001, 40, 2004-2021) is used to modify the nucleotides in the loop. Click chemistry was developed to join together organic molecules under mild conditions in the presence of a diverse range of functional groups. Most click-mediated modifications are performed on the nitrogenous bases by introducing novel base analogues, attaching fluorophores or isotopic elements for molecular imaging, forming inter-strand linkages between oligonucleotides, and for the bioconjugation of molecules. The best example of click chemistry is the Cu¹ catalyzed version of Huisgen's [3+2] azide-alkyne cycloaddition reaction (Angew. Chem., Int. Ed. 1963, 2, 633-645), discovered independently by Sharpless and Meldal (the CuAAC reaction) (Angew. Chem., Int. Ed. 2002, 41, 2596-2599).

According to some embodiments, the introduction of active amino or thiol groups into synthesized oligonucleotides provides acceptors for, e.g., subsequent chemical fluorescent labeling.

According to some embodiments, the stem-loop structure may comprise alternative or modified nucleotides, including, but not limited to, ribonucleic acids (RNA), peptide-nucleic acids (PNA), locked nucleic acids (LNA). According to some embodiments, the loop portion of the stem-loop structure may comprise a chemical structure that does not comprise nucleic acids.

The single-stranded DNA (ssDNA) molecules described herein have no packaging constraints imposed by the limiting space within the viral capsid. This permits the insertion of one or more genetic elements, e.g., a single-stranded enhancer, a single-stranded intron, a single-stranded posttranscriptional regulatory element, a single-stranded polyadenylation signal, and a single-stranded regulatory switch, large transgenes, multiple transgenes etc.

According to some embodiments, the nucleic acid sequence of interest further comprises at least one single-stranded promoter linked to the at least one nucleic acid sequence of interest.

In other aspects of the disclosure, the single-stranded transgene cassettes find use in gene editing applications, as described in more detail herein.

According to some embodiments, the nucleic acid sequence of interest (also referred to as a transgene herein) encodes a protein that is either absent, inactive, or insufficient activity in the recipient subject or a gene that encodes a protein having a desired biological or a therapeutic effect. The transgene can encode a gene product that can function to correct the expression of a defective gene or transcript. In principle, the expression cassette can include any gene that encodes a protein, polypeptide or RNA that is either reduced or absent due to a mutation or which conveys a therapeutic benefit when overexpressed is considered to be within the scope of the disclosure.

The nucleic acid sequence of interest can comprise any sequence that is useful for treating a disease or disorder in a subject. A ssDNA molecule can be used to deliver and express any gene of interest in the subject, which includes but are not limited to, nucleic acids encoding polypeptides, or non-coding nucleic acids (e.g., RNAi, miRs etc.), as well as exogenous genes and nucleotide sequences, including virus sequences in a subjects' genome, e.g., HIV virus sequences and the like. In some embodiments, ssDNA molecules disclosed herein are used for therapeutic purposes (e.g., for medical, diagnostic, or veterinary uses). In certain embodiments, ssDNA molecules are useful to express any gene of interest in the subject, which includes one or more polypeptides, peptides, ribozymes, peptide nucleic acids, siRNAs, RNAis, antisense oligonucleotides, antisense polynucleotides, or RNAs (coding or non-coding; e.g., siRNAs, shRNAs, micro-RNAs, mRNA or gRNA, and their antisense counterparts (e.g., antagoMiR)), antibodies, antigen binding fragments, or any combination thereof.

Sequences can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENEFORGE® codon optimization and custom gene synthesis platform (Aptagen, Inc., 2190 Fox Mill Rd. Suite 300, Herndon, Va. 20171) or another publicly available database.

In some embodiments, a transgene expressed by the ssDNA molecules is a therapeutic gene. In some embodiments, a therapeutic gene is an antibody, or antibody fragment, or antigen-binding fragment thereof, e.g., a neutralizing antibody or antibody fragment and the like.

In particular, a therapeutic gene is one or more therapeutic agent(s), including, but not limited to, for example, protein(s), polypeptide(s), peptide(s), enzyme(s), antibodies, antigen binding fragments, as well as variants, and/or active fragments thereof, for use in the treatment, prophylaxis, and/or amelioration of one or more symptoms of a disease, dysfunction, injury, and/or disorder.

Exemplary therapeutic genes are described herein in the section entitled “Method of Treatment”.

According to any of the above aspects and embodiments, the ssDNA molecules are synthetically produced.

According to any of the above aspects and embodiments, the ssDNA molecules are devoid of any viral capsid protein coding sequences.

According to any of the above aspects the DNA is peptide nucleic acid (PNA) are synthetic mimics of DNA.

As described herein, the present disclosure relates to single-stranded (ssDNA) molecules. In some aspects, the ssDNA molecules are, e.g., synthetic AAV vectors, e.g., single-stranded (ss) synthetic AAV vectors, produced from double stranded closed-ended DNA comprising phosphorothioate (PS) bonds. The PS bond substitutes a sulfur atom for a non-bridging oxygen in the phosphate backbone of an oligonucleotide. Advantageously, this modification renders the internucleotide linkage resistant to nuclease degradation, and provides accuracy for targeting of the exonuclease.

In some aspects, the disclosure provides a single-stranded transgene cassette comprising at least one single-stranded transgene and at least one inverted terminal repeat (ITR) comprising one or more phosphorothioate-modified nucleotides. According to some embodiments, a ssDNA molecule comprises a first ITR and an optional second ITR; wherein at least one of the first ITR and the optional second ITR comprises one or more phosphorothioate-modified nucleotides. In further embodiments, the ssDNA molecule comprises a 3′ terminal fragment that comprises a terminal resolution site (trs) sequence.

According to some aspects, the disclosure provides an isolated, linear, and single-stranded DNA (ssDNA) molecule comprising a single-stranded transgene cassette comprising at least one single-stranded transgene; and a first inverted terminal repeat (ITR) and a second ITR that each flanks the at least one single-stranded transgene cassette; wherein at least one of the first ITR and the second ITR comprises one or more phosphorothioate-modified nucleotides.

As described in more detail herein, the ssDNA molecule is synthetically produced in vitro from dsDNA comprising phosphorothioate (PS) bonds (“starting material”) by removing one DNA strand from a specific nicking site and to a PS bonded site of the dsDNA. According to further embodiments, the ssDNA molecule is synthetically produced in vitro in a cell-free environment.

According to some embodiments, it is a feature of the present disclosure that the 3′ terminal portion of the double stranded DNA molecule (starting material) comprises a nickase recognition sequence. In one embodiment, the 3′ terminal portion of the dsDNA molecule comprises the sequence 5′-CCAA-3′. In some embodiments the 3′ terminal portion of the dsDNA molecule comprises any one or more of the sequences shown in Table 8 below. Further, since these are unique sequences after a double stranded ceDNA with special engineered nick sites has been nicked by a nicking endonuclease as shown in the table, resultant ssDNA molecules also comprise any one or more of the sequences shown in Table 8 below in its 3′ terminal fragment.

TABLE 8

| Sequence | Nicking Endonuclease |
|---|---|
| 5′-CCAA-3′ | (Nb.BtsI) (Nb.BsrDI) |
| | (Nt.CviPII) |
| 5′-CCAAGC-3′ | (Nb.BbvCI) |
| 5′-CCAACC-3′ | (Nt.BbvCI) |
| 5′-CCAAGAGTCNNNN-3′ | (Nt.BstNBI)- |
| | N can be A, G, C, or T |
| 5′-CCAAG-3′ | (Nb.BsmI) |
| 5′-CCAAC-3′ | (Nb.BssSI) |
| 5′-CCAAGGATCNNNN-3′ | (Nt.AlwI) |
| 5′-CCAAGTCTCN-3′ | (Nt.BsmAI) |
| 5′-CCAAGCTCTTCN-3′ | (Nt.BspQI) |

According to some embodiments, the 3′ terminal fragment of the ssDNA molecule comprises a terminal residue that is hydroxylated (—OH) such that it enables polymerase activity once the ssDNA is transported to the nucleus of a host cell in which the ssDNA get convert to regenerated dsDNA that is capable of being expressed.

According to some embodiments, the ssDNA molecule comprises a 3′ terminal fragment that comprises a terminal resolution site (trs) sequence.

The ssDNA molecule described herein is capable of being transported across the nuclear membrane from the cytosol into the nucleus of a host cell, and reached upon by host cell DNA polymerase to generate a double stranded DNA (“regenerated dsDNA) for expression of the transgene in the host cell. Accordingly, in some embodiments, the terminal residue that is hydroxylated (—OH) in the ssDNA molecule is critical to be responsive towards DNA polymerase activity inside the nucleus of a host cell. According to further embodiments, the DNA polymerase generates a dsDNA molecule.

Importantly, the ssDNA molecule does not activate or minimally activates an innate immune pathway inside a host cell. As used herein the term “the innate immune response” refers to the cellular pathways that respond to pathogen associated molecular patterns and activate a defense response through the RIG-I-like receptors, the toll-like receptors, or other pathogen associated molecular pattern receptors to activate interferon, NF-kappa-B, STAT, IRF and other response pathways that protect against pathogen infection. According to some embodiments, the innate immune pathway may be the cGAS/STING pathway, the TLR9 pathway, an inflammasome-mediated pathway, or a combination thereof. Indicators of the activation of the innate immune response include increased expression and/or phosphorylation of IRF family members, increased expression of the RIG-I like receptors, and increased expression of interferons and/or chemokines.

According to some embodiments, the single-stranded transgene cassette further comprises at least one single-stranded promoter operably linked to the at least one single-stranded transgene; and the dsDNA molecule comprises a regenerated double-stranded expression cassette comprising at least one regenerated double-stranded transgene and at least one double-stranded promoter operably linked to the regenerated double-stranded transgene to control expression of the at least one regenerated double-stranded transgene. The double-stranded expression cassette is capable of being expressed in a host cell, for example a host cell in vivo. In some embodiments, the double-stranded expression cassette is capable of being expressed into at least one therapeutic protein or a fragment thereof.

In further embodiments, the single-stranded transgene cassette further comprises one or more genetic elements selected from the group consisting of a single-stranded enhancer, a single-stranded intron, a single-stranded posttranscriptional regulatory element, a single-stranded polyadenylation signal, and a single-stranded regulatory switch.

In other aspects of the disclosure, the single-stranded transgene cassettes find use in gene editing applications.

Accordingly, in some embodiments, the at least one single-stranded transgene cassette is a promoterless transgene cassette; and the dsDNA molecule comprises at least one regenerated promoterless double-stranded transgene. In some embodiments, the at least one regenerated promoterless double-stranded transgene is capable of being inserted at a target locus in the genome of a host cell. In further embodiments, the at least one regenerated promoterless double-stranded transgene is capable of being inserted at a target locus in the genome of a host cell in vivo. In some embodiments, the at least one regenerated promoterless double-stranded transgene is capable of being inserted at the target locus to replace or to supplement at least one target gene. In other embodiments, the at least one regenerated promoterless double-stranded transgene is capable of being inserted at the target locus via homology-directed recombination (HDR) or microhomology-mediated end joining (MMEJ). In other further embodiments, the at least one single-stranded transgene is a single-stranded donor sequence; and the single-stranded transgene cassette further comprises a single-stranded 5′ homology arm and a single-stranded 3′ homology arm flanking the single-stranded donor sequence. The single-stranded 5′ homology arm and the single-stranded 3′ homology arm are each between about 10 to 2000 nt in length, for example about 100 to 2000 nt in length or about 1000 to 2000 nt in length, or about 10 to 1000 nt in length, for example about 100 to 1000 nt in length or about 10 to 500 nt in length, about 50 to 500 nt in length or about 100 to 500 nt in length, about 10 to 50 nt in length, about 50 to 500 nt in length or about 500 to 1000 nt in length, about 500 to 1500 nt in length, about 1500 to 2000 nt in length, about 2 to 1000 nt in length, about 2 to 500 nt in length, about 2 to 100 nt in length, or about 2 to 50 nt in length. In some embodiments, the at least one regenerated promoterless double-stranded transgene is capable of being inserted at the target locus via non-homology end joining (NHEJ). In some embodiments, the at least one single-stranded transgene is a single-stranded donor sequence; and the single-stranded transgene cassette is devoid of a single-stranded 5′ homology arm and a single-stranded 3′ homology arm. In other embodiments, the single-stranded transgene cassette is cleavable and further comprises: at least a first single-stranded guide RNA (gRNA) target sequence (TS); at least a first single-stranded protospacer adjacent motif (PAM); at least a second single-stranded gRNA TS; and at least a second single-stranded PAM.

As described in more detail herein, in some embodiments, the ssDNA molecule described herein is synthetically produced from the dsDNA construct by a method comprising: a) contacting the dsDNA construct with one or more nicking endonucleases that nick one of the single strands of the dsDNA construct at one or more nick sites; and b) contacting the dsDNA construct with an exonuclease capable of removing nucleotides from the nicked strand of the dsDNA construct to thereby produce the ssDNA molecule.

Closed-Ended DNA (ceDNA) Vectors

In some embodiments, LNPs provided by the present disclosure comprise closed-ended DNA (ceDNA).

In some embodiments, the TNA comprises closed-ended linear duplexed (ceDNA) vectors that can express a transgene (e.g. a therapeutic nucleic acid (TNA)). The ceDNA vectors as described herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

ceDNA vectors preferably have a linear and continuous structure rather than a non-continuous structure. The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, it is likely to remain a single molecule. In some embodiments, ceDNA vectors can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects, and also in view of their DNA methylation which is of prokaryotic type for ceDNA-plasmids and of eukaryotic type for the ceDNA vector.

Provided herein are non-viral, capsid-free ceDNA molecules with covalently closed ends (ceDNA). These non-viral capsid free ceDNA molecules can be produced in permissive host cells from an expression construct (e.g., a ceDNA-plasmid, a ceDNA-bacmid, a ceDNA-baculovirus, or an integrated cell-line) containing a heterologous gene (e.g., a transgene, in particular a therapeutic transgene) positioned between two different inverted terminal repeat (ITR) sequences, where the ITRs are different with respect to each other. In some embodiments, one of the ITRs is modified by deletion, insertion, and/or substitution as compared to a wild-type ITR sequence (e.g. AAV ITR); and at least one of the ITRs comprises a functional terminal resolution site (trs) and a Rep binding site. The ceDNA vector is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g., exonuclease I or exonuclease III), e.g., for over an hour at 37° C.

In one aspect, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR. In one embodiment, the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. In one embodiment, the first ITR and the second ITR are both modified but are different sequences, or have different modifications, or are not identical modified ITRs, and have different 3D spatial configurations. Stated differently, a ceDNA vector with asymmetric ITRs have ITRs where any changes in one ITR relative to the WT-ITR are not reflected in the other ITR; or alternatively, where the asymmetric ITRs have a the modified asymmetric ITR pair can have a different sequence and different three-dimensional shape with respect to each other.

In one embodiment, a ceDNA vector comprises, in the 5′ to 3′ direction: a first adeno-associated virus (AAV) inverted terminal repeat (ITR), a nucleotide sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In one embodiment, a modified ITR pair are substantially symmetrical as defined herein, that is, the modified ITR pair can have a different sequence but have corresponding or the same symmetrical three-dimensional shape. In some embodiments, the symmetrical ITRs, or substantially symmetrical ITRs can be wild type ITRs (WT-ITRs) as described herein. That is, both ITRs have a wild-type sequence from the same AAV serotype. In some other embodiments, the two wild-type ITRs can be from different AAV serotypes. For example, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

The wild-type or mutated or otherwise modified ITR sequences provided herein represent DNA sequences included in the expression construct (e.g., ceDNA-plasmid, ceDNA Bacmid, ceDNA-baculovirus) for production of the ceDNA vector. Thus, ITR sequences actually contained in the ceDNA vector produced from the ceDNA-plasmid or other expression construct may or may not be identical to the ITR sequences provided herein as a result of naturally occurring changes taking place during the production process (e.g., replication error).

In one embodiment, a ceDNA vector in the LNPs of the present disclosure comprising the expression cassette with a transgene which is a therapeutic nucleic acid sequence, can be operatively linked to one or more regulatory sequence(s) that allows or controls expression of the transgene. In one embodiment, the polynucleotide comprises a first ITR sequence and a second ITR sequence, wherein the nucleotide sequence of interest is flanked by the first and second ITR sequences, and the first and second ITR sequences are asymmetrical relative to each other, or symmetrical relative to each other.

In one embodiment, an expression cassette is located between two ITRs in the following order with one or more of: a promoter operably linked to a transgene, a posttranscriptional regulatory element, and a polyadenylation and termination signal. In one embodiment, the promoter is regulatable-inducible or repressible. The promoter can be any sequence that facilitates the transcription of the transgene. In one embodiment the promoter is a CAG promoter, or variation thereof. The posttranscriptional regulatory element is a sequence that modulates expression of the transgene, as a non-limiting example, any sequence that creates a tertiary structure that enhances expression of the transgene which is a therapeutic nucleic acid sequence.

In one embodiment, the posttranscriptional regulatory element comprises WPRE. In one embodiment, the polyadenylation and termination signal comprise BGHpolyA. Any cis regulatory element known in the art, or combination thereof, can be additionally used e.g., SV40 late polyA signal upstream enhancer sequence (USE), or other posttranscriptional processing elements including, but not limited to, the thymidine kinase gene of herpes simplex virus, or hepatitis B virus (HBV). In one embodiment, the expression cassette length in the 5′ to 3′ direction is greater than the maximum length known to be encapsidated in an AAV virion. In one embodiment, the length is greater than 4.6 kb, or greater than 5 kb, or greater than 6 kb, or greater than 7 kb. Various expression cassettes are exemplified herein.

In one embodiment, the expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. In some embodiments, the expression cassette can comprise a transgene which is a therapeutic nucleic acid sequence in the range of 500 to 50,000 nucleotides in length. In one embodiment, the expression cassette can comprise a transgene which is a therapeutic nucleic acid sequence in the range of 500 to 75,000 nucleotides in length. In one embodiment, the expression cassette can comprise a transgene which is a therapeutic nucleic acid sequence in the range of 500 to 10,000 nucleotides in length. In one embodiment, the expression cassette can comprise a transgene which is a therapeutic nucleic acid sequence in the range of 1000 to 10,000 nucleotides in length. In one embodiment, the expression cassette can comprise a transgene which is a therapeutic nucleic acid sequence in the range of 500 to 5,000 nucleotides in length. The ceDNA vectors do not have the size limitations of encapsidated AAV vectors, and thus enable delivery of a large-size expression cassette to the host. In one embodiment, the ceDNA vector is devoid of prokaryote-specific methylation.

In one embodiment, the rigid therapeutic nucleic acid can be a plasmid.

In one embodiment, the ceDNA vectors disclosed herein are used for therapeutic purposes (e.g., for medical, diagnostic, or veterinary uses) or immunogenic polypeptides.

The expression cassette can comprise any transgene which is a therapeutic nucleic acid sequence. In certain embodiments, the ceDNA vector comprises any gene of interest in the subject, which includes protein, enzyme, one or more polypeptides, peptides, ribozymes, peptide nucleic acids, siRNAs, gRNA, mRNA, RNAis, antisense oligonucleotides, antisense polynucleotides, antibodies, antigen binding fragments, or any combination thereof.

In one embodiment, the ceDNA expression cassette can include, for example, an expressible exogenous sequence (e.g., open reading frame) that encodes a protein that is either absent, inactive, or insufficient activity in the recipient subject or a gene that encodes a protein having a desired biological or a therapeutic effect. In one embodiment, the exogenous sequence such as a donor sequence can encode a gene product that can function to correct the expression of a defective gene or transcript. In one embodiment, the expression cassette can also encode corrective DNA strands, encode polypeptides, sense or antisense oligonucleotides, or RNAs (coding or non-coding; e.g., siRNAs, shRNAs, micro-RNAs, and their antisense counterparts (e.g., antagoMiR)). In one embodiment, expression cassettes can include an exogenous sequence that encodes a reporter protein to be used for experimental or diagnostic purposes, such as b-lactamase, b-galactosidase (LacZ), alkaline phosphatase, thymidine kinase, green fluorescent protein (GFP), chloramphenicol acetyltransferase (CAT), luciferase, and others well known in the art.

Accordingly, the expression cassette can include any gene that encodes a protein, polypeptide or RNA that is either reduced or absent due to a mutation or which conveys a therapeutic benefit when overexpressed is considered to be within the scope of the disclosure. The ceDNA vector may comprise a template or donor nucleotide sequence used as a correcting DNA strand to be inserted after a double-strand break (or nick) provided by a nuclease. The ceDNA vector may include a template nucleotide sequence used as a correcting DNA strand to be inserted after a double-strand break (or nick) provided by a guided RNA nuclease, meganuclease, or zinc finger nuclease.

### IV. Preparation of Lipid Nanoparticles (LNPs)

Lipid nanoparticles (LNPs) can form spontaneously upon mixing of a therapeutic nucleic acid (e.g., ceDNA, ssDNA, synthetic AAV, etc., as described herein) and a pharmaceutically acceptable excipient that comprises a lipid.

Generally, LNPs can be formed by any method known in the art. For example, the LNPs can be prepared by the methods described, for example, in US2013/0037977, US2010/0015218, US2013/0156845, US2013/0164400, US2012/0225129, and US2010/0130588, content of each of which is incorporated herein by reference in its entirety. In some embodiments, LNPs can be prepared using a continuous mixing method, a direct dilution process, or an in-line dilution process. The processes and apparatuses for preparing lipid nanoparticles using direct dilution and in-line dilution processes are described in US2007/0042031, the content of which is incorporated herein by reference in its entirety. The processes and apparatuses for preparing lipid nanoparticles using step-wise dilution processes are described in US2004/0142025, the content of which is incorporated herein by reference in its entirety.

According to some embodiments, the disclosure provides for an LNP comprising a DNA vector, including a ceDNA vector, ssDNA vector, or synthetic AAV, as described herein and an ionizable lipid. For example, a lipid nanoparticle formulation that is made and loaded with therapeutic nucleic acid like ceDNA obtained by the process as disclosed in International Patent Application No. PCT/US2018/050042, filed on Sep. 7, 2018, which is incorporated by reference in its entirety herein. This can be accomplished by high energy mixing of ethanolic lipids with aqueous ceDNA, ssDNA or mRNA at low pH which protonates the ionizable lipid and provides favorable energetics for synthetic AAV/lipid association and nucleation of particles. The particles can be further stabilized through aqueous dilution and removal of the organic solvent. The particles can be concentrated to the desired level.

Generally, the lipid particles are prepared at a total lipid to synthetic ceDNA, ssDNA or mRNA (mass or weight) ratio of from about 10:1 to 30:1. In some embodiments, the lipid to ssDNA molecule or the dsDNA construct ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and synthetic exeDNA, ssDNA or mRNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo at low pH and to drive membrane association and fusogenicity. Generally, ionizable lipids are lipids comprising at least one amino group that is positively charged or becomes protonated under acidic conditions, for example at pH of 6.5 or lower.

In one embodiment, the LNPs can be prepared by an impinging jet process. Generally, the particles are formed by mixing lipids dissolved in alcohol (e.g., ethanol) with ceDNA, ssDNA or mRNA dissolved in a buffer, e.g., a citrate buffer, a sodium acetate buffer, a sodium acetate and magnesium chloride buffer, a malic acid buffer, a malic acid and sodium chloride buffer, or a sodium citrate and sodium chloride buffer. The mixing ratio of lipids to ceDNA, ssDNA or mRNA can be about 45-55% lipid and about 65-45% ceDNA, ssDNA or mRNA.

The lipid solution can contain an ionizable lipid, a ceramide, a lipid-anchored polymer and a sterol (e.g., cholesterol) at a total lipid concentration of 5-30 mg/mL, more likely 5-15 mg/mL, most likely 9-12 mg/mL in an alcohol, e.g., in ethanol. In the lipid solution, mol ratio of the lipids can range from about 25-98% for the cationic lipid, preferably about 35-65%; about 0-15% for the non-ionic lipid, preferably about 0-12%; about 0-15% for the PEG or PEG conjugated lipid molecule, preferably about 1-6%; and about 0-75% for the sterol, preferably about 30-50%.

The ceDNA solution can comprise the ceDNA at a concentration range from 0.3 to 1.0 mg/mL, preferably 0.3-0.9 mg/mL in buffered solution, with pH in the range of 3.5-5.

For forming the LNPs, in one exemplary but nonlimiting embodiment, the two liquids are heated to a temperature in the range of about 15-40° C., preferably about 30-40° C., and then mixed, for example, in an impinging jet mixer, instantly forming the LNP. The mixing flow rate can range from 10-600 mL/min. The tube ID can have a range from 0.25 to 1.0 mm and a total flow rate from 10-600 mL/min. The combination of flow rate and tubing ID can have the effect of controlling the particle size of the LNPs between 30 and 200 nm. The solution can then be mixed with a buffered solution at a higher pH with a mixing ratio in the range of 1:1 to 1:3 vol:vol, preferably about 1:2 vol:vol. If needed this buffered solution can be at a temperature in the range of 15-40° C. or 30-40° C. The mixed LNPs can then undergo an anion exchange filtration step. Prior to the anion exchange, the mixed LNPs can be incubated for a period of time, for example 30 mins to 2 hours. The temperature during incubating can be in the range of 15-40° C. or 30-40° C. After incubating the solution is filtered through a filter, such as a 0.8 μm filter, containing an anion exchange separation step. This process can use tubing IDs ranging from 1 mm ID to 5 mm ID and a flow rate from 10 to 2000 mL/min.

After formation, the LNPs can be concentrated and diafiltered via an ultrafiltration process where the alcohol is removed and the buffer is exchanged for the final buffer solution, for example, phosphate buffered saline (PBS) at about pH 7, e.g., about pH 6.9, about pH 7.0, about pH 7.1, about pH 7.2, about pH 7.3, or about pH 7.4.

The ultrafiltration process can use a tangential flow filtration format (TFF) using a membrane nominal molecular weight cutoff range from 30-500 kD. The membrane format is hollow fiber or flat sheet cassette. The TFF processes with the proper molecular weight cutoff can retain the LNP in the retentate and the filtrate or permeate contains the alcohol; citrate buffer and final buffer wastes. The TFF process is a multiple step process with an initial concentration to a ceDNA concentration of 1-3 mg/mL. Following concentration, the LNPs solution is diafiltered against the final buffer for 10-20 volumes to remove the alcohol and perform buffer exchange. The material can then be concentrated an additional 1-3-fold. The concentrated LNP solution can be sterile filtered.

### V. Pharmaceutical Compositions and Formulations

The present disclosure also provides a pharmaceutical composition comprising the LNPs of the present disclosure and at least one pharmaceutically acceptable excipient.

According to some embodiments, the TNA (e.g., ceDNA) is encapsulated in the LNP. In one embodiment, the LNPs of the disclosure are provided with full encapsulation, partial encapsulation of therapeutic nucleic acid. In one embodiment, the nucleic acid therapeutics is fully encapsulated in the LNPs to form a nucleic acid containing lipid particle. In one embodiment, the nucleic acid may be encapsulated within the lipid portion of the particle, thereby protecting it from enzymatic degradation.

In one embodiment, encapsulation of TNA (e.g., ceDNA) in the LNPs of the present disclosure can be determined by performing a membrane-impermeable fluorescent dye exclusion assay, which uses a dye that has enhanced fluorescence when associated with nucleic acid, for example, an Oligreen® assay or PicoGreen® assay. Generally, encapsulation is determined by adding the dye to the lipid particle formulation, measuring the resulting fluorescence, and comparing it to the fluorescence observed upon addition of a small amount of nonionic detergent. Detergent-mediated disruption of the lipid bilayer releases the encapsulated TNA (e.g., ceDNA), allowing it to interact with the membrane-impermeable dye. Encapsulation of ceDNA can be calculated as E=(Io−I)/Io, where I and Io refer to the fluorescence intensities before and after the addition of detergent.

Depending on the intended use of the LNPs, the proportions of the components can vary and the delivery efficiency of a particular formulation can be measured using, for example, an endosomal release parameter (ERP) assay.

In one embodiment, the TNA (e.g., ceDNA) can be complexed with the lipid portion of the particle or encapsulated in the lipid position of the lipid particle (e.g., lipid nanoparticle). In one embodiment, the TNA can be fully encapsulated in the lipid position of the lipid particle (e.g., lipid nanoparticle), thereby protecting it from degradation by a nuclease, e.g., in an aqueous solution. In one embodiment, the TNA in the lipid particle (e.g., lipid nanoparticle) is not substantially degraded after exposure of the lipid particle (e.g., lipid nanoparticle) to a nuclease at 37° C. for at least about 20, 30, 45, or 60 minutes. In some embodiments, the TNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In another embodiment, the lipid nanoparticles are kept in a frozen state devoid of TNA. Such lipid nanoparticles are known as empty LNP. The TNA may be combined with the empty LNP, and the TNA is spontaneously taken up by the LNP at room temperature (rt) or higher. Combining empty LNPs with TNA using bedside formulation is advantageous in minimizing waste and promoting increased stability since the TNA and empty LNP can be stored separately under conditions to optimize each component. See WO2021155274A1.

In one embodiment, the LNPs are substantially non-toxic to a subject, e.g., to a mammal such as a human.

In one embodiment, the pharmaceutical composition comprising LNPs of the disclosure is an aqueous solution. In one embodiment, the pharmaceutical composition comprising LNPs of the disclosure is a lyophilized powder.

According to some aspects, the at least one pharmaceutically acceptable excipient in the pharmaceutical compositions of the present disclosure is sucrose, tris, trehalose and/or glycine.

In one embodiment, the pharmaceutical compositions comprising LNPs of the disclosure are suitable for administration to a subject for in vivo delivery to cells, tissues, or organs of the subject. In some embodiments, the pharmaceutical composition is suitable for a desired route of therapeutic administration (e.g., parenteral administration). The pharmaceutical compositions for therapeutic purposes can be formulated as a solution, microemulsion, dispersion, liposomes, empty LNPs and TNA solution for bedside combination, or other ordered structure suitable for high TNA (e.g., ceDNA) vector concentration. Sterile injectable solutions can be prepared by incorporating the TNA (e.g., ceDNA) vector compound in the required amount in an appropriate buffer with one or a combination of ingredients enumerated above, as required, followed by filtered sterilization.

Pharmaceutical compositions comprising LNPs of the disclosure are suitable for topical, systemic, intra-amniotic, intrathecal, intracranial, intraarterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

Pharmaceutical compositions for therapeutic purposes typically must be sterile and stable under the conditions of manufacture and storage.

In one embodiment, LNPs are solid core particles that possess at least one lipid bilayer. In one embodiment, the LNPs have a non-bilayer structure, i.e., a non-lamellar (i.e., non-bilayer) morphology. Without limitations, the non-bilayer morphology can include, for example, three dimensional tubes, rods, cubic symmetries, etc. The non-lamellar morphology (i.e., non-bilayer structure) of the LNPs can be determined using analytical techniques known to and used by those of skill in the art. Such techniques include, but are not limited to, Cryo-Transmission Electron Microscopy (“Cryo-TEM”), Differential Scanning calorimetry (“DSC”), X-Ray Diffraction, and the like. For example, the morphology of the lipid particles (lamellar vs. non-lamellar) can readily be assessed and characterized using, e.g., Cryo-TEM analysis as described in US2010/0130588, the content of which is incorporated herein by reference in its entirety.

In one embodiment, the LNPs having a non-lamellar morphology are electron dense.

In one embodiment, the LNPs provided by the present disclosure are either unilamellar or multilamellar in structure. In some aspects, the pharmaceutical composition of the disclosure comprises multi-vesicular particles and/or foam-based particles. By controlling the composition and concentration of the lipid components, one can control the rate at which the lipid conjugates exchange out of the lipid particle and, in turn, the rate at which the LNP becomes fusogenic. In addition, other variables including, for example, pH, temperature, or ionic strength, can be used to vary and/or control the rate at which the LNP becomes fusogenic. Other methods which can be used to control the rate at which the LNP becomes fusogenic will be apparent to those of ordinary skill in the art based on this disclosure. It will also be apparent that by controlling the composition and concentration of the lipid conjugate, one can control the lipid particle size.

In some embodiments, the reference LNP is an LNP that does not comprise the helper lipid represented by Formula (I), (II), (III), or (IV), or a salt or an ester thereof, or a deuterated analogue of any of the foregoing; or (ii) comprises a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) and a reference lipid-anchored polymer comprising at least two hydrophobic tails each comprise 12 to 15 carbon atoms in a single aliphatic chain backbone.

According to some embodiments, for ophthalmic delivery, interfering RNA-ligand conjugates and nanoparticle-ligand conjugates may be combined with ophthalmologically acceptable preservatives, co-solvents, surfactants, viscosity enhancers, penetration enhancers, buffers, sodium chloride, or water to form an aqueous, sterile ophthalmic suspension or solution.

### Unit Dosage

In one embodiment, the pharmaceutical compositions can be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### VI. Methods of Treatment

In some aspects, the present disclosure provides methods of treating a disorder in a subject that comprise administering to the subject an effective amount of an LNP of the disclosure of the pharmaceutical composition comprising the LNP of the disclosure. In some embodiments, the disorder is a genetic disorder.

As used herein, the term “genetic disease” or “genetic disorder” refers to a disease, partially or completely, directly or indirectly, caused by one or more abnormalities in the genome, including and especially a condition that is present from birth. The abnormality may be a mutation, an insertion or a deletion in a gene. The abnormality may affect the coding sequence of the gene or its regulatory sequence.

Provided herein are methods for treating genetic disorders by administering the LNP of the disclosure or the pharmaceutical composition comprising LNPs of the disclosure. There are a number of inherited diseases in which defective genes are known, and typically fall into two classes: deficiency states, usually of enzymes, which are generally inherited in a recessive manner, and unbalanced states, which may involve regulatory or structural proteins, and which are typically but not always inherited in a dominant manner. For deficiency state diseases, the LNPs and LNP compositions of the disclosure can be used to deliver transgenes to bring a normal gene into affected tissues for replacement therapy, as well, in some embodiments of any of the aspects and embodiments herein, to create animal models for the disease using antisense mutations. For unbalanced disease states, the LNPs and LNP compositions of the disclosure can be used to create a disease state in a model system, which could then be used in efforts to counteract the disease state. Thus, the LNPs or LNP compositions of the disclosure and methods disclosed herein permit the treatment of genetic diseases. As used herein, a disease state is treated by partially or wholly remedying the deficiency or imbalance that causes the disease or makes it more severe.

In general, the LNPs and LNP compositions of the disclosure can be used to deliver any transgene in accordance with the description above to treat, prevent, or ameliorate the symptoms associated with any disorder related to gene expression. Illustrative disease states include, but are not-limited to: cystic fibrosis (and other diseases of the lung), hemophilia A, hemophilia B, thalassemia, anemia and other blood disorders, AIDS, Alzheimer's disease, Parkinson's disease, Huntington's disease, amyotrophic lateral sclerosis, epilepsy, and other neurological disorders, cancer, diabetes mellitus, muscular dystrophies (e.g., Duchenne, Becker), Hurler's disease, adenosine deaminase deficiency, metabolic defects, retinal degenerative diseases (and other diseases of the eye), mitochondriopathies (e.g., Leber's hereditary optic neuropathy (LHON), Leigh syndrome, and subacute sclerosing encephalopathy), myopathies (e.g., facioscapulohumeral myopathy (FSHD) and cardiomyopathies), diseases of solid organs (e.g., brain, liver, kidney, heart), and the like. In some embodiments of any of the aspects and embodiments herein, the ceDNA vectors as disclosed herein can be advantageously used in the treatment of individuals with metabolic disorders (e.g., ornithine transcarbamylase deficiency).

In one embodiment of any of the aspects or embodiments herein, the LNPs of the disclosure or the pharmaceutical compositions comprising the LNPs of the disclosure can be used to treat, ameliorate, and/or prevent a disease or disorder caused by mutation in a gene or gene product. Exemplary diseases or disorders that can be treated with the LNPs or the LNP compositions of the disclosure include, but are not limited to, metabolic diseases or disorders (e.g., Fabry disease, Gaucher disease, phenylketonuria (PKU), glycogen storage disease); urea cycle diseases or disorders (e.g., ornithine transcarbamylase (OTC) deficiency); lysosomal storage diseases or disorders (e.g., metachromatic leukodystrophy (MLD), mucopolysaccharidosis Type II (MPSII; Hunter syndrome)); liver diseases or disorders (e.g., progressive familial intrahepatic cholestasis (PFIC); blood diseases or disorders (e.g., hemophilia A and B, thalassemia, and anemia); cancers and tumors, and genetic diseases or disorders (e.g., cystic fibrosis).

In one embodiment, the LNPs or LNP compositions of the disclosure may be employed to deliver a heterologous nucleotide sequence in situations in which it is desirable to regulate the level of transgene expression (e.g., transgenes encoding hormones or growth factors).

In one embodiment of any of the aspects or embodiments herein, the LNPs or LNP compositions of the disclosure can be used to correct an abnormal level and/or function of a gene product (e.g., an absence of, or a defect in, a protein) that results in the disease or disorder. The LNPs or LNP compositions of the disclosure can produce a functional protein and/or modify levels of the protein to alleviate or reduce symptoms resulting from, or confer benefit to, a particular disease or disorder caused by the absence or a defect in the protein. For example, treatment of OTC deficiency can be achieved by producing functional OTC enzyme; treatment of hemophilia A and B can be achieved by modifying levels of Factor VIII, Factor IX, and Factor X; treatment of PKU can be achieved by modifying levels of phenylalanine hydroxylase enzyme; treatment of Fabry or Gaucher disease can be achieved by producing functional alpha galactosidase or beta glucocerebrosidase, respectively; treatment of MFD or MPSII can be achieved by producing functional arylsulfatase A or iduronate-2-sulfatase, respectively; treatment of cystic fibrosis can be achieved by producing functional cystic fibrosis transmembrane conductance regulator; treatment of glycogen storage disease can be achieved by restoring functional G6Pase enzyme function; and treatment of PFIC can be achieved by producing functional ATP8B1, ABCB11, ABCB4, or TJP2 genes.

In some embodiments, the LNPs or LNP compositions of the disclosure can be used to provide a DNA-based therapeutic to a cell in vitro or in vivo. Examples of DNA-based therapeutics include, but are not limited to, minicircle DNA, minigene, viral DNA (e.g., Lentiviral or AAV genome) or non-viral synthetic DNA vectors, closed-ended linear duplex DNA (ceDNA/CELiD), plasmids, bacmids, Doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

In one embodiment of any of the aspects or embodiments herein, exemplary transgenes encoded by ceDNA in the LNPs or LNP compositions of the disclosure include, but are not limited to: X, lysosomal enzymes (e.g., hexosaminidase A, associated with Tay-Sachs disease, or iduronate sulfatase, associated, with Hunter Syndrome/MPS II), erythropoietin, angiostatin, endostatin, superoxide dismutase, globin, leptin, catalase, tyrosine hydroxylase, as well as cytokines (e.g., a interferon, β-interferon, interferon-γ, interleukin-2, interleukin-4, interleukin 12, granulocyte-macrophage colony stimulating factor, lymphotoxin, and the like), peptide growth factors and hormones (e.g., somatotropin, insulin, insulin-like growth factors 1 and 2, platelet derived growth factor (PDGF), epidermal growth factor (EGF), fibroblast growth factor (FGF), nerve growth factor (NGF), neurotrophic factor-3 and 4, brain-derived neurotrophic factor (BDNF), glial derived growth factor (GDNF), transforming growth factor-a and -b, and the like), receptors (e.g., tumor necrosis factor receptor). In some exemplary embodiments, the transgene encodes a monoclonal antibody specific for one or more desired targets. In some exemplary embodiments, more than one transgene is encoded by the ceDNA vector. In some exemplary embodiments, the transgene encodes a fusion protein comprising two different polypeptides of interest. In some embodiments of any of the aspects and embodiments herein, the transgene encodes an antibody, including a full-length antibody or antibody fragment, as defined herein. In some embodiments of any of the aspects and embodiments herein, the antibody is an antigen-binding domain or an immunoglobulin variable domain sequence, as that is defined herein. Other illustrative transgene sequences encode suicide gene products (thymidine kinase, cytosine deaminase, diphtheria toxin, cytochrome P450, deoxycytidine kinase, and tumor necrosis factor), proteins conferring resistance to a drug used in cancer therapy, and tumor suppressor gene products.

In one embodiment of any of the aspects or embodiments herein, this disclosure provides a method of providing anti-tumor immunity in a subject, the method comprising administering to the subject an effective amount of any embodiment of an LNP contemplated herein or any embodiment of a pharmaceutical composition comprising an LNP contemplated herein. Furthermore, this disclosure provides a method of treating a subject having a disease, disorder or condition associated with an elevated expression of a tumor antigen, the method comprising administering to the subject an effective amount of any embodiment of an LNP contemplated herein or any embodiment of a pharmaceutical composition comprising an LNP contemplated herein. In some embodiments, the TNA is retained in the spleen for at least about 6 hours, or at least about 9 hours, or at least about 12 hours, or at least about 15 hours, or at least about 18 hours, or at least about 21 hours, or at least about 24 hours, or at least about 27 hours, or at least about 30 hours, or at least about 33 hours, or at least about 36 hours after dosing of an LNP of this disclosure, for example, via intravenous or intratumoral administration. In some embodiments, the amount (i.e., number of copies) of the TNA at the start of a 12, 18, or 24-hour time window post-dosing and the amount of the TNA at the end of the time window are within the same order of magnitude (e.g., 10⁻⁷ copies, 10⁻⁶ copies, 10⁻⁵ copies, 10⁻⁴ copies, 10⁻³ copies, 10⁻² copies, 10⁻¹ copies, 10° copies, 101 copies, 102 copies, 103 copies, etc. or any other suitable therapeutic levels). In other words, there is minimal reduction in concentrations of the TNA in the spleen within a 12, 18, or 24-hour time window post-dosing. In some embodiments, the TNA is a messenger RNA (mRNA).

Examples of solid tumors treatable with an LNP disclosed herein or a pharmaceutical composition comprising the same include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. According to some embodiments, the tumor or cancer is a melanoma, e.g., an advanced stage melanoma. Metastatic lesions of the aforementioned cancers can also be treated or prevented using the methods and compositions of the disclosure. Examples of other solid tumors or cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers.

In further embodiments, the present disclosure provides a method of treating a blood disease, disorder or condition in a subject, the method comprising administering to the subject an effective amount of any embodiment of an LNP contemplated herein or any embodiment of a pharmaceutical composition comprising an LNP contemplated herein. Non-limiting examples of the blood disease, disorder or condition include acute myeloid leukemia (AML), acute lymphoblastic leukemia (ALL), chronic lymphocytic leukemia (CLL), chronic myelogenous leukemia (CML), Hodgkin lymphoma (HL), multiple myeloma, a myelodysplastic syndrome (MDS), non-Hodgkin lymphoma (NHL), adrenoleukodystrophy (ALD), Hurler syndrome, Krabbe disease (Globoid-cell leukodystrophy or GLD), metachromatic leukodystrophy (MLD), severe aplastic anemia (SAA), severe combined immunodeficiency (SCID), sickle cell disease (SCD), thalassemia, Wiskott-Aldrich syndrome, Diamond-Blackfan anemia, essential thrombocytosis, Fanconi anemia, hemophagocytic lymphohistiscytosis (HLH), juvenile myelomonocytic leukemia (JMML), myelofibrosis, polycythemia vera, and a combination thereof. In some embodiments, the TNA is a messenger RNA (mRNA). In some embodiments, the TNA is retained in the bone marrow for at least about 6 hours, or at least about 9 hours, or at least about 12 hours, or at least about 15 hours, or at least about 18 hours, or at least about 21 hours, or at least about 24 hours, or at least about 27 hours, or at least about 30 hours, or at least about 33 hours, or at least about 36 hours after dosing of an LNP of this disclosure, for example, via intravenous or intratumoral administration. In some embodiments, the amount (i.e. number of copies) of the TNA at the start of a 12, 18, or 24-hour time window post-dosing and the number of the TNA at the end of the time window are within the same order of magnitude (e.g., 10⁻⁷ copies, 10⁻⁶ copies, 10⁻⁵ copies, 10⁻⁴ copies, 10⁻³ copies, 10⁻² copies, 10⁻¹ copies, 100 copies, 101 copies, 102 copies, 103 copies, etc. or any other suitable therapeutic levels) or are reduced for less than one order of magnitude. In other words, there is minimal or insignificant reduction in concentrations of the TNA in the bone marrow within a 12, 18, or 24-hour time window post-dosing. In some embodiments, the TNA is a messenger RNA (mRNA).

### Administration

In some embodiments, an LNP or an LNP composition of the disclosure can be administered to an organism for transduction of cells in vivo. In some embodiments, an LNP or an LNP composition of the disclosure can be administered to an organism for transduction of cells ex vivo.

Generally, administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route. Exemplary modes of administration of an LNP or an LNP composition of the disclosure include oral, rectal, transmucosal, intranasal, inhalation (e.g., via an aerosol), buccal (e.g., sublingual), vaginal, intrathecal, intraocular, transdermal, intraendothelial, in utero (or in ovo), parenteral (e.g., intravenous, subcutaneous, intradermal, intracranial, intramuscular [including administration to skeletal, diaphragm and/or cardiac muscle], intrapleural, intracerebral, and intraarticular), topical (e.g., to both skin and mucosal surfaces, including airway surfaces, and transdermal administration), intralymphatic, and the like, as well as direct tissue or organ injection (e.g., to liver, eye, skeletal muscle, cardiac muscle, diaphragm muscle or brain).

Administration of the LNP or LNP compositions of the disclosure can be to any site in a subject, including, without limitation, a site selected from the group consisting of the brain, a skeletal muscle, a smooth muscle, the heart, the diaphragm, the airway epithelium, the liver, the kidney, the spleen, the pancreas, the skin, and the eye. The most suitable route in any given case will depend on the nature and severity of the condition being treated, ameliorated, and/or prevented and on the nature of the particular ceDNA LNP that is being used. Additionally, ceDNA permits one to administer more than one transgene in a single vector, or multiple ceDNA vectors (e.g., a ceDNA cocktail).

In one embodiment of any of the aspects or embodiments herein, the LNPs or LNP compositions of the disclosure can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In some embodiments, the LNPs of the disclosure or the pharmaceutical compositions comprising the LNPs of the disclosure, when administered to a subject, is characterized by a lower immunogenicity than a reference LNP or a pharmaceutical composition comprising a reference LNP. In some embodiments, the immunogenicity of the LNP of the disclosure or the pharmaceutical composition comprising the LNP of the disclosure may be measured by measuring levels of one or more proinflammatory cytokines. Accordingly, in some embodiments, the LNPs of the disclosure or the pharmaceutical compositions comprising the LNPs of the disclosure, when administered to a subject, elicits a lower pro-inflammatory cytokine response than a reference LNP or a pharmaceutical composition comprising a reference LNP. The term “elicits a lower pro-inflammatory cytokine response than a reference LNP or a pharmaceutical composition comprising a reference LNP”, as used herein, means that the LNP of the disclosure or the pharmaceutical composition comprising the LNP of the disclosure, when administered to a subject, causes a smaller increase in the levels of one or more pro-inflammatory cytokines as compared to a reference LNP or a pharmaceutical composition comprising a reference LNP. Exemplary pro-inflammatory cytokines include, but are not limited to, granulocyte colony stimulating factor (G-CSF), interleukin 1 alpha (IL-1α), interleukin 1 beta (IL-1β), interleukin 6 (IL-6), interleukin 8 (IL-8 or CXCL8), interleukin 11 (IL-11), interleukin 17 (IL-17), interleukin 18 (IL-18), interferon α (IFN-α), interferon β (IFN-β), interferon γ (IFN-γ), C—X—C motif chemokine ligand 10 (CXCL10 or IP-10), monocyte chemoattractant protein 1 (MCP-1), CD40L, CCL2, CCL3, CCL4, CCL5, CCL11, tumor necrosis factor α (TNF-α), and combinations thereof. In some embodiments, the reference LNP is an LNP that does not comprise a helper lipid (e.g., C₂-C₈ ceramide or sphingomyelin) as described herein. The reference LNP can be an LNP that. (i) does not comprise the helper lipid represented by Formula (I), (II), (III), or (IV), or a salt or an ester thereof, or a deuterated analogue of any of the foregoing; or (ii) comprises a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) and a reference lipid-anchored polymer comprising at least two hydrophobic tails each comprise 12 to 15 carbon atoms in a single aliphatic chain backbone.

In one embodiment, the reference LNP comprises an ionizable lipid, DSPC, cholesterol, a lipid-anchored polymer comprising PEG attached to a lipid moiety which has two hydrophobic tails, each comprising of 14 carbon atoms (e.g., DMG-PEG2000).

### EXAMPLES

The following examples are provided by way of illustration not limitation.

### Example 1. Preparation of Lnps Comprising C18 Ceramide

LNP formulations were prepared using various species of ceramides as helper lipids. For example, the LNP formulations were prepared using C18 ceramide (d18:1/18:0) and C18 dihydroceramide (d18:0/18:0) of the following structures:

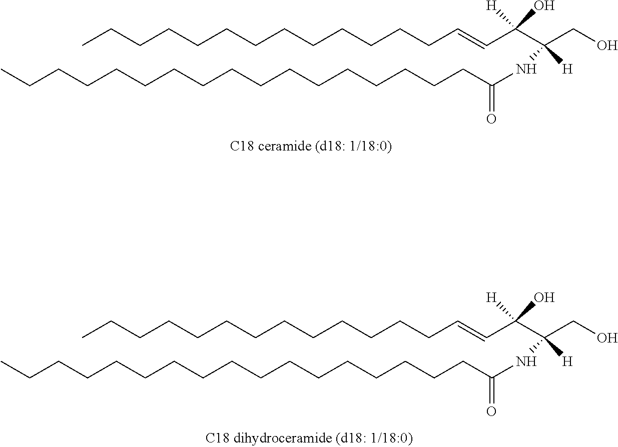 [SMILES: CCCCCCCCCCCCC/C=C/[C@@H](O)[C@H](CO)NC(=O)CCCCCCCCCCCCCCCCC ; CCCCCCCCCCCCC/C=C/[C@@H](O)[C@H](CO)NC(=O)CCCCCCCCCCCCCCCCC]

The specific prepared formulations are shown in Table 10 below:

TABLE 10
LNP formulations comprising C18 ceramide and C18 dihydroceramide:

| | Constituents of LNP | Average | Polydispersity | Encapsulation | |
|---|---|---|---|---|---|
| | Formulation | Diameter | Index | Efficiency | |
| No. | Components (ratios) | (nm) | (PDI) | (EE) % | Notes |
| LNP | Ionizable Lipid No. | N/A | N/A | N/A | Too insoluble |
| 2 | 87:C18 | | | | to |
| | dihydroceramide:Cholesterol:DSPE- | | | | successfully |
| | PEG2000-OH | | | | formulate |
| | (47.5:10:39.5:3) | | | | |
| LNP | Control Ionizable | 116.9 | 0.069 | 91.8% | Large |
| 3 | Lipid Z:C18 | | | | particles and |
| | ceramide:Cholesterol:DSG- | | | | EE that is |
| | PEG2000-OMe:DSPE- | | | | lower than |
| | PEG2000-GalNAc3 | | | | 95% |
| | (47.5:10:39.5:2.5:0.5) | | | | |

It was found that including C18 ceramide or C18 dihydroceramide as a helper lipid in an LNP does not yield an LNP suitable for therapeutic nucleic acid administration. For example, LNP 2 containing C18 dihydroceramide as a helper lipid was insoluble and failed to yield successful LNP formulation. Further, it was also found that LNP 3 containing C18 ceramide as a helper lipid had an average diameter of 116.9 nm, a LNP size generally considered to not be suitable for therapeutic use due to the fenestration size of the target organ such as the liver and specifically, the hepatocytes. The encapsulation efficiency of LNP 3 was also lower than 95%. Accordingly, the results of Example 1 indicate that C18 ceramide or C18 dihydroceramide as a helper lipid in an LNP does not result in LNPs that could be therapeutically useful.

### Example 2. Preparation of LNPs Containing C2 Ceramide and Various Lipid-Anchored Polymers

Additional LNP formulations were prepared using C2 ceramide (d18:1/2:0) as a helper lipid. In this example, LNP formulations were prepared containing different lipid-anchored polymers and C2 ceramide as listed in Table 11 below. The structure of C2 ceramide is also shown below:

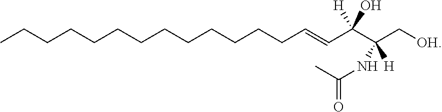 [SMILES: CCCCCCCCCCCCC/C=C/[C@@H](O)[C@H](CO)NC(C)=O]

TABLE 11
LNP formulations comprising C2 ceramide (d18:1/2:0)
and various lipid-anchored polymers.

| | | Average | Polydispersity | Encapsulation |
|---|---|---|---|---|
| LNP | Constituents of LNP Formulation | Diameter | Index | Efficiency |
| No. | Components (ratios) | (nm) | (PDI) | (EE) % |
| LNP33 | Ionizable Lipid 81:C2 | 75.7 | 0.1301 | 90 |
| | ceramide:Cholesterol:DMG- | | | |
| | PEG2000 (47.5:10:39.5:3) | | | |
| LNP44 | Ionizable Lipid 87:C2 | 71.3 | 0.084 | 96.9 |
| | ceramide:Cholesterol:DSG- | | | |
| | PEG2000-OMe (47.5:10:39.5:3) | | | |
| LNP55 | Ionizable Lipid | 76.17 | 0.088 | 97.8 |
| | 87:DSPC:Cholesterol:DMG- | | | |
| | PEG2000:-PEG2K-GalNAc3 | | | |
| | (47.6:10:39.5:2.4:0.5) | | | |
| LNP4 | Ionizable Lipid Z:C2 | 72.2 | 0.042 | 96.7 |
| | ceramide:Cholesterol:DSG- | | | |
| | PEG2000-OMe (47.5:10:39.5:3) | | | |
| LNP33 | Ionizable Lipid 81:C2 | 75.7 | 0.1301 | 90 |
| | ceramide:Cholesterol:DMG- | | | |
| | PEG2000 (47.5:10:39.5:3) | | | |
| LNP44 | Ionizable Lipid 87:C2 | 71.3 | 0.084 | 96.9 |
| | ceramide:Cholesterol:DSG- | | | |
| | PEG2000-OMe (47.5:10:39.5:3) | | | |
| LNP55 | Ionizable Lipid | 76.17 | 0.088 | 97.8 |
| | 87:DSPC:Cholesterol:DMG- | | | |
| | PEG2000:-PEG2K-GalNAc3 | | | |
| | (47.6:10:39.5:2.4:0.5) | | | |
| LNP5 | Ionizable Lipid Z:C2 | 73.5 | 0.085 | 97.6 |
| | ceramide:cholesterol:bis-DSG- | | | |
| | PEG2000 (47.5:10:39.5:3) | | | |
| LNP6 | Ionizable Lipid Z:C2 | 71.0 | 0.051 | 96.4 |
| | ceramide:cholesterol:DODA- | | | |
| | PG46G (47.5:10:39.5:3) | | | |

As indicated in Table 11, using 47 mol % of ionizable lipids, 10 mol % C2 ceramide (d18:1/2:0) as a helper lipid, 40 mol % cholesterol as a structural lipid, and various types of lipid-anchored polymers consistently resulted in LNPs with the average diameter less than 80 nm or 75 nm, and encapsulation efficiencies greater than 90% (mostly above 95%). The reference LNP55 that contains 47 mol % ionizable lipid, 10 mol % DSPC as the helper lipid, 40 mol % cholesterol as a structural lipid, and DMG-PEG2000 (~3 mol %; dissociable lipid-polymer) as the first lipid polymer recorded average diameters that is less than 80 nm. Ionizable Lipid Z (structure not shown) belongs to a different class of ionizable lipids compared to Ionizable Lipid 81 and Ionizable Lipid 87, whereby both the headgroup and lipid tail moieties are structurally distinct from those of Ionizable Lipid 81 and Ionizable Lipid 87. The structures of Ionizable Lipid 81 and Ionizable Lipid 87 are shown in Table 6.

### Example 3. Preparation of LNPs Containing Various Amounts of C2 Ceramide

Additional LNP formulations containing different amounts of a helper lipid (e.g., C2 ceramide (d18:0/2:0) (C18 ceramide (d18:1/18:0) were used as a suboptimal LNP (LNP32)) were prepared. These LNP formulations are listed in Table 12 below.

TABLE 12
LNP formulations containing varying amounts of C2 ceramide.

| | | Average | Polydispersity | Encapsulation |
|---|---|---|---|---|
| LNP | Constituents of LNP Formulation | Diameter | Index | Efficiency |
| No. | Components (ratios, mol %) | (nm) | (PDI) | (EE) % |
| LNP105 | Ionizable Lipid | 155 | 0.13 | 97% |
| | Z:DSPC:Cholesterol:DSG- | | | |
| | PEG2000-OMe (33:40:24:3) | | | |
| LNP31 | Ionizable Lipid Z:C2 | 77.4 | 0.03 | 96.9% |
| | ceramide:Cholesterol:DSG- | | | |
| | PEG2000-OMe:DSPE-PEG2000-GalNAc3 | | | |
| | (36.54:30.77:30.38:1.92:0.38) | | | |
| LNP30 | Ionizable Lipid Z:C2 | 73.3 | 0.023 | 97.2% |
| | ceramide:Cholesterol:DSG-PEG2000- | | | |
| | OMe:DSPE-PEG2000-GalNAc3 | | | |
| | (39.58:25:32.92:2.08:0.42) | | | |
| LNP29 | Ionizable Lipid Z:C2 | 74.7 | 0.014 | 96.9 |
| | ceramide:Cholesterol:DSG-PEG2000- | | | |
| | OMe:DSPE-PEG2000-GalNAc3 | | | |
| | (43.18:18.18:35.91:2.27:0.45) | | | |
| LNP8 | Ionizable Lipid Z:C2 | 79.4 | 0.020 | 95.5 |
| | ceramide:Cholesterol:DSG-PEG2000- | | | |
| | OMe:DSPE-PEG2000-GalNAc3 | | | |
| | (47.5:10:39.5:2.5:0.5) | | | |
| LNP9 | Ionizable Lipid Z:C2 | 86.2 | 0.004 | 94.3 |
| | ceramide:Cholesterol:DSG-PEG2000- | | | |
| | OMe:DSPE-PEG2000-GalNAc3 | | | |
| | (50.0:5.26:41.58:2.63:0.53) | | | |
| LNP10 | Ionizable Lipid Z:C2 | 93.4 | 0.003 | 93.7 |
| | ceramide:Cholesterol:DSG-PEG2000- | | | |
| | OMe:DSPE-PEG2000-GalNAc3 | | | |
| | (51.35:2.7:42.7:2.7:0.54) | | | |
| LNP32 | Ionizable Lipid Z:C18 | 113.0 | 0.026 | 92.2 |
| | ceramide:Cholesterol:DSG-PEG2000- | | | |
| | OMe:DSPE-PEG2000-GalNAc3 | | | |
| | (47.5:10:39.5:2.5:0.5) | | | |

As indicated in Table 12, increasing the amounts of C2 ceramide helper lipid (as indicated by increasing the percentage molar ratio of C2 ceramide) and decreasing the percentage molar ratio of cholesterol results in LNPs with a diameter smaller than 80 nm, and encapsulation efficiencies of greater than 95%. However, it should be noted that, while they were successfully formulated into LNPs, visible precipitates were observed in lipid mixtures containing 30 mol % C2 ceramide and 40 mol % C2 ceramide while in cold storage. A ceramide content of about 10% to about 20% of the total lipid present in an LNP appeared to yield stable particles and with the most desirable analytics in terms of average diameter (e.g., <80 nm) and encapsulation efficiencies of >95%. Consistent with this observation, mol % greater than 40% of a helper lipid (e.g., DSPC) resulted in the average lipid particle size significantly larger (>80 nm in diameter (see LNP105), suggesting that the preferred molar ratio for a helper lipid for small particle size is from about 7 mol % to about 35 mol %. Similarly, the optimal molecular percentage of structural lipid (e.g., cholesterol) ranges from about 30% to about 40% in an LNP. Any reduction of mol % of sterol (e.g., cholesterol) below 30% by increasing molecular ratio of a helper lipid above 40% resulted in significantly larger particle sizes (e.g., >100 nm in diameter as seen in LNP105). Table 12 also shows that when formulated with a C18 ceramide, LNP32 has an average diameter size exceeding 100 nm, indicating that only certain molecular compositions as described above and in combination with certain types of a helper lipid, cholesterol, and lipid-polymer lead to desirable particle sizes for effective delivery (optimal range of particle size for effective deliver; e.g., 60 nm to 80 nm in diameter). The presence of a helper lipid like C18 ceramide at 10 mol % may affect the dynamics of lipid particle.

Furthermore, Table 13 shows that, consistent with the analytics in Table 12, LNPs formulated with 10 mol % C2 ceramide (d18:1/2:0), 40 mol % of sterol, 3 mol % of lipid-anchored polymer, but with a different type of ionizable lipids, i.e., Ionizable Lipid 87, and with or without GalNAc3 conjugated to a second lipid-anchored polymer consistently resulted in particle sizes that are <75 nm in diameter and encapsulation efficiencies that are suitable for in vivo therapeutic applications, confirming the observation made above.

TABLE 13
LNP formulations containing 10 mol % C2 Ceramide
(d18:1/2:0) and Ionizable Lipid 87

| LNP No. | Lipid composition | Zave (nm) | PDI | EE (%) |
|---|---|---|---|---|
| LNP33 | Ionizable Lipid 87:C2 | 73.7 | 0.127 | 92.0 |
| | ceramide:Cholesterol:DODA-PG46G:DSPE- | | | |
| | PEG2000-OMe (47.5:10:39.5:2.5:0.5) | | | |
| LNP34 | Ionizable Lipid 87:C2 | 71.0 | 0.121 | 89.3 |
| | ceramide:Cholesterol:DODA-PG46G:DSPE- | | | |
| | PEG2000-GalNAc3 (47.5:10:39.5:2.5:0.5) | | | |

### Example 4. Preparation of LNPs Containing Different Species of C2-C8 Ceramides

To examine the effect of a helper lipid on particle sizes, additional LNP formulations containing different species of C2-C8 ceramides were prepared. The structures of C2 (d14:1/2:0), C4, C6 and C8 ceramides used in the formulations are shown below:

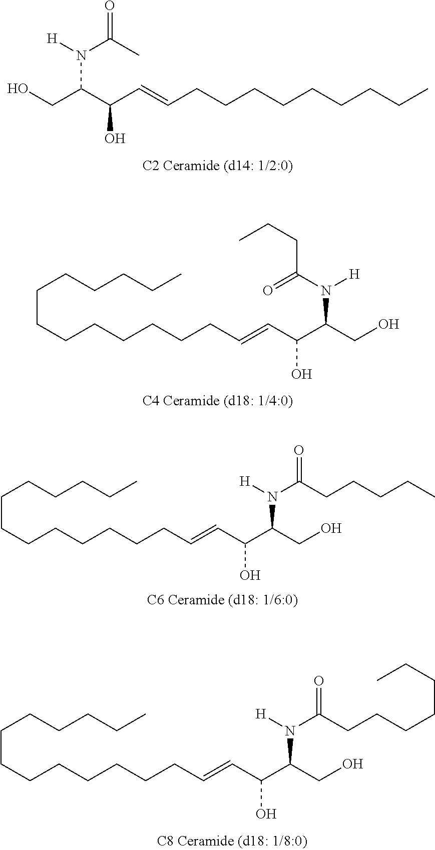

The analytics of these LNP formulations are listed in Table 14 below.

TABLE 14
LNP formulations comprising different species of C2-C8 ceramides

| | Constituents of LNP | Average | Polydispersity | Encapsulation |
|---|---|---|---|---|
| LNP | Formulation | Diameter | Index | Efficiency |
| No. | Components (ratios) | (nm) | (PDI) | (EE) % |
| LNP11 | Ionizable Lipid | 70.9 | 0.029 | 93.9% |
| | Z:C2 ceramide (d18:1/ | | | |
| | 2:0):Cholesterol:DSG- | | | |
| | PEG2000-OMe | | | |
| | (47.5:10:39.5:3) | | | |
| LNP12 | Ionizable Lipid | 82.6 | 0.027 | 93.2% |
| | Z:C2 ceramide (d14:1/ | | | |
| | 2:0):Cholesterol:DSG- | | | |
| | PEG2000-OMe | | | |
| | (47.5:10:39.5:3) | | | |
| LNP13 | Ionizable Lipid Z:C4 | 75.7 | 0.068 | 88.0% |
| | ceramide:Cholesterol:DSG- | | | |
| | PEG2000-OMe | | | |
| | (47.5:10:39.5:3) | | | |
| LNP14 | Ionizable Lipid Z:C6 | 75.4 | 0.039 | 88.8% |
| | ceramide:Cholesterol:DSG- | | | |
| | PEG2000-OMe | | | |
| | (47.5:10:39.5:3) | | | |
| LNP15 | Ionizable Lipid Z:C8 | 76.6 | 0.066 | 93.1% |
| | ceramide:Cholesterol:DSG- | | | |
| | PEG2000-OMe | | | |
| | (47.5:10:39.5:3) | | | |

As indicated in Table 14, using different species of C2-C8 ceramides as helper lipids in LNPs consistently results in LNPs with a diameter smaller than 80 nm, and encapsulation efficiencies of greater than 88%.

### Example 5. LNPs Comprising Ceramide and Sphingomyelin Support In Vivo Expression of Nucleic Acids

The goal of this study was to demonstrate in vivo expression of nucleic acids that are in the exemplary LNPs of the disclosure that comprise C2 ceramide (d18:1/2:0), C8 ceramide, or C2 sphingomyelin as a helper lipid and using the molecular ratio identified above that yields smaller LNP particle sizes (e.g., 60-80 nm in diameter). To this end, various types of LNP as shown in Table 15 were formulated with ceDNA encoding luciferase as a cargo and analyzed for their sizes and encapsulation efficiency. The LNPs were then administered to CD-1 mice (males) intravenously (IV) at a dose of 0.5 mg/kg and 2.0 mg/kg (0 day). The LNPs used in the experiment are shown in Table 15 below.

TABLE 15
LNP profiles dosed intravenously to CD-1 mice

| | | Average | Polydispersity | Encapsulation |
|---|---|---|---|---|
| | Constituents of LNP Formulation | Diameter | Index | Efficiency |
| No. | Components (ratios) | (nm) | (PDI) | (EE) % |
| DPBS | N/A | N/A | N/A | N/A |
| (negative | | | | |
| control) | | | | |
| LNP D | Ionizable Lipid | 82.8 | 0.094 | 94.5% |
| | Z:DSPC:Cholesterol:DMG- | | | |
| | PEG2000:DSPE-PEG2000-GalNAc4 | | | |
| | (47.5:10.0:40.2:1.8:0.5) | | | |
| LNP1 | Ionizable Lipid Z:C2 | 73.5 | 0.046 | 95.5% |
| | ceramide:Cholesterol:DSG-PEG2000- | | | |
| | OMe:DSPE-PEG2000-GalNAc1 | | | |
| | (47.5:10.0:39.5:2.5:0.5) | | | |
| LNP35 | Ionizable Lipid Z:C8 | 77.0 | 0.062 | 94.4 |
| | ceramide:Cholesterol:DSG-PEG2000- | | | |
| | OMe:DSPE-PEG2000-GalNAc3 | | | |
| | (47.5:10.0:39.5:2.5:0.5) | | | |
| LNP36 | Ionizable Lipid Z:C2 | 68.1 | 0.077 | 95.1 |
| | sphingomyelin:Cholesterol:DSG- | | | |
| | PEG2000-OMe:DSPE-PEG2000- | | | |
| | GalNAc3 (47.5:10.0:39.5:2.5:0.5) | | | |

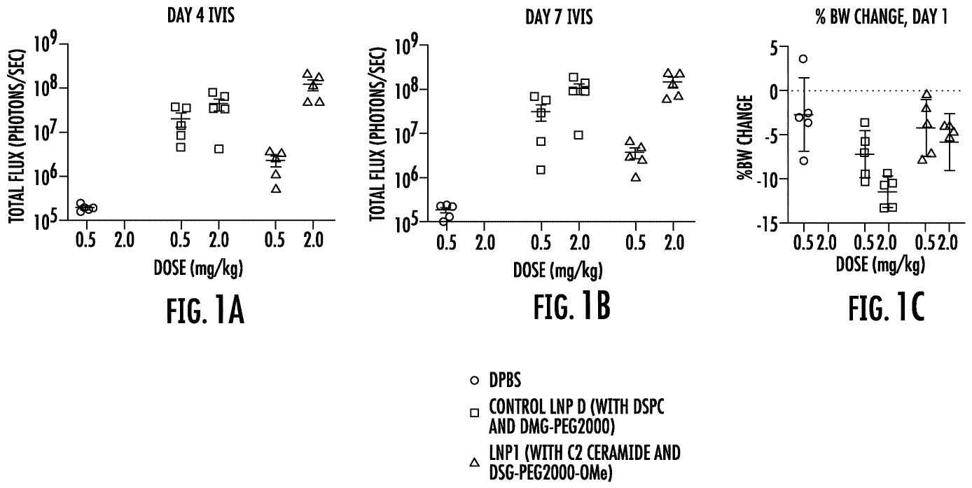

Total fluorescence was measured on Day 3 and Day 7. FIG. 1A shows the amount of total fluorescence measured (IVIS) for both tested LNPs and negative control at Day 4 post-dosing. FIG. 1B shows the amount of total fluorescence measured for both tested LNPs and negative control at Day 7 post-dosing. The results shown in FIG. 1A and FIG. 1B indicate that administration of the exemplary LNP of the disclosure comprising C2 ceramide, i.e., LNP1 results in a dose-dependent expression of nucleic acid in the LNP at both Day 4 and Day 7.

FIG. 1C shows % change in body weight of mice on Day 1. The results shown in FIG. 1C indicate that the tested exemplary LNP of the disclosure comprising C2 ceramide, i.e., LNP1 caused a much milder body weight change in mice as compared to the reference LNP that incorporates DSPC as the helper lipid and DMG-PEG2000 as a C14 tail lipid polymer.

The results presented in Example 5 demonstrate that an exemplary LNP of the disclosure comprising C2 ceramide can be used to encapsulate nucleic acid of large size (>2000 bp) and the LNP can be administered and delivered in vivo to support expression of nucleic acids without triggering any major tolerability issues and other adverse events in mice (e.g., rough hair coat, facial swelling).

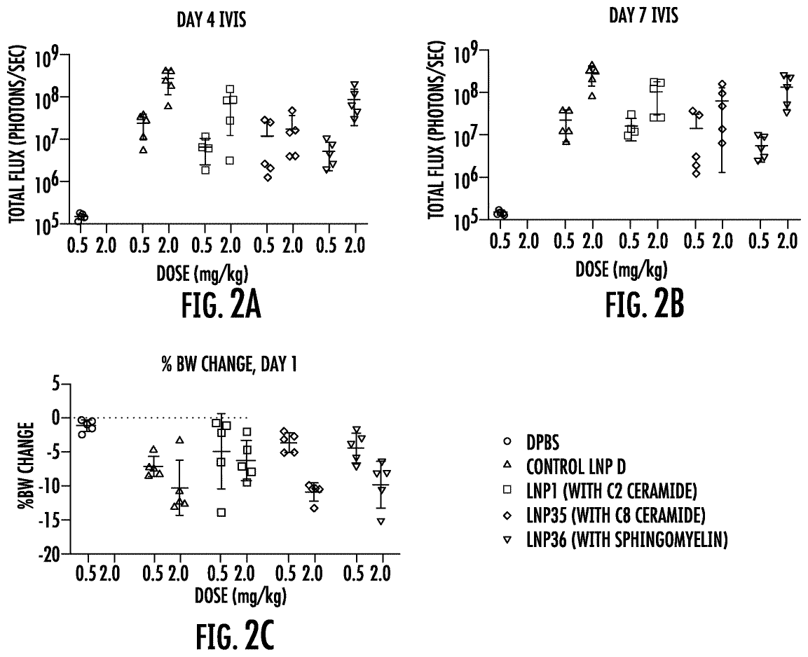

Further, LNPs containing C2 sphingomyelin (d18:1/2:0) as a helper lipid were also tested along with C2 ceramide LNP. Surprisingly, LNPs containing C2 sphingomyelin, which had an average particle diameter size of <70 nm (see Table 14), also exhibited a similar level of expression and tolerability profiles to those of C2 ceramide containing LNPs (FIGS. 2A-2C). This result suggests that C2 ceramide or C2 sphingomyelin-containing LNPs are similar to each other in terms of their overall physical profiles and their applicability, if the other LNP components and their molar ratios remain the same. FIGS. 2A-2C also show that LNPs containing C8 ceramide (d18:1/8:0) exhibited equivalent levels of expression and tolerability profiles as LNPs containing C2 ceramide or C2 sphingomyelin.

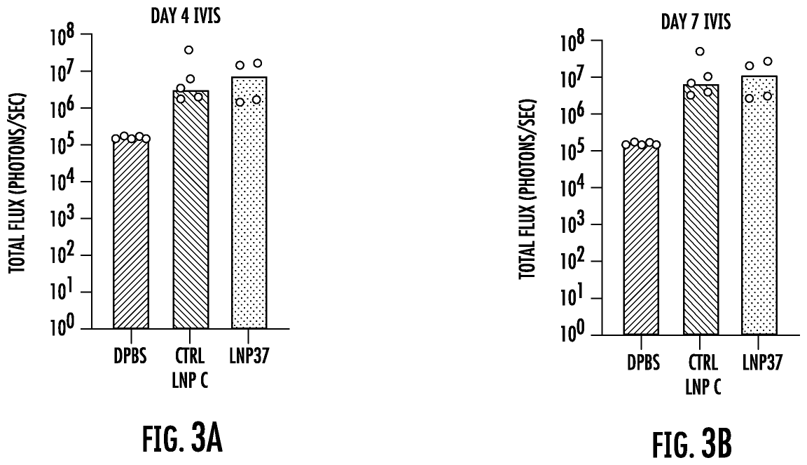

The ceramide or sphingomyelin-containing LNPs discussed above and with analytics, expression and tolerability profiles shown in Table 15, FIGS. 1A-1C and FIGS. 2A-2C, i.e., namely LNP1, LNP35 and LNP36 each contain DSG-PEG2000-OMe as the first lipid-anchored polymer, which has two hydrophobic tails, each with 18 carbon atoms. LNPs that contain C2 ceramide (d18:1/2:0) as the helper lipid and instead DMG-PEG2000 (i.e., having two hydrophobic tails with 14 carbon atoms) as a lipid polymer were also prepared, analyzed, and compared to their DSPC counterpart (i.e., LNPs that contain DSPC instead of C2 ceramide as the helper lipid and DMG-PEG2000 as a C14 lipid polymer). The analytics of these LNPs are presented in Table 16. in vivo luciferase expression levels in CD-1 mice are presented in FIGS. 3A and 3B. FIGS. 3A and 3B demonstrate that on both Day 4 and Day 7 post-dosing, expression in mice was higher with LNP37 formulated with Ionizable Lipid 81 (see lipid structure in Table 6), DMG-PEG2000 as a lipid polymer and C2 ceramide (d18:1/2:0) as a helper lipid, as compared to the LNP C (annotated as “CTRL LNP C” in FIGS. 3A and 3B) that was formulated with Ionizable Lipid 81, DMG-PEG2000 as C14 lipid polymer, but with DSPC as the helper lipid. As expected, however, the average LNP particle size for these two formulations were less than 80 nm in diameter, consistent with the observations made above, e.g, 40-50 mol % ionizable lipid; 10-20 mol % helper lipid; 30-40 mol % sterol; 3-5% lipid-anchored polymer for small particle sizes (<80 nm in diameter).

TABLE 16
LNPs dosed intravenously to CD-1 mice

| | | Average | Polydispersity | Encapsulation |
|---|---|---|---|---|
| | Constituents of LNP Formulation | Diameter | Index | Efficiency |
| LNP No. | Components (ratios) | (nm) | (PDI) | (EE) % |
| DPBS | N/A | N/A | N/A | N/A |
| (negative | | | | |
| control) | | | | |
| LNP C | Ionizable Lipid | 72.1 | 0.118 | 96.2 |
| | 81:DSPC:Cholesterol:DMG- | | | |
| | PEG2000:DSPE-PEG2000-GalNAc3 | | | |
| | (47.5:10.0:39.5:2.5:0.5) | | | |
| LNP37 | Ionizable Lipid 81:C2 | 77.1 | 0.113 | 95.9 |
| | Ceramide:Cholesterol:DMG- | | | |
| | PEG2000:DSPE-PEG2000-GalNAc3 | | | |
| | (47.5:10.0:39.5:2.5:0.5) | | | |

### Example 6. Immunogenicity of Exemplary LNPs of the Disclosure

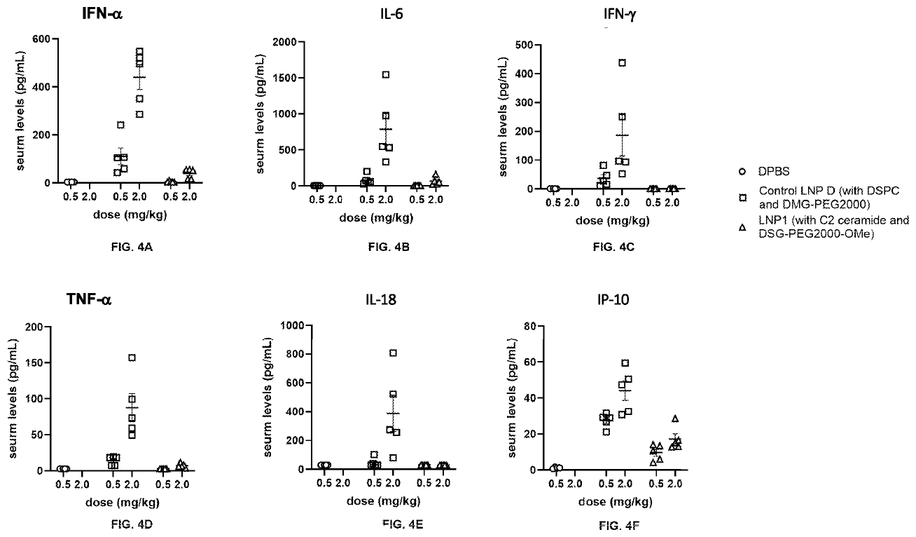

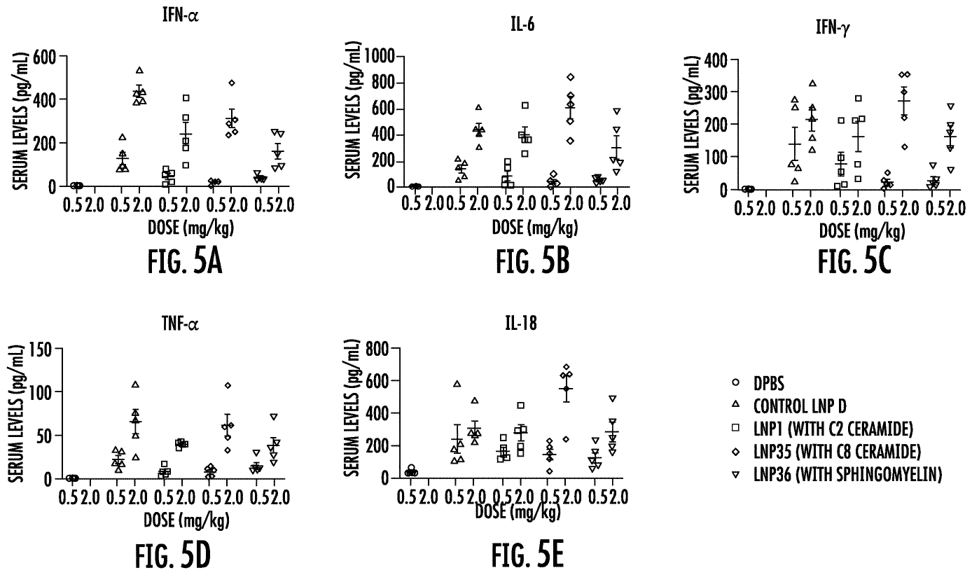

The immunogenicity profiles of the C2 ceramide-containing LNP1 and LNP35, C2 sphingomyelin-containing LNP36, LNP D (see Table 14 for formulation recipes and analytics and FIGS. 1A-1C and FIGS. 2A-2C for in vivo expression levels in mice and Day 1 body weight changes in mice) were compared by analyzing, at 6 hours post-dosing, the blood serum levels of multiple types of cytokines implicated in the regulation of innate immune response, i.e., IFN-alpha, IL-6, IFN-gamma, TNF-alpha, IL-18, and IP-10. FIGS. 4A-4F indicate that at both 0.5 mg/kg and 2.0 mg/kg, the blood serum levels of all six measured cytokines were lower for the C2 ceramide-containing LNP1 as compared to LNP D. In addition, FIGS. 5A-5E indicate that at both 0.5 mg/kg and 2.0 mg/kg, the blood serum levels of IFN-alpha, IL-6, IFN-gamma, TNF-alpha, and IL-18 were lower for the C2 ceramide-containing LNP1 and also C2 sphingomyelin-containing LNP36 as compared to the LNP D.

Additional C2 ceramide-containing LNP formulations, namely LNP23 through LNP28, were prepared to assess the impact of a lipid-anchored polymer on LNP characteristics. Referring to Table 17, each of LNP23, LNP24, and LNP25 was formulated using Ionizable Lipid Z and C2 ceramide (d18:1/2:0) as the helper lipid, but various different species of the first lipid-anchored polymer, e.g., PEG or PG conjugated to lipid having two C18 hydrophobic tails (i.e., DSG-PEG2000-OMe, bis-DSG-PEG2000, DODA-PG46) to assess the impact of a first lipid-anchored polymer on LNP sizes. LNP26, LNP27, and LNP28 were equivalent to LNP23, LNP24, and LNP25, respectively, with the exception that Ionizable Lipid 87 was used instead as a ionizable lipid. LNP A containing DSPC as a helper lipid and a C14 containing DMG-PEG lipid as a lipid polymer was prepared as reference formulations. As shown in Table 17, various lipid-anchored polymers at 3 mol % had little or no impact on particle sizes, as all of tested LNPs exhibited the average diameter of less than 80 nm.

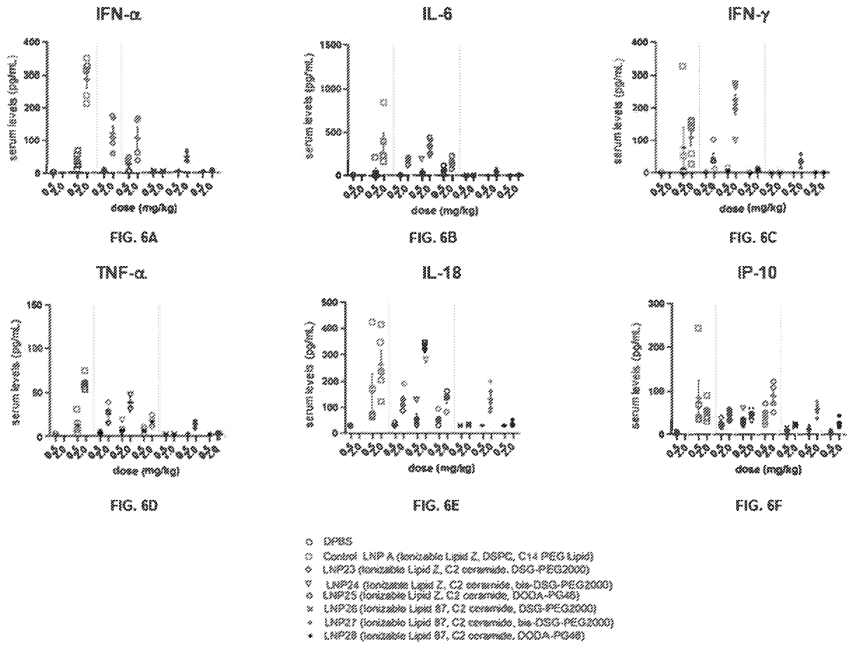

The formulations listed in Table 17 were administered to CD-1 mice and at 6 hours post-dosing, the blood samples were collected. Serum levels of the multiple types of cytokines were measured as described above. FIGS. 6A-6F indicate that at both 0.5 mg/kg and 2.0 mg/kg, the blood serum levels of all of IFN-α, IL-6, IFN-γ, TNF-γ, IL-18, and IP-10 were relatively lower for the C2 ceramide-containing LNP1 as compared to the LNP A. Of note, the blood serum levels for Ionizable Lipid 87 formulations (i.e., LNP26, LNP27, and LNP28) were significantly lower across all six measured cytokines.

TABLE 17
Tested exemplary LNPs of the disclosure

| | | Average | Polydispersity | Encapsulation |
|---|---|---|---|---|
| | Constituents of LNP Formulation | Diameter | Index | Efficiency |
| No. | Components (ratios) | (nm) | (PDI) | (EE) % |
| LNP A | Ionizable Lipid Z:DSPC:Cholesterol:C14 | 79.0 | 0.080 | 92.4 |
| | PEG2000 lipid*:DSPE-PEG2000-GalNAc4 | | | |
| | (47.5:10.0:40.2:1.8:0.5) | | | |
| LNP23 | Ionizable Lipid Z:C2 | 74.6 | 0.067 | 94.6 |
| | ceramide:Cholesterol:DSG-PEG2000- | | | |
| | OMe:DSPE-PEG-2000-GalNAc3 | | | |
| | (47.5:10.0:39.5:2.5:0.5) | | | |
| LNP24 | Ionizable Lipid Z:C2 | 79.3 | 0.084 | 95.9 |
| | ceramide:Cholesterol:bis-DSG- | | | |
| | PEG2000:DSPE-PEG-2000-GalNAc3 | | | |
| | (47.5:10.0:39.5:2.5:0.5) | | | |
| LNP25 | Ionizable Lipid Z:C2 | 74.0 | 0.059 | 94.2 |
| | ceramide:Cholesterol:DODA- | | | |
| | PG46:DSPE-PEG-2000-GalNAc3 | | | |
| | (47.5:10.0:39.5:2.5:0.5) | | | |
| LNP26 | Ionizable Lipid 87:C2 | 63.7 | 0.076 | 93.8 |
| | ceramide:Cholesterol:DSG-PEG2000- | | | |
| | OMe:DSPE-PEG-2000-GalNAc3 | | | |
| | (47.5:10.0:39.5:2.5:0.5) | | | |
| LNP27 | Ionizable Lipid 87:C2 | 74.9 | 0.103 | 93.3 |
| | ceramide:Cholesterol:bis-DSG- | | | |
| | PEG2000:DSPE-PEG-2000-GalNAc3 | | | |
| | (47.5:10.0:39.5:2.5:0.5) | | | |
| LNP28 | Ionizable Lipid 87:C2 | 64.2 | 0.073 | 93.7 |
| | ceramide:Cholesterol:DODA- | | | |
| | PG46:DSPE-PEG-2000-GalNAc3 | | | |
| | (47.5:10.0:39.5:2.5:0.5) | | | |

*C14 PEG2000 Lipid is a lipid polymer having two hydrophobic tails that each contain 14 carbon atoms, conjugated to PEG2000 (DMG-PEG2000), used herein as a reference LNP.

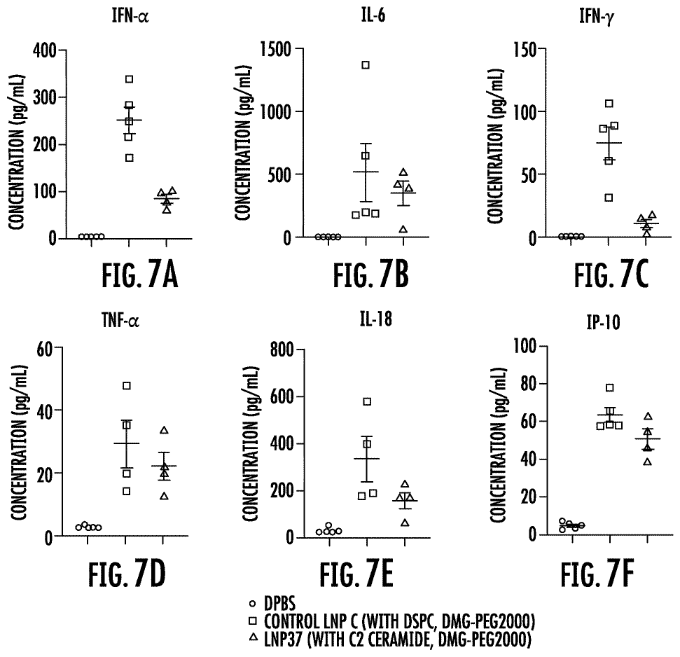

The immunogenicity profiles of the C2 ceramide-containing LNP37 and LNP C (see Table 16 for LNP molecular composition and analytics and FIGS. 3A-3C for in vivo expression and body weight changes in mice) were also tested. FIGS. 7A-7F indicate that at 2.0 mg/kg, the blood serum levels of all six measured cytokines were relatively lower in the C2 ceramide-containing LNP37 group as compared to the LNP C treated group.

Collectively, the blood serum cytokine level data of FIGS. 4A-4F, 5A-5E, 6A-6F and 7A-7F indicate that C2 ceramide and C2 sphingomyelin in particular as helper lipids in LNP formulations may have a positive impact on mitigating proinflammatory immune responses, independent from the type of lipid-anchored polymer (e.g., with C14 DMG-PEG2000 (see FIGS. 4A-4F, 5A-5E, 6A-6F) versus C18 group like DSG-PEG2000-OMe, DSG-PEG2000-OH, bis-DSG-PEG2000, or DODA-PG46 (see FIGS. 7A-7F).

### Example 7. Plasma Clearance of C2 Ceramide-Containing LNP

The pharmacokinetic properties of LNP E (Ionizable Lipid Z:DSPC:Cholesterol:C14 DMG-PEG Lipid:DSG-PEG2000-GalNAc; 47.5:10.0:39.5:2.5:0.5 mol %) and C2-containing LNP1 (Ionizable Lipid Z:C2 ceramide:Cholesterol:DSG-PEG2000-OMe:DSPE-PEG2000-GalNAc3; 47.5:10.0:39.5:2.5:0.5 mol %) were measured and compared with each other. The respective LNPs were formulated as disclosed above. LNP E and LNP1 LNP formulations were injected IV bolus via the tail vein of CD-1 mice and whole blood samples were collected with K₂EDTA as anticoagulant 150 μL/aliquot for qPCR at 2 min, 1 hour, 3 hour and 6-hour timepoints. Body weight, mortality, and clinical observations were recorded. The plasma portion of the blood was separated and collected.

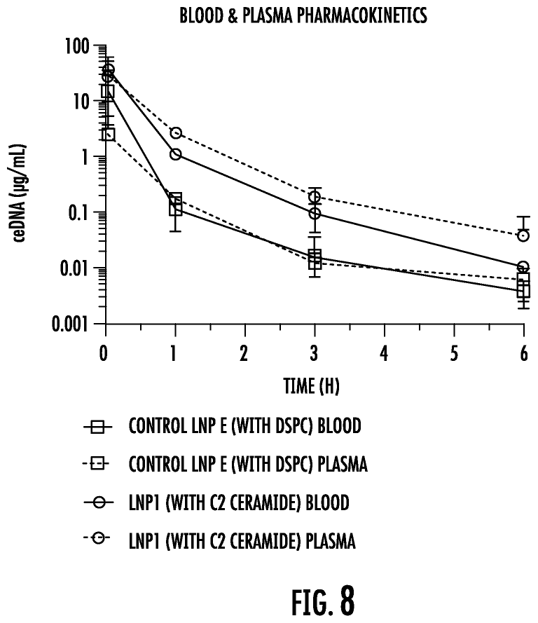

Pharmacokinetic profiles of whole blood and plasma clearance of LNP E with C14 lipid polymer and the C2 ceramide-containing LNP1 with a C18 DSG-PEG lipid-anchored polymer are depicted in FIG. 8. It was observed that whole blood and plasma concentrations of ceDNA in mice treated with the LNP1 were higher than the corresponding concentrations in mice treated with LNP E (with DSPC helper lipid and C14 DMG-PEG). The higher retention of ceDNA in the bloodstream or less rapid clearance of the ceDNA cargo from the bloodstream as delivered by the C2 ceramide-containing LNP1 could be beneficial in that off-target delivery to non-target cells, including but not limited to a red blood cell, and a macrophage, may be mitigated.

### Example 8. LNP Formulations with mRNA Cargo Exhibit In Vitro Expression and Hepatocyte Uptake

All of the LNP formulations described in the foregoing Examples 1-7 were prepared using ceDNA vector as the nucleic acid cargo. The goal of this study was to explore the characteristics of LNP formulations of the invention that carry mRNA as the nucleic acid cargo, such as but not limited to C2 ceramide (d18:1/2:0)-containing LNPs that are formulated with mRNA. mRNA structurally differs from a ceDNA vector at least in that mRNA is single-stranded and is likely less negatively charged than the covalently closed-ended and double-stranded ceDNA vector. mRNA is also known to be less stable than DNA and less rigid than DNA.

To this end, the LNP40 as listed in Table 18 was prepared using luciferase mRNA as the nucleic cargo. LNP40 included the DiD (DiIC18 (5); 1,1′-dioctadecyl-3,3,3′,3′-tetramethylindodicarbocyanine, 4-chlorobenzenesulfonate salt) for the purpose of measuring and analyzing the uptake of the particles by primary mouse hepatocytes. Like the ceDNA/LNP formulations, mRNA/LNP formulations of this disclosure (i.e., containing ceramide and/or sphingomyelin as helper lipid(s)), as exemplified by LNP40, exhibited average diameters and encapsulation efficiencies that are considered to be suitable for therapeutic use, including delivery to the hepatocytes.

TABLE 18
Physicochemical properties of LNP40 having mRNA-luciferase cargo

| | | Average | Polydispersity | Encapsulation |
|---|---|---|---|---|
| LNP | | Diameter | Index | Efficiency |
| Formulation | Lipids and Molar Ratios | (nm) | (PDI) | (%) |
| LNP40 | Ionizable Lipid 87:C2 | 55.2 | 0.023 | 97.6 |
| | ceramide:Cholesterol:DSG- | | | |
| | PEG2000-OMe:DSPE-PEG2000- | | | |
| | GalNAc3:DiD | | | |
| | (47.5:10:39:2.5:0.5:0.5) | | | |

The in vitro expression of mRNA-luciferase as delivered in LNP40 was also investigated. Briefly, primary mouse hepatocytes from C57BL/6 mice were plated at 50,000 cells per well in William's E attachment media (Thermo fisher #A1217601). Four hours after plating, the cells were treated with 200 ng LNP40 or vehicle (negative control) in Hepatocyte Culture Medium. 1 hour after the mRNA/LNP treatments, the cells were washed twice with DPBS and the hepatocyte culture media was added to the wells. Approximately, 18 hours later, relative cell viability was measured by CellTiter-Fluor Cell viability assay (Promega #G6080). Subsequently, luciferase activity was measured using One Step Luciferase assay system (BPS Bioscience #60690-1). To analyze the hepatocyte uptake of the mRNA/LNP, the cells were imaged using Opera High Content imaging for DiD (Ex/Em: 650/665) and Hoechst (Ex/Em: 350/460 nm) signals.

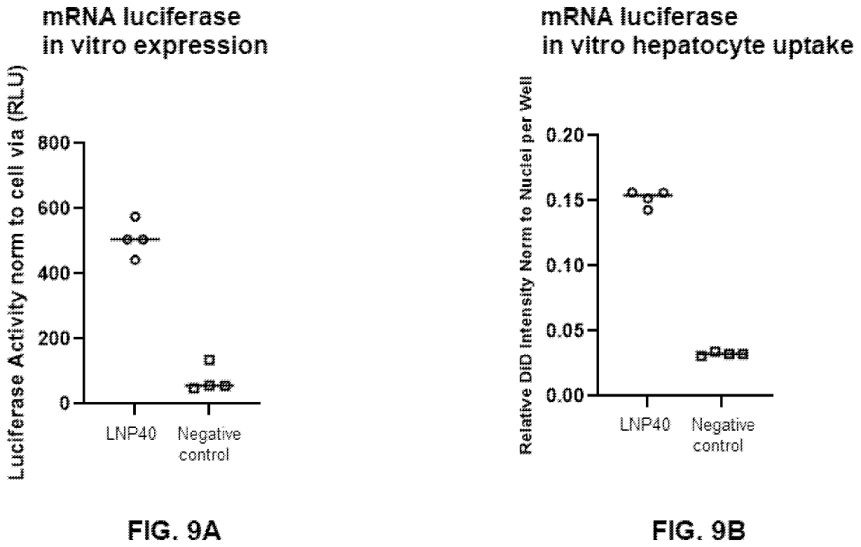

FIG. 9A and FIG. 9B respectively indicate that the luciferase activity and hepatocyte uptake were both detected in primary mouse hepatocytes that had been incubated with LNP40, but not the negative control that contained no LNP.

In addition, LNP formulations incorporating a C14 lipid-anchored polymer, namely DMG-PEG2000, Ionizable Lipid 81 and ceramide helper lipids ranging from 2 to 8 carbon atoms in the fatty acid tail were also prepared using mRNA-luciferase as the nucleic acid cargo (see Table 16). The primary mouse hepatocyte mRNA-luciferase expression assay was set up as described above, but with one additional step, i.e., the LNPs were incubated in cynomolgus monkey serum before they were used to treat the primary mouse hepatocytes. The purpose of this incubation was to facilitate the opsonization of LNPs, if any, i.e., to allow the LNPs to be coated with peptides and/or antibodies found in the cynomolgus monkey serum).

The analytical data for LNP41, LNP42, LNP43, and LNP45 as non-limiting examples of mRNA ceramide- or sphingomyelin-containing LNPs (presented in Table 19) is similar to that of LNP40 shown in Table 18 and indicates that these exemplary mRNA/LNP formulations with mol % of each component disclosed herein consistently exhibited the average particle sizes less than 80 nm in diameter and encapsulation efficiencies that are suitable for therapeutic use.

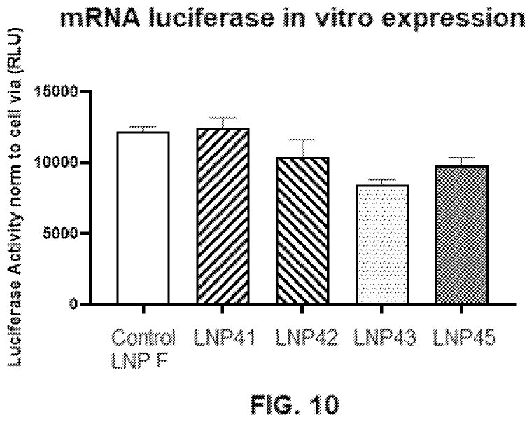

As shown in FIG. 10, LNP41 formulated with C2 ceramide (d18:1/2:0) was characterized by the highest levels of mRNA-luciferase expression. Although increasing the number of carbon atoms in the ceramide fatty acid tails appeared to reduce the mRNA-luciferase expression in primary mouse hepatocytes, the expression levels of LNP42 (having C4 ceramide (d18:1/4:0)), LNP43 (having C6 ceramide (d18:1/6:0), and LNP45 (having C8 ceramide (d18:1/8:0) were nevertheless within the same order of magnitude as the expression level of the LNP F. As shown in FIG. 10, the luciferase expression level of C2 ceramide-containing LNP41 was slightly higher than the luciferase expression level of the LNP F.

TABLE 19
Physicochemical properties of LNP41, LNP42,
LNP43, and LNP45 having mRNA-luciferase cargo

| | | Average | Polydispersity | Encapsulation |
|---|---|---|---|---|
| LNP | | Diameter | Index | Efficiency |
| Formulation | Lipids and Molar Ratios | (nm) | (PDI) | (%) |
| LNP F | Ionizable Lipid | 67.7 | 0.066 | 95.7 |
| | 81:DSPC:Cholesterol:DMG- | | | |
| | PEG2000:DiD | | | |
| | (47.5:10:39:3:0.5) | | | |
| LNP41 | Ionizable Lipid 81:C2 | 75.8 | 0.076 | 95.3 |
| | ceramide:Cholesterol:DMG- | | | |
| | PEG2000:DiD | | | |
| | (47.5:10:39:3:0.5) | | | |
| LNP42 | Ionizable Lipid 81:C4 | 65.2 | 0.070 | 95.7 |
| | ceramide:Cholesterol:DMG- | | | |
| | PEG2000:DiD | | | |
| | (47.5:10:39:3:0.5) | | | |
| LNP43 | Ionizable Lipid 81:C6 | 65.4 | 0.103 | 94.4 |
| | ceramide:Cholesterol:DMG- | | | |
| | PEG2000:DiD | | | |
| | (47.5:10:39:3:0.5) | | | |
| LNP45 | Ionizable Lipid 81:C8 | 66.4 | 0.097 | 95.9 |
| | ceramide:Cholesterol:DMG- | | | |
| | PEG2000:DiD | | | |
| | (47.5:10:39:3:0.5) | | | |

### Example 9. LNP Formulations with mRNA Cargo Exhibit In Vivo Expression, Improved Half-Life in Whole Blood, and Superior Cargo Concentration and Retention in Certain Organs

The following LNP formulations as listed in Table 20 were prepared using luciferase mRNA as the nucleic acid cargo.

TABLE 20
Physicochemical properties of LNP formulations having mRNA-luciferase cargo

| | | Average | Polydispersity | Encapsulation |
|---|---|---|---|---|
| LNP | | Diameter | Index | Efficiency |
| Formulation | Lipids and Molar Ratios | (nm) | (PDI) | (%) |
| LNP G | Ionizable Lipid | 70.3 | 0.059 | 84.7 |
| | Z:DSPC:Cholesterol:DMG- | | | |
| | PEG2000 | | | |
| | (47.5:10:39.5:3) | | | |
| LNP101 | Ionizable Lipid | 89.9 | 0.01 | 87.8 |
| | 87:Cholesterol:DSG- | | | |
| | PEG2000-OMe | | | |
| | (57.6:39.5:3) | | | |
| LNP102 | Ionizable Lipid | 82.6 | 0.019 | 91.4 |
| | 87:DSPC:Cholesterol:DSG- | | | |
| | PEG2000-OMe | | | |
| | (47.5:10:39.5:3) | | | |
| LNP103 | Ionizable Lipid 87:C2 | 84.2 | 0.031 | 88.6 |
| | ceramide:Cholesterol:DSG- | | | |
| | PEG2000-OMe | | | |
| | (47.5:10:39:3) | | | |
| LNP104 | Ionizable Lipid | 74.0 | 0.030 | 90.3 |
| | Z:DSPC:Cholesterol:DSG- | | | |
| | PEG2000-OMe | | | |
| | (47.5:10:39:3) | | | |

CD-1 mice were injected IV bolus via the tail vein at a dose of 0.3 mg/kg of any one of the LNP formulations listed in Table 20. Whole blood samples were collected with K₂EDTA as anticoagulant 150 μL/aliquot for qPCR at 2 min, 1-hour and 6-hour timepoints and also at the 24-hour terminal timepoint. The total fluorescence (IVIS) in the liver was also measured at the 24-hour terminal timepoint. As can be seen in FIG. 10, LNP102, LNP103, and LNP104, which are LNP formulations of the invention, i.e., LNP formulations that include a helper lipid (e.g., C2 ceramide (d18:1/2:0) or DSPC) and a lipid-anchored polymer having two hydrophobic tails that each comprise 16 to 22 carbon atoms in a single aliphatic chain backbone (e.g., DSG-PEG2000-OMe), all showed good in vivo luciferase expression in mice.

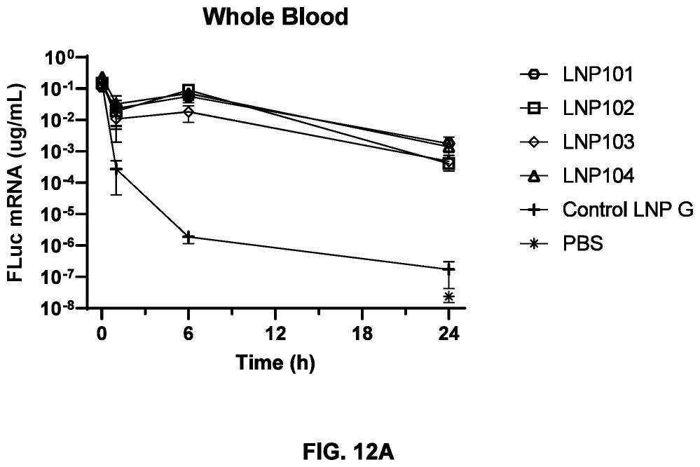

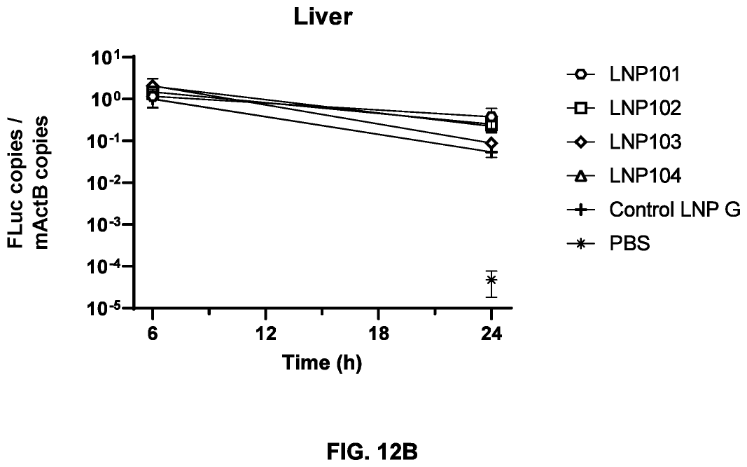

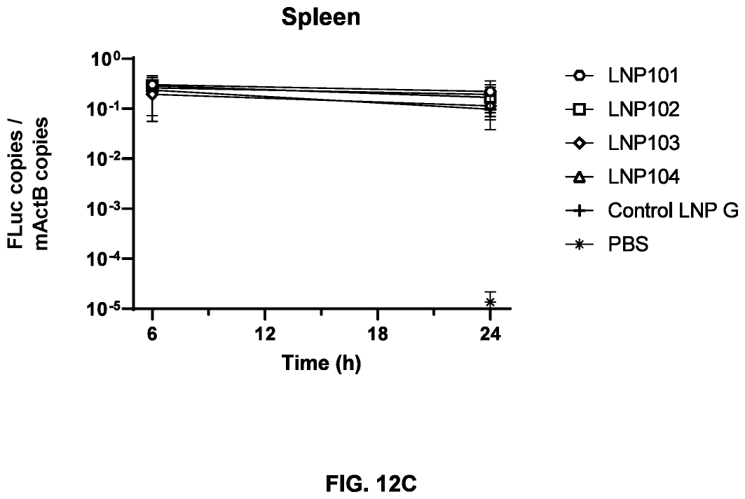

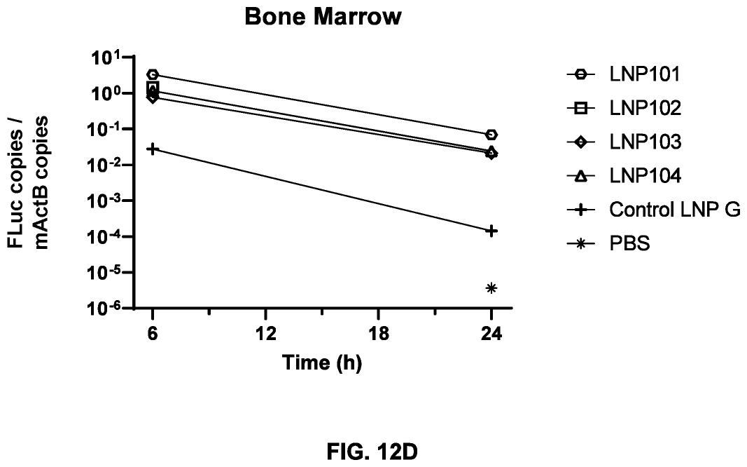

Furthermore, pharmacokinetic (PK) studies investigating luciferase mRNA levels over the 24-hour period in whole blood (FIG. 12A), liver (FIG. 12B), spleen (FIG. 12C), and bone marrow (FIG. 12D) were also conducted. Table 21 below summarizes the half-life (t_{1/2}), area under the curve at the terminal timepoint which is 24 hours (AUCₗₐₛₜ), and clearance rate (Cl) parameters of the tested luciferase mRNA LNP formulations. The following examples examine the t_{1/2} and AUC of stealth versus non-stealth LNPs at 30 minutes.

TABLE 21
Pharmacokinetic (PK) parameters of luciferase mRNA LNP formulations

| LNP | | t_{1/2} | AUCₗₐₛₜ | Cl |
|---|---|---|---|---|
| Formulation | Lipids and Molar Ratios | (hour) | (hour*ng/mL) | (mL/min/kg) |
| LNP G | Ionizable Lipid | 2.56 | 27.2 | 307 |
| | Z:DSPC:Cholesterol:DMG- | | | |
| | PEG2000 | | | |
| | (47.5:10:39.5:3) | | | |
| LNP101 | Ionizable Lipid | 5.20 | 536 | 15.2 |
| | 87:Cholesterol:DSG- | | | |
| | PEG2000-OMe | | | |
| | (57.6:39.5:3) | | | |
| LNP102 | Ionizable Lipid | 3.12 | 573 | 14.5 |
| | 87:DSPC:Cholesterol:DSG- | | | |
| | PEG2000-OMe | | | |
| | (47.5:10:39.5:3) | | | |
| LNP103 | Ionizable Lipid 87:C2 | 4.53 | 221 | 37.2 |
| | ceramide:Cholesterol:DSG- | | | |
| | PEG2000-OMe | | | |
| | (47.5:10:39:3) | | | |
| LNP104 | Ionizable Lipid | 4.38 | 672 | 12.2 |
| | Z:DSPC:Cholesterol:DSG- | | | |
| | PEG2000-OMe | | | |
| | (47.5:10:39:3) | | | |

Overall, the various PK parameters and values shown in Table 21 indicate that the LNP formulations of the invention (each having the C18 DSG-PEG2000-OMe as a lipid-anchored polymer) and LNP G having the combination of DSPC as a helper lipid and C14 DMG-PEG2000 as a lipid-anchored polymer possessed opposite PK profiles: longer half-life (t_{1/2}), higher blood exposure at the terminal timepoint which is 24 hours (AUCₗₐₛₜ) and slower clearance from the systemic circulation in the LNP formulations, as opposed to shorter half-life (t_{1/2}), lower blood exposure at the terminal timepoint (AUCₗₐₛₜ) and faster clearance from the systemic circulation in LNP G. In whole blood (FIG. 12A), LNP formulations of the invention, namely LNP102, LNP103, and LNP104, all exhibited excellent stealth properties in that the half-life (t_{1/2}) of each of these LNP formulations is about 3-6 hours. In contrast, LNP G that incorporates DMG-PEG2000 as a lipid-anchored polymer exhibited a half-life (t_{1/2}) of about 2.5 hours in whole blood.

From a clearance rate (Cl) standpoint, LNP102, LNP103, and LNP104 exhibited Cl rates of about 10-40 mL/min/kg, whereas LNP G had a significantly higher Cl rate of 307 ml/min/kg. A higher clearance rate (Cl) is indicative of a quicker rate of the drug substance (i.e., luciferase mRNA) being cleared from the systemic circulation. Hence, the Cl rates indicated that the luciferase mRNA delivered by LNP G was rapidly cleared from the bloodstream.

High Cl rates correlate with low blood exposures (AUCₗₐₛₜ) of the drug substance at the terminal timepoint, which in this case is 24 hours. Accordingly, the AUCₗₐₛₜ values in Table 21 indicate that a blood exposure (AUCₗₐₛₜ) of only 27.2 hour*ng/mL, as compared to significantly higher AUCₗₐₛₜ values of about 200 hour*ng/ml to about 700 hour*ng/ml of all of the LNP formulations of LNP102, LNP103, and LNP104. The calculated Cl rates and AUCₗₐₛₜ values of the tested LNP formulations are further corroborated by the PK curves of FIG. 12A. OAs shown in FIG. 12A, the luciferase mRNA concentrations as detected by qPCR steadily dropped from 10⁻¹ μg/mL to 10⁻³ μg/mL over the 24-hour period in all of the LNP formulations; whereas in LNP G the luciferase mRNA concentrations dropped from 10⁻¹ μg/mL to almost 10⁻⁴ μg/mL within the first hour, which continued to significantly drop to 10⁻⁶ μg/mL at 6 hours and further, to almost 10⁻⁷ μg/mL at 24 hours. As discussed above in Example 7, the higher retention of ceDNA in the bloodstream or less rapid clearance of the ceDNA cargo from the bloodstream as delivered by the and C2 ceramide-containing LNP1 could be beneficial in that off-target delivery to non-target cells.

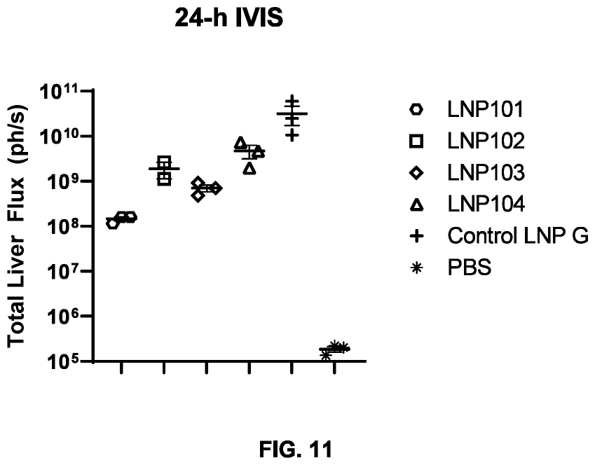

In the liver (FIG. 12B), LNP102, LNP103, and LNP104 all exhibited slightly higher number of copies of luciferase mRNA and at least equivalent or higher retention rates of luciferase mRNA from 6 hours to 24 hours post-dosing, as compared to LNP G. The observation of higher luciferase mRNA amounts in the LNP formulations is surprising and unexpected, considering that the IVIS fluorescence data as shown in FIG. 11 suggested that the luciferase mRNA expression levels of these LNP formulations were lower than that of LNP G.

Similarly, in the spleen (FIG. 12C), LNP formulations of the invention all exhibited excellent retention rates of luciferase mRNA from 6 hours to 24 hours post-dosing with negligible reductions in copy amounts whereby the amount at 6 hours and the amount at 24 hours post-dosing were within the same order of magnitude. In the spleen, T-cells including CD8+ T-cells are primed to generate precursors with an enhanced ability to differentiate into long-lived, stem-like memory T cells. Stem-like T-cells (TSC) are a subpopulation of mature T-cells that display stem cell-like properties, maintaining long-lasting immune effect even among exhausting clones.

In the bone marrow where hematopoietic stems cells (HSC) are present (FIG. 12D), the amounts of luciferase mRNA throughout the 6-24 hour post-dosing period, as delivered by LNP formulations of the invention (i.e., LNP102, LNP103, and LNP104), were consistently found to be about 2 orders of magnitude higher than the amounts of luciferase mRNA as delivered by LNP G. Moreover, as indicated in FIG. 12D, the retention rates of luciferase mRNA in the mice bone marrow during the 6-24-hour post-dosing period, as delivered by LNP102, LNP103, and LNP104, were also superior to the retention rate of the mRNA as delivered by LNP G. Within the 6-24-hour post-dosing period, the amount of copies of luciferase mRNA in the mice bone marrow merely dropped for less than one order of magnitude in mice dosed with the LNP formulations, as compared to a drop of greater than one order of magnitude within the same time window for mice dosed with LNP G.

### Example 10. Blood pK Characteristics of Stealth Versus Non-Stealth LNPs within 30 Minutes of Administration to the Blood in CD-1 Mice

The following study was performed in male CD-1 mice aged 3-5 weeks. Animals were dosed with dose of 1 mg/kg of LNP201, LNP202, and LNP203, all encapsulating ceDNA as a cargo. These LNPs were formulated as described above and their particle sizes and encapsulation efficiency were measured and found to be consistent with previous results (e.g., the average particle size of <80 nm in diameter and 90% EE, respectively). Blood samples were collected through submandibular or tail vein nick at 0 h, 1 h, 3 h, 6 h, and 24 h post dose administration. Blood samples were stored at −80° C. in K₂ EDTA tubes until analysis was performed to determine ceDNA levels (qPCR analysis was performed to determine ceDNA levels in blood).

TABLE 22
Formulations for pK Analysis over 30 minutes in Blood

| Test Article | Compositional Basis (mol %) |
|---|---|
| LNP201 | Lipid Z/DSPC/Chol/DSG-PEG2000-OH/DiD/ |
| | DSPE-PEG5000-N3 (47.5/10.0/39.0/2.8/0.5/0.2) |
| LNP202 | Lipid 87/DSPC/Chol/DSG-PEG2000-OH/DiD/ |
| | DSPE-PEG5000-N3 (47.5/10.0/39.0/2.8/0.5/0.2) |
| LNP203 - non-Stealth | Lipid Z/DSPC/chol/DMG-PEG2000-OH |
| reference LNP | |

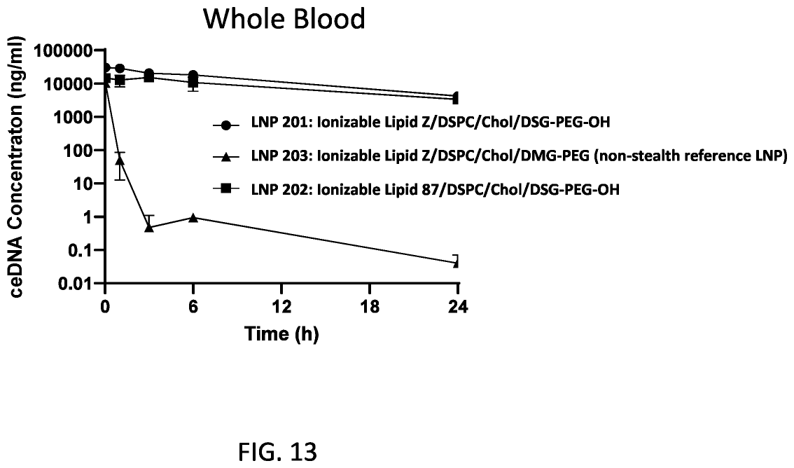

As shown in FIG. 13, LNP201 (ionizable Lipid Z, t_{1/2}=8.91) and LNP202 (ionizable Lipid 87, t_{1/2}=10.4) that contain C18 DSG-PEG as a lipid-anchored polymer exhibited much greater and extended blood half-life (t_{1/2}) as compared to that of the reference LNP, LNP203 (t_{1/2}=0.25 alpha phase), which comprises C14 DMG-PEG as lipid polymer. The detailed results are shown in Table 23 below and FIG. 13:

TABLE 23
Blood pK characteristics for stealth versus non-stealth LNP in the
first 3 hours of blood exposure as measured by t_{1/2} and AUC.

| | Half-life (t_{1/2}) | AUC (hr*ng/ml) |
|---|---|---|
| Formulation | (stealth/non-stealth) | (stealth/non-stealth) |
| LNP201 (Stealth) | 8.91 (35.6-fold) | 311,000 (207-fold) |
| LNP202 (Stealth) | 10.4 (41.6-fold) | 193,000 (128-fold) |
| LNP203 non-stealth Lipid Z | 0.25 (alpha) | 1500 |

For LNP203 and other non-stealth LNPs, 99.9% of the formulation is cleared from systemic circulation by 3 hours, hence only t_{1/2} of the alpha phase (i.e., 0.25 t_{1/2}) is reported in Table 23. When the rapid clearance of 99.9% of the LNP for non-stealth LNP is considered, the ratios of the t_{1/2} and AUC of stealth to non-stealth is very different than shown in the previous example. In this example, the biological effect of stealth versus non-stealth LNPs is much more pronounced. The ratio of the t_{1/2} (hr) alpha phase is 35.6 for LNP201 versus LNP203. Similarly, the ratio of the t_{1/2} (hr) alpha phase is 41.6 for LNP202 versus LNP203.

The effect on AUC was even more intense. The ratio of the AUC (hr*ng/ml) of the alpha phase is 207-fold for LNP201 versus LNP203. Similarly, the ratio of the AUC (hr*ng/ml) of the alpha phase is 128-fold for LNP202 versus LNP203. These data suggest that stealth LNPs comprising C18 lipid-anchored polymer, all formulated using the component ratios disclosed herein consistently exhibited stealth characteristics that are resistant to random off-target delivery to or biological uptake by non-target cells, while possessing average particle sizes effective for therapeutic uses (<80 nm in diameter).

### Example 11. Effect of Increasing Lipid Anchored Polymer Content on Stealth LNPs

In this example, the effect of increasing the amount of lipid anchored polymer into helper lipid containing stealth LNPs was explored. A series of LNPs containing from 1.5% and up to 7.0% lipid-anchored polymers were formulated as shown in Table 24 below:

TABLE 24
Maximum Amount of Lipid Anchored Polymers in Stealth LNPs

| | |
|---|---|
| Lipid-Anchored Polymer | Lipid-Anchored Polymer mol % |

| | | | | |
|---|---|---|---|---|
| DSG-PEG2k-OMe | 1.5 | 3.0 | 5.0 | 7.0 |
| DODA-PG45 | 1.5 | 3.0 | 5.0 | 7.0 |
| DSPE-PCB1 | 1.5 | 3.0 | 5.0 | 7.0 |
| DSPE-PMPC | 1.5 | 3.0 | 5.0 | 7.0 |

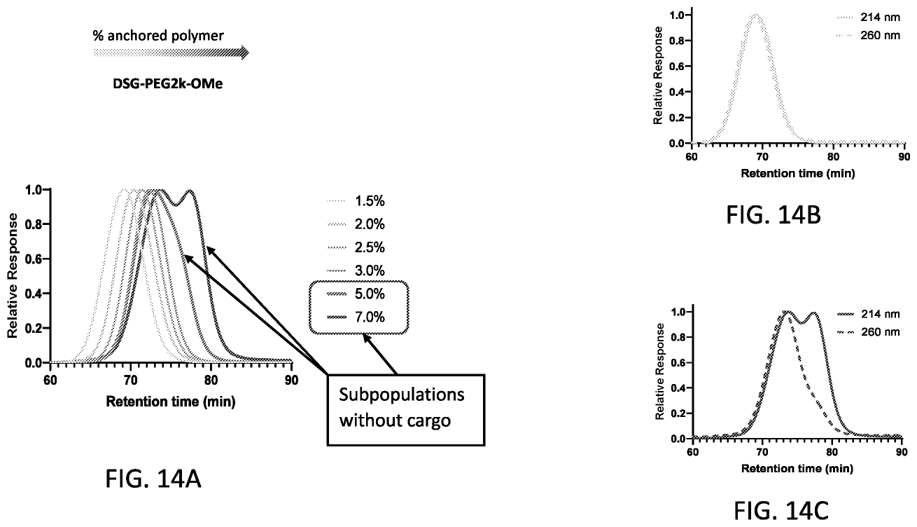

The polymer composition was Lipid Z, DSPC (helper lipid), cholesterol, and 1.5-7.0% lipid anchored polymer in 47.5:10:(35.5-41):1.5-7.0 mol % ratios or Lipid Z, DSPC, cholesterol, DiD, DSPE-PEG5000-N3, second lipid-anchored polymer in mol % ratios 47.5:10:(35.0-40.5):0.5:0.2:(1.3-6.8). Polymer hydrophilicity of the lipid-anchored polymers of Table 24 decreases as the list goes down the rows. The results showed that increasing polymer density up to 5 and 7 mol % significantly increased LNP thermal stability to elevated temperatures ranging from 20-80° C. As shown in FIG. 14A, using HPLC-SEC analysis of the listed LNPs, it was found that increasing the total lipid-anchored polymer content either with a first and second lipid-anchored polymer or any of a single lipid-anchored polymer of Table 24 greater than 5 mol % resulted in subpopulations of LNP particles without cargo encapsulated. FIG. 14B shows the uniform retention time of LNPs with cargo at 1.5 mol % lipid-anchored polymer (measured at 214 nm to track lipid and 260 nm to track nucleic acid cargo) and FIG. 14C shows the non-uniform retention time of LNPs with cargo at 7 mol % lipid-anchored polymer, suggesting that the presence of a subpopulation of LNPs without cargo at higher polymer mol % (e.g., 7%). Therefore, in the present disclosure, 1.5 mol %, 2 mol %, 2.5 mol %, 3 mol %, and up to 5 mol % of a lipid-anchored polymer was found to be an ideal target range for most stealth LNP formulations. See FIGS. 14A-C.

### Example 12. Designing Stealthy and Targetable LNPs with Stability

A useful enhancement of the functionality of the stealth LNPs disclosed herein is to add the ability of the LNP first to evade rapid opsonization/destabilization and also to target the cargo to specific cells and tissues by adding a targeting moiety to the LNP through conjugation to, e.g., a lipid anchored polymer. The goal was to first create stealthy LNPs by using between 3-5 mol % lipid-anchored polymer in combination with ionizable lipids (35-50 mol %), helper lipids (~10 mol %), and sterols (~30-40 mol %) where the LNP can be sufficiently stable, small, and stealthy to transport any cargo such as mRNA, dsDNA, ssDNA or other gene editing or gene silencing components. The basis of this design was that a combination of one or two lipid anchored polymers can be divided into two primary functions. Those functions include first to provide stealth character to an LNP by avoiding rapid opsonization and remaining in blood circulation long enough for the second, and critically important targeting function to be carried out. This second function, which is a targeting function, should be achieved through a sub-population of the total lipid anchored polymer content in mol % on the surface of the LNP. The targeting function occurs by inclusion of a conjugation moiety (“handle”) to a subpopulation of the total lipid-anchored polymers (“second lipid-anchored polymer”) which can be conjugated to a targeting moiety such as an scFv, VHH or one or more other specific binding ligand moieties. This disclosure provides such stealth LNPs with a first lipid anchored polymer as the main driver of LNP stealth and stability through employing linker-lipid portion for the first anchored lipid that do not allow rapid dissociation and function to enable stealthiness (e.g., C18 DODA, C18 DSPE, C18 DSG, etc.). Next, this disclosure provides a second lipid anchored polymer functionalized to contain a conjugation handle to conjugate a targeting moiety to the LNP. The population or subpopulation of lipid anchored polymer conveying the targeting function can also contribute to the stealth characteristic of the LNP by carefully selecting a lipid component that resists rapid disassociation from the LNP surface.

In this example, stealth LNPs containing a second lipid polymer with a conjugation handle attached were formulated. The size of the resulting LNP and % encapsulation of an mRNA cargo was measured.

The results in Table 25 show twenty working examples of formulated stealth LNPs with an azide conjugation handle covalently attached to a second lipid anchored polymer and where all the LNPs encapsulate an mRNA cargo expressing luciferase. In Table 25, LNPs 301-310 do not include a helper lipid and thus contain a higher level (57.6%) of the ionizable lipid, which generally led to larger particle sizes as compared to corresponding LNPs having ~10 mol % helper lipid (e.g., DSPC, C2 ceramide, etc.). In Table 25, LNPs 311-320 contain 10% DSPC helper lipid and 47.5% of various ionizable lipids and 40 mol % of cholesterol. All of the LNP formulations Table 25, encapsulated the cargo in an acceptable manner. The vast majority of the formulated LNPs encapsulated the cargo with greater than 90% effectiveness. The particle size after formulation was acceptable in most cases (e.g., 60-80 nm in diameter) with only two formulations that had no helper lipid showing an average size greater than 100 nm in diameter.

TABLE 25
Composition with Reactive Azide on Second Lipid Anchored Polymer as Conjugation Handle

| LNP | Composition with Reactive Azide on Second Lipid Anchored Polymer | Size (nm) | EE(%) |
|---|---|---|---|
| 301 | LIPID 87:Chol:DSG-PEG2000-OMe:DSPE-PEG5000-N3:DiD | 85.3 | 92.0% |
| | (57.6:39.45:2.4:0.5:0.05) | | |
| 302 | LIPID Z:Chol:DSG-PEG2000-OMe:DSPE-PEG5000-N3:DiD | 101.8 | 93.7% |
| | (57.6:39.45:2.4:0.5:0.05) | | |
| 303 | Lipid 58:Chol:DSG-PEG2000-OMe:DSPE-PEG5000-N3:DiD | 101.4 | 93.1% |
| | (57.6:39.45:2.4:0.5:0.05) | | |
| 304 | MC3:Chol:DSG-PEG2000-OMe:DSPE-PEG5000-N3:DiD | 87.5 | 90.9% |
| | (57.6:39.45:2.4:0.5:0.05) | | |
| 305 | L369:Chol:DSG-PEG2000-OMe:DSPE-PEG5000-N3:DiD | 87.1 | 92.9% |
| | (57.6:39.45:2.4:0.5:0.05) | | |
| 306 | LP01:Chol:DSG-PEG2000-OMe:DSPE-PEG5000-N3:DiD | 78.6 | 96.0% |
| | (57.6:39.45:2.4:0.5:0.05) | | |
| 307 | SM102:Chol:DSG-PEG2000-OMe:DSPE-PEG5000-N3:DiD | 78.1 | 92.8% |
| | (57.6:39.45:2.4:0.5:0.05) | | |
| 308 | DOTAP:Chol:DSG-PEG2000-OMe:DSPE-PEG5000-N3:DiD | 64.1 | 98.8% |
| | (57.6:39.45:2.4:0.5:0.05) | | |
| 309 | LIPID 81:Chol:DSG-PEG2000-OMe:DSPE-PEG5000-N3:DiD | 88.8 | 94.2% |
| | (57.6:39.45:2.4:0.5:0.05) | | |
| 310 | ALC-0315:Chol:DSG-PEG2000-Ome:DSPE-PEG5000-N3:DiD | 88.4 | 89.2% |
| | (57.6:39.45:2.4:0.5:0.05) | | |
| 311 | LIPID 87:DSPC:Chol:DSG-PEG2000-Ome:DSPE-PEG5000-N3:DiD | 70.6 | 99.0% |
| | (47.5:10:39.45:2.5:0.5:0.05) | | |
| 312 | Lipid Z:DSPC:Chol:DSG-PEG2000-Ome:DSPE-PEG5000- | 67.7 | 99.0% |
| | N3:DiD (47.5:10:39.45:2.5:0.5:0.05) | | |
| 313 | LIPID 58:DSPC:Chol:DSG-PEG2000-Ome:DSPE-PEG5000- | 70.5 | 97.6% |
| | N3:DiD (47.5:10:39.45:2.5:0.5:0.05) | | |
| 314 | MC3:DSPC:Chol:DSG-PEG2000-Ome:DSPE-PEG5000-N3:DiD | 65.8 | 97.2% |
| | (47.5:10:39.45:2.5:0.5:0.05) | | |
| 315 | L369:DSPC:Chol:DSG-PEG2000-Ome:DSPE-PEG5000-N3:DiD | 68.5 | 99.2% |
| | (47.5:10:39.45:2.5:0.5:0.05) | | |
| 316 | LP01:DSPC:Chol:DSG-PEG2000-Ome:DSPE-PEG5000-N3:DiD | 68.9 | 98.0% |
| | (47.5:10:39.45:2.5:0.5:0.05) | | |
| 317 | SM102:DSPC:Chol:DSG-PEG2000-Ome:DSPE-PEG5000- | 63.9 | 99.3% |
| | N3:DiD (47.5:10:39.45:2.5:0.5:0.05) | | |
| 318 | DOTAP:DSPC:Chol:DSG-PEG2000-Ome:DSPE-PEG5000-N3:DiD | 74.8 | 99.9% |
| | (47.5:10:39.45:2.5:0.5:0.05) | | |
| 319 | LIPID 81:DSPC:Chol:DSG-PEG2000-Ome:DSPE-PEG5000-N3:DiD | 80.8 | 98.7% |
| | (47.5:10:39.45:2.5:0.5:0.05) | | |
| 320 | ALC-0315:DSPC:Chol:DSG-PEG2000-Ome:DSPE-PEG5000- | 62.8 | 96.9% |
| | N3:DiD (47.5:10:39.45:2.5:0.5:0.05) | | |

In summary, it was found that most of the LNPs formulated with different ionizable lipids and the azide-containing second lipid-anchored polymer showed useful LNP characteristics for effective conjugation to any potential targeting moiety, thereby significantly enhancing the functionality as stealthy LNP.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

What is claimed is:

1. A lipid nanoparticle (LNP) comprising:
a therapeutic nucleic acid (TNA);
an ionizable lipid;
a sterol;
a first lipid-anchored polymer; wherein the first lipid-anchored polymer comprises:
i) a polymer;
ii) a lipid moiety comprising at least one hydrophobic tail; and
iii) optionally a linker connecting the polymer to the lipid moiety;
wherein the at least one hydrophobic tail comprises 12 to 22 carbon atoms in a single aliphatic chain backbone; and
a helper lipid represented by Formula (I): 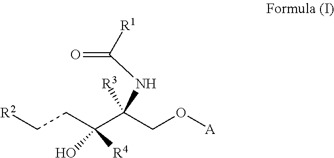
or a salt or an ester thereof, wherein: [symbol]
is a single bond or a double bond;
A is hydrogen, 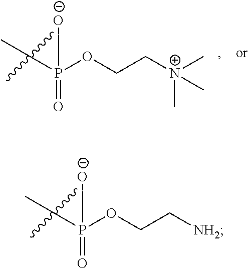 [SMILES: *P(=O)([O-])OCC[N+](C)(C)C ; *P(=O)([O-])OCCN]
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

2. A lipid nanoparticle (LNP) comprising:
a therapeutic nucleic acid (TNA);
an ionizable lipid;
a sterol;
a first lipid-anchored polymer; wherein the first lipid-anchored polymer comprises:
i) a polymer;
ii) a lipid moiety comprising at least two hydrophobic tails; and
iii) optionally a linker connecting the polymer to the lipid moiety;
wherein the at least two hydrophobic tails each comprise 16 to 22 carbon atoms in a single aliphatic chain backbone; and
a helper lipid represented by Formula (I): 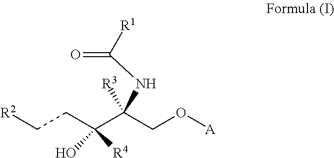
or a salt or an ester thereof, wherein: [symbol]
is a single bond or a double bond;
A is hydrogen, 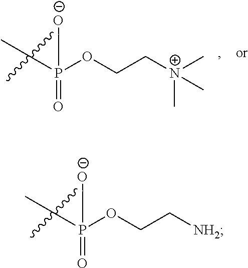 [SMILES: *P(=O)([O-])OCC[N+](C)(C)C ; *P(=O)([O-])OCCN]
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

3. A lipid nanoparticle (LNP) comprising:
a therapeutic nucleic acid (TNA);
an ionizable lipid;
a sterol;
a first lipid-anchored polymer; wherein the first lipid-anchored polymer comprises:
i) a polymer;
ii) a lipid moiety comprising at least two hydrophobic tails; and
iii) optionally a linker connecting the polymer to the lipid moiety;
wherein the at least two hydrophobic tails each comprise 12 to 15 carbon atoms in a single aliphatic chain backbone; and
a helper lipid represented by Formula (I): 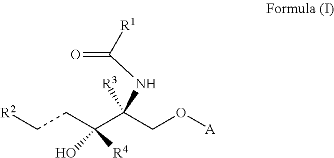
or a salt or an ester thereof, wherein: [symbol]
is a single bond or a double bond;
A is hydrogen, 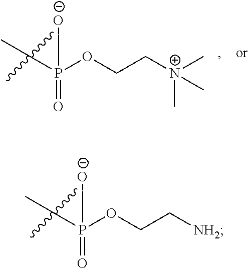 [SMILES: *P(=O)([O-])OCC[N+](C)(C)C ; *P(=O)([O-])OCCN]
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

4. A lipid nanoparticle (LNP) comprising:
a therapeutic nucleic acid (TNA);
an ionizable lipid;
a sterol;
a first lipid-anchored polymer; wherein the first lipid-anchored polymer comprises:
i) a polymer;
ii) a lipid moiety comprising a single hydrophobic tail; and
iii) optionally a linker connecting the polymer to the lipid moiety;
wherein the single hydrophobic tail comprises 12 to 22 carbon atoms in a single aliphatic chain backbone; and
a helper lipid represented by Formula (I): 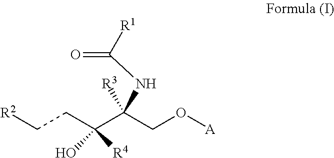
or a salt or an ester thereof, or a deuterated analogue of any of the foregoing, wherein: [symbol]
is a single bond or a double bond;
A is hydrogen, 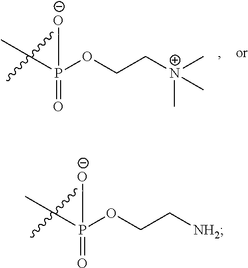 [SMILES: *P(=O)([O-])OCC[N+](C)(C)C ; *P(=O)([O-])OCCN]
R¹ is C₁-C₁₇ alkyl or C₂-C₁₇ alkenyl;
R² is C₁-C₂₂ alkyl or C₂-C₂₂ alkenyl;
R³ is hydrogen or C₁-C₂ alkyl; and
R⁴ is hydrogen or C₁-C₂ alkyl.

5. The lipid nanoparticle (LNP) of any one of claims 1 to 4, wherein the helper lipid is represented by Formula (II): 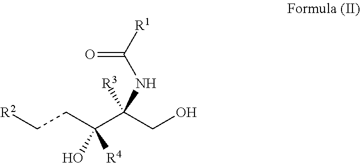
or a salt or an ester thereof, or a deuterated analogue of any of the foregoing.

6. The lipid nanoparticle (LNP) of any one of claims 1 to 4, wherein the helper lipid is represented by Formula (III): 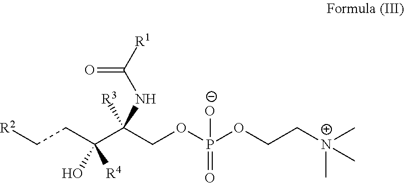
or a salt or an ester thereof, or a deuterated analogue of any of the foregoing.

7. The lipid nanoparticle (LNP) of any one of claims 1 to 4, wherein the helper lipid is represented by Formula (IV): 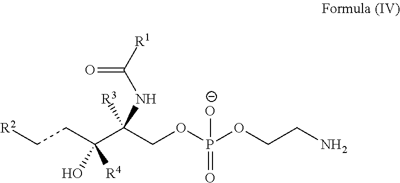
or a salt or an ester thereof, or a deuterated analogue of any of the foregoing.

8. The lipid nanoparticle (LNP) of any one of claims 1 to 7, wherein the LNP does not comprise distearoylphosphatidylcholine (DSPC), provided that a helper lipid represented by (I), (II), (III), or (IV), or a salt or an ester thereof, or a deuterated analogue of any of the foregoing is present.

9. The lipid nanoparticle (LNP) of any one of claims 1 to 8, wherein the LNP does not comprise 1,2-dioleoyl-sn-glycero-3-phosphocholine (DOPC) or 1,2-dioleoyl-sn-glycero-3-phosphoethanolamine (DOPE), provided that a helper lipid represented by (I), (II), (III), or (IV), or a salt or an ester thereof, or a deuterated analogue of any of the foregoing is present.

10. The lipid nanoparticle (LNP) of any one of claims 1 to 9, wherein the LNP does not comprise a phosphatidylcholine that is not conjugated to a polymer.

11. The lipid nanoparticle (LNP) of any one of claims 1 to 7, wherein R¹ is C₁-C₁₀ alkyl or C₂-C₁₀ alkenyl.

12. The lipid nanoparticle (LNP) of any one of claims 1 to 11, wherein [symbol] is a double bond.

13. The lipid nanoparticle (LNP) of any one of claims 1 to 11, wherein R¹ is C₁-C₈ alkyl or C₂-C₈ alkenyl.

14. The lipid nanoparticle (LNP) of claim 13, wherein R¹ is C₁-C₇ alkyl or C₂-C₇ alkenyl.

15. The lipid nanoparticle (LNP) of claim 14, wherein R¹ is C₁ alkyl, C₃ alkyl, C₅ alkyl, or C₇ alkyl.

16. The lipid nanoparticle (LNP) of claim 15, wherein R¹ is C₁ alkyl.

17. The lipid nanoparticle (LNP) of any one of claims 1 to 16, wherein R² is C₃-C₁₅ alkyl or C₃-C₁₅ alkenyl.

18. The lipid nanoparticle (LNP) of claim 17, wherein R² is C₉ alkyl, C₁₁ alkyl, C₁₂ alkyl, C₁₃ alkyl, C₁₄ alkyl or C₁₅ alkyl.

19. The lipid nanoparticle (LNP) of claim 18, wherein R² is C₁₂ alkyl, C₁₃ alkyl, or C₁₄ alkyl.

20. The lipid nanoparticle (LNP) of claim 19, wherein R² is C₁₃ alkyl.

21. The lipid nanoparticle (LNP) of any one of claims 1 to 20, wherein R³ is hydrogen.

22. The lipid nanoparticle (LNP) of any one of claims 1 to 20, wherein R³ is C₁ alkyl.

23. The lipid nanoparticle (LNP) of any one of claims 1 to 22, wherein R⁴ is hydrogen.

24. The lipid nanoparticle (LNP) of any one of claims 1 to 22, wherein R⁴ is C₁ alkyl.

25. The lipid nanoparticle (LNP) of claim 1, wherein the helper lipid represented by Formula (I) is selected from any of the helper lipids listed in Table 8, or a salt or an ester thereof, or a deuterated analogue of any of the foregoing; or the helper lipid represented by Formula (I) is selected from: 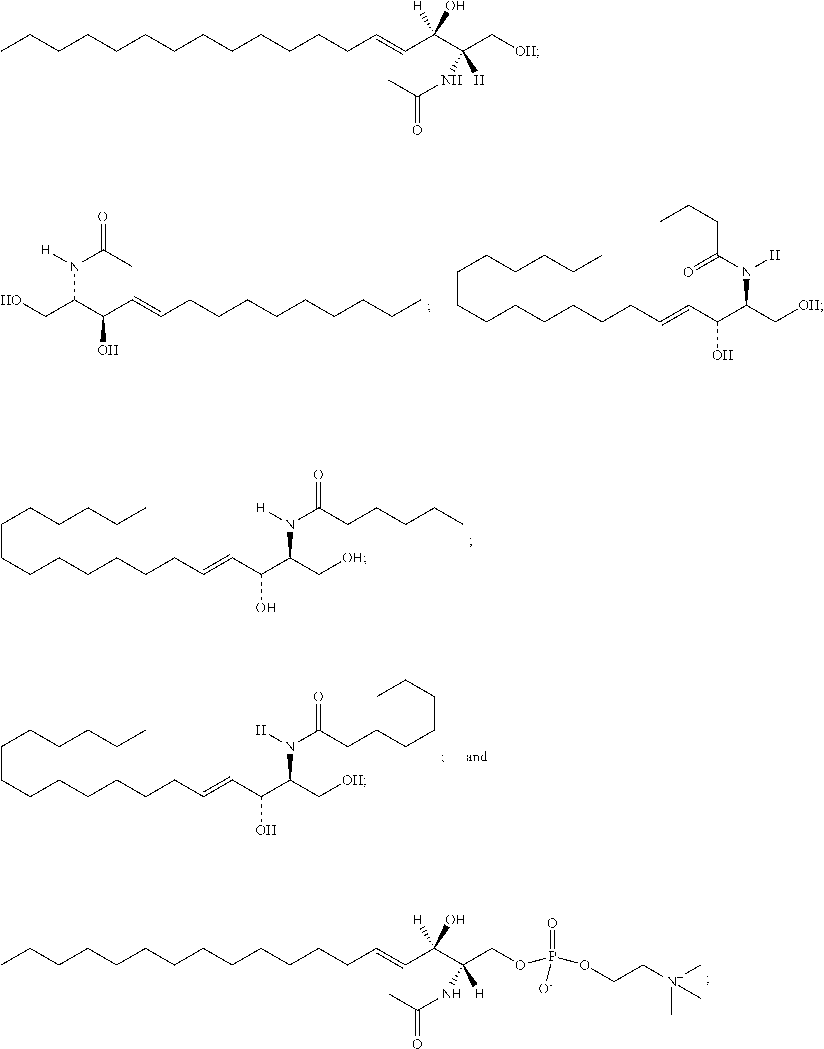
or a salt or an ester thereof, or a deuterated analogue of any of the foregoing.

26. The lipid nanoparticle (LNP) of claim 25, wherein the helper lipid represented by Formula (I) is 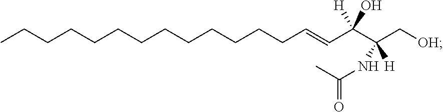 [SMILES: CCCCCCCCCCCCC/C=C/[C@@H](O)[C@H](CO)NC(C)=O]
or a salt or an ester thereof, or a deuterated analogue of any of the foregoing.

27. The lipid nanoparticle (LNP) of claim 25, wherein the lipid represented by Formula (I) is: 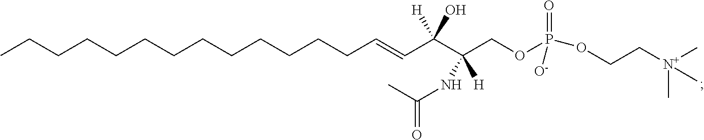 [SMILES: CCCCCCCCCCCCC/C=C/[C@@H](O)[C@H](COP(=O)([O-])OCC[N+](C)(C)C)NC(C)=O]
or a salt or an ester thereof, or a deuterated analogue of any of the foregoing.

28. A lipid nanoparticle (LNP) comprising:
a therapeutic nucleic acid (TNA);
an ionizable lipid;
a sterol;
a first lipid-anchored polymer; wherein the first lipid-anchored polymer comprises:
i) a polymer;
ii) a lipid moiety comprising at least two hydrophobic tails; and
iii) optionally a linker connecting the polymer to the lipid moiety;
wherein the at least two hydrophobic tails each comprise 16 to 22 carbon atoms in a single aliphatic chain backbone; and
a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE);
wherein the LNP has a whole blood half-life (t_{1/2}) of at least about 3 hours.

29. The lipid nanoparticle (LNP) of claim 28, wherein the LNP has a whole blood half-life (t_{1/2}) of about 3 hours to about 8 hours, or about 3 hours to about 7.5 hours, or about 3 hours to about 7 hours, or about 3 hours to about 6.5 hours, or about 3 hours to about 6 hours, or about 3 hours to about 9 hours, or about 3 hours to about 10 hours, or about 3 hours to about 11 hours, or about 3 hours to about 12 hours, or about 3 hours to about 13 hours, or about 3 hours to abouts 14 hours, or about 3 hours to about 15 hours, or about 3 hours to about 16 hours, or about 3 hours to about 24 hours.

30. The lipid nanoparticle (LNP) of claim 29, wherein the LNP has a whole blood half-life (t_{1/2}) of about 3 hours to about 3.5 hours, or about 3 hours to about 4 hours, or about 3 hours to about 4.5 hours, or about 3 hours to about 5 hours, or about 3 hours to about 5.5 hours, or about 3.5 hours to about 4 hours, or about 3.5 hours to about 4.5 hours, or about 3.5 hours to about 5 hours, or about 3.5 hours to about 5.5 hours, or about 4 hours to about 4.5 hours, or about 4 hours to about 5 hours, or about 4 hours to about 5.5 hours, or about 4.5 hours to about 5 hours, or about 4.5 hours to about 5.5 hours, or about 5 hours to about 5.5 hours.

31. The lipid nanoparticle (LNP) of any one of claims 28 to 30, wherein the LNP has a whole blood clearance rate (Cl) of about 10 mL/min/kg to about 50 mL/min/kg, or about 10 mL/min/kg to about 45 mL/min/kg, or about 10 mL/min/kg to about 40 mL/min/kg.

32. The lipid nanoparticle (LNP) of claim 31, wherein the LNP has a whole blood clearance rate (Cl) of about 30 mL/min/kg to about 40 mL/min/kg, or about 35 mL/min/kg to about 40 mL/min/kg, or about 10 mL/min/kg to about 20 mL/min/kg, or about 10 mL/min/kg to about 18 mL/min/kg, or about 10 mL/min/kg to about 15 mL/min/kg.

33. The lipid nanoparticle (LNP) of any one of claims 28 to 32, wherein the LNP has a whole blood terminal timepoint exposure (AUCₗₐₛₜ) of at least 5 times greater than that of a reference LNP having C14-15 lipid polymer.

34. The lipid nanoparticle (LNP) of claim 33, wherein the C14-15 lipid polymer is DMA PEG.

35. The lipid nanoparticle (LNP) of claim 33, wherein the LNP has a whole blood terminal timepoint exposure (AUCₗₐₛₜ) of about 200 hour*ng/ml to about 250 hour*ng/ml, or about 200 hour*ng/ml to about 300 hour*ng/mL, or about 500 hour*ng/mL to about 700 hour*ng/ml, or about 500 hour*ng/ml to about 550 hour*ng/ml, or about 500 hour*ng/ml to about 600 hour*ng/mL, or about 550 hour*ng/ml to about 600 hour*ng/mL, or about 600 hour*ng/ml to about 700 hour*ng/ml, or about 600 hour*ng/mL to about 650 hour*ng/ml, or about 650 hour*ng/ml to about 700 hour*ng/mL.

36. The lipid nanoparticle (LNP) of claim 34 or claim 35, wherein the terminal timepoint is 24 hours.

37. The lipid nanoparticle (LNP) of any one of claims 1 to 27, wherein the first lipid-anchored polymer comprises a lipid moiety comprising a single or two hydrophobic tails.

38. The lipid nanoparticle (LNP) of any one of claims 28 to 30, wherein the first lipid-anchored polymer comprises a lipid moiety comprising two hydrophobic tails.

39. The lipid nanoparticle (LNP) of claim 38, wherein the two hydrophobic tails are each a fatty acid.

40. The lipid nanoparticle (LNP) of any one of claims 27 to 36, 38, and 39, wherein the two hydrophobic tails each independently comprise 16, 17, 18, 19, 20, 21, or 22 carbon atoms.

41. The lipid nanoparticle (LNP) of claim 40, wherein each of the two hydrophobic tails each independently comprise 16, 17, 18, 19, 20, or 21 carbon atoms.

42. The lipid nanoparticle (LNP) of claim 41, wherein each of the two hydrophobic tails each independently comprise 16, 17, 18, 19, or 20 carbon atoms.

43. The lipid nanoparticle (LNP) of claim 42, wherein the two hydrophobic tails each independently comprise 16, 17, 18, or 19 carbon atoms.

44. The lipid nanoparticle (LNP) of claim 43, wherein the two hydrophobic tails each independently comprise 16, 17, or 18 carbon atoms.

45. The lipid nanoparticle (LNP) of claim 44, wherein the two hydrophobic tails each comprise 16 carbon atoms.

46. The lipid nanoparticle (LNP) of claim 44, wherein the two hydrophobic tails each comprise 18 carbon atoms.

47. The lipid nanoparticle (LNP) of claim 42, wherein the two hydrophobic tails each comprise 20 carbon atoms.

48. The lipid nanoparticle (LNP) of any one of claims 38 to 47, wherein the two hydrophobic tails are each independently selected from the group consisting of octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

49. The lipid nanoparticle (LNP) of claim 38, wherein one of the two hydrophobic tails comprises 12, 13, 14, or 15 carbon atoms.

50. The lipid nanoparticle (LNP) of claim 49, wherein one of the two hydrophobic tails comprises 12, 13, or 14 carbon atoms.

51. The lipid nanoparticle (LNP) of claim 50, wherein one of the two hydrophobic tails comprises 12 carbon atoms.

52. The lipid nanoparticle (LNP) of claim 50, wherein one of the two hydrophobic tails each comprise 14 carbon atoms.

53. The lipid nanoparticle (LNP) of any one of claims 49 to 52, wherein one of the two hydrophobic tails is selected from the group consisting of lauric acid, myristic acid, myristoleic acid, and a derivative thereof.

54. The lipid nanoparticle (LNP) of claim 37, wherein the first lipid-anchored polymer comprises a lipid moiety comprising a single hydrophobic tail.

55. The lipid nanoparticle (LNP) of claim 54, wherein the single hydrophobic tail is a fatty acid.

56. The lipid nanoparticle (LNP) of claim 55, wherein the single hydrophobic tail comprises 16, 17, 18, 19, 20, 21, or 22 carbon atoms.

57. The lipid nanoparticle (LNP) of claim 56, wherein the single hydrophobic tail comprises 16, or 18 carbon atoms.

58. The lipid nanoparticle (LNP) of claim 56, wherein the single hydrophobic tail comprises 20 carbon atoms.

59. The lipid nanoparticle (LNP) of claim 56, wherein the single hydrophobic tail comprises 18 carbon atoms.

60. The lipid nanoparticle (LNP) of claim 56, wherein the single hydrophobic tail comprises 16 carbon atoms.

61. The lipid nanoparticle (LNP) of claim 55, wherein the single hydrophobic tail is selected from the group consisting of octadecylamine, palmitic acid, stearic acid, arachidic acid, palmitoleic acid, sapienic acid, oleic acid, elaidic acid, vaccenic acid, linoleic acid, linoelaidic acid, α-linolenic acid, arachidonic acid, eicosapentaenoic acid, and a derivative thereof.

62. The lipid nanoparticle (LNP) of any one of claims 1 to 61, wherein the first lipid-anchored polymer is a glycerolipid.

63. The lipid nanoparticle (LNP) of any one of claims 1 to 61, wherein the first lipid-anchored polymer is a phospholipid.

64. The lipid nanoparticle (LNP) of any one of claims 1 to 48 and 62 to 63, wherein the first lipid-anchored polymer comprises a linker-lipid moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), distearoyl-rac-glycerol (DSG), 1,2-dipalmitoyl-rac-glycerol (DPG), a derivative thereof, and a combination any of the foregoing.

65. The lipid nanoparticle (LNP) of claim 64, wherein the first lipid-anchored polymer comprises a linker-lipid moiety selected from the group consisting of DOPE, DSPE, DSG, DODA, DPG, a derivative thereof, and a combination of any of the foregoing.

66. The lipid nanoparticle (LNP) of any one of claims to 1 to 38, 49 to 53, 62, and 68, wherein the first lipid-anchored polymer comprises a linker-lipid moiety selected from the group consisting of 1,2-dimyristoyl-rac-glycero-3-methoxy (DMG), R-3-[(ω-methoxycarbamoyl)]-1,2-dimyristyloxl-propyl-3-amine, a derivative thereof, and a combination of any of the foregoing.

67. The lipid nanoparticle (LNP) of claim 66, wherein the first lipid-anchored polymer comprises DMG.

68. The lipid nanoparticle (LNP) of any one of claims 1 to 67, wherein the polymer is selected from the group consisting of polyethylene (PE), polypropylene (PP), polyethylene glycol (PEG), polyglycerol (PG), polyvinyl alcohol (PVOH), polysarcosine (pSar), and a combination thereof.

69. The lipid nanoparticle (LNP) of claim 68, wherein the polymer is selected from the group consisting of polyethylene glycol (PEG), polyglycerol (PG), polysarcosine (pSar), and a combination thereof.

70. The lipid nanoparticle (LNP) of any one of claims 1 to 69, wherein the polymer has an average molecular weight of between about 1000 Da and about 5000 Da.

71. The lipid nanoparticle (LNP) of claim 70, wherein the polymer has an average molecular weight of between about 2000 Da and about 5000 Da.

72. The lipid nanoparticle (LNP) of claim 71, wherein the polymer has an average molecular weight of about 2000 Da.

73. The lipid nanoparticle (LNP) of claim 71, wherein the polymer has an average molecular weight of about 3200 Da to about 3500 Da.

74. The lipid nanoparticle (LNP) of any one of claims 69 to 73, wherein the polymer is polyethylene glycol (PEG).

75. The lipid nanoparticle (LNP) of any one of claims 1 to 74, wherein the sterol is selected from the group consisting of cholesterol, beta-sitosterol, stigmasterol, beta-sitostanol, campesterol, brassicasterol, and a derivative of thereof, and a combination thereof.

76. The lipid nanoparticle (LNP) of claim 75, wherein the sterol is cholesterol.

77. The lipid nanoparticle (LNP) of claim 75, wherein the sterol is beta-sitosterol.

78. The lipid nanoparticle (LNP) of any one of claims to 1 to 77, wherein the ionizable lipid is a lipid represented by:
a) Formula (A): 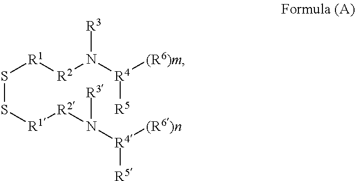
or a pharmaceutically acceptable salt thereof, wherein:
R¹ and R^{1′} are each independently optionally substituted linear or branched C₁₋₃ alkylene;
R² and R^{2′} are each independently optionally substituted linear or branched C₁₋₆ alkylene;
R³ and R^{3′} are each independently optionally substituted linear or branched C₁₋₆ alkyl;
or alternatively, when R² is optionally substituted branched C₁₋₆ alkylene, R² and R³, taken together with their intervening N atom, form a 4- to 8-membered heterocyclyl;
or alternatively, when R^{2′} is optionally substituted branched C₁₋₆ alkylene, R^{2′} and R^{3′}, taken together with their intervening N atom, form a 4- to 8-membered heterocyclyl;
R⁴ and R^{4′} are each independently —CR^{a}, —C(R^{a})₂CR^{a}, or —[C(R^{a})₂]₂CR^{a};
R^{a}, for each occurrence, is independently H or C₁₋₃ alkyl;
or alternatively, when R⁴ is —C(R^{a})₂CR^{a}, or —[C(R^{a})₂]₂CR^{a} and when R^{a} is C₁₋₃ alkyl, R³ and R⁴, taken together with their intervening N atom, form a 4- to 8-membered heterocyclyl;
or alternatively, when R^{4′} is —C(R^{a})₂CR^{a}, or —[C(R^{a})₂]₂CR^{a} and when R^{a} is C₁₋₃ alkyl, R^{3′} and R⁴, taken together with their intervening N atom, form a 4- to 8-membered heterocyclyl;
R⁵ and R^{5′} are each independently hydrogen, C₁₋₂₀ alkylene or C₂₋₂₀ alkenylene;
R⁶ and R^{6′}, for each occurrence, are independently C₁₋₂₀ alkylene, C₃₋₂₀ cycloalkylene, or C₂₋₂₀ alkenylene; and
m and n are each independently an integer selected from 1, 2, 3, 4, and 5; or
b) Formula (B): 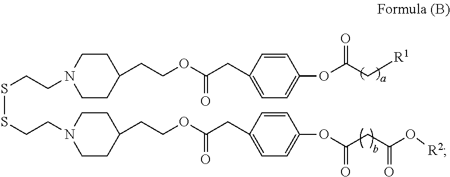
or a pharmaceutically acceptable salt thereof, wherein:
a is an integer ranging from 1 to 20;
b is an integer ranging from 2 to 10;
R¹ is absent or is selected from (C₂-C₂₀)alkenyl, —C(O)O(C₂-C₂₀)alkyl, and cyclopropyl substituted with (C₂-C₂₀)alkyl; and
R² is (C₂-C₂₀)alkyl; or
c) Formula (C): 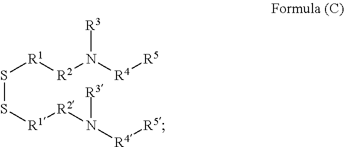
or a pharmaceutically acceptable salt thereof, wherein:
R¹ and R^{1′} are each independently (C₁-C₆)alkylene optionally substituted with one or more groups selected from R^{a};
R² and R^{2′} are each independently (C₁-C₂)alkylene;
R³ and R^{3′} are each independently (C₁-C₆)alkyl optionally substituted with one or more groups selected from R^{b};
or alternatively, R² and R³ and/or R^{2′} and R^{3′} are taken together with their intervening N atom to form a 4- to 7-membered heterocyclyl;
R⁴ and R^{4′} are each a (C₂-C₆)alkylene interrupted by —C(O)O—;
R⁵ and R^{5′} are each independently a (C₂-C₃₀)alkyl or (C₂-C₃₀)alkenyl, each of which are optionally interrupted with —C(O)O— or (C₃-C₆) cycloalkyl; and
R^{a} and R^{b} are each halo or cyano; or
d) Formula (D): 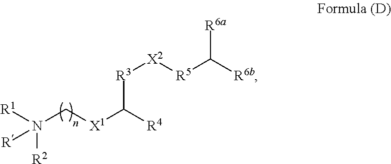
or a pharmaceutically acceptable salt thereof, wherein:
R′ is absent, hydrogen, or C₁-C₆ alkyl; provided that when R′ is hydrogen or C₁-C₆ alkyl, the nitrogen atom to which R′, R¹, and R² are all positively charged;
R¹ and R² are each independently hydrogen, C₁-C₆ alkyl, or C₂-C₆ alkenyl;
R³ is C₁-C₁₂ alkylene or C₂-C₁₂ alkenylene;
R⁴ is C₁-C₁₈ unbranched alkyl, C₂-C₁₈ unbranched alkenyl, or 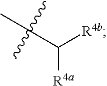
wherein: R^{4a} and R^{4b} are each independently C₁-C₁₆ unbranched alkyl or C₂-C₁₆ unbranched alkenyl; R⁵ is absent, C₁-C₈alkylene, or C₂-C₈ alkenylene;
R^{6a} and R^{6b} are each independently C₇-C₁₆ alkyl or C₇-C₁₆ alkenyl; provided that the total number of carbon atoms in R^{6a} and R^{6b} as combined is greater than 15;
X¹ and X² are each independently —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N═C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6.
e) Formula (E): 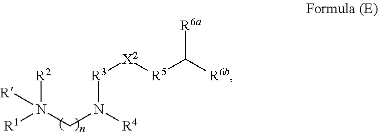
or a pharmaceutically acceptable salt thereof, wherein:
R′ is absent, hydrogen, or C₁-C₃ alkyl; provided that when R′ is hydrogen or C₁-C₃ alkyl, the nitrogen atom to which R′, R¹, and R² are all attached is positively charged;
R¹ and R² are each independently hydrogen or C₁-C₃ alkyl;
R³ is C₃-C₁₀ alkylene or C₃-C₁₀ alkenylene; 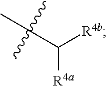
R⁴ is C₁-C₁₆ unbranched alkyl, C₂-C₁₆ unbranched alkenyl, or wherein: R^{4a} and R^{4b} are each independently C₁-C₁₆ unbranched alkyl or C₂-C₁₆ unbranched alkenyl; R⁵ is absent, C₁-C₆ alkylene, or C₂-C₆ alkenylene;
R^{6a} and R^{6b} are each independently C₇-C₁₄ alkyl or C₇-C₁₄ alkenyl;
X is —OC(═O)—, —SC(═O)—, —OC(═S)—, —C(═O)O—, —C(═O)S—, —S—S—, —C(R^{a})═N—, —N═C(R^{a})—, —C(R^{a})═NO—, —O—N—C(R^{a})—, —C(═O)NR^{a}—, —NR^{a}C(═O)—, —NR^{a}C(═O)NR^{a}—, —OC(═O)O—, —OSi(R^{a})₂O—, —C(═O)(CR^{a}₂)C(═O)O—, or OC(═O)(CR^{a}₂)C(═O)—; wherein:
R^{a}, for each occurrence, is independently hydrogen or C₁-C₆ alkyl; and
n is an integer selected from 1, 2, 3, 4, 5, and 6; or
f) an ionizable lipid selected from any of the ionizable lipids in Table 1, 4, 5, 6 or 7.

79. The lipid nanoparticle (LNP) of any one of claims 1 to 78, wherein the LNP further comprises a targeting moiety.

80. The lipid nanoparticle (LNP) of claim 79, wherein the LNP comprises a second lipid-anchored polymer and the targeting moiety is conjugated to the second lipid-anchored polymer.

81. The lipid nanoparticle (LNP) of claim 80, wherein the second lipid-anchored polymer comprises a linker-lipid moiety selected from the group consisting of 1,2-dipalmitoyl-sn-glycero-3-phosphocholine (DPPC), 1-palmitoyl-2-oleoyl-glycero-3-phosphocholine (POPC), 1-palmitoyl-2-oleoyl-sn-glycero-3-phosphoethanolamine (POPE), 1-palmitoyl-2-oleoyl-sn-glycero-3-phospho-(1′-rac-glycerol) (POPG), 1,2-dipalmitoyl-sn-glycero-3-phosphoethanolamine (DPPE), 1,2-distearoyl-sn-glycero-3-phosphoethanolamine (DSPE), 1,2-dielaidoyl-sn-phosphatidylethanolamine (DEPE), 1-stearoyl-2-oleoyl-sn-glycero-3-phosphoethanolamine (SOPE), 1,2-dioleoyl-sn-glycero-3-phosphoglycerol (DOPG), 1,2-dipalmitoyl-sn-glycero-3-phosphoglycerol (DPPG), 18-1-trans PE, 1,2-dioleoyl-sn-glycero-3-phospho-L-serine (DOPS), 1,2-diphytanoyl-sn-glycero-3-phosphoethanolamine (DPHyPE); and dioctadecylamine (DODA), distearoyl-rac-glycerol (DSG), 1,2-dipalmitoyl-rac-glycerol (DPG), a derivative thereof, and a combination any of the foregoing.

82. The lipid nanoparticle (LNP) of claim 81, wherein the second lipid-anchored polymer comprises a linker-lipid moiety selected from the group consisting of DSPE, DSG, DODA, DPG, a derivative thereof, and a combination of any of the foregoing.

83. The lipid nanoparticle of (LNP) of claim 82, wherein the first and the second lipid-anchored polymers are different lipid-anchored polymers; and wherein the linker-lipid of the first and the second lipid-anchored polymers comprise one of the following combinations:
DSG (the first lipid-anchored polymer) and DSPE (the second lipid-anchored polymer);
DSPE (the first lipid-anchored polymer) and DSG (the second lipid-anchored polymer);
DODA (the first lipid-anchored polymer) and DSPE (the second lipid-anchored polymer);
DPG (the first lipid-anchored polymer) and DSPE (the second lipid-anchored polymer);
DMG (the first lipid-anchored polymer) and DSPE (the second lipid-anchored polymer);
DODA (the first lipid-anchored polymer) and DSG (the second lipid-anchored polymer);
DPG (the first lipid-anchored polymer) and DSG (the second lipid-anchored polymer);
DMG (the first lipid-anchored polymer) and DSG (the second lipid-anchored polymer);
DPG (the first lipid-anchored polymer) and DODA (the second lipid-anchored polymer);
DMG (the first lipid-anchored polymer) and DODA (the second lipid-anchored polymer); or
DMG (the first lipid-anchored polymer) and DPG (the second lipid-anchored polymer).

84. The lipid nanoparticle (LNP) of claim 82, wherein the first and the second lipid-anchored polymers are the same lipid-anchored polymers; and wherein the linker-lipid of the first and the second lipid-anchored polymers comprise one of the following combinations:
DSG (the first lipid-anchored polymer) and DSG (the second lipid-anchored polymer);
DSPE (the first lipid-anchored polymer) and DSPE (the second lipid-anchored polymer);
DODA (the first lipid-anchored polymer) and DODA (the second lipid-anchored polymer); or
DPG (the first lipid-anchored polymer) and DPG (the second lipid-anchored polymer).

85. The lipid nanoparticle (LNP) of claim 80, wherein the targeting moiety is conjugated to a DSPE-anchored polymer.

86. The lipid nanoparticle (LNP) of claim 85, wherein the DSPE-anchored polymer is DSPE-PEG or a derivative thereof.

87. The lipid nanoparticle (LNP) of claim 80, wherein the targeting moiety is conjugated to a DSG-anchored polymer.

88. The lipid nanoparticle (LNP) of claim 87, wherein the DSG-anchored polymer is DSG-PEG or a derivative thereof.

89. The lipid nanoparticle (LNP) of claim 80, wherein the targeting moiety is capable of binding to a liver cell.

90. The lipid nanoparticle (LNP) of claim 89, wherein the liver cell is a hepatocyte.

91. The lipid nanoparticle (LNP) of claim 80, wherein the targeting moiety is N-acetyl galactosamine (GalNAc) or a GalNAc derivative.

92. The lipid nanoparticle (LNP) of claim 91, wherein the targeting moiety is a tri-antennary GalNAc conjugate or a tetra-antennary GalNAc conjugate.

93. The lipid nanoparticle (LNP) of claim 80, wherein the targeting moiety is selected from the group consisting of an ApoE protein, an ApoE polypeptide, an ApoB protein, an ApoB polypeptide, a fragment thereof, and a derivative of any of the foregoing.

94. The lipid nanoparticle (LNP) of claim 93, wherein the targeting moiety is selected from the group consisting of an ApoE protein conjugate, an ApoE peptide conjugate, an ApoB protein conjugate, and an ApoB peptide conjugate.

95. The lipid nanoparticle (LNP) of claim 94, wherein the targeting moiety is an ApoE protein conjugate.

96. The lipid nanoparticle (LNP) of any one of claims 1, 3 to 27, 37 to 39, and 49 to 95, wherein the ionizable lipid is Ionizable Lipid 81: 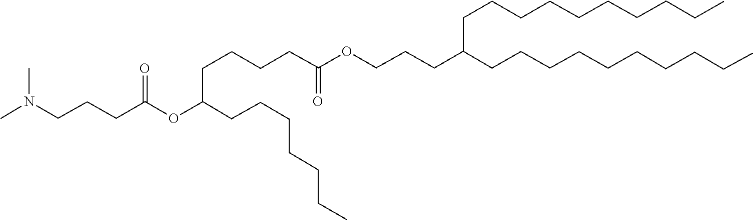 [SMILES: CCCCCCCCCCC(CCCCCCCCCC)CCCOC(=O)CCCCC(CCCCCCC)OC(=O)CCCN(C)C]
4-decyltetradecyl 6-((4-(dimethylamino)butanoyl)oxy)tridecanoate
or a pharmaceutically acceptable salt thereof.

97. The lipid nanoparticle (LNP) of any one of claims 1, 3 to 27, 37 to 39, and 49 to 95, wherein the ionizable lipid is Ionizable Lipid 89: 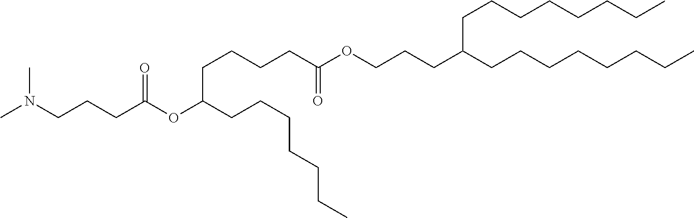 [SMILES: CCCCCCCCC(CCCCCCCC)CCCOC(=O)CCCCC(CCCCCCC)OC(=O)CCCN(C)C]
4-octyldodecyl 6-((4-(dimethylamino)butanoyl)oxy)tridecanoate
or a pharmaceutically acceptable salt thereof.

98. The lipid nanoparticle (LNP) of any one of claims 1 to 48 and 62 to 95, wherein the ionizable lipid is Ionizable Lipid 87: 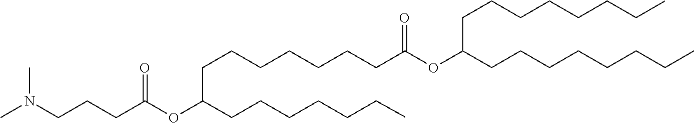 [SMILES: CCCCCCCCC(CCCCCCCC)OC(=O)CCCCCCCC(CCCCCCC)OC(=O)CCCN(C)C]
heptadecan-9-yl 9-((4-(dimethylamino)butanoyl)oxy)hexadecanoate
or a pharmaceutically acceptable salt thereof.

99. The lipid nanoparticle (LNP) of any one of claims 1 to 98, wherein the ionizable lipid is present in the LNP in an amount of about 30 mol % to about 60 mol % of the total lipid present in the LNP.

100. The lipid nanoparticle (LNP) of any one of claims 1 to 99, wherein the ionizable lipid is present in the LNP in an amount of about 35 mol % to about 50 mol % of the total lipid present in the LNP.

101. The lipid nanoparticle (LNP) of any one of claims 1 to 100, wherein the sterol is present in the LNP in an amount of about 20 mol % to about 45 mol % of the total lipid present in the LNP.

102. The lipid nanoparticle (LNP) of claim 101, wherein the sterol is present in the LNP in an amount of about 30 mol % to about 40 mol % of the total lipid present in the LNP.

103. The lipid nanoparticle (LNP) of any one of claims 1 to 102, wherein the first lipid-anchored polymer is present in the LNP in an amount of about 2 mol % to about 5 mol % of the total lipid present in the LNP.

104. The lipid nanoparticle (LNP) of any one of claims 80 to 102, wherein the second lipid-anchored polymer is present in the LNP in an amount of about 0.005 mol % to about 5 mol % of the total lipid present in the LNP.

105. The lipid nanoparticle (LNP) of claim 104, wherein the second lipid-anchored polymer is present in the LNP in an amount of about 0.05 mol % to about 2 mol % of the total lipid present in the LNP.

106. The lipid nanoparticle (LNP) of claim 105, wherein the second lipid-anchored polymer is present in the LNP in an amount of about 0.1 mol % to about 1 mol % of the total lipid present in the LNP.

107. The lipid nanoparticle (LNP) of claim 106, wherein the second lipid-anchored polymer is present in the LNP in an amount of about 0.5 mol % of the total lipid present in the LNP.

108. The lipid nanoparticle (LNP) of any one of claims 103 to 107, wherein the first lipid-anchored polymer and the second lipid anchored polymer are present in the LNP in an amount of about 2.5 mol % and 0.5 mol %, respectively, of the total lipid present in the LNP.

109. The lipid nanoparticle (LNP) of any one of claims 1 to 27, and 37 to 108, wherein the helper lipid represented by Formula (I), (II), (III), or (IV), or a salt or an ester thereof, or a deuterated analogue of any of the foregoing, is present in the LNP in an amount of about 2 mol % to about 40 mol % of the total lipid present in the LNP.

110. The lipid nanoparticle (LNP) of claim 109, wherein the helper lipid represented by Formula (I), (II), (III), or (IV), or a salt or an ester thereof, or a deuterated analogue of any of the foregoing, is present in the LNP in an amount of about 5 mol % to about 30 mol % of the total lipid present in the LNP.

111. The lipid nanoparticle (LNP) of claim 110, wherein the helper lipid represented by Formula (I), (II), (III), or (IV), or a salt or an ester thereof, or a deuterated analogue of any of the foregoing, is present in the LNP in an amount of about 10 mol % to about 20 mol % of the total lipid present in the LNP.

112. The lipid nanoparticle (LNP) of claim 111, wherein the helper lipid represented by Formula (I), (II), (III), or (IV), or a salt or an ester thereof, or a deuterated analogue of any of the foregoing, is present in the LNP in an amount of about 10 mol % of the total lipid present in the LNP.

113. The lipid nanoparticle (LNP) of claim 111, wherein the helper lipid represented by Formula (I), (II), (III), or (IV), or a salt or an ester thereof, or a deuterated analogue of any of the foregoing, is present in the LNP in an amount of about 15 mol % of the total lipid present in the LNP.

114. The lipid nanoparticle (LNP) of claim 111, wherein the helper lipid represented by Formula (I), (II), (III), or (IV), or a salt or an ester thereof, or a deuterated analogue of any of the foregoing, is present in the LNP in an amount of about 20 mol % of the total lipid present in the LNP.

115. The lipid nanoparticle (LNP) of any one of claims 1 to 114, wherein the helper lipid is present in the LNP in an amount of about 2 mol % to about 40 mol %, or about 5 mol % to about 35 mol %, or about 5 mol % to about 30 mol %, or about 5 mol % to about 25 mol %, or about 5 mol % to about 20 mol %, or about 5 mol % to about 15 mol %, or about 5 mol % to about 10 mol %, or about 10 mol % to about 15 mol % of the total lipid present in the LNP.

116. The lipid nanoparticle (LNP) of any one of claims 1 to 115, wherein the LNP is suitable for intravenous administration.

117. The lipid nanoparticle (LNP) of claim 116, wherein the LNP is less immunogenic than a reference LNP; wherein the reference LNP: (i) does not comprise the helper lipid represented by Formula (I), (II), (III), or (IV), or a salt or an ester thereof, or a deuterated analogue of any of the foregoing; or (ii) comprises a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE) and a reference lipid polymer comprising at least two hydrophobic tails each comprise 12 to 15 carbon atoms in a single aliphatic chain backbone.

118. The lipid nanoparticle (LNP) of claim 117, wherein the reference lipid polymer is 1,2-dimyristoyl-rac-glycero-3-methoxypolyethylene glycol (DMG-PEG).

119. The lipid nanoparticle (LNP) of claim 116 or claim 118, wherein the LNP results in an expression level of TNA in a target cell that is equivalent to or higher than the reference LNP.

120. The lipid nanoparticle (LNP) of any one of claims 116 to 119, wherein the LNP elicits lower pro-inflammatory cytokine response than the reference LNP.

121. The lipid nanoparticle (LNP) of claim 119 or 120, wherein the LNP results in a lower uptake of the TNA by a blood cell than of the reference LNP.

122. The lipid nanoparticle (LNP) of claim 121, wherein said blood cell is a red blood cell.

123. The lipid nanoparticle (LNP) of any one of claims 1 to 122, wherein the therapeutic nucleic acid (TNA) is selected from the group consisting of a minigene, a plasmid, a minicircle, a small interfering RNA (siRNA), a microRNA (miRNA), a guide RNA (gRNA), an antisense oligonucleotide (ASO), a ribozyme, a closed-ended DNA (ceDNA), single-stranded DNA (ssDNA), a ministring, a Doggybone™, a protelomere closed ended DNA, a dumbbell linear DNA, a dicer-substrate dsRNA, a small hairpin RNA (shRNA), an asymmetrical interfering RNA (aiRNA), mRNA, tRNA, rRNA, gRNA, a DNA viral vector, a viral RNA vector, a non-viral vector and any combination thereof.

124. The lipid nanoparticle (LNP) of any one of claims 1 to 123, wherein the TNA is greater than about 200 bp or greater than about 200 nt in length.

125. The lipid nanoparticle (LNP) of claim 124, wherein the TNA is greater than about 500 bp or greater than about 500 nt in length.

126. The lipid nanoparticle (LNP) of claim 125, wherein the TNA is greater than about 1000 bp or greater than about 1000 nt in length.

127. The lipid nanoparticle (LNP) of claim 126, wherein the TNA is greater than about 4000 bp or greater than about 4000 nt in length.

128. The lipid nanoparticle (LNP) of any one of claims 1 to 127, wherein the TNA is a closed-ended DNA (ceDNA).

129. The lipid nanoparticle (LNP) of any one of claims 1 to 127, wherein the TNA is a messenger RNA (mRNA).

130. The lipid nanoparticle (LNP) of any one of claims 1 to 129, wherein the TNA is a single-stranded nucleic acid.

131. The lipid nanoparticle (LNP) of any one of claims 1 to 129, wherein the TNA is a double-stranded nucleic acid.

132. A pharmaceutical composition comprising the lipid nanoparticle (LNP) of any one of claims 1 to 131 and a pharmaceutically acceptable carrier.

133. A method of producing the lipid nanoparticle (LNP) of any one of claims 1 to 131, comprising combining:
the therapeutic nucleic acid (TNA);
the ionizable lipid;
the sterol;
the first lipid-anchored polymer;
the helper lipid represented by Formula (I), (II), (III), or (IV), or a salt or an ester thereof, or a deuterated analogue of any of the foregoing; or a helper lipid selected from the group consisting of distearoylphosphatidylcholine (DSPC), 1,2-dioleoyl-sn-glycero-3-phosphocholine (DOPC), and 1,2-dioleoyl-sn-glycero-3-phosphoethanolamine (DOPE);
optionally the second lipid-anchored polymer; and
optionally the targeting moiety.

134. A method of treating a genetic disorder in a subject, said method comprising administering to said subject an effective amount of the lipid nanoparticle (LNP) of any one of claims 1 to 131 or the pharmaceutical composition of claim 132.

135. The method of claim 134, wherein said subject is a human.

136. The method any claim 134 or 135, wherein the genetic disorder is selected from the group consisting of sickle cell anemia; melanoma; hemophilia A (clotting factor VIII (FVIII) deficiency); hemophilia B (clotting factor IX (FIX) deficiency); cystic fibrosis (CFTR); familial hypercholesterolemia (LDL receptor defect); hepatoblastoma; Wilson's disease; phenylketonuria (PKU); congenital hepatic porphyria; an inherited disorder of hepatic metabolism; Lesch Nyhan syndrome; a thalassaemia; xeroderma pigmentosum; Fanconi's anemia; retinitis pigmentosa; ataxia telangiectasia; Bloom's syndrome; retinoblastoma; a mucopolysaccharide storage disease; a Niemann-Pick Disease; Fabry disease; Schindler disease; GM2-gangliosidosis Type II (Sandhoff Disease); Tay-Sachs disease; Metachromatic Leukodystrophy; Krabbe disease; a mucolipidosis (ML); Sialidosis Type II, a glycogen storage disease (GSD); Gaucher disease; cystinosis; Batten disease; Aspartylglucosaminuria; Salla disease; Danon disease (LAMP-2 deficiency); Lysosomal Acid Lipase (LAL) deficiency; a neuronal ceroid lipofuscinoses (NCL); a sphingolipidoses, galactosialidosis; amyotrophic lateral sclerosis (ALS); Parkinson's disease; Alzheimer's disease; Huntington's disease; spinocerebellar ataxia; spinal muscular atrophy (SMA); Friedreich's ataxia; Duchenne muscular dystrophy (DMD); a Becker muscular dystrophy (BMD), dystrophic epidermolysis bullosa (DEB); ectonucleotide pyrophosphatase 1 deficiency; generalized arterial calcification of infancy (GACI); Leber Congenital Amaurosis; Stargardt disease; wet macular degeneration (wet AMD); ornithine transcarbamylase (OTC) deficiency; Usher syndrome; alpha-1 antitrypsin deficiency; a progressive familial intrahepatic cholestasis (PFIC); and Cathepsin A deficiency.

137. The method of claim 136, wherein said genetic disorder is phenylketonuria (PKU).

138. The method of claim 136, wherein the genetic disorder is hemophilia A (Factor VIII deficiency).

139. The method of claim 136, wherein said genetic disorder is Wilson's disease.

140. The method of claim 136, wherein said genetic disorder is Gaucher disease.

141. The method of claim 136, wherein said genetic disorder is Gaucher disease Type I, Gaucher disease Type II or Gaucher disease type III.

142. The method of claim 136, wherein said genetic disorder is Leber congenital amaurosis (LCA).

143. The method of claim 136, wherein said LCA is LCA10.

144. The method of claim 136, wherein said genetic disorder is Stargardt disease.

145. The method of claim 136, wherein said genetic disorder is wet macular degeneration (wet AMD).

146. A method of treating providing anti-tumor immunity in a subject, the method comprising administering to the subject an effective amount of the LNP of any one of claims 1 to 131 or the pharmaceutical composition of claim 132.

147. A method of treating a subject having a disease, disorder or condition associated with an elevated expression of a tumor antigen, the method comprising administering to the subject an effective amount of the LNP of any one of claims 1 to 131 or the pharmaceutical composition of claim 132.

148. The method of any one of claim 146 or claim 147, wherein the TNA is retained in the spleen for at least about 6 hours, or at least about 9 hours, or at least about 12 hours, or at least about 15 hours, or at least about 18 hours, or at least about 21 hours, or at least about 24 hours, or at least about 27 hours, or at least about 30 hours, or at least about 33 hours, or at least about 36 hours after dosing.

149. The method of claim 148, wherein the amount of the TNA at the start of a 12, 18, or 24-hour time window post-dosing and the amount of the TNA at the end of the time window are within the same order of magnitude.

150. A method of treating a blood disease, disorder or condition in a subject, the method comprising administering to the subject an effective amount of the LNP of any one of claims 1 to 131 or the pharmaceutical composition of claim 132.

151. The method of claim 150, wherein the blood disease, disorder or condition is selected from the group consisting of acute myeloid leukemia (AML), acute lymphoblastic leukemia (ALL), chronic lymphocytic leukemia (CLL), chronic myelogenous leukemia (CML), Hodgkin lymphoma (HL), multiple myeloma, a myelodysplastic syndrome (MDS), non-Hodgkin lymphoma (NHL), adrenoleukodystrophy (ALD), Hurler syndrome, Krabbe disease (Globoid-cell leukodystrophy or GLD), metachromatic leukodystrophy (MLD), severe aplastic anemia (SAA), severe combined immunodeficiency (SCID), sickle cell disease (SCD), thalassemia, Wiskott-Aldrich syndrome, Diamond-Blackfan anemia, essential thrombocytosis, Fanconi anemia, hemophagocytic lymphohistiscytosis (HLH), juvenile myelomonocytic leukemia (JMML), myelofibrosis, polycythemia vera, and a combination thereof.

152. The method of any one of claims 146 to 151, wherein the TNA is a messenger RNA (mRNA).

153. The method of any one of claims 146 to 151, wherein the TNA is a single stranded DNA (ssDNA).

154. A lipid nanoparticle (LNP) comprising:
a therapeutic nucleic acid (TNA);
an ionizable lipid;
a helper lipid;
a sterol;
a lipid-anchored polymer; wherein the lipid-anchored polymer comprises:
i) a polymer;
ii) a lipid moiety comprising at least one hydrophobic tail; and
wherein the polymer is linked to the lipid moiety;
wherein the at least one hydrophobic tail comprises between 18 to 22 carbon atoms in a single aliphatic chain backbone;
wherein the sterol is present in the LNP in an amount of about 30 mol % to about 40 mol % of the total lipid present in the LNP;
wherein the lipid-anchored polymer is present in the LNP in an amount of about 2 mol % to about 7 mol % of the total lipid present in the LNP; and
wherein the LNP has an average particle size of 50-100 nm in diameter.

155. The lipid nanoparticle (LNP) of claim 154, wherein the LNP has an average particle size of 60-80 nm in diameter.

156. The lipid nanoparticle (LNP) of claim 155, wherein the LNP further comprises a second lipid-anchored polymer.

157. The lipid nanoparticle (LNP) of claim 156, wherein the second lipid-anchored polymer comprises conjugation reactive moiety.

158. The lipid nanoparticle (LNP) of claim 156, wherein the second lipid-anchored polymer comprises a targeting moiety.

159. The lipid nanoparticle (LNP) of claim 158, wherein the targeting moiety is selected from the group of IgG, Fab, VHH, scFv, a peptide ligand and sugar ligand.

160. The lipid nanoparticle (LNP) of claim 154, wherein the lipid-anchored polymer is present in the LNP in an amount of about 2 mol % to about 5 mol % of the total lipid present in the LNP.

161. A lipid nanoparticle (LNP) comprising:
a therapeutic nucleic acid (TNA);
an ionizable lipid;
a helper lipid;
a sterol;
a first lipid-anchored polymer; wherein the first lipid-anchored polymer comprises:
i) a polymer; and
ii) a lipid moiety comprising at least one hydrophobic tail,
wherein the polymer is linked to the lipid moiety; and
a second lipid-anchored polymer; wherein the second lipid-anchored polymer comprises:
i) a polymer;
ii) a reactive moiety for conjugation to a targeting moiety; and
iii) a lipid moiety comprising at least one hydrophobic tail,
wherein the polymer is linked to the lipid moiety;
wherein the first lipid anchored polymer comprises at least one hydrophobic tail comprising 18 to 22 carbon atoms in a single aliphatic chain backbone;
wherein the second lipid anchored polymer comprises at least one hydrophobic tail comprising 18 to 22 carbon atoms in a single aliphatic chain backbone;
wherein the sterol is present in the LNP in an amount of about 30 mol % to about 40 mol % of the total lipid present in the LNP;
wherein the first lipid-anchored polymer is present in the LNP in an amount of about 2 mol % to about 7 mol % of the total lipid present in the LNP;
wherein the second lipid-anchored polymer is present in the LNP in an amount of about 0.2 mol % to about 2 mol % of the total lipid present in the LNP; and
wherein the LNP has an average particle size of 50-100 nm in diameter.

162. A lipid nanoparticle (LNP) comprising:
an ionizable lipid;
a helper lipid;
a sterol;
a first lipid-anchored polymer; wherein the first lipid-anchored polymer comprises:
i) a polymer; and
ii) a lipid moiety comprising at least one hydrophobic tail,
wherein the polymer is linked to the lipid moiety; and
a second lipid-anchored polymer; wherein the second lipid-anchored polymer comprises:
i) a polymer;
ii) a reactive moiety for conjugation to a targeting moiety; and
iii) a lipid moiety comprising at least one hydrophobic tail,
wherein the polymer is linked to the lipid moiety;
wherein the first lipid anchored polymer comprises at least one hydrophobic tail comprising 18 to 22 carbon atoms in a single aliphatic chain backbone;
wherein the second lipid anchored polymer comprises at least one hydrophobic tail comprising 18 to 22 carbon atoms in a single aliphatic chain backbone;
wherein the sterol is present in the LNP in an amount of about 30 mol % to about 40 mol % of the total lipid present in the LNP;
wherein the first lipid-anchored polymer is present in the LNP in an amount of about 2 mol % to about 7 mol % of the total lipid present in the LNP;
wherein the second lipid-anchored polymer is present in the LNP in an amount of about 0.2 mol % to about 2 mol % of the total lipid present in the LNP; and
wherein the LNP has an average particle size of 50-100 nm in diameter.

163. A lipid nanoparticle (LNP) comprising:
a therapeutic nucleic acid (TNA);
an ionizable lipid;
a helper lipid;
a sterol;
a first lipid-anchored polymer; wherein the first lipid-anchored polymer comprises:
i) a polymer; and
ii) a lipid moiety comprising at least one hydrophobic tail,
wherein the polymer is linked to the lipid moiety; and
a second lipid-anchored polymer; wherein the second lipid-anchored polymer comprises:
i) a polymer; and
ii) a lipid moiety comprising at least one hydrophobic tail, and
iii) optionally a reactive moiety for conjugation to a targeting moiety or a targeting moiety,
wherein the polymer is linked to the lipid moiety;
wherein the first lipid anchored polymer comprises at least one hydrophobic tail comprising 18 to 22 carbon atoms in a single aliphatic chain backbone;
wherein the second lipid anchored polymer comprises at least one hydrophobic tail comprising 18 to 22 carbon atoms in a single aliphatic chain backbone;
wherein the sterol is present in the LNP in an amount of about 30 mol % to about 40 mol % of the total lipid present in the LNP;
wherein the first lipid-anchored polymer is present in the LNP in an amount of about 2 mol % to about 7 mol % of the total lipid present in the LNP;
wherein the second lipid-anchored polymer is present in the LNP in an amount of about 0.2 mol % to about 2 mol % of the total lipid present in the LNP; and
wherein the LNP has an average particle size of 50-100 nm in diameter.
